(19) United States
(12) Patent Application Publication
(10) Pub. No.: US 2018/0186897 A1
(43) Pub. Date: Jul. 5, 2018

(54) NOVEL VACCINES IN PREVENTION AND TREATMENT OF MALARIA

(71) Applicant: Institute for Research in Biomedicine, Bellinzona (CH)

(72) Inventors: Antonio Lanzavecchia, Porza (CH); Roger Geiger, Bellinzona (CH); Luca Piccoli, Bellinzona (CH)

(21) Appl. No.: 15/738,828

(22) PCT Filed: Jun. 24, 2016
(86) PCT No.: PCT/EP2016/064757

§ 371 Date: Dec. 21, 2017

(30) Foreign Application Priority Data
Jun. 26, 2015 (EP) PCT/EP2015/001292
Dec. 22, 2015 (EP) PCT/EP2015/002599

(51) Int. Cl. C07K 16/44; A61P 33/06; C07K 14/705; G01N 33/569

(52) U.S. Cl. CPC C07K 16/44; A61P 33/06; C07K 14/70503; G01N 2800/26; C07K 2319/33; C07K 2317/21; G01N 33/569

(57) ABSTRACT

The present invention provides a pharmaceutical composition, for example a vaccine, which comprises a RIFIN, which is able to bind to a mutated LAIR-1 fragment, which broadly binds to erythrocytes infected with Plasmodium falciparum. Such a RIFIN may be useful in the prevention and/or treatment of malaria.

The present invention relates to the field of malaria medication, in particular to Plasmodium falciparum surface antigens.

The virulence of Plasmodium falciparum and other Plasmodia that cause malaria is attributed to the adhesion of infected erythrocytes to the vascular endothelium or to uninfected erythrocytes to form rosettes. The key to the survival of P. falciparum in the human host is its ability to undergo antigenic variation, by switching expression among protein variants encoded by multigene families, such as var, rif and stevor. About 60 var and 150 rif genes are clonally expressed by P. falciparum and encode a diverse and polymorphic set of molecules displayed on the surface of infected erythrocytes that mediate adhesion to different substrates. It is well established that the antibody response to P. falciparum-infected erythrocytes protects from lethal disease and, consequently, the discovery of specific antibodies and conserved antigens has practical relevance.

In particular, surface antigens of P. falciparum-infected erythrocytes were suggested as immune targets (for review see Chan, J.-A. et al., 2014, Cell. Mol. Life Sci. 71:3633-3657). Surface antigens of infected erythrocytes (IEs), which are also known as “variant surface antigens” or “VSA”, include PfEMP1 (P. falciparum erythrocyte membrane protein 1), RIFIN (repetitive interspersed family proteins), STEVOR (sub-telomeric variable open reading frame proteins) and SURFIN (surface-associated interspersed gene family proteins), whereby the most important immune target appeared to be PfEMP1, which is a major ligand for vascular adhesion and sequestration of IEs. Studies are beginning to identify specific variants of PfEMP1 linked to disease pathogenesis that may be suitable for vaccine development, but overcoming antigenic diversity in PfEMP1 remains a major challenge (for review see Chan, J.-A. et al., 2014, Cell. Mol. Life Sci. 71:3633-3657).

The RIFINSs, another family of antigens found on the surface of IEs, represent the largest family of antigenically variable molecules in P. falciparum. These polypeptides are encoded by 150 rif genes whose expression is upregulated in rosetting parasites. It has been recently shown that RIFINs bind preferentially to erythrocytes of blood group A to form large rosettes and to mediate vascular sequestration of IEs, indicating that they may play an important role in the development of severe malaria (Goel S. et al., 2015, Nat Med. 21(4):314-7).

Recently, there has been considerable technological progress for the isolation of broadly neutralizing human monoclonal antiviral antibodies against highly variable pathogens, such as HIV-1 and influenza virus. These antibodies can be used for passive immunotherapy but also to drive the design of immunogens capable of inducing antibodies of the same type in active vaccination (Burton D. R. et al., Cell Host Microbe, 2012, Oct. 18; 12(4):396-407). However, in spite of these successes, there is little expectation that it would be possible to find antibodies capable of recognizing the huge number of different P. falciparum strains that can infect erythrocytes, considering the extensive polymorphism and the large number of surface molecules. Similarly, it has been difficult so far to identify a structural basis for the design of a vaccine capable of eliciting antibodies that can protect against the highly variable P. falciparum strains.

In view of the above, it is the object of the present invention to overcome the drawbacks of current malaria medications, in particular vaccines, outlined above. In particular, it is the object of the present invention to provide a conserved Plasmodium falciparum antigen, which may be used for example in a pharmaceutical composition, in particular in a vaccine or to identify broadly binding antibodies. Thus, it is also an object of the present invention to provide a pharmaceutical composition, in particular a vaccine, which is able to induce a strong and broad antibody response to infected erythrocytes. In this context, it is furthermore an object of the present invention to provide a pharmaceutical composition, in particular a vaccine, which additionally may also inhibit transmission of P. falciparum.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements may be listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration and/or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits.

Preferably, the subject or patient is a human.

The terms “peptide”, “polypeptide”, and “protein” are used herein interchangeably. As used herein, the terms “peptide”, “polypeptide”, and “protein” and variations of these terms refer to peptide, oligopeptide, oligomer, polypeptide or protein including fusion protein, respectively, comprising at least two amino acids joined to each other by a normal peptide bond, or by a modified peptide bond, such as for example in the cases of isosteric peptides.

For example, a “classical” peptide, polypeptide or protein is typically composed of amino acids selected from the 20 amino acids defined by the genetic code, linked to each other by a normal peptide bond. A peptide, polypeptide or protein can be composed of L-amino acids and/or D-amino acids. Preferably, a peptide, polypeptide or protein is either (entirely) composed of L-amino acids or (entirely) of D-amino acids, thereby forming “retro-inverso peptide sequences”. The term “retro-inverso (peptide) sequences” refers to an isomer of a linear peptide sequence in which the direction of the sequence is reversed and the chirality of each amino acid residue is inverted (see e.g. Jameson et al., Nature, 368, 744-746 (1994); Brady et al., Nature, 368, 692-693 (1994)). In particular, the terms “peptide”, “polypeptide”, “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide or protein may comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” preferably include for example lipopeptides, lipoproteins, glycopeptides, glycoproteins and the like.

The term “recombinant polypeptide”, as used herein, refers to any polypeptide which is prepared, expressed, created or isolated by recombinant means, and which is not naturally occurring.

As used herein, the term “antibody” encompasses various forms of antibodies, preferably an antibody is a monoclonal antibody. Antibodies include, without being limited to, whole antibodies, antibody fragments, human antibodies, chimeric antibodies, humanized antibodies and genetically engineered antibodies (variant or mutant antibodies) as long as the characteristic properties according to the invention are retained. Especially preferred are human or humanized monoclonal antibodies, especially as recombinant human monoclonal antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol, 147 (1991) 86-95). The term “human antibody” as used herein also comprises such antibodies which are modified, e.g. in the variable region, to generate the properties according to the invention.

As used herein, the term “variable region” (variable region of a light chain (V_{L}), variable region of a heavy chain (V_{H})) denotes each of the pair of light and heavy chains which is involved directly in binding the antibody to the antigen.

As used herein, the term “constant domain” (also referred to as “constant region”) refers to a domain of an antibody which is not involved directly in binding an antibody to an antigen, but exhibits various effector functions. For example, antibodies or immunoglobulins may be divided in the classes: IgA, IgD, IgE, IgG and IgM, depending on the amino acid sequence of the constant region of their heavy chains. Several of these may be further divided into subclasses, e.g. IgG1, IgG2, IgG3, and IgG4, IgA1 and IgA2. The heavy chain constant regions that correspond to the different classes of immunoglobulins may be called α, ε, γ, and μ, respectively.

As used herein, the terms “nucleic acid”, “nucleic acid molecule” and “polynucleotide” are used interchangeably and are intended to include DNA molecules and RNA molecules. A nucleic acid molecule may be single-stranded or double-stranded, but preferably is double-stranded DNA.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The terms “binding” and, in particular, “specifically binding” and similar reference does not encompass non-specific sticking.

As used herein, the term “sequence variant” refers to any alteration in a reference sequence, whereby a reference sequence is any of the sequences listed in the “Table of Sequences and SEQ ID Numbers” (sequence listing), i.e. SEQ ID NO: 1 to SEQ ID NO: 639. Thus, the term “sequence variant” includes nucleotide sequence variants and amino acid sequence variants. In particular, in a “sequence variant” the functionality (of the reference sequence) is preserved, i.e. the sequence variant is functional (also referred to as “functional sequence variant”). A “sequence variant” as used herein typically has a sequence which is at least 70% identical to the reference sequence, preferably at least 80% identical to the reference sequence, more preferably at least 90% identical, even more preferably at least 95% identical, and particularly preferably at least 99% identical to the reference sequence.

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” in the context of a nucleotide sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleic acid (nucleotide) sequence can either result in a change in the respective reference amino acid sequence, i.e. in a “sequence variant” of the respective amino acid sequence or not. Preferred sequence variants are such nucleotide sequence variants, which do not result in amino acid sequence variants (silent mutations), but other non-silent mutations are within the scope as well, in particular mutant nucleotide sequences, which result in an amino acid sequence, which is at least 70% identical to the reference sequence, preferably at least 80% identical to the reference sequence, more preferably at least 90% identical, even more preferably at least 95% identical, and particularly preferably at least 99% identical to the reference sequence.

An “sequence variant” in the context of an amino acid has an altered sequence in which one or more of the amino acids in the reference sequence is deleted or substituted, or one or more amino acids are inserted into the sequence of the reference amino acid sequence. As a result of the alterations, the amino acid sequence variant has an amino acid sequence which is at least 70% identical to the reference sequence, preferably at least 80% identical to the reference sequence, more preferably at least 90% identical, even more preferably at least 95% identical, and particularly preferably at least 99% identical to the reference sequence. Variant sequences which are at least 90% identical have no more than 10 alterations, i.e. any combination of deletions, insertions or substitutions, per 100 amino acids of the reference sequence.

In the context of (poly-)peptides/proteins, a “linear sequence” or a “sequence” is the order of amino acids in a peptide/protein in an amino to carboxyl terminal direction in which residues that neighbor each other in the sequence are contiguous in the primary structure of the peptide/protein.

While it is possible to have non-conservative amino acid substitutions in a “sequence variant”, it is preferred in a “sequence variant” that the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acid, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Importantly, the sequence variants are functional sequence variants, i.e. the alterations in the sequence variants do not abolish the functionality of the respective reference sequence, in the present case, preferably, the functionality of a RIFIN, of an N-terminal semi-conserved domain of a RIFIN and/or of second variable (V2) domain of a RIFIN to bind to the same binding site of a mutated LAIR-1 fragment. Guidance in determining which nucleotides and amino acid residues, respectively, may be substituted, inserted or deleted without abolishing such functionality are found by using computer programs well known in the art.

As used herein, a nucleic acid sequence or an amino acid sequence “derived from” a designated nucleic acid, peptide, polypeptide or protein refers to the origin of the polypeptide. Preferably, the nucleic acid sequence or amino acid sequence which is derived from a particular sequence has an amino acid sequence that is essentially identical to that sequence or a portion thereof, from which it is derived, whereby “essentially identical” includes sequence variants as defined above. Preferably, the nucleic acid sequence or amino acid sequence which is derived from a particular peptide or protein, is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins and nucleic acids are thus easily identifiable to one of ordinary skill in the art, e.g. by the use of computer programs, which are able to predict protein domains, such as transmembrane domains, signal domains, binding domains, or the like. Likewise, sequences “derived from” other sequence are usually easily identifiable to one of ordinary skill in the art as having its origin in the sequence.

Preferably, a nucleic acid sequence or an amino acid sequence derived from another nucleic acid, peptide, polypeptide or protein may be identical to the starting nucleic acid, peptide, polypeptide or protein (from which it is derived). However, a nucleic acid sequence or an amino acid sequence derived from another nucleic acid, peptide, polypeptide or protein may also have one or more mutations relative to the starting nucleic acid, peptide, polypeptide or protein (from which it is derived), in particular a nucleic acid sequence or an amino acid sequence derived from another nucleic acid, peptide, polypeptide or protein may be a functional sequence variant as described above of the starting nucleic acid, peptide, polypeptide or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, preferably a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

The present invention is based, amongst other findings, on the surprising finding that a fragment of LAIR-1, which is about 100 amino acids long and carries at least one mutation as outlined below and in the appended claims, is able to bind broadly to erythrocytes infected with different Plasmodium falciparum strains. In the next step, present inventors have also identified the target to which the broadly binding mutated LAIR-1 domain binds to, which is surprisingly a RIFIN and, thus, a Plasmodium falciparum surface antigen showing huge antigenic variation. In particular, it could not be expected that the mutated LAIR-1 domain, which is able to bind to different Plasmodium falciparum strains, binds to a RIFIN, and thus to a protein of a family known for their antigenic variation. This RIFIN can be used for a vaccine, which is able to induce a strong and broad antibody response to infected erythrocytes. Moreover, since RIFINs have been found also on sporozoites and gametocytes, this vaccine can also inhibit transmission.

### Pharmaceutical Composition

In a first aspect the present invention provides a pharmaceutical composition comprising a polypeptide, which comprises or consists of a second variable (V2) domain and/or an N-terminal semi-conserved domain of a RIFIN, which is/are able to bind to a LAIR-1 fragment, wherein the LAIR-1 fragment has an amino acid sequence according to SEQ ID NO: 1:

XXLPRPXXSXXXXXXXXLGSXXTXVCRGPXGXXTFRLXXXXXXX₁YX₂XX
EXVXXX₃XPXXSEARFRXXSVXXGXXGXXRCXYYXX₄X₅XWSXXSXXXXX
XVK

wherein
X is any amino acid or no amino acid (deletion mutation);
X₁ is T, L, G, I, R, K or no amino acid; however, if X₂ is N, X₃ is A, X₄ is P and X₅ is P, then X₁, is L, G, I, R, K or no amino acid;
X₂ is N, S or T; however, if X₁ is T, X₃ is A, X₄ is P and X₅ is P, then X₂ is S or T;
X₃ is A, T, P, or V; however, if X₁ is T, X₂ is N, X₄ is P and X₅ is P, then X₃ is T, P, or V;
X₄ is P, 5, A, or D; however, if X₁ is T, X₂ is N, X₃ is A and X₅ is P, then X₄ is S, A, or D; and
X₅ is P, R, or S; however, if X₁, is T, X₂ is N, X₃ is A and X₄ is P, then X₅ is R or S;
   and wherein the LAIR-1 fragment has at least 70% amino acid sequence identity to amino acids 24 to 121 of native human LAIR-1 (SEQ ID NO: 10). Preferably, the pharmaceutical composition according to the present invention comprises a polypeptide, which comprises or consists of a second variable (V2) domain, which is able to bind to a LAIR-1 fragment as described above.

Thus, the mutated LAIR-1 fragment as described herein (i.e. the mutated LAIR-1 fragment to which the second variable (V2) domain of a RIFIN/N-terminal semi-conserved domain of a RIFIN is/are able to bind to) comprises at least 1, 2, 3, 4, or 5 mutations at one or more of the following five positions (in comparison to human native LAIR-1):
T67 (referred to as “X₁” in SEQ ID NO: 1),
N69 (referred to as “X₂” in SEQ ID NO: 1),
A77 (referred to as “X₃” in SEQ ID NO: 1),
P106 (referred to as “X₄” in SEQ ID NO: 1), and
P107 (referred to as “X₅” in SEQ ID NO: 1).

One or more of these mutations enable binding of the mutated LAIR-1 fragment to a RIFIN, i.e. to a surface antigen of Plasmodium falciparum.

Optionally, the mutated LAIR-1 fragment as described herein may comprise further mutations at positions different from T67, N69, A77, P106, and P107 (i.e. in addition to one or more mutation(s) at one or more of the following five positions: T67, N69, A77, P106, and P107), with the proviso that the LAIR-1 fragment shows at least 70% amino acid sequence identity to amino acids 67 to 107 of native human LAIR-1 (SEQ ID NO: 10). Thus, one or more of such further mutations may occur in the LAIR-1 fragment as described herein.

Amino acid sequence identity may be calculated as described above. In particular, the expression “LAIR-1 fragment” refers to fragment (i.e. to a stretch of consecutive amino acids linked in particular by a peptide bond), which shows at least 70% amino acid sequence identity to amino acids 24 to 121 of native human LAIR-1 as described below (SEQ ID NO: 10). Thus, such a “LAIR-1 fragment” in particular comprises no more than 29 amino acid mutations (in total, i.e. comprising the 1-5 mutation(s) at any of positions T67, N69, A77, P106, and P107 and the mutation(s) at other position(s)) in comparison to amino acids 24 to 121 of native human LAIR-1 (i.e. in comparison to an amino acid sequence according to SEQ ID NO: 10, which has a length of 98 amino acids).

Preferably, the mutated LAIR-1 fragment shows at least 75% amino acid sequence identity to amino acids 24 to 121 of native human LAIR-1 as described below (SEQ ID NO: 10). In other words, the mutated LAIR-1 fragment comprises preferably no more than 24 amino acid mutations in comparison to amino acids 24 to 121 of native human LAIR-1 (i.e. in comparison to an amino acid sequence according to SEQ ID NO: 10, which has a length of 98 amino acids).

More preferably, the mutated LAIR-1 fragment shows at least 80% amino acid sequence identity to amino acids 24 to 121 of native human LAIR-1 as described below (SEQ ID NO: 10). In other words, the mutated LAIR-1 fragment comprises preferably no more than 19 amino acid mutations in comparison to amino acids 24 to 121 of native human LAIR-1 (i.e. in comparison to an amino acid sequence according to SEQ ID NO: 10, which has a length of 98 amino acids).

Even more preferably, the mutated LAIR-1 fragment shows at least 85% amino acid sequence identity to amino acids 24 to 121 of native human LAIR-1 as described below (SEQ ID NO: 10). In other words, the mutated LAIR-1 fragment comprises preferably no more than 14 amino acid mutations in comparison to amino acids 24 to 121 of native human LAIR-1 (i.e. in comparison to an amino acid sequence according to SEQ ID NO: 10, which has a length of 98 amino acids).

Particularly preferably, the mutated LAIR-1 fragment shows at least 87% amino acid sequence identity to amino acids 24 to 121 of native human LAIR-1 as described below (SEQ ID NO: 10). In other words, the mutated LAIR-1 fragment comprises preferably no more than 12 amino acid mutations in comparison to amino acids 24 to 121 of native human LAIR-1 (i.e. in comparison to an amino acid sequence according to SEQ ID NO: 10, which has a length of 98 amino acids).

As described above, the optional one or more further mutations at a position different from T67, N69, A77, P106, and P107 are preferably a deletion and/or a substitution, whereby a substitution is more preferred. For an amino acid substitution at a position different from T67, N69, A77, P106, and P107 it is preferred that such a substitution is a conservative amino acid substitution. In a conservative amino acid substitution the substituting amino acid has similar structural and/or chemical properties as the corresponding substituted amino acid (i.e. the amino acid in the original sequence which was substituted). By way of example, conservative amino acid substitutions involve substitution of one aliphatic or hydrophobic amino acid, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; substitution of one amide-containing residue, e.g. asparagine and glutamine, with another; substitution of one aromatic residue, e.g. phenylalanine and tyrosine, with another; substitution of one basic residue, e.g. lysine, arginine and histidine, with another; and substitution of one small amino acid, e.g., alanine, serine, threonine, methionine, and glycine, with another.

As used herein, the term “LAIR-1” refers to the protein “Leukocyte-associated immunoglobulin-like receptor 1”, which is also known as CD305. LAIR-1 is an inhibitory receptor widely expressed throughout the immune system, i.e. on peripheral mononuclear cells, including NK cells, T cells, and B cells. LAIR-1 regulates the immune response, in particular to prevent lysis of cells recognized as self. Collagens and C1q were found to be high-affinity functional ligands of LAIR-1.

LAIR-1 was implicated in various functions, including reduction of the increase of intracellular calcium evoked by B-cell receptor ligation; modulation of cytokine production in CD4+ T-cells, thereby down-regulating IL-2 and IFN-gamma production while inducing secretion of transforming growth factor-beta; down-regulation of IgG and IgE production in B-cells as well as IL-8, IL-10 and TNF secretion; inhibition of proliferation and induction of apoptosis in myeloid leukemia cell lines as well as prevention of nuclear translocation of NF-kappa-B p65 subunit/RELA and phosphorylation of I-kappa-B alpha/CHUK in these cells; and inhibition of differentiation of peripheral blood precursors towards dendritic cells. Activation by Tyr phosphorylation results in recruitment and activation of the phosphatases PTPN6 and PTPN11. A more detailed overview over the various functions of LAIR-1 is provided by Meyaard L., 2008, J Leukoc Biol. 83(4):799-803.

The gene LAIR1, which encodes the protein LAIR-1, is a member of both the immunoglobulin superfamily and the leukocyte-associated inhibitory receptor family. LAIR1 consists of 10 exons and shows considerable homology to LAIR2. The LAIR-2 gene encodes a protein hLAIR-2 that is about 84% homologous to hLAIR-1 but lacks a transmembrane and an intracellular domain (cf. Meyaard L., 2008, J Leukoc Biol. 83(4):799-803). In particular, the mutated LAIR-1 fragment as described herein may thus also be a corresponding “mutated LAIR-2 fragment”, which is mutated accordingly, i.e. in respect to the 1, 2, 3, 4, or 5 mutations at one or more of the five positions corresponding to T67, N69, A77, P106, and P107 in native human LAIR-1.

Human LAIR-1 is a type I transmembrane glycoprotein of 287 amino acids containing a single extracellular C2-type Ig-like domain and two ITIMs in its cytoplasmic tail. An ITIM is an immunoreceptor tyrosine-based inhibition motif (ITIM), which is a conserved sequence of amino acids (S/I/V/LxYxxI/V/L) that is found in the cytoplasmic tails of many inhibitory receptors of the immune system. LAIR-1 is structurally related to several other inhibitory Ig superfamily members localized to the leukocyte receptor complex (LRC) on human chromosome 19q13.4, suggesting that these molecules have evolved from a common ancestral gene.

Of the 287 amino acids of human native LAIR-1, in the order from N- to C-terminus, amino acids 1 to 21 represent a signal peptide, amino acids 22 to 165 represent an extracellular domain, amino acids 166 to 186 represent a transmembrane domain, and amino acids 187 to 287 represent a cytoplasmic domain. In mature LAIR-1, the signal peptide is typically removed, i.e. mature LAIR-1 typically comprises amino acids 22 to 287.

Several different splice variants of the LAIR-family have been cloned. LAIR-1 b lack 17 amino acids in the stalk region between the transmembrane domain and Ig-like domain as compared with the full-length LAIR-1a, which may affect their glycosylation (for review see Meyaard L., 2008, J Leukoc Biol. 83(4):799-803). LAIR-1a and LAIR-1 b might be differentially expressed in NK and T cells, but the relevance of this has not been studied extensively. LAIR-1c is identical to LAIR-1b except for a single amino acid deletion in the extracellular domain, namely, one of the glutamic acid residues at positions E23 and E24 of LAIR-1a, LAIR-1 b, and LAIR-1 d is deleted in LAIR-1c. LAIR-1d lacks part of the intracellular tail (for review see Meyaard L., 2008, J Leukoc Biol. 83(4):799-803). Genebank accession codes of the cloned cDNAs are: AF013249 (human LAIR-1a), AF109683 (human LAIR-1b), AF251509 (human LAIR-1c), AF251510 (human LAIR-1d).

In the following, the sequences of the four human LAIR-1 splice variants are provided (amino acid sequences and cDNA sequences). The five amino acid positions T67, N69, A77, P106, and P107, which are particularly relevant for the mutations in the LAIR-1 fragment according to the present invention, are shown in bold.

hLAIR-1a amino acid sequence, cf. GenBank
accession code AF013249 - “translatedprotein”:

SEQ ID NO: 2

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPYR
CIYYKPPKWSEQSDYLELLVKETSGGPDSPDTEPGSSAGPTQRPSDNSHN
EHAPASQGLKAEHLYILIGVSVVFLFCLLLLVLFCLHRQNQIKQGPPRSK
DEEQKPQQRPDLAVDVLERTADKATVNGLPEKDRETDTSALAAGSSQEVT
YAQLDHWALTQRTARAVSPQSTKPMAESITYAAVARH
hLAIR-1a nucleotide sequence, cf. GenBank
accession code AF013249 - “CDS”:

SEQ ID NO: 3

atgtctccccaccccaccgccctcctgggcctagtgctctgcctggccca
gaccatccacacgcaggaggaagatctgcccagaccctccatctcggctg
agccaggcaccgtgatccccctggggagccatgtgactttcgtgtgccgg
ggcccggttggggttcaaacattccgcctggagagggagagtagatccac
atacaatgatactgaagatgtgtctcaagctagtccatctgagtcagagg
ccagattccgcattgactcagtaagtgaaggaaatgccgggccttatcgc
tgcatctattataagccccctaaatggtctgagcagagtgactacctgga
gctgctggtgaaagaaacctctggaggcccggactccccggacacagagc
ccggctcctcagctggacccacgcagaggccgtcggacaacagtcacaat
gagcatgcacctgcttcccaaggcctgaaagctgagcatctgtatattct
catcggggtctcagtggtcttcctcttctgtctcctcctcctggtcctct
tctgcctccatcgccagaatcagataaagcaggggccccccagaagcaag
gacgaggagcagaagccacagcagaggcctgacctggctgttgatgttct
agagaggacagcagacaaggccacagtcaatggacttcctgagaaggaca
gagagacggacacctcggccctggctgcagggagttcccaggaggtgacg
tatgctcagctggaccactgggccctcacacagaggacagcccgggctgt
gtccccacagtccacaaagcccatggccgagtccatcacgtatgcagccg
ttgccagacactga
hLAIR-1b amino acid sequence, cf. GenBank
accession code AF109683 - “translated protein”:

SEQ ID NO: 4

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPY
CIYYKPPKWSEQSDYLELLVKGPTQRPSDNSHNEHAPASQGLKAEHLYIL
IGVSVVFLFCLLLLVLFCLHRQNQIKQGPPRSKDEEQKPQQRPDLAVDVL
ERTADKATVNGLPEKDRETDTSALAAGSSQEVTYAQLDHWALTQRTARAV
SPQSTKPMAESITYAAVARH
hLAIR-1b nucleotide sequence, cf. GenBank
accession code AF109683 - “CDS”:

SEQ ID NO: 5

atgtctccccaccccaccgccctcctgggcctagtgctctgcctggccca
gaccatccacacgcaggaggaagatctgcccagaccctccatctcggctg
agccaggcaccgtgatccccctggggagccatgtgactttcgtgtgccgg
ggcccggttggggttcaaacattccgcctggagagggagagtagatccac
atacaatgatactgaagatgtgtctcaagctagtccatctgagtcagagg
ccagattccgcattgactcagtaagtgaaggaaatgccgggccttatcgc
tgcatctattataagccccctaaatggtctgagcagagtgactacctgga
gctgctggtgaaaggacccacgcagaggccgtcggacaacagtcacaatg
agcatgcacctgcttcccaaggcctgaaagctgagcatctgtatattctc
atcggggtctcagtggtcttcctcttctgtctcctcctcctggtcctctt
ctgcctccatcgccagaatcagataaagcaggggccccccagaagcaagg
acgaggagcagaagccacagcagaggcctgacctggctgttgatgttcta
gagaggacagcagacaaggccacagtcaatggacttcctgagaaggacag
agagacggacacctcggccctggctgcagggagttcccaggaggtgacgt
atgctcagctggaccactgggccctcacacagaggacagcccgggctgtg
tccccacagtccacaaagcccatggccgagtccatcacgtatgcagccgt
tgccagacactga
hLAIR-1c amino acid sequence, cf. GenBank
accession code AF251509 - “translated protein”:

SEQ ID NO: 6

MSPHPTALLGLVLCLAQTIHTQEDLPRPSISAEPGTVIPLGSHVTFVCRG
PVGVQTFRLERESRSTYNDTEDVSQASPSESEARFRIDSVSEGNAGPYRC
IYYKPPKWSEQSDYLELLVKGPTQRPSDNSHNEHAPASQGLKAEHLYILI
GVSVVFLFCLLLLVLFCLHRQNQIKQGPPRSKDEEQKPQQRPDLAVDVLE
RTADKATVNGLPEKDRETDTSALAAGSSQEVTYAQLDHWALTQRTARAVS
PQSTKPMAESITYAAVARH
hLAIR-1c nucleotide sequence, cf. GenBank
accession code AF251509 - “CDS”:

SEQ ID NO: 7

atgtctccccaccccaccgccctcctgggcctagtgctctgcctggccca
gaccatccacacgcaggaggatctgcccagaccctccatctcggctgagc
caggcaccgtgatccccctggggagccatgtgactttcgtgtgccggggc
ccggttggggttcaaacattccgcctggagagggagagtagatccacata
caatgatactgaagatgtgtctcaagctagtccatctgagtcagaggcca
gattccgcattgactcagtaagtgaaggaaatgccgggccttatcgctgc
atctattataagccccctaaatggtctgagcagagtgactacctggagct
gctggtgaaaggacccacgcagaggccgtcggacaacagtcacaatgagc
atgcacctgcttcccaaggcctgaaagctgagcatctgtatattctcatc
ggggtctcagtggtcttcctcttctgtctcctcctcctggtcctcttctg
cctccatcgccagaatcagataaagcaggggccccccagaagcaaggacg
aggagcagaagccacagcagaggcctgacctggctgttgatgttctagag
aggacagcagacaaggccacagtcaatggacttcctgagaaggacagaga
gacggacacctcggccctggctgcagggagttcccaggaggtgacgtatg
ctcagctggaccactgggccctcacacagaggacagcccgggctgtgtcc
ccacagtccacaaagcccatggccgagtccatcacgtatgcagccgttgc
cagacactga
hLAIR-1d amino acid sequence, cf. GenBank
accession code AF251510 - “translated protein”:

SEQ ID NO: 8

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPYR
CIYYKPPKWSEQSDYLELLVKETSGGPDSPDTEPGSSAGPTQRPSDNSHN
EHAPASQGLKAEHLYILIGVSVVFLFCLLLLVLFCLHRQNQIKQGPPRSK
DEEQKPQQR
hLAIR-1d nucleotide sequence, cf. GenBank
accession code AF251510 - “CDS”:

SEQ ID NO: 9

atgtctccccaccccaccgccctcctgggcctagtgctctgcctggccca
gaccatccacacgcaggaggaagatctgcccagaccctccatctcggctg
agccaggcaccgtgatccccctggggagccatgtgactttcgtgtgccgg
ggcccggttggggttcaaacattccgcctggagagggagagtagatccac
atacaatgatactgaagatgtgtctcaagctagtccatctgagtcagagg
ccagattccgcattgactcagtaagtgaaggaaatgccgggccttatcgc
tgcatctattataagccccctaaatggtctgagcagagtgactacctgga
gctgctggtgaaagaaacctctggaggcccggactccccggacacagagc
ccggctcctcagctggacccacgcagaggccgtcggacaacagtcacaat
gagcatgcacctgcttcccaaggcctgaaagctgagcatctgtatattct
catcggggtctcagtggtcttcctcttctgtctcctcctcctggtcctct
tctgcctccatcgccagaatcagataaagcaggggccccccagaagcaag
gacgaggagcagaagccacagcagaggtga

Of note, all of the four isoforms of human native LAIR-1 comprise the identical sequence motif according to SEQ ID NO: 10 as shown below, which comprises the five amino acid positions at which a mutation may occur in the LAIR-1 fragment (shown in bold):

(SEQ ID NO: 10)

EDLPRPSISAEPGTVIPLGSHVTFVCRGPVGVQTFRLERESRSTYNDTED
VSQASPSESEARFRIDSVSEGNAGPYRCIYYKPPKWSEQSDYLELLVK

This motif is shown underlined in the above amino acid sequences of the four isoforms of native human LAIR-1 (cf. SEQ ID NOs 2, 4, 6 and 8).

This sequence motif of native human LAIR-1 (amino acids 24-121 of native human LAIR-1) is in particular the polypeptide encoded by the third exon of native human LAIR-1. Namely, the gene LAIR-1 (identifier: ENSG00000167613) is located on human chromosome 19: 54,351,384-54,370,558 reverse strand. The “third exon” of native human LAIR-1 comprises, in particular consists of, amino acids 23-120 in case of the third exon (identifier: ENSE00003538434) of the LAIR-1 isoform hLAIR-1c, while the “third exon” of native human LAIR-1 comprises, in particular consists of, amino acids 24-121 in case of the third exon of the other LAIR-1 isoforms (identifier: ENSE00003554448).

Of note, the positions T67, N69, A77, P106, and P107 are identical in human LAIR-1a, hLAIR-1b, and hLAIR-1d, while in hLAIR-1c (SEQ ID NO: 5) these positions are shifted—due to the deletion of one of E23 and E24—to the positions T66, N68, A76, P105, and P106. It is understood that the expressions “at one or more of the following five positions: T67, N69, A77, P106, and P107” and “at a position different from T67, N69, A77, P106, and P107” as used herein, thus refers to exactly these positions of hLAIR-1a, hLAIR-1b, and hLAIR-1d—whereas it refers to positions T66, N68, A76, P105, and P106 in hLAIR-1c.

Moreover, the above sequence motif according to SEQ ID NO: 10 thus corresponds to amino acids 24-121 in hLAIR-1a, hLAIR-1b, and hLAIR-1d, but to amino acids 23—120 in hLAIR-1c.

In the present invention it is preferred that the LAIR-1 fragment as described herein (i) includes at least a mutation at the position T67; or (ii) includes at least a mutation at the position N69; or (iii) includes at least a mutation at the position A77; or (iv) includes at least a mutation at the position P106; or (v) includes at least a mutation at the position P107. Preferably, the LAIR-1 fragment as described herein includes at least a mutation at the position N69, more preferably the LAIR-1 fragment as described herein includes at least a mutation at the position N69 selected from the group consisting of N69S and N69T, even more preferably the LAIR-1 fragment as described herein includes at least the mutation N69S.

It is also preferred that the LAIR-1 fragment as described herein includes a mutation at least two of the following five positions: T67, N69, A77, P106, and P107. Thereby, the LAIR-1 fragment as described herein may preferably include (i) at least a mutation at the position T67 and at the position N69; or (ii) at least a mutation at the position T67 and at the position A77; or (iii) at least a mutation at the position T67 and at the position P106; or (iv) at least a mutation at the position T67 and at the position P107; or (v) at least a mutation at the position N69 and at the position A77; or (vi) at least a mutation at the position N69 and at the position P106; or (vii) at least a mutation at the position N69 and at the position P107; or (viii) at least a mutation at the position A77 and at the position P106; or (ix) at least a mutation at the position A77 and at the position P107; or (x) at least a mutation at the position P106 and at the position P107.

More preferably, the LAIR-1 fragment as described herein includes (i) at least a mutation at the position T67 and at the position N69, (ii) at least a mutation at the position T67 and at the position A77, or (iii) at least a mutation at the position A77 and at the position N69; even more preferably the LAIR-1 fragment as described herein includes (i) at least a mutation at the position T67 selected from the group consisting of T67G, T67I, T67L, T67R, and T67K and at the position N69 selected from the group consisting of N69S and N69T, (ii) at least a mutation at the position T67 selected from the group consisting of T67G, T67I, T67L, T67R, and T67K and at the position A77 selected from the group consisting of A77T, A77P and A77V, or (iii) at least a mutation at the position A77 selected from the group consisting of A77T, A77P and A77V and at the position N69 selected from the group consisting of N69S and N69T; and particularly preferably the LAIR-1 fragment as described herein includes (i) at least the mutations T67L and N69S, (ii) at least the mutations T67L and A77T, or (iii) at least the mutations N69S and A77T.

Preferably, the LAIR-1 fragment as described herein includes a mutation at least three of the following five positions: T67, N69, A77, P106, and P107. Thereby, the LAIR-1 fragment as described herein may preferably include (i) at least a mutation at the position T67, at the position N69 and at the position A77; or (ii) at least a mutation at the position T67, at the position N69 and at the position P106; or (iii) at least a mutation at the position T67, at the position N69 and at the position P107; or (iv) at least a mutation at the position T67, at the position A77 and at the position P106; or (v) at least a mutation at the position T67, at the position A77 and at the position P107; or (vi) at least a mutation at the position T67, at the position P106 and at the position P107; or (vii) at least a mutation at the position N69, at the position A77 and at the position P106; or (viii) at least a mutation at the position N69, at the position A77 and at the position P107; or (ix) at least a mutation at the position N69, at the position P106 and at the position P107; or (x) at least a mutation at the position A77, at the position P106 and at the position P107.

More preferably, the LAIR-1 fragment as described herein includes (i) at least a mutation at the position T67, at the position N69 and at the position A77, (ii) at least a mutation at the position T67, at the position N69 and at the position P107 or (iii) at least a mutation at the position T67, at the position A77 and at the position P107; even more preferably the LAIR-1 fragment as described herein includes (i) at least a mutation at the position T67 selected from the group consisting of T67G, T67I, I67L, I67R, and I67K, at the position N69 selected from the group consisting of N69S and N69T and at the position A77 selected from the group consisting of A77T, A77P and A77V, (ii) at least a mutation at the position T67 selected from the group consisting of T67G, T67I, T67L, T67R, and T67K, at the position N69 selected from the group consisting of N69S and N69T and at the position P107 selected from the group consisting of P107S and P107R or (iii) at least a mutation at the position T67 selected from the group consisting of T67G, T67I, T67L, T67R, and T67K, at the position A77 selected from the group consisting of A771, A77P and A77V and at the position P107 selected from the group consisting of P107S and P107R; and particularly preferably the LAIR-1 fragment as described herein includes (i) at least the mutations T67L, N69S and A77T, (ii) at least the mutations T67L, N69S and P107R, or (iii) at least the mutations T67L, A77T and P107R.

It is also preferred that the LAIR-1 fragment as described herein includes a mutation at at least four of the following five positions: T67, N69, A77, P106, and P107. Thereby, the LAIR-1 fragment as described herein may preferably include (i) at least a mutation at the position T67, at the position N69, at the position A77 and at the position P106; or (ii) at least a mutation at the position T67, at the position N69, at the position A77 and at the position P107; or (iii) at least a mutation at the position T67, at the position N69, at the position P106 and at the position P107; or (iv) at least a mutation at the position T67, at the position A77, at the position P106 and at the position P107; or (v) at least a mutation at the position N69, at the position A77, at the position P106 and at the position P107.

More preferably, the LAIR-1 fragment as described herein includes (i) at least a mutation at the position T67, at the position N69, at the position A77, and at position P107 or (ii) at least a mutation at the position T67, at the position N69, at the position P106, and at position P107; even more preferably the LAIR-1 fragment as described herein includes (i) at least a mutation at the position T67 selected from the group consisting of T67G, T67I, T67L, T67R, and T67K, at the position N69 selected from the group consisting of N69S and N69T, at the position A77 selected from the group consisting of A77T, A77P and A77V, and at the position P107 selected from the group consisting of P107S and P107R or (ii) at least a mutation at the position T67 selected from the group consisting of T67G, T67I, T67L, T67R, and T67K, at the position N69 selected from the group consisting of N69S and N69T, at the position P106 selected from the group consisting of P106S, P106A, and P106D, and at the position P107 selected from the group consisting of P107S and P107R; and particularly preferably the LAIR-1 fragment as described herein includes (i) at least the mutations T67L, N69S, A77T and P107R or (ii) at least the mutations T67L, N69S, P106S and P107R.

Preferably, the LAIR-1 fragment as described herein includes a mutation at each of the following five positions: T67, N69, A77, P106, and P107; more preferably the LAIR-1 fragment as described herein includes a mutation at the position T67 selected from the group consisting of T67G, T67I, T67L, T67R, and T67K, at the position N69 selected from the group consisting of N69S and N69T, at the position A77 selected from the group consisting of A77T, A77P and A77V, at the position P106 selected from the group consisting of P106S, P106A, and P106D and at the position P107 selected from the group consisting of P107S and P107R; and particularly preferably the LAIR-1 fragment as described herein includes the mutations T67L, N69S, A77T, P106S and P107R.

In the present invention, it is preferred that the mutation is a deletion or a substitution, preferably the mutation is a substitution as described above.

Preferably, the pharmaceutical composition according to the present invention comprises a polypeptide, which comprises or consists of a second variable (V2) domain and/or an N-terminal semi-conserved domain of a RIFIN, which is/are able to bind to a LAIR-1 fragment, wherein the LAIR-1 fragment has an amino acid sequence according to SEQ ID NO: 11 as shown below and wherein the LAIR-1 fragment has at least 70% amino acid sequence identity to amino acids 24 to 121 of native human LAIR-1 (SEQ ID NO: 10).

SEQ ID NO: 11

XXLPRPXXSXXXXXXXXLGSXXTXVCRGPXGXXTFRLXXXXXXX₁YX₂XX
EXVXXX₃XPXXSEARFRXXSVXXGXXGXXRCXYYXX₄X₅XWSXXSXXXXX
XVK

wherein
X is any amino acid or no amino acid (deletion);
X₁ is T or L; however, if X₂ is N, X₃ is A, X₄ is P and X₅ is P, then X₁, is L;
X₂ is N or 5; however, if X₁ is T, X₃ is A, X₄ is P and X₅ is P, then X₂ is S;
X₃ is A or T; however, if X₁, is T, X₂ is N, X₄ is P and X₅ is P, then X₃ is T;
X₄ is P or 5; however, if X₁ is T, X₂ is N, X₃ is A and X₅ is P, then X₄ is S; and
X₅ is P or R; however, if X₁ is T, X₂ is N, X₃ is A and X₄ is P, then X₅ is R.

Preferably, the LAIR-1 fragment as described herein comprises at least the following mutation in comparison to native human LAIR-1 T67L and/or N69S.

In the present invention, it is particularly preferred that the LAIR-1 fragment, to which the second variable (V2) domain of a RIFIN and/or the N-terminal semi-conserved domain of a RIFIN is able to bind to, comprises at least the following mutations in comparison to native human LAIR-1: T67L, N69S, A77T, P106S, and P107R.

Preferably, the second variable (V2) domain of a RIFIN/N-terminal semi-conserved domain of a RIFIN is/are able to bind to a LAIR-1 fragment having an amino acid sequence according to any of SEQ ID NOs 12, 14, 16, 18, 20, 22, 24, 26, 28, 30, 32, 34, 36, 38, 40, 42, 44, 46, 48, 50, 52, and 54 or the second variable (V2) domain of a RIFIN/N-terminal semi-conserved domain of a RIFIN is/are able to bind to a functional sequence variant of these exemplified amino acid sequences. These exemplified amino acid sequences of the LAIR-1 fragment are shown below in Table 1. Moreover, Table 1 also shows preferred examples of nucleic acid sequences encoding said amino acid sequences.

TABLE 1
Sequences and SEQ ID NOs of preferred exemplary LAIR1 fragments as described
herein.

| SEQ | | |
|---|---|---|
| ID | | |
| NO | Description | Sequence |
| 12 | MGC1/MGC32_EXON | EDLPRPSISAAEGTVIPLGSHVTFVCRGPVGVQTFRLEKDSRSIY |
| | aa | NDTENVSQPSPSESEARFRIDSVSEGNAGLYRCVYYKAPKWSA |
| | | QSDYLELLVK |
| 13 | MGC1/MGC32_EXON | Gaagatctgcccagaccctccatctcggctgccgaaggcaccgtgatccccctgggg |
| | nucl | agccatgtgactttcgtgtgccggggcccggttggggttcaaacattccgcctggagaa |
| | | ggacagtagatccatatacaatgatactgaaaatgtgtctcaacctagtccatctgagtc |
| | | agaggccagatttcgcattgactcagtgagtgaaggaaatgccggactttatcggtgcgt |
| | | ctattataaggcccctaaatggtctgcgcagagtgattacctggagctgctggtgaaa |
| 14 | MGC2_EXON | EHLPRPSISPEPGTVITLGSHVTFVCRGPVGVQTFRLEKDSRSTY |
| | aa | NDTEDVSQPSPSESEARFRIDSVSEGYAGLYRCLYYKPPKWSEQ |
| | | SDYLELLVK |
| 15 | MGC2_EXON | gagcatctgcccagaccctccatctcgcctgagccaggcaccgtgatcaccctgggg |
| | nucl | agccatgtgactttcgtgtgccggggcccggttggggttcaaacattccgcctggagaa |
| | | ggacagtagatccacatacaatgatactgaagatgtgtctcaacctagtccatctgagtc |
| | | agaggccagattccgcattgactcagtaagtgaaggatatgccgggctttatcgctgcct |
| | | ctattataagccccctaaatggtctgagcagagtgactacctggagctgctggtgaaa |
| 16 | MGC4_EXON | EDLPRPSISAEPDTVIPLGSHVTFVCRGPVGVHTFRLERGWRYN |
| | aa | DTEDVSQAGPSESEARFRIDSVREGNAGLYRCIYYIAPKWSEQS |
| | | DYLELRVK |
| 17 | MGC4_EXON | Gaagatctgcccagaccctccatctcggctgagccagacaccgtaatccccctgggg |
| | nucl | agccatgtgactttcgtgtgccggggcccggttggggttcacacattccgcctggagag |
| | | ggggtggaggtacaacgacactgaagatgtgtctcaagctggtccatctgagtcagagg |
| | | ccagattccgcattgactcggtaagggaaggaaatgccgggctttatcgatgcatctatt |
| | | acatagcccctaaatggtctgagcagagtgactacctggagctgcgggtgaaa |
| 18 | MGC5/MGC29_EXON | EDLPRPSISAEPGTVIPLGSHVTFVCRGPVGVHTFRLERGWRYN |
| | aa | DTEDVSQAGPSQSEARFRIDSVREGNAGLYRCLYYIPPKWSEQ |
| | | SDYLELRVK |
| 19 | MGC5/MGC29_EXON | Gaagatctgcccagaccctccatctcggctgagccaggcaccgtgatccccctgggg |
| | nucl | agccatgtgactttcgtgtgccggggccccgttggggttcacacattccgcctggagag |
| | | ggggtggagatacaacgacactgaagatgtgtctcaagctggtccatctcagtcagagg |
| | | ccagattccgcattgactcggtaagggaaggaaatgccgggctttatcgatgcctctatt |
| | | acataccccctaaatggtctgagcagagtgactacctggaactgcgggtgaaa |
| 20 | MGC7/MGC37_EXON | DDLPRPSISPEPGTVIPLGSHVTFVCRGPVGVQTFRLEKDRRST |
| | aa | YNDTEDVSQPSPSESEARFRIDSVTEGNAGLYRCVYYKPPKWS |
| | | DQSDFLELLVK |
| 21 | MGC7/MGC37_EXON | Gatgatctgcccagaccctctatctcgcctgagccaggcaccgtgatccccctgggga |
| | nucl | gccatgtgactttcgtgtgtcggggcccggttggggttcaaacattccgcctggagaagg |
| | | acagaagatccacatacaatgatactgaagatgtgtctcaacctagtccatctgagtca |
| | | gaggccagattccgcattgactcagtaactgaaggaaatgccgggctttatcgctgcgt |
| | | ctattataagccccctaaatggtctgaccagagtgacttcctggagttgctggtgaag |
| 22 | MGC17_EXON | EDLPRPSISAEEGTVIPLGSRLTFVCRGPVGVHTFRLERDRRSTY |
| | aa | NDTEDVSHPSPSESEARFRIDSVSEGNAGLYRCVYYKSPEWSK |
| | | QSDYLELLVK |
| 23 | MGC17_EXON | Gaagatctgcccagaccctccatctcggctgaggaaggcaccgtgattcccctgggg |
| | nucl | agccgtctgactttcgtgtgccggggcccggttggggttcacacattccgcctggagag |
| | | ggaccgtagatccacatacaatgatactgaagatgtgtctcaccctagtccatctgagtc |
| | | tgaggccagatttcgcattgactcagtgagtgaaggaaatgccgggctttatcgctgcgt |
| | | ctattataagtcccctgaatggtctaagcagagtgattacctggagctgctggtgaaa |
| 24 | MGC26_EXON | EDLPRPSISPEPATVIPLGSHVTIVCRGPVGVETFRLQKESRSLYN |
| | aa | DTEDVSQPSPSESEARFRIDSVSEGHGGLYRCLYYKSSKWSEQS |
| | | DYLEMLVK |
| 25 | MGC26_EXON | Gaagatctgcccagaccctccatctcgccggagccagccaccgtgatccccctggg |
| | nucl | gagccatgtgactatcgtgtgccggggcccggttggggttgaaacattccgcctgcaga |
| | | aggagagtagatccctgtacaatgacactgaagatgtgtctcaacctagtccatctgagt |
| | | cagaggccagattccgcattgactcagtaagtgaagggcatggcgggctttatcgctgc |
| | | ctctattataagtcttctaaatggtctgagcagagtgactacctggagatgctggtgaaa |
| 26 | MGC28/MGC33_EXON | EDLPRPTISAETGTVISLGSHVTFVCRGPLGVQTFRLERESRSRYS |
| | aa | ETEDVSQVGPSESEARFRIDSVSEGNAGLYRCIYYKPPKWSEQS |
| | | DYLELRVK |
| 27 | MGC28/MGC33_EXON | Gaagatctgcccagacccaccatctcggctgagacaggcaccgtgatctccctgggg |
| | nucl | agccatgtgactttcgtgtgccggggcccacttggggtgcaaacattccgcctggagag |
| | | ggagagtaggtccagatacagtgaaactgaagatgtgtctcaagttggtccatctgagtc |
| | | agaggccagattccgcattgactcagtgagtgaaggaaatgccgggctttatcgatgca |
| | | tctattacaaaccccctaaatggtctgagcagagtgactacctggagctgcgggtgaaa |
| 28 | MGC34_EXON | EDLPRPSISAEPGTVIPLGSHVTFVCRGPVGIHTFRLERESRSLYT |
| | aa | ETEDVTQVSPSESEARFRIESVTEGNAGLYRCVYYKPPKWSEQS |
| | | DYLELLVK |
| 29 | MGC34_EXON | Gaagatctgcccagaccctccatctcggctgagccaggcaccgtgatccccctgggg |
| | nucl | agtcatgtgaccttcgtgtgccggggcccggttgggattcacacattccgcctggagag |
| | | ggagagtagatccctatacactgaaactgaagatgtgactcaagtaagtccttctgagtc |
| | | agaggccagattccgcattgagtcagtaactgaaggaaatgccgggctttatcgctgcg |
| | | tctattataagccccctaaatggtctgagcagagtgactacctggagctgctggtgaaa |
| 30 | MGC35_EXON | EDLPRPSISAEPGSVIPLGSLVTFVCRGPVGVHTFRLERGWTYN |
| | aa | DTEDVSQAGPSESEARFRMDSVREGNAGLYRCIYYKPPKWSE |
| | | QSAYLELRVK |
| 31 | MGC35_EXON | Gaagatctgcccagaccctccatctcggctgagccaggctccgtgatccccctgggg |
| | nucl | agccttgtgactttcgtgtgccggggcccggttggggttcacacattccgcctcgagagg |
| | | gggtggacatacaacgacactgaagatgtgtctcaagctggtccatctgagtcagaggc |
| | | cagattccgcatggactcggtaagggaaggaaatgccgggctttatcgatgcatctatta |
| | | caaaccccctaaatggtctgagcagagtgcctacctggaactgcgggtgaaa |
| 32 | MGC36_EXON | EEDLPRPSISAEPDTVIPLGSHVTFVCRGPVGVHTFRLERGWRY |
| | aa | NDTEDVSQAGPSESEARFRIDSVREGNAGLYRCIYYIAPKWSE |
| | | QSDYLELRVK |
| 33 | MGC36_EXON | Gaagaagatctgcccagaccctccatctcggctgagccagacaccgtaatccccctg |
| | nucl | gggagccatgtgactttcgtgtgccggggcccggttggggttcacacattccgcctgga |
| | | gagggggtggaggtacaacgacactgaagatgtgtctcaagctggtccatctgagtcag |
| | | aggccagattccgcattgactcggtaagggaaggaaatgccgggctttatcgatgcatc |
| | | tattacatagcccctaaatggtctgagcagagtgactacctggagctgcgggtgaaa |
| 34 | MGD21_EXON | DLPRPSISAEPGTVIPLGSHVTFVCRGPVGVQTFRLERERNYLYS |
| | aa | DTEDVSQTSPSESEARFRIDSVNAGNAGLFRCIYYKSRKWSEQS |
| | | DYLELWK |
| 35 | MGD21_EXON | Gatctgcccagaccctccatctcggctgagccgggcaccgtgatccccctggggagc |
| | nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaa |
| 36 | MGD23_EXON | EDLPRPSLSAEPGTVIPLGSHVTFVCRGPAGVETFRLERESRFTY |
| | aa | NDTEDVSQASPSESEARFRIDSVSEGNAGPYRCLYYKARKWSD |
| | | QSDYLELLVK |
| 37 | MGD23_EXON | Gaagatctgcccagaccctccctctcggctgaaccaggcaccgtgatccccctgggg |
| | nucl | agtcacgtgactttcgtgtgccggggcccggctggggtcgaaacattccgcctggagag |
| | | ggagagtagattcacttacaacgatactgaagatgtgtctcaagcgagtccatctgagtc |
| | | agaggccagattccgcattgactcagtaagtgaaggaaatgccgggccttatcgctgcc |
| | | tctattataaggcccgtaaatggtctgaccagagtgactacttggaattgctggtgaag |
| 38 | MGD30_EXON | EKLPRPSISAEPGTVIPLGSRVTFVCRGPVGVQTFRLERETSFTYN |
| | aa | DTEDVSQVSPSESEARFRIDSVSEGYAGPYRCVYYKAPKWSEQ |
| | | SDYLDLLVK |
| 39 | MGD30_EXON | Gaaaaactgcccagaccctccatctcggctgagccgggcaccgtgatccccctggg |
| | nucl | gagccgtgtgactttcgtgtgccggggcccggttggggttcaaacattccgcctagaga |
| | | gggagactagctttacatataatgatactgaagatgtgtctcaggttagtccgtctgagtca |
| | | gaggccagattccgcattgactcagtgagtgagggatatgccgggccttatcgctgcgt |
| | | ctattataaggcccctaagtggtccgagcagagtgactacctggacctgctggtgaaa |
| 40 | MGD33_EXON | EKLPRPSISAEPGTVIPLGSRVTFVCRGPVGVQTFRLERETRSTY |
| | aa | NDTEDVSQVSPSESEARFRIDSVSEGYAGPYRCVYYKAPKWSE |
| | | QSDYLDLLVK |
| 41 | MGD33_EXON | gaaaaactgcccagaccctccatctcggctgagccgggcaccgtgatccccctgggg |
| | nucl | agccgtgtgactttcgtgtgccggggcccggttggggttcaaacattccgcctagagag |
| | | ggagactagatctacatataatgatactgaagatgtgtctcaggttagtccgtctgagtca |
| | | gaggccagattccgcattgactcagtgagtgagggatatgccgggccttatcgctgcgt |
| | | ctattataaggcccctaagtggtccgagcagagtgactacctggacctgctggtgaaa |
| 42 | MGD34_EXON | EDLPRPSISAEPGTVIPLGSHVTFVCRGPVGVQTFRLERERNYLY |
| | aa | SDTEDVSQTSPSESEARFRIDSVNAGNAGLFRCIYYKSRKWSEQ |
| | | SDYLELVVK |
| 43 | MGD34_EXON | Gaagatctgcccagaccctccatctcggctgagccgggcaccgtgatccccctgggg |
| | nucl | agccatgtgactttcgtgtgccggggcccggttggggttcaaacattccgcctggagag |
| | | ggagaggaattatttatacagtgatactgaagatgtgtctcaaactagtccatctgagtcg |
| | | gaggccagattccgcattgactcagtaaatgcaggcaatgccgggctttttcgctgcatc |
| | | tattacaagtcccgtaaatggtctgagcagagtgactacctggagctggtggtgaaa |
| 44 | MGD35_EXON | NLPRPSLSAEPGTVIPLGSPVTFVCRGPVGVHTFRLERAGRSTY |
| | aa | NDTEDVSHPSPSESEARFRIDSVSEGNAGPYRCVYYKSSKWSEE |
| | | SYCLDLLVK |
| 45 | MGD35_EXON | aatttgcccagaccctccctctcggcggagccaggcaccgtgatccccctggggagc |
| | nucl | cctgtgactttcgtgtgccggggcccggttggggttcacacattccgcctggagagggc |
| | | gggtagatccacatacaatgatactgaagatgtgtctcatcctagtccatctgagtcaga |
| | | ggccagattccgcattgactcagtgagtgagggaaatgccgggccttatcgctgcgtct |
| | | attataagtcctctaaatggtccgaggagagttactgcctggacctgctggtcaaa |
| 46 | MGD39_EXON | DDLPRPSISAEPGTVIPLGSHVTFVCRGPIGVQTFRLERERRSLYS |
| | aa | DTEDVSQVSPFASEARFRIDSVSEGNAGPYRCIYYKDRKWSDQ |
| | | SDYLELLVK |
| 47 | MGD39_EXON | Gacgatctgcccagaccctccatctcggctgagccaggcaccgtgatccccctgggg |
| | nucl | agccatgtgaccttcgtgtgccggggcccaattggggttcaaacattccgcctggagag |
| | | ggagagaagatccttatacagtgatactgaagatgtgtctcaagttagtccatttgcgtca |
| | | gaggccagattccgcattgactcagtaagtgaaggaaatgccgggccatatcgctgcat |
| | | ctattataaggaccggaaatggtctgaccagagtgactacctggagttgctggtgaaa |
| 48 | MGD41_EXON | EDLPRPSLSAEPGTVVPLGSHVTFVCRGPVGVQTFRLERESRST |
| | aa | YNDTEDVSQPSPFESEARFRIDSVSEGNAGPYRCIYYKSPKWSD |
| | | QSDYVELLVK |
| 49 | MGD41_EXON | Gaagatctgcccagaccctccctctcggctgagccaggcaccgtggtccccctgggg |
| | nucl | agccatgtgactttcgtgtgccggggcccggttggggttcaaacattccgcctggagag |
| | | ggagagcagatccacatacaatgatactgaagatgtgtctcaacctagtccatttgagtc |
| | | agaggccagatttcgcattgactcagtaagtgaaggaaatgccgggccttatcgctgca |
| | | tctattataagtcccctaaatggtctgaccagagtgactacgtggagttgctggtgaaa |
| 50 | MGD47_EXON | GDLPRPSISAEPGTAIPLGSQVTFVCRGPIGVQTFRLERESRALY |
| | aa | NDSEDVSQVSPSASEARFRIDSVSEGNAGPYRCIYYKARRWSD |
| | | QSDYLELLVK |
| 51 | MGD47_EXON | ggagatctgcccagaccctccatctcggctgagccaggcaccgcgatccccctgggg |
| | nucl | agccaagtgactttcgtgtgccggggcccaattggggttcaaacattccgcctggagag |
| | | ggagagtcgcgccttatataatgattctgaagatgtgtctcaagttagtccatctgcgtcag |
| | | aggccagattccgcattgactcagtaagtgaaggcaatgccgggccttatcgctgtatct |
| | | attataaggcccgcagatggtctgaccagagtgactatttggagttgttggtgaaa |
| 52 | MGD55_EXON | DDLPRPSISAEPGTVIPLGSHVTFVCRGPIGVQTFRLERESRSLYS |
| | aa | DTEDVSQVSPFASEARFRIDSVSEGNAGPYRCIYYKDRKWSDQ |
| | | SDYLELLVK |
| 53 | MGD55_EXON | gacgatctgcccagaccctccatctcggctgagccaggcaccgtgatccccctgggg |
| | nucl | agccatgtgactttcgtgtgccggggcccaattggggttcaaacattccgcctggagag |
| | | ggagagtagatccttatacagtgatactgaagatgtgtctcaagttagtccatttgcgtcag |
| | | aggccagattccgcattgactcagtaagtgaaggaaatgccgggccatatcgctgcatc |
| | | tattataaggaccggaaatggtctgaccagagtgactacctggagttgctggtgaaa |
| 54 | MGD56_EXON | KDLPRPSLSAEPGTVIPLGSHVTFVCRGPVGVQTFRLQRESRSL |
| | aa | YNDTEDVSHPSPSESEARFRIDSVSEGNAGPYRCVYYKSSKWSE |
| | | ESDCLELLVK |
| 55 | MGD56_EXON | aaagatttgcccagaccctccctctcggctgagccaggcaccgtgatccccctgggga |
| | nucl | gtcatgtgactttcgtgtgccggggcccggttggggttcagacartccgcctgcagaggg |
| | | agagtagatccctttacaatgatactgaagatgtgtctcatcctagtccatctgagtcaga |
| | | ggccagattccgcattgactcagtgagtgagggaaatgccgggccttatcgctgcgtct |
| | | attataagtcctctaaatggtccgaggagagtgactgcctggagctgctggtcaaa |

More preferably, the second variable (V2) domain of a RIFIN/N-terminal semi-conserved domain of a RIFIN is/are able to bind to a LAIR-1 fragment having an amino acid sequence according to any of SEQ ID NO: 28, 34, 42, 46, 50, and 52 or to a functional sequence variant thereof.

Even more preferably, the second variable (V2) domain of a RIFIN/N-terminal semi-conserved domain of a RIFIN is/are able to bind to a LAIR-1 fragment having an amino acid sequence according to SEQ ID NO: 34 or according to a functional sequence variant thereof.

It is also preferred that the second variable (V2) domain of a RIFIN/N-terminal semi-conserved domain of a RIFIN is/are able to bind to an antibody comprising such a LAIR-1 fragment as described above. As used herein, the term “antibody” encompasses various forms of antibodies including, without being limited to, whole antibodies, antibody fragments, human antibodies, chimeric antibodies, humanized antibodies and genetically engineered antibodies (variant or mutant antibodies) as long as the characteristic properties according to the invention are retained. Especially preferred are human or humanized monoclonal antibodies, especially as recombinant human monoclonal antibodies.

The antibody comprising such a LAIR-1 fragment as described above can be of any isotype (e.g., IgA, IgG, IgM i.e. an α, γ or μ heavy chain), but will preferably be IgG. Within the IgG isotype, antibodies may be IgG1, IgG2, IgG3 or IgG4 subclass, whereby IgG1 is preferred. Antibodies of the invention may have a κ or a λ light chain.

Exemplified antibodies comprising such a LAIR-1 fragment as described above, which are preferably of the IgG1 type, are shown below in Table 2. Thus, it is preferred that the second variable (V2) domain of a RIFIN/N-terminal semi-conserved domain of a RIFIN is/are able to bind to an exemplified antibody as shown in Table 2, which is preferably of the IgG1 type having amino acid sequences for the constant region as shown below in Table 2, namely according to (i) SEQ ID NOs 524 and 525 or (ii) SEQ ID NOs 524 and 526, or functional sequence variants thereof.

TABLE 2
Sequences and SEQ ID Numbers of preferred exemplary antibodies

| | | |
|---|---|---|
| SEQ | | |
| ID | | |
| NO | Description | Sequence |

MGC1 ANTIBODY

| | | |
|---|---|---|
| 56 | CDRH1 aa | GFNFRKSW |
| 57 | CDRH2 aa | IREDGSES |
| 58 | CDRH3 aa | ARDRFCNDDEIHRHGQEDLPRPSISAAEGTVIPLGSHVTFVCR |
| | | GPVGVQTFRLEKDSRSIYNDTENVSQPSPSESEARFRIDSVSEG |
| | | NAGLYRCVYYKAPKWSAQSDYLELLVKGQEVTWALFTSCGG |
| | | DGEEPDYDMDV |
| 59 | CDRL1 aa | QSVLYRSKNKNY |
| 60 | CDRL2 aa | STS |
| 61 | CDRL2 long aa | YYCLQYYITPYTFGQ |
| 62 | CDRL3 aa | LQYYITPYT |
| 63 | CDRH1 nuc | gggttcaactttagaaagtcttgg |
| 64 | CDRH2 nuc | ataagagaagatggaagtgagagt |
| 65 | CDRH3 nuc | gcgagagatagattctgcaatgatgatgagattcacagacacggacaagaagatctgc |
| | | ccagaccctccatctcggctgccgaaggcaccgtgatccccctggggagccatgtga |
| | | ctttcgtgtgccggggcccggttggggttcaaacattccgcctggagaaggacagtaga |
| | | tccatatacaatgatactgaaaatgtgtctcaacctagtccatctgagtcagaggccaga |
| | | tttcgcattgactcagtgagtgaaggaaatgccggactttatcggtgcgtctattataagg |
| | | cccctaaatggtctgcgcagagtgattacctggagctgctggtgaaaggtcaggaagtc |
| | | acctgggccctgtttacctcctgtggtggtgatggagaggaacccgactacgacatgga |
| | | cgtc |
| 66 | CDRL1 nuc | cagagtgttttatacaggtccaagaataagaactac |
| 67 | CDRL2 nuc | tcgacatct |
| 68 | CDRL2 long nuc | ctcatttactcgacatctactcgggcg |
| 69 | CDRL3 nuc | ctgcaatattatattactccctacact |
| 70 | heavy chain aa | EVQLVESGGGLVQPGGSLRLSCVASGFNFRKSWMGWVRQA |
| | | PGKGLEWVANIREDGSESFYADSVKGRFTVSRDNAKKSLYLHI |
| | | NSLRAEDTAVYYCARDRFCNDDEIHRHGQEDLPRPSISAAEG |
| | | TVIPLGSHVTFVCRGPVGVQTFRLEKDSRSIYNDTENVSQPSPS |
| | | ESEARFRIDSVSEGNAGLYRCVYYKAPKWSAQSDYLELLVKGQ |
| | | EVTWALFTSCGGDGEEPDYDMDVRGKGTTVTVSS |
| 71 | light chain aa | DIVMTQSPDSLAVSLGERATINCKSSQSVLYRSKNKNYLAWF |
| | | QQKPGQPPKVLIYSTSTRASGVPDRFTGSGSGTDFTLTISSLQA |
| | | EDVAVYYCLQYYITPYTFGQGTKLEIK |
| 72 | heavy chain nuc | gaggtgcagctggtggagtctgggggaggcttggtccagccgggggggtccctgaga |
| | | ctctcctgtgtagcctctgggttcaactttagaaagtcttggatgggttgggtccgccagg |
| | | ctccagggaaggggctggagtgggtggcaaacataagagaagatggaagtgagagtt |
| | | tctatgcggactctgtgaagggccgcttcaccgtctccagagacaacgccaagaaatc |
| | | actgtatctccatatcaacagcctgagagccgaggacacggctgtctattactgtgcga |
| | | gagatagattctgcaatgatgatgagattcacagacacggacaagaagatctgcccag |
| | | accctccatctcggctgccgaaggcaccgtgatccccctggggagccatgtgactttc |
| | | gtgtgccggggcccggttggggttcaaacattccgcctggagaaggacagtagatcca |
| | | tatacaatgatactgaaaatgtgtctcaacctagtccatctgagtcagaggccagatttc |
| | | gcattgactcagtgagtgaaggaaatgccggactttatcggtgcgtctattataaggccc |
| | | ctaaatggtctgcgcagagtgattacctggagctgctggtgaaaggtcaggaagtcacc |
| | | tgggccctgtttacctcctgtggtggtgatggagaggaacccgactacgacatggacgt |
| | | ccggggcaaagggaccacggtcaccgtctcctca |
| 73 | light chain nuc | gacatcgtgatgacccagtctccagactccctggctgtgtctctgggcgagagggcca |
| | | ccatcaactgcaagtccagtcagagtgttttatacaggtccaagaataagaactacttag |
| | | cttggttccagcagaaaccaggacagcctcctaaggtgctcatttactcgacatctactc |
| | | gggcgtccggggtccctgaccgattcactggcagcgggtctgggacagatttcactctc |
| | | accatcagcagcctgcaggctgaagatgtggcagtttattactgtctgcaatattatatta |
| | | ctccctacacttttggccaggggaccaagttggagatcaaa |

MGC2 ANTIBODY

| | | |
|---|---|---|
| 74 | CDRH1 aa | GFTFSNFW |
| 75 | CDRH2 aa | IKEDGSEK |
| 76 | CDRH3 aa | VRERFCSNHIHKEEHLPRPSISPEPGTVITLGSHVTFVCRGPVGV |
| | | QTFRLEKDSRSTYNDTEDVSQPSPSESEARFRIDSVSEGYAGLY |
| | | RCLYYKPPKWSEQSDYLELLVKGDDVTWALYPSCGGDGEAS |
| | | DYNMDV |
| 77 | CDRL1 aa | QRFSGW |
| 78 | CDRL2 aa | KAS |
| 79 | CDRL2 long aa | LIYKASPLA |
| 80 | CDRL3 aa | QHYSNYSYT |
| 81 | CDRH1 nuc | ggattcacctttagtaacttttgg |
| 82 | CDRH2 nuc | ataaaggaagatggaagtgagaaa |
| 83 | CDRH3 nuc | gtgagagagagattctgcagtaatcatatccacaaagaagagcatctgcccagaccct |
| | | ccatctcgcctgagccaggcaccgtgatcaccctggggagccatgtgactttcgtgtgc |
| | | cggggcccggttggggttcaaacattccgcctggagaaggacagtagatccacatac |
| | | aatgatactgaagatgtgtctcaacctagtccatctgagtcagaggccagattccgcatt |
| | | gactcagtaagtgaaggatatgccgggctttatcgctgcctctattataagccccctaaa |
| | | tggtctgagcagagtgactacctggagctgctggtgaaaggtgacgacgtcacctggg |
| | | ccctgtacccctcttgtggtggtgatggagaggcttccgactacaacatggacgtc |
| 84 | CDRL1 nuc | cagcgttttagtggctgg |
| 85 | CDRL2 nuc | aaggcgtct |
| 86 | CDRL2 long nuc | ctgatctataaggcgtctcctttagca |
| 87 | CDRL3 nuc | caacactacagtaattattcatatact |
| 88 | heavy chain aa | EVQLVESGGGLVQPGGSLRLSCAASGFTFSNFWMGWVRQT |
| | | PGKGLEWVANIKEDGSEKYYVDSVRGRFTISRDSAKNSLYLQ |
| | | MNSLRAEDTAVYYCVRERFCSNHIHKEEHLPRPSISPEPGTVITL |
| | | GSHVTFVCRGPVGVQTFRLEKDSRSTYNDTEDVSQPSPSESEA |
| | | RFRIDSVSEGYAGLYRCLYYKPPKWSEQSDYLELLVKGDDVT |
| | | WALYPSCGGDGEASDYNMDVWGKGTTVTVSS |
| 89 | light chain aa | DIQMTQSPSTLSASVGDRVTISCRASQRFSGWLAWYQQKPG |
| | | KAPNLLIYKASPLAGGGPSRFSGSGSGTDFTLTISSLQPDDSAT |
| | | YYCQHYSNYSYTFGQGTKLEIR |
| 90 | heavy chain nuc | gaggtgcagctggtggagtctgggggaggcttggtccagcctggggggtccctgagac |
| | | tctcctgtgcagcctctggattcacctttagtaacttttggatgggttgggtccgccagact |
| | | ccagggaaggggctggagtgggtggccaatataaaggaagatggaagtgagaaata |
| | | ctatgtggactctgtgaggggccgattcaccatctccagagacagcgccaagaactca |
| | | ctttatctgcagatgaacagcctgagagccgaggacacggctgtctattattgtgtgaga |
| | | gagagattctgcagtaatcatatccacaaagaagagcatctgcccagaccctccatct |
| | | cgcctgagccaggcaccgtgatcaccctggggagccatgtgactttcgtgtgccgggg |
| | | cccggttggggttcaaacattccgcctggagaaggacagtagatccacatacaatgat |
| | | actgaagatgtgtctcaacctagtccatctgagtcagaggccagattccgcattgactca |
| | | gtaagtgaaggatatgccgggctttatcgctgcctctattataagccccctaaatggtctg |
| | | agcagagtgactacctggagctgctggtgaaaggtgacgacgtcacctgggccctgta |
| | | cccctcttgtggtggtgatggagaggcttccgactacaacatggacgtctggggcaaag |
| | | ggaccacggtcaccgtctcctca |
| 91 | light chain nuc | gacatccagatgacccagtctccttccaccctgtctgcatctgtgggagacagagtcac |
| | | catctcttgccgggccagtcagcgttttagtggctggttggcctggtatcagcagaaacc |
| | | agggaaagcccctaacctcctgatctataaggcgtctcctttagcaggtgggggcccat |
| | | caaggttcagcggcagtggatctgggacagacttcactctcaccatcagcagcctgca |
| | | gcctgatgattctgcaacttattactgccaacactacagtaattattcatatacttttggcca |
| | | ggggaccaagctggagatcaga |

MGC4 ANTIBODY

| | | |
|---|---|---|
| 92 | CDRH1 aa | GFNSRSYW |
| 93 | CDRH2 aa | INQDGTEK |
| 94 | CDRH3 aa | ARDRFCGGESHLHGEEDLPRPSISAEPDTVIPLGSHVTFVCRGP |
| | | VGVHTFRLERGWRYNDTEDVSQAGPSESEARFRIDSVREGNA |
| | | GLYRCIYYIAPKWSEQSDYLELRVKGGDVTWALLTYCGGDGE |
| | | ESDYPMDV |
| 95 | CDRL1 aa | TGPVTSAYY |
| 96 | CDRL2 aa | SIN |
| 97 | CDRL2 long aa | LIYSINKKH |
| 98 | CDRL3 aa | LLSCGGAQPVW |
| 99 | CDRH1 nuc | ggattcaactctcgtagttattgg |
| 100 | CDRH2 nuc | ataaatcaagatgggactgagaaa |
| 101 | CDRH3 nuc | gcgagagacagattctgtggtggtgagagtcacttgcacggagaagaagatctgccca |
| | | gaccctccatctcggctgagccagacaccgtaatccccctggggagccatgtgacttt |
| | | cgtgtgccggggcccggttggggttcacacattccgcctggagagggggtggaggtac |
| | | aacgacactgaagatgtgtctcaagctggtccatctgagtcagaggccagattccgcat |
| | | tgactcggtaagggaaggaaatgccgggctttatcgatgcatctattacatagcccctaa |
| | | atggtctgagcagagtgactacctggagctgcgggtgaaaggtggggacgtcacctgg |
| | | gccctgttaacgtactgtggcggtgatggagaggaatccgactaccccatggacgtc |
| 102 | CDRL1 nuc | actggacctgtcaccagtgcttactat |
| 103 | CDRL2 nuc | agtataaac |
| 104 | CDRL2 long nuc | cttatttatagtataaacaaaaaacac |
| 105 | CDRL3 nuc | ctgctctcctgtggtggtgctcagccttgggtg |
| 106 | heavy chain aa | EVQLVESGGGLVQPGGSLRLSCEGSGFNSRSYWMTWVRQA |
| | | PGKGLEWVASINQDGTEKNYVDSVKGRFTISRDSAKNSLYLQ |
| | | MSSLRADDTAVYYCARDRFCGGESHLHGEEDLPRPSISAEPDT |
| | | VIPLGSHVTFVCRGPVGVHTFRLERGWRYNDTEDVSQAGPSE |
| | | SEARFRIDSVREGNAGLYRCIYYIAPKWSEQSDYLELRVKGGD |
| | | VTWALLTYCGGDGEESDYPMDVWGKGTTVTVSS |
| 107 | light chain aa | QTVVTQEPSLTVSPGGTVTLTCASSTGPVTSAYYPNWFQQKP |
| | | GQAPRSLIYSINKKHSWTPARFSGSLLGGKAALTLSGVQPEDE |
| | | ADYYCLLSCGGAQPWVFGGGTKLTVQ |
| 108 | heavy chain nuc | gaggtgcagctggtggagtctgggggaggcttggtacagcctggggggtccctgagac |
| | | tctcctgtgaaggctctggattcaactctcgtagttattggatgacctgggtccgccaggc |
| | | tccagggaaggggctggagtgggtggccagtataaatcaagatgggactgagaaaaa |
| | | ttatgtggactctgtgaagggccggttcaccatctccagagactccgccaagaactcac |
| | | tgtatctgcaaatgagcagcctgagagccgacgacacggctgtatattactgtgcgaga |
| | | gacagattctgtggtggtgagagtcacttgcacggagaagaagatctgcccagaccct |
| | | ccatctcggctgagccagacaccgtaatccccctggggagccatgtgactttcgtgtgc |
| | | cggggcccggttggggttcacacattccgcctggagagggggtggaggtacaacgac |
| | | actgaagatgtgtctcaagctggtccatctgagtcagaggccagattccgcattgactcg |
| | | gtaagggaaggaaatgccgggctttatcgatgcatctattacatagcccctaaatggtct |
| | | gagcagagtgactacctggagctgcgggtgaaaggtggggacgtcacctgggccctg |
| | | ttaacgtactgtggcggtgatggagaggaatccgactaccccatggacgtctggggca |
| | | aagggaccacggtcaccgtctcctca |
| 109 | light chain nuc | cagactgtggttactcaggagccctcactgactgtgtccccaggagggacagtcactct |
| | | cacctgtgcttccagcactggacctgtcaccagtgcttactatccaaactggttccagca |
| | | gaagcctggacaagcacccaggtctcttatttatagtataaacaaaaaacactcctgga |
| | | cccctgcccggttctcaggctccctccttgggggcaaagctgccctgacactgtcaggt |
| | | gtacagcctgaggacgaggctgactattactgcctgctctcctgtggtggtgctcagcct |
| | | tgggtgttcggcggagggaccaagctgaccgtccaag |

MGC5 ANTIBODY

| | | |
|---|---|---|
| 110 | CDRH1 aa | GFNSRSYW |
| 111 | CDRH2 aa | INQDGTEK |
| 112 | CDRH3 aa | ARDRFCGGESHLHGEEDLPRPSISAEPGTVIPLGSHVTFVCRGP |
| | | VGVHTFRLERGWRYNDTEDVSQAGPSQSEARFRIDSVREGN |
| | | AGLYRCLYYIPPKWSEQSDYLELRVKGGDVTWALLTYCGGD |
| | | GEESDYPMDV |
| 113 | CDRL1 aa | TGPVTSAYY |
| 114 | CDRL2 aa | NIN |
| 115 | CDRL2 long aa | LIYNINKH |
| 116 | CDRL3 aa | LLSCGGAQPWV |
| 117 | CDRH1 nuc | ggattcaactctcgtagttattgg |
| 118 | CDRH2 nuc | ataaatcaagatggaactgagaaa |
| 119 | CDRH3 nuc | gcgagagacagattctgtggtggtgagagtcacttgcacggagaagaagatctgccca |
| | | gaccctccatctcggctgagccaggcaccgtgatccccctggggagccatgtgacttt |
| | | cgtgtgccggggccccgttggggttcacacattccgcctggagagggggtggagatac |
| | | aacgacactgaagatgtgtctcaagctggtccatctcagtcagaggccagattccgcat |
| | | tgactcggtaagggaaggaaatgccgggctttatcgatgcctctattacataccccctaa |
| | | atggtctgagcagagtgactacctggaactgcgggtgaaaggtggggacgtcacctgg |
| | | gccctgttaacgtactgtggtggtgatggagaggaatccgactaccccatggacgtc |
| 120 | CDRL1 nuc | actggacctgtcaccagtgcttactat |
| 121 | CDRL2 nuc | aatataaac |
| 122 | CDRL2 long nuc | cttatttataatataaacaaaaaacac |
| 123 | CDRL3 nuc | ctgctctcctgtggtggtgctcagccttgggtg |
| 124 | heavy chain aa | EVQLVESGGGLVQPGGSLRLSCEASGFNSRSYWMTWVRQAP |
| | | GKGLEWVATINQDGTEKNYVDSVRGRFTISRDTAKNSLFLQ |
| | | MNSLRAEDTAVYYCARDRFCGGESHLHGEEDLPRPSISAEPGT |
| | | VIPLGSHVTFVCRGPVGVHTFRLERGWRYNDTEDVSQAGPS |
| | | QSEARFRIDSVREGNAGLYRCLYYIPPKWSEQSDYLELRVKGG |
| | | DVTWALLTYCGGDGEESDYPMDVWGKGTTVTVSS |
| 125 | light chain aa | QTVVTQEPSLTVSPGGTVTLTCASNTGPVTSAYYPNWFQQKP |
| | | GQAPRSLIYNINKKHSWTPARFSGSLLGGKAALTLSGVQPEDE |
| | | ADYYCLLSCGGAQPWVFGGGTKLTVQ |
| 126 | heavy chain nuc | gaggtgcagctggtggagtctgggggaggcttggtccagcctggggggtcactgagac |
| | | tctcctgtgaagcctctggattcaactctcgtagttattggatgacctgggtccgccaggc |
| | | tccagggaaggggctggagtgggtggccactataaatcaagatggaactgagaaaaa |
| | | ttatgtggactctgtgaggggccggttcaccatctccagagacaccgccaagaactca |
| | | ctgtttctgcaaatgaacagcctgagagccgaggacacggctgtatattactgcgcgag |
| | | agacagattctgtggtggtgagagtcacttgcacggagaagaagatctgcccagaccc |
| | | tccatctcggctgagccaggcaccgtgatccccctggggagccatgtgactttcgtgtg |
| | | ccggggccccgttggggttcacacattccgcctggagagggggtggagatacaacga |
| | | cactgaagatgtgtctcaagctggtccatctcagtcagaggccagattccgcattgactc |
| | | ggtaagggaaggaaatgccgggctttatcgatgcctctattacataccccctaaatggtc |
| | | tgagcagagtgactacctggaactgcgggtgaaaggtggggacgtcacctgggccct |
| | | gttaacgtactgtggtggtgatggagaggaatccgactaccccatggacgtctggggca |
| | | aagggaccacggtcaccgtctcctca |
| 127 | light chain nuc | cagactgtggtgactcaggagccctcactgactgtgtccccaggagggacagtcactc |
| | | tcacctgtgcttccaacactggacctgtcaccagtgcttactatccaaactggttccagc |
| | | agaagcctggacaagcacccaggtctcttatttataatataaacaaaaaacactcctgg |
| | | acccctgcccggttctcaggctccctccttgggggcaaagctgccctgacactgtcag |
| | | gtgtacagcctgaggacgaggctgactattactgcctgctctcctgtggtggtgctcagc |
| | | cttgggtgttcggcggagggaccaagctgaccgtccaa |

MGC7 ANTIBODY

| | | |
|---|---|---|
| 128 | CDRH1 aa | GFTFRNYW |
| 129 | CDRH2 aa | IRQDGSEK |
| 130 | CDRH3 aa | VRDKFCSDENHMHVADDLPRPSISPEPGTVIPLGSHVTFVCRG |
| | | PVGVQTFRLEKDRRSTYNDTEDVSQPSPSESEARFRIDSVTEGN |
| | | AGLYRCVYYKPPKWSDQSDFLELLVKGEDVTWALFPHCGAD |
| | | GEDSDYYMDV |
| 131 | CDRL1 aa | QGLSTW |
| 132 | CDRL2 aa | AAS |
| 133 | CDRL2 long aa | LIYAASSLQ |
| 134 | CDRL3 aa | QQANSFPLT |
| 135 | CDRH1 nuc | ggattcaccttcagaaattattgg |
| 136 | CDRH2 nuc | ataaggcaagatggaagtgagaag |
| 137 | CDRH3 nuc | gtgagagataaattctgcagtgatgagaatcacatgcacgtagcagatgatctgcccag |
| | | accctctatctcgcctgagccaggcaccgtgatccccctggggagccatgtgactttcg |
| | | tgtgtcggggcccggttggggttcaaacattccgcctggagaaggacagaagatccac |
| | | atacaatgatactgaagatgtgtctcaacctagtccatctgagtcagaggccagattccg |
| | | cattgactcagtaactgaaggaaatgccgggctttatcgctgcgtctattataagccccc |
| | | taaatggtctgaccagagtgacttcctggagttgctggtgaagggtgaggacgtcacctg |
| | | ggccctgttcccccattgtggtgctgatggagaggactccgactactacatggacgtc |
| 138 | CDRL1 nuc | cagggtcttagtacctgg |
| 139 | CDRL2 nuc | gctgcatcc |
| 140 | CDRL2 long nuc | tattattgtcaacaggctaacagtttccctctcactttcggcgga |
| 141 | CDRL3 nuc | caacaggctaacagtttccctctcact |
| 142 | heavy chain aa | EVQLVESGGDLVQPCGSLRLSCAASGFTFRNYWMSWVRQTP |
| | | GKGLEWVANIRQDGSEKYYVDSVKGRFTISRDNAKNLLYLQ |
| | | MNSLRAEDTAVYYCVRDKFCSDENHMHVADDLPRPSISPEPG |
| | | TVIPLGSHVTFVCRGPVGVQTFRLEKDRRSTYNDTEDVSQPSP |
| | | SESEARFRIDSVTEGNAGLYRCVYYKPPKWSDQSDFLELLVKG |
| | | EDVTWALFPHCGADGEDSDYYMDVWGKGTTVTVSS |
| 143 | light chain aa | DIQMTQSPSSVSASVGDRVTITCRASQGLSTWLAWYQQKPG |
| | | KAPKILIYAASSLQSGVPSRFSGSGSGTDFTLTISSLQPEDFATYY |
| | | CQQANSFPLTFGGGTKVEIK |
| 144 | heavy chain nuc | gaggtgcagctggtggagtctgggggagacttggtccagcctggggggtccctgagac |
| | | tctcctgtgcagcctctggattcaccttcagaaattattggatgagttgggtccgccagac |
| | | tccagggaagggactggagtgggtggccaacataaggcaagatggaagtgagaagt |
| | | attatgtggactctgtgaagggccgattcaccatctccagagacaacgccaagaactta |
| | | ttatatctacaaatgaacagcctgagagccgaggacacggctgtgtattactgtgtgaga |
| | | gataaattctgcagtgatgagaatcacatgcacgtagcagatgatctgcccagaccctc |
| | | tatctcgcctgagccaggcaccgtgatccccctggggagccatgtgactttcgtgtgtcg |
| | | gggcccggttggggttcaaacattccgcctggagaaggacagaagatccacatacaa |
| | | tgatactgaagatgtgtctcaacctagtccatctgagtcagaggccagattccgcattga |
| | | ctcagtaactgaaggaaatgccgggctttatcgctgcgtctattataagccccctaaatg |
| | | gtctgaccagagtgacttcctggagttgctggtgaagggtgaggacgtcacctgggccc |
| | | tgttcccccattgtggtgctgatggagaggactccgactactacatggacgtctggggca |
| | | aagggaccacggtcaccgtctcctca |
| 145 | light chain nuc | gacatccagatgacccagtctccatcttccgtgtctgcatctgtaggagacagagtcac |
| | | catcacttgtcgggcgagtcagggtcttagtacctggttagcctggtatcagcagaaacc |
| | | agggaaagcccctaagatcctgatctatgctgcatccagtttgcaaagtggggtcccat |
| | | caaggttcagcggcagtggatctgggacagatttcactctcaccatcagcagcctgca |
| | | gcctgaagattttgcaacttattattgtcaacaggctaacagtttccctctcactttcggcg |
| | | gagggaccaaggtggagatcaaa |

MGC17 ANTIBODY

| | | |
|---|---|---|
| 146 | CDRH1 aa | GFNFRKSW |
| 147 | CDRH2 aa | IREDGSKA |
| 148 | CDRH3 aa | ARDRFCSDDEDHSHGAEDLPRPSISAEEGTVIPLGSRLTFVCRG |
| | | PVGVHTFRLERDRRSTYNDTEDVSHPSPSESEARFRIDSVSEGN |
| | | AGLYRCVYYKSPEWSKQSDYLELLVKGQEVTWALFTSCGGD |
| | | GEVPDYDMDV |
| 149 | CDRL1 aa | QSVLYRSKNKKY |
| 150 | CDRL2 aa | WTS |
| 151 | CDRL2 long aa | LIYWTSTRA |
| 152 | CDRL3 aa | QQYFIFPYT |
| 153 | CDRH1 nuc | gggttcaattttagaaagtcttgg |
| 154 | CDRH2 nuc | ataagagaagatggaagtaaggca |
| 155 | CDRH3 nuc | gcgagagatagattctgcagtgatgatgaggatcacagccacggagcagaagatctg |
| | | cccagaccctccatctcggctgaggaaggcaccgtgattcccctggggagccgtctg |
| | | actttcgtgtgccggggcccggttggggttcacacattccgcctggagagggaccgtag |
| | | atccacatacaatgatactgaagatgtgtctcaccctagtccatctgagtctgaggccag |
| | | atttcgcattgactcagtgagtgaaggaaatgccgggctttatcgctgcgtctattataagt |
| | | cccctgaatggtctaagcagagtgattacctggagctgctggtgaaaggtcaggaagtc |
| | | acctgggccctgtttacttcttgtggtggtgatggagaggtacccgactacgacatggac |
| | | gtc |
| 156 | CDRL1 nuc | cagagtgttttatacaggtccaagaataagaaatat |
| 157 | CDRL2 nuc | tggacatct |
| 158 | CDRL2 long nuc | ctcatttactggacatctactcgggcg |
| 159 | CDRL3 nuc | cagcagtattttatttttccgtacact |
| 160 | heavy chain aa | EVQLVESGGGLVQPGGSLKLSCVASGFNFRKSWMSWVRQA |
| | | PGKGLEWVANIREDGSKAYYVDSVKGRFTVSRDNAKNSLYL |
| | | QINSLRADDTAVYYCARDRFCSDDEDHSHGAEDLPRPSISAEE |
| | | GTVIPLGSRLTFVCRGPVGVHTFRLERDRRSTYNDTEDVSHPS |
| | | PSESEARFRIDSVSEGNAGLYRCVYYKSPEVVSKQSDYLELLVKG |
| | | QEVTWALFTSCGGDGEVPDYDMDVRGKGTTVTVSS |
| 161 | light chain aa | DIVMTQSPDSLAVSLGERATINCKSSQSVLYRSKNKKYLAWFQ |
| | | QRPGQPPKVLIYWTSTRASGVPDRFSGSGSGTDFTLTISSLQA |
| | | DDVAVYYCQQYFIFPYTFGQGTKLEIR |
| 162 | heavy chain nuc | gaggtgcagctggtggagtcggggggaggcttggtccagcctggggggtccctgaaa |
| | | ctgtcctgtgtagcctctgggttcaattttagaaagtcttggatgagttgggtccgccaggc |
| | | tccagggaaggggctggagtgggtggcaaacataagagaagatggaagtaaggcat |
| | | actatgtggactctgtcaagggccgattcaccgtctccagagacaacgccaagaactc |
| | | gctgtatctgcagatcaacagcctgagagccgacgacacggctgtctattactgtgcga |
| | | gagatagattctgcagtgatgatgaggatcacagccacggagcagaagatctgccca |
| | | gaccctccatctcggctgaggaaggcaccgtgattcccctggggagccgtctgactttc |
| | | gtgtgccggggcccggttggggttcacacattccgcctggagagggaccgtagatcca |
| | | catacaatgatactgaagatgtgtctcaccctagtccatctgagtctgaggccagatttc |
| | | gcattgactcagtgagtgaaggaaatgccgggctttatcgctgcgtctattataagtccc |
| | | ctgaatggtctaagcagagtgattacctggagctgctggtgaaaggtcaggaagtcacc |
| | | tgggccctgtttacttcttgtggtggtgatggagaggtacccgactacgacatggacgtc |
| | | cggggcaaagggaccacggtcaccgtctcttca |
| 163 | light chain nuc | gacatcgtgatgacccaatctcctgactccctggctgtgtctctgggcgagagggccac |
| | | catcaactgcaagtccagccagagtgttttatacaggtccaagaataagaaatatttagc |
| | | ttggttccagcagagaccaggacagcctcctaaggttctcatttactggacatctactcg |
| | | ggcgtccggggtccctgaccgattcagtggcagcgggtctgggacagatttcactctca |
| | | ccatcagcagcctgcaggctgatgatgtggcagtttattattgtcagcagtattttatttttcc |
| | | gtacacttttggccaggggaccaagttggagatcaga |

MGC26 ANTIBODY

| | | |
|---|---|---|
| 164 | CDRH1 aa | GFTFSTYW |
| 165 | CDRH2 aa | IKQDGTER |
| 166 | CDRH3 aa | VRDRFCRDHMHIEEDLPRPSISPEPATVIPLGSHVTIVCRGPVG |
| | | VETFRLQKESRSLYNDTEDVSQPSPSESEARFRIDSVSEGHGGL |
| | | YRCLYYKSSKWSEQSDYLEMLVKGEDVTWALFPYCGGDGEES |
| | | DYYMDV |
| 167 | CDRL1 aa | QRLSRS |
| 168 | CDRL2 aa | KAS |
| 169 | CDRL2 long aa | LIYKASPLE |
| 170 | CDRL3 aa | QQYSNYSYS |
| 171 | CDRH1 nuc | ggattcacctttagtacttattgg |
| 172 | CDRH2 nuc | ataaagcaagatggaactgagaga |
| 173 | CDRH3 nuc | gtgagagacagattctgcagagatcacatgcacatagaagaagatctgcccagaccc |
| | | tccatctcgccggagccagccaccgtgatccccctggggagccatgtgactatcgtgt |
| | | gccggggcccggttggggttgaaacattccgcctgcagaaggagagtagatccctgta |
| | | caatgacactgaagatgtgtctcaacctagtccatctgagtcagaggccagattccgca |
| | | ttgactcagtaagtgaagggcatggcgggctttatcgctgcctctattataagtcttctaaa |
| | | tggtctgagcagagtgactacctggagatgctggtgaaaggtgaggacgtcacctggg |
| | | ccctgttcccctattgtggtggtgatggagaggaatccgactactacatggacgtc |
| 174 | CDRL1 nuc | cagcgtcttagtcgctcg |
| 175 | CDRL2 nuc | aaggcgtct |
| 176 | CDRL2 long nuc | ctgatctataaggcgtctcctttagaa |
| 177 | CDRL3 nuc | caacaatacagtaattattcatatagt |
| 178 | heavy chain aa | EVQLVDSGGGLVQPGGSLRLSCAASGFTFSTYWMTWVRQTP |
| | | GKGLEWVASIKQDGTERYYVDSVKGRFIISRDNAKNSLYLQM |
| | | HSLRAEDTAVYYCVRDRFCRDHMHIEEDLPRPSISPEPATVIPL |
| | | GSHVTIVCRGPVGVETFRLQKESRSLYNDTEDVSQPSPSESEAR |
| | | FRIDSVSEGHGGLYRCLYYKSSKWSEQSDYLEMLVKGEDVTW |
| | | ALFPYCGGDGEESDYYMDVWGKGTTVTVSS |
| 179 | light chain aa | DIQLTQSPSTLSASVGDRVTISCRASQRLSRSLAWYQQRPRKA |
| | | PNLLIYKASPLEIGGPSRFTGSGSGTEFTLTISSLQPDDSATYYC |
| | | QQYSNYSYSFGQGTKLEIR |
| 180 | heavy chain nuc | gaggtgcagctggtggattctgggggaggcttggtccagcctggggggtccctgagact |
| | | ctcctgtgcagcctctggattcacctttagtacttattggatgacctgggtccgccagact |
| | | ccagggaaggggctggagtgggtggccagcataaagcaagatggaactgagagata |
| | | ctatgtggactctgtgaagggccgattcattatctccagagacaacgccaagaactcac |
| | | tatatttgcaaatgcacagcctgagagccgaggacacggctgtgtattattgtgtgagag |
| | | acagattctgcagagatcacatgcacatagaagaagatctgcccagaccctccatctc |
| | | gccggagccagccaccgtgatccccctggggagccatgtgactatcgtgtgccgggg |
| | | cccggttggggttgaaacattccgcctgcagaaggagagtagatccctgtacaatgac |
| | | actgaagatgtgtctcaacctagtccatctgagtcagaggccagattccgcattgactca |
| | | gtaagtgaagggcatggcgggctttatcgctgcctctattataagtcttctaaatggtctga |
| | | gcagagtgactacctggagatgctggtgaaaggtgaggacgtcacctgggccctgttc |
| | | ccctattgtggtggtgatggagaggaatccgactactacatggacgtctggggcaaagg |
| | | gaccacggtcaccgtctcctca |
| 181 | light chain nuc | gacatccagctgacccagtctccttccaccctgtctgcatctgtaggagacagagtcac |
| | | catctcttgccgggccagtcagcgtcttagtcgctcgttggcctggtatcagcagagacc |
| | | acggaaagcccctaacctcctgatctataaggcgtctcctttagaaattgggggcccat |
| | | caaggttcaccggcagtggatctgggacagaattcactctcaccatcagcagcctgca |
| | | gcctgatgattctgcaacttattactgccaacaatacagtaattattcatatagttttggcca |
| | | ggggaccaagctggagatcaga |

MGC28 ANTIBODY

| | | |
|---|---|---|
| 182 | CDRH1 aa | GFTFSSYW |
| 183 | CDRH2 aa | INQDGSER |
| 184 | CDRH3 aa | ARQRFCSDGSLFHGEDLPRPTISAETGTVISLGSHVTFVCRGPL |
| | | GVQTFRLERESRSRYSETEDVSQVGPSESEARFRIDSVSEGNAG |
| | | LYRCIYYKPPKWSEQSDYLELRVKGEDVTWALLTYCGGDRDE |
| | | SDYYMDV |
| 185 | CDRL1 aa | TGSVTSGSF |
| 186 | CDRL2 aa | STT |
| 187 | CDRL2 long aa | LIYSTTKKH |
| 188 | CDRL3 aa | LLYCGGGQPWV |
| 189 | CDRH1 nuc | ggattcacgtttagttcttattgg |
| 190 | CDRH2 nuc | ataaaccaagatggaagtgagaga |
| 191 | CDRH3 nuc | gcgagacaaagattctgcagtgatgggagtctctttcacggagaagatctgcccagac |
| | | ccaccatctcggctgagacaggcaccgtgatctccctggggagccatgtgactttcgtg |
| | | tgccggggcccacttggggtgcaaacattccgcctggagagggagagtaggtccaga |
| | | tacagtgaaactgaagatgtgtctcaagttggtccatctgagtcagaggccagattccgc |
| | | attgactcagtgagtgaaggaaatgccgggctttatcgatgcatctattacaaaccccct |
| | | aaatggtctgagcagagtgactacctggagctgcgggtgaaaggtgaggacgtcacct |
| | | gggccctgttaacctattgtggtggtgatagagacgaatccgactactacatggacgtc |
| 192 | CDRL1 nuc | actggatcagtcaccagtggttccttt |
| 193 | CDRL2 nuc | agtacaacc |
| 194 | CDRL2 long nuc | ctgatttatagtacaaccaaaaaacac |
| 195 | CDRL3 nuc | ctactctactgtggtggtggtcaaccttgggtg |
| 196 | heavy chain aa | EVQLVESGGGLVQPGGSLRLSCEASGFTFSSYWMTWVRQAP |
| | | GKGLEWVANINQDGSERYYVDSVKGRFTISRDTVKNSLYLQ |
| | | MNNLRAEDTAVYYCARQRFCSDGSLFHGEDLPRPTISAETGT |
| | | VISLGSHVTFVCRGPLGVQTFRLERESRSRYSETEDVSQVGPSE |
| | | SEARFRIDSVSEGNAGLYRCIYYKPPKWSEQSDYLELRVKGEDV |
| | | TWALLTYCGGDRDESDYYMDVWGKGTTVTVSS |
| 197 | light chain aa | QTVVTQEPSLTVSPGGTVTLTCASSTGSVTSGSFPNWFQQTP |
| | | GQAPRSLIYSTTKKHSWTPARFSGSLLGGKAALTVSDTQPEDE |
| | | AEYYCLLYCGGGQPWVFGGGTKLTVL |
| 198 | heavy chain nuc | gaggtgcagctggtggagtctgggggaggcttggtccagccgggggggtccctgaga |
| | | ctctcctgtgaagcctctggattcacgtttagttcttattggatgacctgggtccgccaggc |
| | | tccagggaaggggctggagtgggtggccaatataaaccaagatggaagtgagagata |
| | | ttatgtggactctgtgaagggccggttcaccatctccagagacaccgtcaagaactcac |
| | | tgtatttgcaaatgaacaacctgagagccgaggacacggctgtatattactgcgcgaga |
| | | caaagattctgcagtgatgggagtctctttcacggagaagatctgcccagacccaccat |
| | | ctcggctgagacaggcaccgtgatctccctggggagccatgtgactttcgtgtgccggg |
| | | gcccacttggggtgcaaacattccgcctggagagggagagtaggtccagatacagtg |
| | | aaactgaagatgtgtctcaagttggtccatctgagtcagaggccagattccgcattgact |
| | | cagtgagtgaaggaaatgccgggctttatcgatgcatctattacaaaccccctaaatggt |
| | | ctgagcagagtgactacctggagctgcgggtgaaaggtgaggacgtcacctgggccc |
| | | tgttaacctattgtggtggtgatagagacgaatccgactactacatggacgtctggggca |
| | | aagggaccacggtcaccgtctcctca |
| 199 | light chain nuc | cagactgtggtgactcaggagccctcactgactgtgtccccaggagggacagtcactc |
| | | tcacctgtgcttccagtactggatcagtcaccagtggttcctttccaaactggttccagca |
| | | gacacctggacaagcacccaggtcactgatttatagtacaaccaaaaaacactcttgg |
| | | acccctgcccggttctcaggctctctccttgggggcaaagctgccctgacagtgtcaga |
| | | tacacagccggaggacgaggctgagtattactgcctactctactgtggtggtggtcaac |
| | | cttgggtgttcggcggagggaccaagctgaccgtccta |

MGC29 ANTIBODY

| | | |
|---|---|---|
| 200 | CDRH1 aa | GFNSRSYW |
| 201 | CDRH2 aa | INQDGTEK |
| 202 | CDRH3 aa | ARDRFCGGESHLHGEEDLPRPSISAEPGTVIPLGSHVTFVCRGP |
| | | VGVHTFRLERGWRYNDTEDVSQAGPSQSEARFRIDSVREGN |
| | | AGLYRCLYYIPPKWSEQSDYLELRVKGGDVTWALLTYCGGD |
| | | GEESDYPMDV |
| 203 | CDRL1 aa | TGPVTSAYY |
| 204 | CDRL2 aa | NIN |
| 205 | CDRL2 long aa | LIYNINKKH |
| 206 | CDRL3 aa | LLSCGGAQPWV |
| 207 | CDRH1 nuc | ggattcaactctcgtagttattgg |
| 208 | CDRH2 nuc | ataaatcaagatggaactgagaaa |
| 209 | CDRH3 nuc | gcgagagacagattctgtggtggtgagagtcacttgcacggagaagaagatctgccca |
| | | gaccctccatctcggctgagccaggcaccgtgatccccctggggagccatgtgacttt |
| | | cgtgtgccggggccccgttggggttcacacattccgcctggagagggggtggagatac |
| | | aacgacactgaagatgtgtctcaagctggtccatctcagtcagaggccagattccgcat |
| | | tgactcggtaagggaaggaaatgccgggctttatcgatgcctctattacataccccctaa |
| | | atggtctgagcagagtgactacctggaactgcgggtgaaaggtggggacgtcacctgg |
| | | gccctgttaacgtactgtggtggtgatggagaggaatccgactaccccatggacgtc |
| 210 | CDRL1 nuc | actggacctgtcaccagtgcttactat |
| 211 | CDRL2 nuc | aatataaac |
| 212 | CDRL2 long nuc | cttatttataatataaacaaaaaacac |
| 213 | CDRL3 nuc | ctgctctcctgtggtggtgctcagccttgggtg |
| 214 | heavy chain aa | EVQLVESGGGLVQPGGSLRLSCEASGFNSRSYWMTWVRQAP |
| | | GKGLEWVATINQDGTEKNYVDSVRGRFTISRDTAKNSLFLQ |
| | | MNSLRAEDTAVYYCARDRFCGGESHLHGEEDLPRPSISAEPGT |
| | | VIPLGSHVTFVCRGPVGVHTFRLERGWRYNDTEDVSQAGPS |
| | | QSEARFRIDSVREGNAGLYRCLYYIPPKWSEQSDYLELRVKGG |
| | | DVTWALLTYCGGDGEESDYPMDVWGKGTTVTVSS |
| 215 | light chain aa | QTVVTQEPSLTVSPGGTVTLTCASNTGPVTSAYYPNWFQQKP |
| | | GQAPRSLIYNINKKHSWTPARFSGSLLGGKAALTLSGVQPEDE |
| | | ADYYCLLSCGGAQPWVFGGGTKLTVQ |
| 216 | heavy chain nuc | gaggtgcagctggtggagtctgggggaggcttggtccagcctggggggtccctgagac |
| | | tctcctgtgaagcctctggattcaactctcgtagttattggatgacctgggtccgccaggc |
| | | tccagggaaggggctggagtgggtggccactataaatcaagatggaactgagaaaaa |
| | | ttatgtggactctgtgaggggccggttcaccatctccagagacaccgccaagaactca |
| | | ctgtttctgcaaatgaacagcctgagagccgaggacacggctgtatattactgcgcgag |
| | | agacagattctgtggtggtgagagtcacttgcacggagaagaagatctgcccagaccc |
| | | tccatctcggctgagccaggcaccgtgatccccctggggagccatgtgactttcgtgtg |
| | | ccggggccccgttggggttcacacattccgcctggagagggggtggagatacaacga |
| | | cactgaagatgtgtctcaagctggtccatctcagtcagaggccagattccgcattgactc |
| | | ggtaagggaaggaaatgccgggctttatcgatgcctctattacataccccctaaatggtc |
| | | tgagcagagtgactacctggaactgcgggtgaaaggtggggacgtcacctgggccct |
| | | gttaacgtactgtggtggtgatggagaggaatccgactaccccatggacgtctggggca |
| | | aagggaccacggtcaccgtctcctca |
| 217 | light chain nuc | cagactgtggtgactcaggagccctcactgactgtgtccccaggagggacagtcactc |
| | | tcacctgtgcttccaacactggacctgtcaccagtgcttactatccaaactggttccagc |
| | | agaagcctggacaagcacccaggtctcttatttataatataaacaaaaaacactcctgg |
| | | acccctgcccggttctcaggctccctccttgggggcaaagctgccctgacactgtcag |
| | | gtgtacagcctgaggacgaggctgactattactgcctgctctcctgtggtggtgctcagc |
| | | cttgggtgttcggcggagggaccaagctgaccgtccaa |

MGC32 ANTIBODY

| | | |
|---|---|---|
| 218 | CDRH1 aa | GFNFRKSW |
| 219 | CDRH2 aa | IREDGSES |
| 220 | CDRH3 aa | ARDRFCNDDEIHRHGQEDLPRPSISAAEGTVIPLGSHVTFVCR |
| | | GPVGVQTFRLEKDSRSIYNDTENVSQPSPSESEARFRIDSVSEG |
| | | NAGLYRCVYYKAPKWSAQSDYLELLVKGQEVTWALFTSCGG |
| | | DGEEPDYDMDV |
| 221 | CDRL1 aa | QSVLYRSKNKNY |
| 222 | CDRL2 aa | STS |
| 223 | CDRL2 long aa | LIYSTSTRA |
| 224 | CDRL3 aa | LQYYITPYT |
| 225 | CDRH1 nuc | gggttcaactttagaaagtcttgg |
| 226 | CDRH2 nuc | ataagagaagatggaagtgagagt |
| 227 | CDRH3 nuc | gcgagagatagattctgcaatgatgatgagattcacagacacggacaagaagatctgc |
| | | ccagaccctccatctcggctgccgaaggcaccgtgatccccctggggagccatgtga |
| | | ctttcgtgtgccggggcccggttggggttcaaacattccgcctggagaaggacagtaga |
| | | tccatatacaatgatactgaaaatgtgtctcaacctagtccatctgagtcagaggccaga |
| | | tttcgcattgactcagtgagtgaaggaaatgccggactttatcggtgcgtctattataagg |
| | | cccctaaatggtctgcgcagagtgattacctggagctgctggtgaaaggtcaggaagtc |
| | | acctgggccctgtttacctcctgtggtggtgatggagaggaacccgactacgacatgga |
| | | cgtc |
| 228 | CDRL1 nuc | cagagtgttttatacaggtccaagaataagaactac |
| 229 | CDRL2 nuc | tcgacatct |
| 230 | CDRL2 long nuc | ctcatttactcgacatctactcgggcg |
| 231 | CDRL3 nuc | ctgcaatattatattactccctacact |
| 232 | heavy chain aa | EVQLVESGGGLVQPGGSLRLSCVASGFNFRKSWMGWVRQA |
| | | PGKGLEWVANIREDGSESFYADSVKGRFTVSRDNAKKSLYLHI |
| | | NSLRAEDTAVYYCARDRFCNDDEIHRHGQEDLPRPSISAAEG |
| | | TVIPLGSHVTFVCRGPVGVQTFRLEKDSRSIYNDTENVSQPSPS |
| | | ESEARFRIDSVSEGNAGLYRCVYYKAPKWSAQSDYLELLVKGQ |
| | | EVTWALFTSCGGDGEEPDYDMDVRGKGTTVTVSS |
| 233 | light chain aa | DILMTQSPDSLAVSLGERATINCKSSQSVLYRSKNKNYLAWFQ |
| | | QKPGQPPKVLIYSTSTRASGVPDRFTGSGSGTDFTLTISSLQAE |
| | | DVAVYYCLQYYITPYTFGQGTKLEIK |
| 234 | heavy chain nuc | gaggtgcagctggtggagtctgggggaggcttggtccagccgggggggtccctgaga |
| | | ctctcctgtgtagcctctgggttcaactttagaaagtcttggatgggttgggtccgccagg |
| | | ctccagggaaggggctggagtgggtggcaaacataagagaagatggaagtgagagtt |
| | | tctatgcggactctgtgaagggccgcttcaccgtctccagagacaacgccaagaaatc |
| | | actgtatctccatatcaacagcctgagagccgaggacacggctgtctattactgtgcga |
| | | gagatagattctgcaatgatgatgagattcacagacacggacaagaagatctgcccag |
| | | accctccatctcggctgccgaaggcaccgtgatccccctggggagccatgtgactttc |
| | | gtgtgccggggcccggttggggttcaaacattccgcctggagaaggacagtagatcca |
| | | tatacaatgatactgaaaatgtgtctcaacctagtccatctgagtcagaggccagatttc |
| | | gcattgactcagtgagtgaaggaaatgccggactttatcggtgcgtctattataaggccc |
| | | ctaaatggtctgcgcagagtgattacctggagctgctggtgaaaggtcaggaagtcacc |
| | | tgggccctgtttacctcctgtggtggtgatggagaggaacccgactacgacatggacgt |
| | | ccggggcaaagggaccacggtcaccgtctcctca |
| 235 | light chain nuc | gacatcctcatgacccagtctccagactccctggctgtgtctctgggcgagagggcca |
| | | ccatcaactgcaagtccagtcagagtgttttatacaggtccaagaataagaactacttag |
| | | cttggttccagcagaaaccaggacagcctcctaaggtgctcatttactcgacatctactc |
| | | gggcgtccggggtccctgaccgattcactggcagcgggtctgggacagatttcactctc |
| | | accatcagcagcctgcaggctgaagatgtggcagtttattactgtctgcaatattatatta |
| | | ctccctacacttttggccaggggaccaagttggagatcaaa |

MGC33 ANTIBODY

| | | |
|---|---|---|
| 236 | CDRH1 aa | GFTFSSYW |
| 237 | CDRH2 aa | INQDGSER |
| 238 | CDRH3 aa | ARQRFCSDGSLFHGEDLPRPTISAETGTVISLGSHVTFVCRGPL |
| | | GVQTFRLERESRSRYSETEDVSQVGPSESEARFRIDSVSEGNAG |
| | | LYRCIYYKPPKWSEQSDYLELRVKGEDVTWALLTYCGGDRDE |
| | | SDYYMDV |
| 239 | CDRL1 aa | TGSVTSGSF |
| 240 | CDRL2 aa | STT |
| 241 | CDRL2 long aa | LIYSTTKKH |
| 242 | CDRL3 aa | LLYCGGGQPWV |
| 243 | CDRH1 nuc | ggattcacgtttagttcttattgg |
| 244 | CDRH2 nuc | ataaaccaagatggaagtgagaga |
| 245 | CDRH3 nuc | gcgagacaaagattctgcagtgatgggagtctctttcacggagaagatctgcccagac |
| | | ccaccatctcggctgagacaggcaccgtgatctccctggggagccatgtgactttcgtg |
| | | tgccggggcccacttggggtgcaaacattccgcctggagagggagagtaggtccaga |
| | | tacagtgaaactgaagatgtgtctcaagttggtccatctgagtcagaggccagattccgc |
| | | attgactcagtgagtgaaggaaatgccgggctttatcgatgcatctattacaaaccccct |
| | | aaatggtctgagcagagtgactacctggagctgcgggtgaaaggtgaggacgtcacct |
| | | gggccctgttaacctattgtggtggtgatagagacgaatccgactactacatggacgtc |
| 246 | CDRL1 nuc | actggatcagtcaccagtggttccttt |
| 247 | CDRL2 nuc | agtacaacc |
| 248 | CDRL2 long nuc | ctgatttatagtacaaccaaaaaacac |
| 249 | CDRL3 nuc | ctactctactgtggtggtggtcaaccttgggtg |
| 250 | heavy chain aa | EVHLVESGCGLVQPGGSLRLSCEASGFTFSSYWMTWVRQAP |
| | | GKGLEWVANINQDGSERYYVDSVKGRFTISRDTVKNSLYLQ |
| | | MNNLRAEDTAVYYCARQRFCSDGSLFHGEDLPRPTISAETGT |
| | | VISLGSHVTFVCRGPLGVQTFRLERESRSRYSETEDVSQVGPSE |
| | | SEARFRIDSVSEGNAGLYRCIYYKPPKWSEQSDYLELRVKGEDV |
| | | TWALLTYCGGDRDESDYYMDVWGKGTTVTVSS |
| 251 | light chain aa | QTVVTQEPSLTVSPGGTVTLTCASSTGSVTSGSFPNWFQQTP |
| | | GQAPRSLIYSTTKKHSWTPARFSGSLLGGKAALTVSDTQPEDE |
| | | AEYYCLLCGGGQPWVFGGGTKLTVQ |
| 252 | heavy chain nuc | gaggtgcacctggtggagtctgggggaggcttggtccagccgggggggtccctgaga |
| | | ctctcctgtgaagcctctggattcacgtttagttcttattggatgacctgggtccgccaggc |
| | | tccagggaaggggctggagtgggtggccaatataaaccaagatggaagtgagagata |
| | | ttatgtggactctgtgaagggccggttcaccatctccagagacaccgtcaagaactcac |
| | | tgtatttgcaaatgaacaacctgagagccgaggacacggctgtatattactgcgcgaga |
| | | caaagattctgcagtgatgggagtctctttcacggagaagatctgcccagacccaccat |
| | | ctcggctgagacaggcaccgtgatctccctggggagccatgtgactttcgtgtgccggg |
| | | gcccacttggggtgcaaacattccgcctggagagggagagtaggtccagatacagtg |
| | | aaactgaagatgtgtctcaagttggtccatctgagtcagaggccagattccgcattgact |
| | | cagtgagtgaaggaaatgccgggctttatcgatgcatctattacaaaccccctaaatggt |
| | | ctgagcagagtgactacctggagctgcgggtgaaaggtgaggacgtcacctgggccc |
| | | tgttaacctattgtggtggtgatagagacgaatccgactactacatggacgtctggggca |
| | | aagggaccacggtcaccgtctcctca |
| 253 | light chain nuc | cagactgtggtgactcaggagccctcactgactgtgtccccaggagggacagtcactc |
| | | tcacctgtgcttccagtactggatcagtcaccagtggttcctttccaaactggttccagca |
| | | gacacctggacaagcacccaggtcactgatttatagtacaaccaaaaaacactcttgg |
| | | acccctgcccggttctcaggctctctccttgggggcaaagctgccctgacagtgtcaga |
| | | tacacagccggaggacgaggctgagtattactgcctactctactgtggtggtggtcaac |
| | | cttgggtgttcggcggagggaccaagctgaccgtccaa |

MGC34 ANTIBODY

| | | |
|---|---|---|
| 254 | CDRH1 aa | GFTFSSYW |
| 255 | CDRH2 aa | INQDGSQK |
| 256 | CDRH3 aa | ARERLCTDDSHMHGEEDLPRPSISAEPGTVIPLGSHVTFVCRG |
| | | PVGIHTFRLERESRSLYTETEDVTQVSPSESEARFRIESVTEGNAG |
| | | LYRCVYYKPPKWSEQSDYLELLVKCEDVTRALFTHCGGDGKE |
| | | SDYHMDV |
| 257 | CDRL1 aa | TGAVTSGYY |
| 258 | CDRL2 aa | STS |
| 259 | CDRL2 long aa | LIYSTSKTH |
| 260 | CDRL3 aa | LLYYGGPQPWV |
| 261 | CDRH1 nuc | ggattcacctttagtagttattgg |
| 262 | CDRH2 nuc | ataaaccaagatggaagtcagaaa |
| 263 | CDRH3 nuc | gcgagagaaagattgtgcactgatgatagtcacatgcacggagaagaagatctgccc |
| | | agaccctccatctcggctgagccaggcaccgtgatccccctggggagtcatgtgacct |
| | | tcgtgtgccggggcccggttgggattcacacattccgcctggagagggagagtagatc |
| | | cctatacactgaaactgaagatgtgactcaagtaagtccttctgagtcagaggccagatt |
| | | ccgcattgagtcagtaactgaaggaaatgccgggctttatcgctgcgtctattataagcc |
| | | ccctaaatggtctgagcagagtgactacctggagctgctggtgaaaggtgaggacgtc |
| | | acccgggccctgttcacccactgtggtggtgatggaaaggagtccgactaccacatgg |
| | | acgtc |
| 264 | CDRL1 nuc | actggagcagtcaccagtggttactat |
| 265 | CDRL2 nuc | agtacaagc |
| 266 | CDRL2 long nuc | ctgatttatagtacaagcaaaacacac |
| 267 | CDRL3 nuc | ctgctctattatggtggtcctcagccttgggtg |
| 268 | heavy chain aa | EVQLVESGGGLVQPGGSLRLSCEASGFTFSSYWMSWVRQAP |
| | | GKGLEWVANINQDGSQKDYVDSVKGRFTISRDTAKNSLYLQ |
| | | MNSLRAEDTAVYYCARERLCTDDSHMHGEEDLPRPSISAEPG |
| | | TVIPLGSHVTFVCRGPVGIHTFRLERESRSLYTETEDVTQVSPSE |
| | | SEARFRIESVTEGNAGLYRCVYYKPPKWSEQSDYLELLVKGED |
| | | VTRALFTHCGGDGKESDYHMDVWGKGTTVTVSS |
| 269 | light chain aa | QTVVTQEPSLTVSPGGTVTLTCASSTGAVTSGYYPSWFHQKP |
| | | GQPVRALIYSTSKTHSWTPARFSGSLLGGKAALTLSNVQPEDE |
| | | ADYYCLLYYGGPQPVWFGGGTKLTVQ |
| 270 | heavy chain nuc | gaggtgcagctggtggagtctgggggaggcttggtccagcctggggggtcactgagac |
| | | tctcctgtgaagcctccggattcacctttagtagttattggatgagctgggtccgccaggc |
| | | tccagggaaggggctggagtgggtggccaatataaaccaagatggaagtcagaaag |
| | | attatgtggattctgtgaagggccgattcaccatctccagagacaccgccaagaattcat |
| | | tatatctccaaatgaacagcctgagagccgaggacacggctgtttactactgtgcgaga |
| | | gaaagattgtgcactgatgatagtcacatgcacggagaagaagatctgcccagaccct |
| | | ccatctcggctgagccaggcaccgtgatccccctggggagtcatgtgaccttcgtgtgc |
| | | cggggcccggttgggattcacacattccgcctggagagggagagtagatccctataca |
| | | ctgaaactgaagatgtgactcaagtaagtccttctgagtcagaggccagattccgcattg |
| | | agtcagtaactgaaggaaatgccgggctttatcgctgcgtctattataagccccctaaat |
| | | ggtctgagcagagtgactacctggagctgctggtgaaaggtgaggacgtcacccggg |
| | | ccctgttcacccactgtggtggtgatggaaaggagtccgactaccacatggacgtctgg |
| | | ggcaaagggaccacggtcaccgtctcctca |
| 271 | light chain nuc | cagactgtggtgactcaggagccctcactgactgtgtccccaggagggacagtcactc |
| | | tcacctgtgcttctagcactggagcagtcaccagtggttactatccaagctggttccacc |
| | | agaaacctggacaaccagtcagggcactgatttatagtacaagcaaaacacactcct |
| | | ggacccctgcccgcttctcaggctccctccttgggggcaaagctgccctgacactgtc |
| | | aaatgtccagcctgaggacgaggctgactattactgcctgctctattatggtggtcctca |
| | | gccttgggtgttcggcggagggaccaagctgaccgtccaa |

MGC35 ANTIBODY

| | | |
|---|---|---|
| 272 | CDRH1 aa | GFNSRSYW |
| 273 | CDRH2 aa | INQDATEK |
| 274 | CDRH3 aa | ARDRFCGGESHLHGQEDLPRPSISAEPGSVIPLGSLVTFVCRGP |
| | | VGVHTFRLERGWTYNDTEDVSQAGPSESEARFRMDSVREGN |
| | | AGLYRCIYYKPPKWSEQSAYLELRVKGGDVTWALLTYCGGD |
| | | GEESDYPMDV |
| 275 | CDRL1 aa | TGPVTSAYY |
| 276 | CDRL2 aa | NIN |
| 277 | CDRL2 long aa | LIYNINKKH |
| 278 | CDRL3 aa | LLSCGGAQPWV |
| 279 | CDRH1 nuc | ggattcaactctcgtagttattgg |
| 280 | CDRH2 nuc | ataaatcaagatgcaactgagaaa |
| 281 | CDRH3 nuc | gcgagagacagattctgtggtggtgagagtcacttgcacggacaagaagatctgccca |
| | | gaccctccatctcggctgagccaggctccgtgatccccctggggagccttgtgactttc |
| | | gtgtgccggggcccggttggggttcacacattccgcctcgagagggggtggacataca |
| | | acgacactgaagatgtgtctcaagctggtccatctgagtcagaggccagattccgcatg |
| | | gactcggtaagggaaggaaatgccgggctttatcgatgcatctattacaaaccccctaa |
| | | atggtctgagcagagtgcctacctggaactgcgggtgaaaggtggggacgtcacctgg |
| | | gccctgttaacgtactgtggtggtgatggagaggaatccgactaccccatggacgtc |
| 282 | CDRL1 nuc | actggacctgtcaccagtgcttactat |
| 283 | CDRL2 nuc | aatataaac |
| 284 | CDRL2 long nuc | cttatttataatataaacaaaaaacac |
| 285 | CDRL3 nuc | ctgctctcctgtggtggtgctcagccttgggtg |
| 286 | heavy chain aa | EVQLVESGGGLVQPGGSLRLSCEASGFNSRSYWMTWVRQAP |
| | | GKGLEWVASINQDATEKNYVDSVKGRFTISRDTAKNSLYLQM |
| | | NSLRAEDTAVYYCARDRFCGGESHLHGQEDLPRPSISAEPGSV |
| | | IPLGSLVTFVCRGPVGVHTFRLERGWTYNDTEDVSQAGPSESE |
| | | ARFRMDSVREGNAGLYRCIYYKPPKWSEQSAYLELRVKGGDV |
| | | TWALLTYCGGDGEESDYPMDVWGKGTTVTVSS |
| 287 | light chain aa | QTVVTQEPSLTVSPGGTVTLTCASSTGPVTSAYYPNWFQQKP |
| | | GQAPRSLIYNINKKHSWTPDRFSGSLLGGKAALTLSGVQPEDE |
| | | ADYYCLLSCGGAQPWVFGGGTKLTVQ |
| 288 | heavy chain nuc | gaggtgcaactggtggagtctgggggaggcttggtccagcctggggggtccctgagac |
| | | tctcctgtgaagcctctggattcaactctcgtagttattggatgacctgggtccgccaggc |
| | | tccagggaaggggctggagtgggtggccagtataaatcaagatgcaactgagaaaaa |
| | | ttatgtggactctgtgaagggccggttcaccatctccagagacaccgccaagaactca |
| | | ctgtatctgcaaatgaacagcctgagagccgaggacacggctgtatattactgcgcga |
| | | gagacagattctgtggtggtgagagtcacttgcacggacaagaagatctgcccagacc |
| | | ctccatctcggctgagccaggctccgtgatccccctggggagccttgtgactttcgtgtg |
| | | ccggggcccggttggggttcacacattccgcctcgagagggggtggacatacaacga |
| | | cactgaagatgtgtctcaagctggtccatctgagtcagaggccagattccgcatggact |
| | | cggtaagggaaggaaatgccgggctttatcgatgcatctattacaaaccccctaaatgg |
| | | tctgagcagagtgcctacctggaactgcgggtgaaaggtggggacgtcacctgggcc |
| | | ctgttaacgtactgtggtggtgatggagaggaatccgactaccccatggacgtctgggg |
| | | caaagggaccacggtcaccgtctcctca |
| 289 | light chain nuc | cagactgtggtgactcaggagccctcactgactgtgtccccaggagggacagtcactc |
| | | tcacctgtgcttccagcactggacctgtcaccagtgcttactatccaaactggttccagc |
| | | agaagcctggacaagcacccaggtctcttatttataatataaacaaaaaacactcctgg |
| | | acccctgaccggttctcaggctccctccttgggggcaaagctgccctgacactgtcag |
| | | gtgtacagcctgaggacgaggctgactattactgcctgctctcctgtggtggtgctcagc |
| | | cttgggtgttcggcggagggaccaagctgaccgtccaa |

MGC36 ANTIBODY

| | | |
|---|---|---|
| 290 | CDRH1 aa | GFNSRSYW |
| 291 | CDRH2 aa | INQDGTEK |
| 292 | CDRH3 aa | ARDRFCGGESHLHGEEDLPRPSISAEPDTVIPLGSHVTFVCRGP |
| | | VGVHTFRLERGWRYNDTEDVSQAGPSESEARFRIDSVREGNA |
| | | GLYRCIYYIAPKWSEQSDYLELRVKGGDVTWALLTYCGGDGE |
| | | ESDYPMDV |
| 293 | CDRL1 aa | TGPVTSAYY |
| 294 | CDRL2 aa | SIN |
| 295 | CDRL2 long aa | LIYSINKKH |
| 296 | CDRL3 aa | LLSCGGAQPVW |
| 297 | CDRH1 nuc | ggattcaactctcgtagttattgg |
| 298 | CDRH2 nuc | ataaatcaagatgggactgagaaa |
| 299 | CDRH3 nuc | gcgagagacagattctgtggtggtgagagtcacttgcacggagaagaagatctgccca |
| | | gaccctccatctcggctgagccagacaccgtaatccccctggggagccatgtgacttt |
| | | cgtgtgccggggcccggttggggttcacacattccgcctggagagggggtggaggtac |
| | | aacgacactgaagatgtgtctcaagctggtccatctgagtcagaggccagattccgcat |
| | | tgactcggtaagggaaggaaatgccgggctttatcgatgcatctattacatagcccctaa |
| | | atggtctgagcagagtgactacctggagctgcgggtgaaaggtggggacgtcacctgg |
| | | gccctgttaacgtactgtggcggtgatggagaggaatccgactaccccatggacgtc |
| 300 | CDRL1 nuc | actggacctgtcaccagtgcttactat |
| 301 | CDRL2 nuc | agtataaac |
| 302 | CDRL2 long nuc | cttatttatagtataaacaaaaaacac |
| 303 | CDRL3 nuc | ctgctctcctgtggtggtgctcagccttgggtg |
| 304 | heavy chain aa | EVVLVESGGGLVQPGGSLRLSCEASGFNSRSYWMTWVRQAP |
| | | GKGLEWVASINQDGTEKNYVDSVKGRFTISRDSAKNSLYLQ |
| | | MSSLRADDTAVYYCARDRFCGGESHLHGEEDLPRPSISAEPDT |
| | | VIPLGSHVTFVCRGPVGVHTFRLERGWRYNDTEDVSQAGPSE |
| | | SEARFRIDSVREGNAGLYRCIYYIAPKWSEQSDYLELRVKGGD |
| | | VTWALLTYCGGDGEESDYPMDVWGKGTTVTVSS |
| 305 | light chain aa | QTVVTQEPSLTVSPGGTVTLTCASSTGPVTSAYYPNWFQQKP |
| | | GQAPRSLIYSINKKHSWTPARFSGSLLGGKAALTLSGVQPEDE |
| | | ADYYCLLSCGGAQPWVFGGGTKLTVQ |
| 306 | heavy chain nuc | gaggtggtactggtggagtctgggggaggcttggtccagcctggggggtccctgagact |
| | | ctcctgtgaagcctctggattcaactctcgtagttattggatgacctgggtccgccaggct |
| | | ccagggaaggggctggagtgggtggccagtataaatcaagatgggactgagaaaaat |
| | | tatgtggactctgtgaagggccggttcaccatctccagagactccgccaagaactcact |
| | | gtatctgcaaatgagcagcctgagagccgacgacacggctgtatattactgtgcgaga |
| | | gacagattctgtggtggtgagagtcacttgcacggagaagaagatctgcccagaccct |
| | | ccatctcggctgagccagacaccgtaatccccctggggagccatgtgactttcgtgtgc |
| | | cggggcccggttggggttcacacattccgcctggagagggggtggaggtacaacgac |
| | | actgaagatgtgtctcaagctggtccatctgagtcagaggccagattccgcattgactcg |
| | | gtaagggaaggaaatgccgggctttatcgatgcatctattacatagcccctaaatggtct |
| | | gagcagagtgactacctggagctgcgggtgaaaggtggggacgtcacctgggccctg |
| | | ttaacgtactgtggcggtgatggagaggaatccgactaccccatggacgtctggggca |
| | | aagggaccacggtcaccgtctcctca |
| 307 | light chain nuc | cagactgtggttactcaggagccctcactgactgtgtccccaggagggacagtcactct |
| | | cacctgtgcttccagcactggacctgtcaccagtgcttactatccaaactggttccagca |
| | | gaagcctggacaagcacccaggtctcttatttatagtataaacaaaaaacactcctgga |
| | | cccctgcccggttctcaggctccctccttgggggcaaagctgccctgacactgtcaggt |
| | | gtacagcctgaggacgaggctgactattactgcctgctctcctgtggtggtgctcagcct |
| | | tgggtgttcggcggagggaccaagctgaccgtccaa |

MGC37 ANTIBODY

| | | |
|---|---|---|
| 308 | CDRH1 aa | GFTFRNYW |
| 309 | CDRH2 aa | IRQDGSEK |
| 310 | CDRH3 aa | VRDKFCSDENHMHVADDLPRPSISPEPGTVIPLGSHVTFVCRG |
| | | PVGVQTFRLEKDRRSTYNDTEDVSQPSPSESEARFRIDSVTEGN |
| | | AGLYRCVYYKPPKWSDQSDFLELLVKGEDVTWALFPHCGAD |
| | | GEDSDYYMDV |
| 311 | CDRL1 aa | QRLSRS |
| 312 | CDRL2 aa | KAS |
| 313 | CDRL2 long aa | LIYKASPLE |
| 314 | CDRL3 aa | QQYSNYSYS |
| 315 | CDRH1 nuc | ggattcaccttcagaaattattgg |
| 316 | CDRH2 nuc | ataaggcaagatggaagtgagaag |
| 317 | CDRH3 nuc | gtgagagataaattctgcagtgatgagaatcacatgcacgtagcagatgatctgcccag |
| | | accctctatctcgcctgagccaggcaccgtgatccccctggggagccatgtgactttcg |
| | | tgtgtcggggcccggttggggttcaaacattccgcctggagaaggacagaagatccac |
| | | atacaatgatactgaagatgtgtctcaacctagtccatctgagtcagaggccagattccg |
| | | cattgactcagtaactgaaggaaatgccgggctttatcgctgcgtctattataagccccc |
| | | taaatggtctgaccagagtgacttcctggagttgctggtgaagggtgaggacgtcacctg |
| | | ggccctgttcccccattgtggtgctgatggagaggactccgactactacatggacgtc |
| 318 | CDRL1 nuc | cagcgtcttagtcgctcg |
| 319 | CDRL2 nuc | aaggcgtct |
| 320 | CDRL2 long nuc | ctgatctataaggcgtctcctttagaa |
| 321 | CDRL3 nuc | caacaatacagtaattattcatatagt |
| 322 | heavy chain aa | EVQLVESGGDLVQPGGSLRLSCAASGFTFRNYWMSWVRQTP |
| | | GKGLEWVANIRQDGSEKYYVDSVKGRFTISRDNAKNLLYLQ |
| | | MNSLRAEDTAVYYCVRDKFCSDENHMHVADDLPRPSISPEPG |
| | | TVIPLGSHVTFVCRGPVGVQTFRLEKDRRSTYNDTEDVSQPSP |
| | | SESEARFRIDSVTEGNAGLYRCVYYKPPKWSDQSDFLELLVKG |
| | | EDVTWALFPHCGADGEDSDYYMDVWGKGTTVTVSS |
| 323 | light chain aa | DIQLTQSPSTLSASVGDRVTISCRASRLSRSLAWYQQRPRKA |
| | | PNLLIYKASPLEIGGPSRFTGSGSGTEFTLTISSLQPDDSATYYC |
| | | QQYSNYSYSFGQGTKLEIR |
| 324 | heavy chain nuc | gaggtgcagctggtggagtctgggggagacttggtccagcctggggggtccctgagac |
| | | tctcctgtgcagcctctggattcaccttcagaaattattggatgagttgggtccgccagac |
| | | tccagggaagggactggagtgggtggccaacataaggcaagatggaagtgagaagt |
| | | attatgtggactctgtgaagggccgattcaccatctccagagacaacgccaagaactta |
| | | ttatatctacaaatgaacagcctgagagccgaggacacggctgtgtattactgtgtgaga |
| | | gataaattctgcagtgatgagaatcacatgcacgtagcagatgatctgcccagaccctc |
| | | tatctcgcctgagccaggcaccgtgatccccctggggagccatgtgactttcgtgtgtcg |
| | | gggcccggttggggttcaaacattccgcctggagaaggacagaagatccacatacaa |
| | | tgatactgaagatgtgtctcaacctagtccatctgagtcagaggccagattccgcattga |
| | | ctcagtaactgaaggaaatgccgggctttatcgctgcgtctattataagccccctaaatg |
| | | gtctgaccagagtgacttcctggagttgctggtgaagggtgaggacgtcacctgggccc |
| | | tgttcccccattgtggtgctgatggagaggactccgactactacatggacgtctggggca |
| | | aagggaccacggtcaccgtctcctca |
| 325 | light chain nuc | gacatccagctgacccagtctccttccaccctgtctgcatctgtaggagacagagtcac |
| | | catctcttgccgggccagtcagcgtcttagtcgctcgttggcctggtatcagcagagacc |
| | | acggaaagcccctaacctcctgatctataaggcgtctcctttagaaattgggggcccat |
| | | caaggttcaccggcagtggatctgggacagaattcactctcaccatcagcagcctgca |
| | | gcctgatgattctgcaacttattactgccaacaatacagtaattattcatatagttttggcca |
| | | ggggaccaagctggagatcaga |

MGD21 ANTIBODY

| | | |
|---|---|---|
| 326 | CDRH1 aa | GDYVNTNRR |
| 327 | CDRH2 aa | VHQSGRT |
| 328 | CDRH3 aa | ARASPLKSQRDTDLPRPSISAEPGTVIPLGSHVTFVCRGPVGV |
| | | QTFRLERERNYLYSDTEDVSQTSPSESEARFRIDSVNAGNAGLF |
| | | RCIYYKSRKWSEQSDYLELWKGEDVTWALSQSQDDPRACP |
| | | QGELPISTDIYYVDV |
| 329 | CDRL1 aa | QHINDS |
| 330 | CDRL2 aa | GAS |
| 331 | CDRL2 long aa | LIYGASNLH |
| 332 | CDRL3 aa | QQCNCFPPD |
| 333 | CDRH1 nuc | ggtgactacgtcaatactaataggagg |
| 334 | CDRH2 nuc | gttcatcaaagtgggagaacc |
| 335 | CDRH3 nuc | gcgagagcgtctccactcaaatctcagagggacaccgatctgcccagaccctccatc |
| | | tcggctgagccgggcaccgtgatccccctggggagccatgtgactttcgtgtgccggg |
| | | gcccggttggggttcaaacattccgcctggagagggagaggaattatttatacagtgata |
| | | ctgaagatgtgtctcaaactagtccatctgagtcggaggccagattccgcattgactcag |
| | | taaatgcaggcaatgccgggctttttcgctgcatctattacaagtcccgtaaatggtctga |
| | | gcagagtgactacctggagctggtggtgaaaggtgaggacgtcacctgggccctgtcc |
| | | cagtctcaagacgaccctcgagcttgtccccagggggagctccccataagtaccgat |
| | | atttactacgtggacgtc |
| 336 | CDRL1 nuc | caacatattaatgattct |
| 337 | CDRL2 nuc | ggtgcatcc |
| 338 | CDRL2 long nuc | ctgatatatggtgcatccaatttgcac |
| 339 | CDRL3 nuc | caacagtgtaattgtttccctccggac |
| 340 | heavy chain aa | EVQLVETGPGLMKTSGTLSLTCAVSGDYVNTNRRWSWVRQ |
| | | APGKGLEWIGEVHQSGRTNYNPSLKSRVTISVDKSKNQFSLKV |
| | | DSVTAADTAVYYCARASPLKSQRDTDLPRPSISAEPGTVIPLGS |
| | | HVTFVCRGPVGVQTFRLERERNYLYSDTEDVSQTSPSESEARF |
| | | RIDSVNAGNAGLFRCIYYKSRKWSEQSDYLELWKGEDVTWA |
| | | LSQSQDDPRACPQGELPISTDIYYVDVWGNGTTVTVSS |
| 341 | light chain aa | AIRMTQSPSSLSASPGDKVSITCRASQHINDSLAWFQQRPGK |
| | | APKLLIYGASNLHSGVPSRFSGIGSGTDFTLTITGLQSEDFATY |
| | | FCQQCNCFPPDFGQGTRLEIK |
| 342 | heavy chain nuc | gaggtgcagctggtggagacgggcccaggactgatgaagacttcggggaccctgtcc |
| | | ctcacgtgcgctgtgtctggtgactacgtcaatactaataggaggtggagttgggtccgc |
| | | caggccccagggaagggcctggagtggattggagaggttcatcaaagtgggagaac |
| | | caattacaacccgtccctcaagagccgagtcaccatatcagtagacaagtctaagaat |
| | | cagttctctctgaaggtggactctgtgaccgccgcggacacggccgtgtattactgtgcg |
| | | agagcgtctccactcaaatctcagagggacaccgatctgcccagaccctccatctcg |
| | | gctgagccgggcaccgtgatccccctggggagccatgtgactttcgtgtgccggggcc |
| | | cggttggggttcaaacattccgcctggagagggagaggaattatttatacagtgatactg |
| | | aagatgtgtctcaaactagtccatctgagtcggaggccagattccgcattgactcagtaa |
| | | atgcaggcaatgccgggctttttcgctgcatctattacaagtcccgtaaatggtctgagc |
| | | agagtgactacctggagctggtggtgaaaggtgaggacgtcacctgggccctgtccca |
| | | gtctcaagacgaccctcgagcttgtccccagggggagctccccataagtaccgatattt |
| | | actacgtggacgtctggggcaacgggaccacggtcaccgtctcctca |
| 343 | light chain nuc | gccatccggatgacccagtctccatcctcactctctgcatcaccaggggacaaagtca |
| | | gcatcacttgtcgggcgagtcaacatattaatgattctttggcctggtttcaacaaaggcc |
| | | agggaaagccccaaaactcctgatatatggtgcatccaatttgcacagtggggtcccat |
| | | cgaggttcagcggcactgggtcagggacagatttcactctcactatcaccggcctgca |
| | | gtctgaagattttgcaacttatttctgtcaacagtgtaattgtttccctccggacttcggcca |
| | | agggacacgactggagattaaa |

MGD23 ANTIBODY

| | | |
|---|---|---|
| 344 | CDRH1 aa | GGSISSNKW |
| 345 | CDRH2 aa | VYQTGIT |
| 346 | CDRH3 aa | ATISQLRPQGDTEDLPRPSLSAEPGTVIPLGSHVTFVCRGPAGV |
| | | ETFRLERESRFTYNDTEDVSQASPSESEARFRIDSVSEGNAGPYR |
| | | CLYYKARKWSDQSDYLELLVKGADVTWALPQSQLAPRACPQ |
| | | GELRISTDVFSMNV |
| 347 | CDRL1 aa | QYVGNY |
| 348 | CDRL2 aa | GVS |
| 349 | CDRL2 long aa | LIHGVSTLQ |
| 350 | CDRL3 aa | QQYYTSPPD |
| 351 | CDRH1 nuc | ggtggctccattagtagtaataagtgg |
| 352 | CDRH2 nuc | gtgtatcagactggtattacc |
| 353 | CDRH3 nuc | gcgacaatttctcaactgaggccgcagggggacaccgaagatctgcccagaccctc |
| | | cctctcggctgaaccaggcaccgtgatccccctggggagtcacgtgactttcgtgtgcc |
| | | ggggcccggctggggtcgaaacattccgcctggagagggagagtagattcacttaca |
| | | acgatactgaagatgtgtctcaagcgagtccatctgagtcagaggccagattccgcatt |
| | | gactcagtaagtgaaggaaatgccgggccttatcgctgcctctattataaggcccgtaa |
| | | atggtctgaccagagtgactacttggaattgctggtgaagggtgcggacgtcacctggg |
| | | ccctgccccagtctcagctcgcccctcgagcttgtccccagggagaactccgcattag |
| | | taccgatgttttctccatgaacgtc |
| 354 | CDRL1 nuc | caatatgttgggaattat |
| 355 | CDRL2 nuc | ggtgtatcc |
| 356 | CDRL2 long nuc | ctcattcacggtgtatccactttgcaa |
| 357 | CDRL3 nuc | cagcagtattatacttcccctccggac |
| 358 | heavy chain aa | QVQLQESGPGLVKPSGTLSLTCSVSGGSISSNKWWSWVRQS |
| | | PCKGLEWIGEVYQTGITNYNPSLKGRVTMSVDKSKNQFSLRL |
| | | TSVTAADTAVYYCATISQLRPQGDTEDLPRPSLSAEPGTVIPLG |
| | | SHVTFVCRGPAGVETFRLERESRFTYNDTEDVSQASPSESEARF |
| | | RIDSVSEGNAGPYRCLYYKARKWSDQSDYLELLVKGADVTW |
| | | ALPQSQLAPRACPQGELRISTDVFSMNVWGNGTTVTVSS |
| 359 | light chain aa | AVRVTQSPTSLSASTGDRVTITCRTSQYVGNYLDWYQQKPG |
| | | KAPKLLIHGVSTLQNGVPSRFSGSASGTDFTLNITCLQSEDSAT |
| | | YYCQQYYTSPPDFGQGTRLEIK |
| 360 | heavy chain nuc | caggtgcagctgcaggagtcgggcccaggactggtgaagccttcgggaaccctgtcc |
| | | ctcacctgcagtgtctctggtggctccattagtagtaataagtggtggagttgggtccgcc |
| | | agtccccagggaagggcctggagtggattggggaggtgtatcagactggtattaccaa |
| | | ctacaacccgtccctcaagggtcgagtcaccatgtcagtggacaagtccaagaacca |
| | | attctccctgagactgacttctgtgaccgccgcggacacggccgtgtattactgtgcgac |
| | | aatttctcaactgaggccgcagggggacaccgaagatctgcccagaccctccctctc |
| | | ggctgaaccaggcaccgtgatccccctggggagtcacgtgactttcgtgtgccggggc |
| | | ccggctggggtcgaaacattccgcctggagagggagagtagattcacttacaacgata |
| | | ctgaagatgtgtctcaagcgagtccatctgagtcagaggccagattccgcattgactca |
| | | gtaagtgaaggaaatgccgggccttatcgctgcctctattataaggcccgtaaatggtct |
| | | gaccagagtgactacttggaattgctggtgaagggtgcggacgtcacctgggccctgc |
| | | cccagtctcagctcgcccctcgagcttgtccccagggagaactccgcattagtaccga |
| | | tgttttctccatgaacgtctggggcaacgggaccacggtcaccgtctcttca |
| 361 | light chain nuc | gccgtccgggtgacccagtctccaacctcactgtctgcatctacaggagacagagtca |
| | | ccatcacttgtcggacgagtcaatatgttgggaattatttagattggtatcagcaaaaacc |
| | | agggaaagcccctaaactcctcattcacggtgtatccactttgcaaaatggggtcccat |
| | | caaggttcagtggcagtgcctccgggacagacttcactctcaacatcacctgcctaca |
| | | gtctgaagattctgcaacttattactgtcagcagtattatacttcccctccggacttcggcc |
| | | aagggacacgcctggaaattaag |

MGD30 ANTIBODY

| | | |
|---|---|---|
| 362 | CDRH1 aa | GGSITSSKW |
| 363 | CDRH2 aa | IYHNGTT |
| 364 | CDRH3 aa | ATASPFKSHHRTTEKLPRPSISAEPGTVIPLGSRVTFVCRGPVGV |
| | | QTFRLERETSFTYNDTEDVSQVSPSESEARFRIDSVSEGYAGPYR |
| | | CVYYKAPKWSEQSDYLDLLVKGEDVTWALTQPQLDPRACPQ |
| | | GDLRMSTDIYCMDV |
| 365 | CDRL1 aa | QDISTF |
| 366 | CDRL2 aa | AAS |
| 367 | CDRL2 long aa | LIFAASTLQ |
| 368 | CDRL3 aa | QQYYCFPPD |
| 369 | CDRH1 nuc | ggtggctccatcaccagtagtaagtgg |
| 370 | CDRH2 nuc | atctatcataatgggaccacc |
| 371 | CDRH3 nuc | gcaacggcgtctcccttcaagtctcatcacaggaccaccgaaaaactgcccagacc |
| | | ctccatctcggctgagccgggcaccgtgatccccctggggagccgtgtgactttcgtgt |
| | | gccggggcccggttggggttcaaacattccgcctagagagggagactagctttacatat |
| | | aatgatactgaagatgtgtctcaggttagtccgtctgagtcagaggccagattccgcatt |
| | | gactcagtgagtgagggatatgccgggccttatcgctgcgtctattataaggcccctaag |
| | | tggtccgagcagagtgactacctggacctgctggtgaaaggtgaggacgtcacttggg |
| | | ccctgacccagcctcagctcgaccctcgagcttgtccccagggggacctccgcatga |
| | | gcaccgatatttactgcatggacgtc |
| 372 | CDRL1 nuc | caggatattagcactttt |
| 373 | CDRL2 nuc | gctgcatct |
| 374 | CDRL2 long nuc | ctaatctttgctgcatctactttacaa |
| 375 | CDRL3 nuc | caacagtattattgtttccctccggac |
| 376 | heavy chain aa | QVQLQESGPGLVKPSETLSLSCAVTGGSITSSKWWTWVRQG |
| | | PDKGLEWIGKIYHNGTTNYNPSLKSRVAMSVDKSRNQFSLRL |
| | | TSVTAADTALYYCATASPFKSHHRTTEKLPRPSISAEPGTVIPLG |
| | | SRVTFVCRGPVGVQTFRLERETSFTYNDTEDVSQVSPSESEARF |
| | | RIDSVSEGYAGPYRCVYYKAPKWSEQSDYLDLLVKGEDVTWA |
| | | LTQPQLDPRACPQGDLRMSTDIYCMDVWGKGTTVTVSS |
| 377 | light chain aa | AIRLTQSPSSLSASIGDRVTITCRASQDISTFLAWYQQESGKAP |
| | | RLLIFAASTLQTGVPSRFSGSGSGTDFTLTISGLQSEDFATYYCQ |
| | | QYYCFPPDFGQGTRLDIK |
| 378 | heavy chain nuc | caggtgcagctgcaggagtcgggcccaggactggtgaagccttcagaaaccctgtcc |
| | | ctctcctgcgctgtcactggtggctccatcaccagtagtaagtggtggacttgggtccgc |
| | | cagggcccagataaggggctggagtggattgggaaaatctatcataatgggaccacc |
| | | aactacaatccgtccctcaagagtcgagtcgccatgtcggtggacaagtccaggaac |
| | | cagttctccctgagactgacctccgtgaccgccgcggacacggccttgtattactgtgc |
| | | aacggcgtctcccttcaagtctcatcacaggaccaccgaaaaactgcccagaccctc |
| | | catctcggctgagccgggcaccgtgatccccctggggagccgtgtgactttcgtgtgcc |
| | | ggggcccggttggggttcaaacattccgcctagagagggagactagctttacatataat |
| | | gatactgaagatgtgtctcaggttagtccgtctgagtcagaggccagattccgcattgac |
| | | tcagtgagtgagggatatgccgggccttatcgctgcgtctattataaggcccctaagtgg |
| | | tccgagcagagtgactacctggacctgctggtgaaaggtgaggacgtcacttgggccc |
| | | tgacccagcctcagctcgaccctcgagcttgtccccagggggacctccgcatgagca |
| | | ccgatatttactgcatggacgtctggggcaaagggaccacggtcaccgtctcctca |
| 379 | light chain nuc | gccatccggttgacccaatctccatcctcactctctgcatctataggagacagagtcac |
| | | catcacttgtcgggcgagtcaggatattagcacttttttggcctggtatcaacaagagtca |
| | | ggtaaagccccaaggctcctaatctttgctgcatctactttacaaactggggtcccttca |
| | | aggttcagcggcagtggatctgggacagatttcactctcaccatcagcggcctgcaatc |
| | | tgaagattttgcaacttattactgtcaacagtattattgtttccctccggacttcggccaagg |
| | | gacacgactggacattaaa |

MGD33 ANTIBODY

| | | |
|---|---|---|
| 380 | CDRH1 aa | GGSITSSKW |
| 381 | CDRH2 aa | IYHNGTT |
| 382 | CDRH3 aa | ATASPFKSHHRTTEKLPRPSISAEPGTVIPLGSRVTFVCRGPVGV |
| | | QTFRLERETRSTYNDTEDVSQVSPSESEARFRIDSVSEGYAGPY |
| | | RCVYYKAPKWSEQSDYLDLLVKGEDVTWALTQPQLDPRACP |
| | | QGDLRMSTDIYCMDV |
| 383 | CDRL1 aa | QDISTY |
| 384 | CDRL2 aa | AAS |
| 385 | CDRL2 long aa | LIFAASSLQ |
| 386 | CDRL3 aa | QQYYCFPPD |
| 387 | CDRH1 nuc | ggtggctccatcaccagtagtaagtgg |
| 388 | CDRH2 nuc | atctatcataatgggaccacc |
| 389 | CDRH3 nuc | gcaacggcgtctcccttcaagtctcatcacaggaccaccgaaaaactgcccagacc |
| | | ctccatctcggctgagccgggcaccgtgatccccctggggagccgtgtgactttcgtgt |
| | | gccggggcccggttggggttcaaacattccgcctagagagggagactagatctacata |
| | | taatgatactgaagatgtgtctcaggttagtccgtctgagtcagaggccagattccgcatt |
| | | gactcagtgagtgagggatatgccgggccttatcgctgcgtctattataaggcccctaag |
| | | tggtccgagcagagtgactacctggacctgctggtgaaaggtgaggacgtcacttggg |
| | | ccctgacccagcctcagctcgaccctcgagcttgtccccagggggacctccgcatga |
| | | gcaccgatatttactgcatggacgtc |
| 390 | CDRL1 nuc | caggatattagcacttat |
| 391 | CDRL2 nuc | gctgcatct |
| 392 | CDRL2 long nuc | ctaatctttgctgcatctagtttacaa |
| 393 | CDRL3 nuc | caacaatattattgtttccctccggac |
| 394 | heavy chain aa | HVQLQESGPGLVKPSETLSLSCAVTGGSITSSKWWTWVRQGP |
| | | DKGLEWIGKIYHNGTTNYNPSLKSRVAMSVDKSKNQFSLRLT |
| | | SVTAADTAVYYCATASPFKSHHRTTEKLPRPSISAEPGTVIPLGS |
| | | RVTFVCRGPVGVQTFRLERETRSTYNDTEDVSQVSPSESEARFR |
| | | IDSVSEGYAGPYRCVYYKAPKWSEQSDYLDLLVKGEDVTWAL |
| | | TQPQLDPRACPQGDLRMSTDIYCMDVWGKGTTVTVSS |
| 395 | light chain aa | AIRLTQSPSSLSASIGDRVTITCRASQDISTYLAWYQQESGKAP |
| | | RLLIFAASSLQTGVPSRFSGSGSGTDFTLTISGLQSEDFATYYCQ |
| | | QYYCFPPDFGQGTRLDIK |
| 396 | heavy chain nuc | cacgtgcagctgcaggagtcgggcccaggactggtgaagccttcagaaaccctgtcc |
| | | ctctcctgcgctgtcactggtggctccatcaccagtagtaagtggtggacttgggtccgc |
| | | cagggcccagataaggggctggagtggattgggaaaatctatcataatgggaccacc |
| | | aactacaatccgtccctcaagagtcgagtcgccatgtcggtggacaagtccaagaac |
| | | cagttctccctgagactgacctccgtgaccgccgcggacacggccgtgtattactgtgc |
| | | aacggcgtctcccttcaagtctcatcacaggaccaccgaaaaactgcccagaccctc |
| | | catctcggctgagccgggcaccgtgatccccctggggagccgtgtgactttcgtgtgcc |
| | | ggggcccggttggggttcaaacattccgcctagagagggagactagatctacatataat |
| | | gatactgaagatgtgtctcaggttagtccgtctgagtcagaggccagattccgcattgac |
| | | tcagtgagtgagggatatgccgggccttatcgctgcgtctattataaggcccctaagtgg |
| | | tccgagcagagtgactacctggacctgctggtgaaaggtgaggacgtcacttgggccc |
| | | tgacccagcctcagctcgaccctcgagcttgtccccagggggacctccgcatgagca |
| | | ccgatatttactgcatggacgtctggggcaaagggaccacggtcaccgtctcctca |
| 397 | light chain nuc | gccatccggttgacccaatctccatcctcactctctgcatctataggagacagagtcac |
| | | catcacttgtcgggcgagtcaggatattagcacttatttggcctggtatcaacaagagtc |
| | | aggtaaagccccaaggctcctaatctttgctgcatctagtttacaaactggggtcccttc |
| | | aaggttcagcggcagtggatctgggacagatttcactctcaccatcagcggcctgcagt |
| | | ctgaagattttgcaacttattactgtcaacaatattattgtttccctccggacttcggccaag |
| | | ggacacgactggacattaaa |

MGD34 ANTIBODY

| | | |
|---|---|---|
| 398 | CDRH1 aa | GDYVNTNRR |
| 399 | CDRH2 aa | VHQSGRT |
| 400 | CDRH3 aa | ARASPLKSQRDTEDLPRPSISAEPGTVIPLGSHVTFVCRGPVGV |
| | | QTFRLERERNYLYSDTEDVSQTSPSESEARFRIDSVNAGNAGLF |
| | | RCIYYKSRKWSEQSDYLELVVKGEDVTWALSQSQVDPRACP |
| | | QGELPISTDIYYVDV |
| 401 | CDRL1 aa | QHINDS |
| 402 | CDRL2 aa | GAS |
| 403 | CDRL2 long aa | LIYGASNLH |
| 404 | CDRL3 aa | QQCNCFPPD |
| 405 | CDRH1 nuc | ggtgactacgtcaatactaataggagg |
| 406 | CDRH2 nuc | gttcatcaaagtgggagaacc |
| 407 | CDRH3 nuc | gcgagagcgtctccactcaaatctcagagggacaccgaagatctgcccagaccctc |
| | | catctcggctgagccgggcaccgtgatccccctggggagccatgtgactttcgtgtgcc |
| | | ggggcccggttggggttcaaacattccgcctggagagggagaggaattatttatacagt |
| | | gatactgaagatgtgtctcaaactagtccatctgagtcggaggccagattccgcattgac |
| | | tcagtaaatgcaggcaatgccgggctttttcgctgcatctattacaagtcccgtaaatggt |
| | | ctgagcagagtgactacctggagctggtggtgaaaggtgaggacgtcacctgggccct |
| | | gtcccagtctcaaGTCGACcctcgagcttgtccccagggggagctccccataagt |
| | | accgatatttactacgtggacgtc |
| 408 | CDRL1 nuc | caacatattaatgattct |
| 409 | CDRL2 nuc | ggtgcatcc |
| 410 | CDRL2 long nuc | ctgatatatggtgcatccaatttgcac |
| 411 | CDRL3 nuc | caacagtgtaattgtttccctccggac |
| 412 | heavy chain aa | EVQLVESGPGLMKTSGTLSLTCAVSGDYVNTNRRWSWVRQA |
| | | PGKGLEWIGEVHQSGRTNYNPSLKSRVTISVDKSKNQFSLKV |
| | | DSVTAADTAVYYCARASPLKSQRDTEDLPRPSISAEPGTVIPLG |
| | | SHVTFVCRGPVGVQTFRLERERNYLYSDTEDVSQTSPSESEARF |
| | | RIDSVNAGNAGLFRCIYYKSRKWSEQSDYLELWKGEDVTWA |
| | | LSQSQVDPRACPQGELPISTDIYYVDVWGNGTTFTVSS |
| 413 | light chain aa | AIRMTQSPSSLSASPGDKVSITCRASQHINDSLAWFQQRPGK |
| | | APKLLIYGASNLHSGVPSRFSGTGSGTDFTLTITGLQSEDFATY |
| | | FCQQCNCFPPDFGQGTRLEIK |
| 414 | heavy chain nuc | gaggtgcagctggtggagtcgggcccaggactgatgaagacttcggggaccctgtcc |
| | | ctcacgtgcgctgtgtctggtgactacgtcaatactaataggaggtggagttgggtccgc |
| | | caggccccagggaagggcctggagtggattggagaggttcatcaaagtgggagaac |
| | | caattacaacccgtccctcaagagccgagtcaccatatcagtagacaagtctaagaat |
| | | cagttctctctgaaggtggactctgtgaccgccgcggacacggccgtgtattactgtgcg |
| | | agagcgtctccactcaaatctcagagggacaccgaagatctgcccagaccctccatc |
| | | tcggctgagccgggcaccgtgatccccctggggagccatgtgactttcgtgtgccggg |
| | | gcccggttggggttcaaacattccgcctggagagggagaggaattatttatacagtgata |
| | | ctgaagatgtgtctcaaactagtccatctgagtcggaggccagattccgcattgactcag |
| | | taaatgcaggcaatgccgggctttttcgctgcatctattacaagtcccgtaaatggtctga |
| | | gcagagtgactacctggagctggtggtgaaaggtgaggacgtcacctgggccctgtcc |
| | | cagtctcaaGTCGACcctcgagcttgtccccagggggagctccccataagtaccg |
| | | atatttactacgtggacgtctggggcaacgggaccacgttcaccgtctcctca |
| 415 | light chain nuc | gccatccggatgacccagtctccatcctcactctctgcatcaccaggggacaaagtca |
| | | gcatcacttgtcgggcgagtcaacatattaatgattctttggcctggtttcaacaaaggcc |
| | | agggaaagccccaaaactcctgatatatggtgcatccaatttgcacagtggggtcccat |
| | | cgaggttcagcggcactgggtcagggacagatttcactctcactatcaccggcctgca |
| | | gtctgaagattttgcaacttatttctgtcaacagtgtaattgtttccctccggacttcggcca |
| | | agggacacgactggagattaaa |

MGD35 ANTIBODY

| | | |
|---|---|---|
| 416 | CDRH1 aa | GASISSINW |
| 417 | CDRH2 aa | IHHNGST |
| 418 | CDRH3 aa | ATASSLKSQRDTNLPRPSLSAEPGTVIPLGSPVTFVCRGPVGVH |
| | | TFRLERAGRSTYNDTEDVSHPSPSESEARFRIDSVSEGNAGPYR |
| | | CVYYKSSKWSEESYCLDLLVKTEDVTWARPQPQLDPRACPQG |
| | | DLRISTDFYYMDV |
| 419 | CDRL1 aa | QAIGTY |
| 420 | CDRL2 aa | NAS |
| 421 | CDRL2 long aa | LIYNASTLQ |
| 422 | CDRL3 aa | QHYYNYPPA |
| 423 | CDRH1 nuc | ggtgcctccatcagtagtattaattgg |
| 424 | CDRH2 nuc | atccatcataatgggagcacc |
| 425 | CDRH3 nuc | gcgactgcctcttcattgaagtctcagagggacaccaatttgcccagaccctccctctc |
| | | ggcggagccaggcaccgtgatccccctggggagccctgtgactttcgtgtgccgggg |
| | | cccggttggggttcacacattccgcctggagagggcgggtagatccacatacaatgat |
| | | actgaagatgtgtctcatcctagtccatctgagtcagaggccagattccgcattgactca |
| | | gtgagtgagggaaatgccgggccttatcgctgcgtctattataagtcctctaaatggtcc |
| | | gaggagagttactgcctggacctgctggtcaaaactgaggacgtcacgtgggcccgg |
| | | ccccagcctcagctcgaccctcgagcttgtccccagggggacctccgcattagcacc |
| | | gatttttactacatggacgtc |
| 426 | CDRL1 nuc | caggctattggcacttat |
| 427 | CDRL2 nuc | aatgcttcc |
| 428 | CDRL2 long nuc | ctgatctataatgcttccactttgcaa |
| 429 | CDRL3 nuc | caacactattataattatcctccggcc |
| 430 | heavy chain aa | QVQLQESGPGLVKPSGTLSLTCAVSGASISSINWWSWVRQTP |
| | | EKGLEWIGQIHHNGSTNYNPSLKSRVAISVDKSKNQFSLKLTS |
| | | FTAADTAVYYCATASSLKSQRDTNLPRPSLSAEPGTVIPLGSPV |
| | | TFVCRGPVGVHTFRLERAGRSTYNDTEDVSHPSPSESEARFRID |
| | | SVSEGNAGPYRCVYYKSSKWSEESYCLDLLVKTEDVTWARPQ |
| | | PQLDPRACPQGDLRISTDFYYMDVWGKGTTVTVSS |
| 431 | light chain aa | AIRMTQSPSSLSASTGDRVTITCRTSQAIGTYLAWYQQNPGK |
| | | APNLLIYNASTLQSGVPSRFSASGSGTDFTLTISGLQSDDFVTY |
| | | FCQHYYNYPPAFGQGTRLEIQ |
| 432 | heavy chain nuc | caggtgcagctgcaggagtcgggcccaggactggtgaagccttcggggaccctgtcc |
| | | ctcacctgcgctgtctctggtgcctccatcagtagtattaattggtggagttgggtccgtca |
| | | gaccccagaaaaggggctggagtggattggacaaatccatcataatgggagcacca |
| | | actacaacccgtccctcaagagtcgggtcgccatatcagttgacaagtccaagaacc |
| | | agttctccctgaagttgacttctttcaccgccgcggacacggccgtgtattattgtgcgac |
| | | tgcctcttcattgaagtctcagagggacaccaatttgcccagaccctccctctcggcgg |
| | | agccaggcaccgtgatccccctggggagccctgtgactttcgtgtgccggggcccggt |
| | | tggggttcacacattccgcctggagagggcgggtagatccacatacaatgatactgaa |
| | | gatgtgtctcatcctagtccatctgagtcagaggccagattccgcattgactcagtgagt |
| | | gagggaaatgccgggccttatcgctgcgtctattataagtcctctaaatggtccgaggag |
| | | agttactgcctggacctgctggtcaaaactgaggacgtcacgtgggcccggccccag |
| | | cctcagctcgaccctcgagcttgtccccagggggacctccgcattagcaccgattttta |
| | | ctacatggacgtctggggcaaagggaccacggtcaccgtctcttca |
| 433 | light chain nuc | gccatccggatgacccagtctccatcctcactctctgcatctacgggagacagagtca |
| | | ccatcacttgtcggacgagtcaggctattggcacttatttagcgtggtatcagcagaacc |
| | | cagggaaagcccctaacctcctgatctataatgcttccactttgcaaagtggggtcccat |
| | | caaggttcagcgccagtggctctgggacagatttcactctcaccatcagcggcctgca |
| | | gtctgacgattttgtcacttatttctgccaacactattataattatcctccggccttcggcca |
| | | agggacacgactggagattcaa |

MGD39 ANTIBODY

| | | |
|---|---|---|
| 434 | CDRH1 aa | GGSISAYRW |
| 435 | CDRH2 aa | VYNDGNT |
| 436 | CDRH3 aa | ATISPLRPQSDTDDLPRPSISAEPGTVIPLGSHVTFVCRGPIGVQ |
| | | TFRLERERRSLYSDTEDVSQVSPFASEARFRIDSVSEGNAGPYRC |
| | | IYYKDRKWSDQSDYLELLVKGEDVTWALPQSQLAPRACPQEE |
| | | LNISTDIFSMNV |
| 437 | CDRL1 aa | HDVGNY |
| 438 | CDRL2 aa | GAS |
| 439 | CDRL2 long aa | LIHGASTLQ |
| 440 | CDRL3 aa | QQYYSSPPG |
| 441 | CDRH1 nuc | ggtggctccatcagtgcttataggtgg |
| 442 | CDRH2 nuc | gtctataatgatggcaatacc |
| 443 | CDRH3 nuc | gcgacaatttctccactgaggcctcagagtgacaccgacgatctgcccagaccctcc |
| | | atctcggctgagccaggcaccgtgatccccctggggagccatgtgaccttcgtgtgcc |
| | | ggggcccaattggggttcaaacattccgcctggagagggagagaagatccttatacag |
| | | tgatactgaagatgtgtctcaagttagtccatttgcgtcagaggccagattccgcattgac |
| | | tcagtaagtgaaggaaatgccgggccatatcgctgcatctattataaggaccggaaatg |
| | | gtctgaccagagtgactacctggagttgctggtgaaaggtgaggacgtcacctgggcc |
| | | ctgccccagtctcagctcgcccctcgcgcttgtccccaggaagaattgaacattagtac |
| | | cgatattttctccatgaacgtc |
| 444 | CDRL1 nuc | catgatgttggtaattat |
| 445 | CDRL2 nuc | ggtgcgtcc |
| 446 | CDRL2 long nuc | ctgatccacggtgcgtccactttgcaa |
| 447 | CDRL3 nuc | caacaatattacagttcccctccgggc |
| 448 | heavy chain aa | QVRLQESGPGLVKPSGTLSLTCTVSGGSISAYRWWSWVRQA |
| | | PGKGLEWIGQVYNDGNTNYNPSLKGRVAMSVDKSKNRFSLR |
| | | LASVTAADTAVYYCATISPLRPQSDTDDLPRPSISAEPGTVIPLG |
| | | SHVTFVCRGPIGVQTFRLERERRSLYSDTEDVSQVSPFASEARF |
| | | RIDSVSEGNAGPYRCIYYKDRKWSDQSDYLELLVKGEDVTWA |
| | | LPQSQLAPRACPQEELNISTDIFSMNVWGKGTTVTVSS |
| 449 | light chain aa | AIRMTQSPASLSASIGDRVTITCRTSHDVGNYLDWYQQKPGK |
| | | APKLLIHGASTLQTGVPSRFSGSGAGTDFTLNITCLQSGDFAM |
| | | YYCQQYYSSPPGFGQGTRLEIK |
| 450 | heavy chain nuc | caggtgcggctgcaggagtcgggcccaggactggtgaagccttcggggaccctgtcc |
| | | ctcacctgcactgtctctggtggctccatcagtgcttataggtggtggagttgggtccgcc |
| | | aggccccaggcaagggcctggagtggattggacaggtctataatgatggcaatacca |
| | | actacaacccgtccctcaagggtcgagtcgccatgtcagtggacaagtccaagaatc |
| | | gattttccctgagattagcgtctgtgaccgccgcggacacggccgtgtattactgtgcga |
| | | caatttctccactgaggcctcagagtgacaccgacgatctgcccagaccctccatctc |
| | | ggctgagccaggcaccgtgatccccctggggagccatgtgaccttcgtgtgccgggg |
| | | cccaattggggttcaaacattccgcctggagagggagagaagatccttatacagtgata |
| | | ctgaagatgtgtctcaagttagtccatttgcgtcagaggccagattccgcattgactcagt |
| | | aagtgaaggaaatgccgggccatatcgctgcatctattataaggaccggaaatggtctg |
| | | accagagtgactacctggagttgctggtgaaaggtgaggacgtcacctgggccctgcc |
| | | ccagtctcagctcgcccctcgcgcttgtccccaggaagaattgaacattagtaccgata |
| | | ttttctccatgaacgtctggggcaaagggaccacggtcaccgtctcctca |
| 451 | light chain nuc | gccatccggatgacccagtctccagcgtctctgtctgcatctataggagacagagtcac |
| | | catcacttgtcggacgagtcatgatgttggtaattatttagattggtatcaacaaaaacca |
| | | ggaaaagcccctaaactcctgatccacggtgcgtccactttgcaaactggggtcccat |
| | | cacggttcagcggcagtggagccgggacagatttcactctcaacatcacctgcctgca |
| | | gtctggagatttcgcaatgtattattgtcaacaatattacagttcccctccgggcttcggcc |
| | | aagggacacgactggagattaaa |

MGD41 ANTIBODY

| | | |
|---|---|---|
| 452 | CDRH1 aa | GGSINTDKW |
| 453 | CDRH2 aa | VLHTGST |
| 454 | CDRH3 aa | ATISTLRPQRDIEDLPRPSLSAEPGTVVPLGSHVTFVCRGPVGV |
| | | QTFRLERESRSTYNDTEDVSQPSPFESEARFRIDSVSEGNAGPY |
| | | RCIYYKSPKWSDQSDYVELLVKGEDVTWAPPQSQLAPRACP |
| | | QGELRTSTDIFSMNV |
| 455 | CDRL1 aa | QDIGNY |
| 456 | CDRL2 aa | GAS |
| 457 | CDRL2 long aa | LIHGASTLL |
| 458 | CDRL3 aa | LQYYSSPPA |
| 459 | CDRH1 nuc | ggtggctccatcaacactgataagtgg |
| 460 | CDRH2 nuc | gtccttcatactgggagcacc |
| 461 | CDRH3 nuc | gcgactatttctacattgaggcctcagcgggacatcgaagatctgcccagaccctccct |
| | | ctcggctgagccaggcaccgtggtccccctggggagccatgtgactttcgtgtgccgg |
| | | ggcccggttggggttcaaacattccgcctggagagggagagcagatccacatacaat |
| | | gatactgaagatgtgtctcaacctagtccatttgagtcagaggccagatttcgcattgact |
| | | cagtaagtgaaggaaatgccgggccttatcgctgcatctattataagtcccctaaatggt |
| | | ctgaccagagtgactacgtggagttgctggtgaaaggtgaggacgtcacctgggcccc |
| | | gccccagtctcagctcgcccctcgagcttgtccccagggagaactccgcactagcac |
| | | cgatattttctccatgaacgtc |
| 462 | CDRL1 nuc | caggatattggtaattac |
| 463 | CDRL2 nuc | ggtgcatcc |
| 464 | CDRL2 long nuc | ctgatccatggtgcatccactttgctg |
| 465 | CDRL3 nuc | ctacaatattacagttcccctccggcc |
| 466 | heavy chain aa | EVQLVESGPGLVKPSGTLSVTCTISGGSINTDKWWTWVRQPP |
| | | GKGLEWVGEVLHTGSTNYNPSLRGRVTISVDKSKNQFSLRLSS |
| | | VTAADTAVYYCATISTLRPQRDIEDLPRPSLSAEPGTVVPLGSH |
| | | VTFVCRGPVGVQTFRLERESRSTYNDTEDVSQPSPFESEARFRI |
| | | DSVSEGNAGPYRCIYYKSPKWSDQSDYVELLVKGEDVTWAPP |
| | | QSQLAPRACPQGELRTSTDIFSMNVWGKGTTVTVSS |
| 467 | light chain aa | AIRMTQSPSSLSAFTGDRVTISCRASQDIGNYLDWYHQKPGR |
| | | APKLLIHGASTLLTGVPSRFSGSGSGTDFTLNITCLQSGDFGIY |
| | | YCLQYYSSPPAFGPGTRLEIK |
| 468 | heavy chain nuc | gaggtgcagctggtggagtcgggcccaggactggtgaagccttcggggaccctgtcc |
| | | gtcacctgcactatctctggtggctccatcaacactgataagtggtggacttgggtccgc |
| | | cagcccccagggaagggccttgagtgggtaggggaagtccttcatactgggagcacc |
| | | aactacaacccgtccctgaggggtcgagtcaccatatcagtggacaagtccaagaac |
| | | cagttctccctgaggctgagttctgtgaccgccgcggacacggccgtatattattgtgcg |
| | | actatttctacattgaggcctcagcgggacatcgaagatctgcccagaccctccctctc |
| | | ggctgagccaggcaccgtggtccccctggggagccatgtgactttcgtgtgccggggc |
| | | ccggttggggttcaaacattccgcctggagagggagagcagatccacatacaatgata |
| | | ctgaagatgtgtctcaacctagtccatttgagtcagaggccagatttcgcattgactcagt |
| | | aagtgaaggaaatgccgggccttatcgctgcatctattataagtcccctaaatggtctga |
| | | ccagagtgactacgtggagttgctggtgaaaggtgaggacgtcacctgggccccgcc |
| | | ccagtctcagctcgcccctcgagcttgtccccagggagaactccgcactagcaccga |
| | | tattttctccatgaacgtctggggcaaagggaccacggtcaccgtctcctca |
| 469 | light chain nuc | gccatccggatgacccagtctccatcctcactgtctgcatttacaggagacagagtcac |
| | | catctcttgccgggcgagtcaggatattggtaattacttagattggtatcaccaaaagcc |
| | | aggaagagcccctaagctcctgatccatggtgcatccactttgctgactggggtcccat |
| | | cacgattcagcggcagtggatccggaacagatttcactctcaacatcacctgcctgca |
| | | gtctggagattttggaatttattactgtctacaatattacagttcccctccggccttcggccc |
| | | agggacacggctggagattaaga |

MGD47 ANTIBODY

| | | |
|---|---|---|
| 470 | CDRH1 aa | GGSISGYKW |
| 471 | CDRH2 aa | VYDDGDT |
| 472 | CDRH3 aa | ATISPLRPQSDTGDLPRPSISAEPGTAIPLGSQVTFVCRGPIGVQ |
| | | TFRLERESRALYNDSEDVSQVSPSASEARFRIDSVSEGNAGPYR |
| | | CIYYKARRWSDQSDYLELLVKGEDVTWALPQSQLAPRACPQE |
| | | DLNISTDIFSTNV |
| 473 | CDRL1 aa | QDVGNY |
| 474 | CDRL2 aa | GAS |
| 475 | CDRL2 long aa | LIHGASTLQ |
| 476 | CDRL3 aa | QQYYTSPPV |
| 477 | CDRH1 nuc | ggtggctccatcagtggttacaagtgg |
| 478 | CDRH2 nuc | gtctatgatgatggcgacacc |
| 479 | CDRH3 nuc | gcgacaatttctccactgaggcctcagagtgacaccggagatctgcccagaccctcc |
| | | atctcggctgagccaggcaccgcgatccccctggggagccaagtgactttcgtgtgcc |
| | | ggggcccaattggggttcaaacattccgcctggagagggagagtcgcgccttatataat |
| | | gattctgaagatgtgtctcaagttagtccatctgcgtcagaggccagattccgcattgact |
| | | cagtaagtgaaggcaatgccgggccttatcgctgtatctattataaggcccgcagatggt |
| | | ctgaccagagtgactatttggagttgttggtgaaaggtgaggacgtcacctgggccctgc |
| | | cccagtctcagctcgcccctcgcgcttgtccccaggaagatttgaacattagtaccgat |
| | | attttctctacgaacgtc |
| 480 | CDRL1 nuc | caggatgttggaaattat |
| 481 | CDRL2 nuc | ggtgcgtcc |
| 482 | CDRL2 long nuc | ctcatccacggtgcgtccactttgcaa |
| 483 | CDRL3 nuc | caacaatattacacttcccctccggtc |
| 484 | heavy chain aa | QVRLQESGPGLVKPSGTLSLTCTVSGGSISGYKWWSWVRQA |
| | | PGKGLEWIGQVYDDGDTNYNPDLKGRVALSVDKSKSRFSLSL |
| | | ASVTAADTAIYFCATISPLRPQSDTGDLPRPSISAEPGTAIPLGS |
| | | QVTFVCRGPIGVQTFRLERESRALYNDSEDVSQVSPSASEARFR |
| | | IDSVSEGNAGPYRCIYYKARRWSDQSDYLELLVKGEDVTWAL |
| | | PQSQLAPRACPQEDLNISTDIFSTNVWGKGTTVTVSS |
| 485 | light chain aa | AIRMTQSPASLSASVGDRVTITCRTSQDVGNYLDWYQQKPG |
| | | KAPKLLIHGASTLQAGVPSRFNGSGSGTDFTGISCVQSGDFA |
| | | IYYCQQYYTSPPVFGQGTRLEIK |
| 486 | heavy chain nuc | caggtgcggctgcaggagtcgggcccaggactggtgaagccttcggggaccctgtcc |
| | | ctcacctgcactgtctcgggtggctccatcagtggttacaagtggtggagttgggtccgc |
| | | caggccccaggcaagggcctggagtggattggacaggtctatgatgatggcgacacc |
| | | aactacaatccggacctgaagggtcgagtcgccctgtcagtggacaagtccaagagt |
| | | cgattttccctcagcctagcgtctgtgaccgccgcggacacggccatatacttctgtgcg |
| | | acaatttctccactgaggcctcagagtgacaccggagatctgcccagaccctccatct |
| | | cggctgagccaggcaccgcgatccccctggggagccaagtgactttcgtgtgccggg |
| | | gcccaattggggttcaaacattccgcctggagagggagagtcgcgccttatataatgatt |
| | | ctgaagatgtgtctcaagttagtccatctgcgtcagaggccagattccgcattgactcag |
| | | taagtgaaggcaatgccgggccttatcgctgtatctattataaggcccgcagatggtctg |
| | | accagagtgactatttggagttgttggtgaaaggtgaggacgtcacctgggccctgccc |
| | | cagtctcagctcgcccctcgcgcttgtccccaggaagatttgaacattagtaccgatatt |
| | | ttctctacgaacgtctggggcaaagggacaacggtcaccgtctcttca |
| 487 | light chain nuc | gccatccggatgacccagtctccagcgtccctgtctgcatctgtaggagacagagtca |
| | | ccatcacttgtcggacgagtcaggatgttggaaattatttagattggtatcaacaaaaac |
| | | caggaaaagcccctaaactcctcatccacggtgcgtccactttgcaagctggggtccc |
| | | atcacgtttcaacggcagtggatccgggacagatttcactctcggcatcagttgtgtgca |
| | | gtctggagatttcgcgatctattactgtcaacaatattacacttcccctccggtcttcggcc |
| | | aagggacacgactggagattaaa |

MGD55 ANTIBODY

| | | |
|---|---|---|
| 488 | CDRH1 aa | GGSISAYKW |
| 489 | CDRH2 aa | VYHNGNT |
| 490 | CDRH3 aa | ATISPLRPQSDTDDLPRPSISAEPGTVIPLGSHVTFVCRGPIGVQ |
| | | TFRLERESRSLYSDTEDVSQVSPFASEARFRIDSVSEGNAGPYRC |
| | | IYYKDRKWSDQSDYLELLVKGEDVTWALPQSQLAPRACPQEE |
| | | LNISTDIFSMNV |
| 491 | CDRL1 aa | QDVGNY |
| 492 | CDRL2 aa | GAS |
| 493 | CDRL2 long aa | LIHGASTLQ |
| 494 | CDRL3 aa | QQYYSSPPG |
| 495 | CDRH1 nuc | ggtggctccatcagtgcttataagtgg |
| 496 | CDRH2 nuc | gtctatcataatggcaacacc |
| 497 | CDRH3 nuc | gcgacaatttctccactgaggcctcagagtgacaccgacgatctgcccagaccctcc |
| | | atctcggctgagccaggcaccgtgatccccctggggagccatgtgactttcgtgtgccg |
| | | gggcccaattggggttcaaacattccgcctggagagggagagtagatccttatacagtg |
| | | atactgaagatgtgtctcaagttagtccatttgcgtcagaggccagattccgcattgactc |
| | | agtaagtgaaggaaatgccgggccatatcgctgcatctattataaggaccggaaatggt |
| | | ctgaccagagtgactacctggagttgctggtgaaaggtgaggacgtcacctgggccct |
| | | gccccagtctcagctcgcccctcgcgcttgtccccaggaagaattgaacattagtacc |
| | | gatattttctccatgaacgtc |
| 498 | CDRL1 nuc | caggatgttggtaattat |
| 499 | CDRL2 nuc | ggtgcgtcc |
| 500 | CDRL2 long nuc | ctgatccacggtgcgtccactttgcaa |
| 501 | CDRL3 nuc | caacaatattacagttcccctccgggc |
| 502 | heavy chain aa | QVQLQESGPGLVKPSGTLSLTCTVSGGSISAYKWWSWVRQA |
| | | PGKGLEWIGQVYHNGNTNYNPSLKGRVAMSVDKSKNRFSLR |
| | | LASVTAADTAVYYCATISPLRPQSDTDDLPRPSISAEPGTVIPLG |
| | | SHVTFVCRGPIGVQTFRLERESRSLYSDTEDVSQVSPFASEARF |
| | | RIDSVSEGNAGPYRCIYYKDRKWSDQSDYLELLVKGEDVTWA |
| | | LPQSQLAPRACPQEELNISTDIFSMNVWGKGTTVTVSS |
| 503 | light chain aa | AIRMTQSPASLSASIGDRVTITCRTSQDVGNYLDWYQQKPGK |
| | | APKLLIHGASTLQTGVPSRFSGSGAGTDFTLNITCLQSGDFAM |
| | | YYCQQYYSSPPGFGQGTRLEIK |
| 504 | heavy chain nuc | caggtgcagctgcaggagtcgggcccaggactggtgaagccttcggggaccctgtcc |
| | | ctcacctgcactgtctctggtggctccatcagtgcttataagtggtggagttgggtccgcc |
| | | aggccccaggcaagggcctggagtggattggacaggtctatcataatggcaacacca |
| | | actacaacccgtccctcaagggtcgagtcgccatgtcagtggacaagtccaagaatc |
| | | gattttccctgagactagcgtctgtgaccgccgcggacacggccgtgtattactgtgcga |
| | | caatttctccactgaggcctcagagtgacaccgacgatctgcccagaccctccatctc |
| | | ggctgagccaggcaccgtgatccccctggggagccatgtgactttcgtgtgccggggc |
| | | ccaattggggttcaaacattccgcctggagagggagagtagatccttatacagtgatact |
| | | gaagatgtgtctcaagttagtccatttgcgtcagaggccagattccgcattgactcagta |
| | | agtgaaggaaatgccgggccatatcgctgcatctattataaggaccggaaatggtctga |
| | | ccagagtgactacctggagttgctggtgaaaggtgaggacgtcacctgggccctgccc |
| | | cagtctcagctcgcccctcgcgcttgtccccaggaagaattgaacattagtaccgatatt |
| | | ttctccatgaacgtctggggcaaagggaccacggtcaccgtctcctca |
| 505 | light chain nuc | gccatccggatgacccagtctccagcgtctctgtctgcatctataggagacagagtcac |
| | | catcacttgtcggacgagtcaggatgttggtaattatttagattggtatcaacaaaaacca |
| | | ggaaaagcccctaaactcctgatccacggtgcgtccactttgcaaactggggtcccat |
| | | cacggttcagcggcagtggagccgggacagatttcactctcaacatcacctgcctgca |
| | | gtctggagatttcgcaatgtattactgtcaacaatattacagttcccctccgggcttcggc |
| | | caagggacacgactggaaattaaga |

MGD56 ANTIBODY

| | | |
|---|---|---|
| 506 | CDRH1 aa | GGSITTNNW |
| 507 | CDRH2 aa | IFRSGTT |
| 508 | CDRH3 aa | ATASPFKSQRDTKDLPRPSLSAEPGTVIPLGSHVTFVCRGPVGV |
| | | QTFRLQRESRSLYNDTEDVSHPSPSESEARFRIDSVSEGNAGPY |
| | | RCVYYKSSKWSEESDCLELLVKTEDVTWARPQPQLDPRACPR |
| | | GDLRISTDVYYMDV |
| 509 | CDRL1 aa | QAITSY |
| 510 | CDRL2 aa | NAS |
| 511 | CDRL2 long aa | LIYNASTLQ |
| 512 | CDRL3 aa | QHYYTYPPA |
| 513 | CDRH1 nuc | ggtggctccatcactactaataattgg |
| 514 | CDRH2 nuc | atctttcgtagtgggaccacc |
| 515 | CDRH3 nuc | gcgacagcctctccattcaagtctcagagggacaccaaagatttgcccagaccctcc |
| | | ctctcggctgagccaggcaccgtgatccccctggggagtcatgtgactttcgtgtgccg |
| | | gggcccggttggggttcagacattccgcctgcagagggagagtagatccctttacaatg |
| | | atactgaagatgtgtctcatcctagtccatctgagtcagaggccagattccgcattgact |
| | | cagtgagtgagggaaatgccgggccttatcgctgcgtctattataagtcctctaaatggt |
| | | ccgaggagagtgactgcctggagctgctggtcaaaactgaggacgtcacctgggccc |
| | | ggccccagcctcagctcgaccctcgagcttgtccccggggggacctccgcattagca |
| | | ccgatgtttactacatggacgtc |
| 516 | CDRL1 nuc | caggctattaccagttat |
| 517 | CDRL2 nuc | aatgcttcc |
| 518 | CDRL2 long nuc | ctgatctataatgcttccactttgcaa |
| 519 | CDRL3 nuc | caacactattatacttaccctccggcc |
| 520 | heavy chain aa | QVQLQESGPGLVKPSGTLSLTCAVSGGSITTNNWWSWVRQT |
| | | PGKGLEWIGEIFRSGTTNYNPSLKSRVAISLDKSKNQFSLKLTSV |
| | | TAADTAVYYCATASPFKSQRDTKDLPRPSLSAEPGTVIPLGSHV |
| | | TFVCRGPVGVQTFRLQRESRSLYNDTEDVSHPSPSESEARFRID |
| | | SVSEGNAGPYRCVYYKSSKWSEESDCLELLVKTEDVTWARPQ |
| | | PQLDPRACPRGDLRISTDVYYMDVWGKGTTVTVSS |
| 521 | light chain aa | AIRMTQSPSSLSASTGDRVTITCRASQAITSYLAWYRQKPGKA |
| | | PDLLIYNASTLQSGVPSRFSASGSGTDFALTITGLQSEDFVIYFC |
| | | QHYYTYPPAFGQGTRLEIK |
| 522 | heavy chain nuc | caggtgcagctgcaggagtcgggcccaggactggtgaagccttcggggaccctgtcc |
| | | ctcacctgcgctgtctctggtggctccatcactactaataattggtggagttgggtccgtc |
| | | agaccccaggaaaggggctggagtggattggagaaatctttcgtagtgggaccacca |
| | | actacaacccgtccctcaagagtcgggtcgccatttcattagacaagtccaagaacca |
| | | gttctccctgaagttgacttctgtgaccgccgcggacacggccgtgtattactgtgcgac |
| | | agcctctccattcaagtctcagagggacaccaaagatttgcccagaccctccctctcg |
| | | gctgagccaggcaccgtgatccccctggggagtcatgtgactttcgtgtgccggggcc |
| | | cggttggggttcagacattccgcctgcagagggagagtagatccctttacaatgatactg |
| | | aagatgtgtctcatcctagtccatctgagtcagaggccagattccgcattgactcagtga |
| | | gtgagggaaatgccgggccttatcgctgcgtctattataagtcctctaaatggtccgagg |
| | | agagtgactgcctggagctgctggtcaaaactgaggacgtcacctgggcccggcccc |
| | | agcctcagctcgaccctcgagcttgtccccggggggacctccgcattagcaccgatgt |
| | | ttactacatggacgtctggggcaaagggaccacggtcaccgtctcctca |
| 523 | light chain nuc | gccatccggatgacccagtctccatcctcactctctgcatctacaggggacagagtca |
| | | ccatcacttgtcgggcgagtcaggctattaccagttatttagcctggtatcggcagaaac |
| | | cagggaaagcccctgacctcctgatctataatgcttccactttgcaaagtggggtcccat |
| | | caagattcagcgccagtggctctgggacagatttcgctctcaccatcaccggcctgca |
| | | gtctgaggattttgtaatttatttctgccaacactattatacttaccctccggccttcggcca |
| | | agggacacgactggagattaaa |

Constant regions

| | | |
|---|---|---|
| 524 | IgG1 CH1—CH2—CH3 | ASTKGPSVFPLAPSSKSTSGGTAALGCLVKDYFPEPVTVSWNS |
| | aa | GALTSGVHTFPAVLQSSGLYSLSSVVTVPSSSLGTQTYICNVN |
| | | HKPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALPA |
| | | PIEKTISKAKGQPREPQVYTLPPSREEMTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 525 | IgG CK aa | RTVAAPSVFIFPPSDEQLKSGTASVVCLLNNFYPREAKVQWKV |
| | | DNALQSGNSQESVTEQDSKDSTYSLSSTLTLSKADYEKHKVYA |
| | | CEVTHQGLSSPVTKSFNRGEC |
| 526 | IgG CL aa | GQPKAAPSVTLFPPSSEELQANKATLVCLISDFYPGAVTVAWK |
| | | ADSSPVKAGVETTTPSKQSNNKYAASSYLSLTPEQWKSHRSYS |
| | | CQVTHEGSTVEKTVAPTECS |
| 527 | IgGI CH1—CH2—CH3 | gcgtcgaccaagggcccatcggtcttccccctggcaccctcctccaagagcacctct |
| | nucl | gggggcacagcggccctgggctgcctggtcaaggactacttccccgaacctgtgacg |
| | | gtctcgtggaactcaggcgccctgaccagcggcgtgcacaccttcccggctgtcctac |
| | | agtcctcaggactctactccctcagcagcgtggtgaccgtgccctccagcagcttggg |
| | | cacccagacctacatctgcaacgtgaatcacaagcccagcaacaccaaggtggaca |
| | | agagagttgagcccaaatcttgtgacaaaactcacacatgcccaccgtgcccagcac |
| | | ctgaactcctggggggaccgtcagtcttcctcttccccccaaaacccaaggacaccct |
| | | catgatctcccggacccctgaggtcacatgcgtggtggtggacgtgagccacgaAga |
| | | Ccctgaggtcaagttcaactggtacgtggacggcgtggaggtgcataatgccaagac |
| | | aaagccgcgggaggagcagtacaacagcacgtaccgtgtggtcagcgtcctcaccg |
| | | tcctgcaccaggactggctgaatggcaaggagtacaagtgcaaggtctccaacaaag |
| | | ccctcccagcccccatcgagaaaaccatctccaaagccaaagggcagccccgaga |
| | | accacaggtgtacaccctgcccccatcccgggaggagatgaccaagaaccaggtca |
| | | gcctgacctgcctggtcaaaggcttctatcccagcgacatcgccgtggagtgggagag |
| | | caatgggcagccggagaacaactacaagaccacgcctcccgtgctggactccgacg |
| | | gctccttcttcctctatagcaagctcaccgtggacaagagcaggtggcagcaggggaa |
| | | cgtcttctcatgctccgtgatgcatgaggctctgcacaaccactacacgcagaagagc |
| | | ctctccctgtccccgggtaaa |
| 528 | IgG CK nucl | cgTacGgtggctgcaccatctgtcttcatcttcccgccatctgatgagcagttgaaatct |
| | | ggaactgcctctgttgtgtgcctgctgaataacttctatcccagagaggccaaagtacag |
| | | tggaaggtggataacgccctccaatcgggtaactcccaggagagtgtcacagagcag |
| | | gacagcaaggacagcacctacagcctcagcagcaccctgacgctgagcaaagcag |
| | | actacgagaaacacaaagtctacgcctgcgaagtcacccatcagggcctgagctcg |
| | | cccgtcacaaagagcttcaacaggggagagtgt |
| 529 | IgG CL nucl | ggtcagcccaaggctgccccctcggtcactctgttcccgccctcctctgaggagcttca |
| | | agccaacaaggccacactggtgtgtctcataagtgacttctacccgggagccgtgaca |
| | | gtggcttggaaagcagatagcagccccgtcaaggcgggagtggagaccaccacacc |
| | | ctccaaacaaagcaacaacaagtacgcggccagcagctatctgagcctgacgcctg |
| | | agcagtggaagtcccacagaagctacagctgccaggtcacgcatgaagggagcacc |
| | | gtggagaagacagtggcccctacagaatgttca |

Preferably, the second variable (V2) domain of a RIFIN/N-terminal semi-conserved domain of a RIFIN is/are able to bind to an antibody having (i) a heavy chain variable region comprising the amino acid sequence of SEQ ID NO: 70 or a functional sequence variant thereof and a light chain variable region comprising the amino acid sequence of SEQ ID NO: 71 or a functional sequence variant thereof; or (ii) a heavy chain variable region comprising the amino acid sequence of SEQ ID NO: 88 or a functional sequence variant thereof and a light chain variable region comprising the amino acid sequence of SEQ ID NO: 89 or a functional sequence variant thereof; or (iii) a heavy chain variable region comprising the amino acid sequence of SEQ ID NO: 106 or a functional sequence variant thereof and a light chain variable region comprising the amino acid sequence of SEQ ID NO: 107 or a functional sequence variant thereof; or (iv) a heavy chain variable region comprising the amino acid sequence of SEQ ID NO: 124 or a functional sequence variant thereof and a light chain variable region comprising the amino acid sequence of SEQ ID NO: 125 or a functional sequence variant thereof; or (v) a heavy chain variable region comprising the amino acid sequence of SEQ ID NO: 142 or a functional sequence variant thereof and a light chain variable region comprising the amino acid sequence of SEQ ID NO: 143 or a functional sequence variant thereof; or (vi) a heavy chain variable region comprising the amino acid sequence of SEQ ID NO: 160 or a functional sequence variant thereof and a light chain variable region comprising the amino acid sequence of SEQ ID NO: 161 or a functional sequence variant thereof; or (vii) a heavy chain variable region comprising the amino acid sequence of SEQ ID NO: 178 or a functional sequence variant thereof and a light chain variable region comprising the amino acid sequence of SEQ ID NO: 179 or a functional sequence variant thereof; or (viii) a heavy chain variable region comprising the amino acid sequence of SEQ ID NO: 196 or a functional sequence variant thereof and a light chain variable region comprising the amino acid sequence of SEQ ID NO: 197 or a functional sequence variant thereof; or (ix) a heavy chain variable region comprising the amino acid sequence of SEQ ID NO: 214 or a functional sequence variant thereof and a light chain variable region comprising the amino acid sequence of SEQ ID NO: 215 or a functional sequence variant thereof; or (x) a heavy chain variable region comprising the amino acid sequence of SEQ ID NO: 232 or a functional sequence variant thereof and a light chain variable region comprising the amino acid sequence of SEQ ID NO: 233 or a functional sequence variant thereof; or (xi) a heavy chain variable region comprising the amino acid sequence of SEQ ID NO: 250 or a functional sequence variant thereof and a light chain variable region comprising the amino acid sequence of SEQ ID NO: 251 or a functional sequence variant thereof; or (xii) a heavy chain variable region comprising the amino acid sequence of SEQ ID NO: 268 or a functional sequence variant thereof and a light chain variable region comprising the amino acid sequence of SEQ ID NO: 269 or a functional sequence variant thereof; or (xiii) a heavy chain variable region comprising the amino acid sequence of SEQ ID NO: 286 or a functional sequence variant thereof and a light chain variable region comprising the amino acid sequence of SEQ ID NO: 287 or a functional sequence variant thereof; or (xiv) a heavy chain variable region comprising the amino acid sequence of SEQ ID NO: 304 or a functional sequence variant thereof and a light chain variable region comprising the amino acid sequence of SEQ ID NO: 305 or a functional sequence variant thereof; or (xv) a heavy chain variable region comprising the amino acid sequence of SEQ ID NO: 322 or a functional sequence variant thereof and a light chain variable region comprising the amino acid sequence of SEQ ID NO: 323 or a functional sequence variant thereof; or (xvi) a heavy chain variable region comprising the amino acid sequence of SEQ ID NO: 340 or a functional sequence variant thereof and a light chain variable region comprising the amino acid sequence of SEQ ID NO: 341 or a functional sequence variant thereof; or (xvii) a heavy chain variable region comprising the amino acid sequence of SEQ ID NO: 358 or a functional sequence variant thereof and a light chain variable region comprising the amino acid sequence of SEQ ID NO: 359 or a functional sequence variant thereof; or (xviii) a heavy chain variable region comprising the amino acid sequence of SEQ ID NO: 376 or a functional sequence variant thereof and a light chain variable region comprising the amino acid sequence of SEQ ID NO: 377 or a functional sequence variant thereof; or (xix) a heavy chain variable region comprising the amino acid sequence of SEQ ID NO: 394 or a functional sequence variant thereof and a light chain variable region comprising the amino acid sequence of SEQ ID NO: 395 or a functional sequence variant thereof; or (xx) a heavy chain variable region comprising the amino acid sequence of SEQ ID NO: 412 or a functional sequence variant thereof and a light chain variable region comprising the amino acid sequence of SEQ ID NO: 413 or a functional sequence variant thereof; or (xxi) a heavy chain variable region comprising the amino acid sequence of SEQ ID NO: 430 or a functional sequence variant thereof and a light chain variable region comprising the amino acid sequence of SEQ ID NO: 431 or a functional sequence variant thereof; or (xxii) a heavy chain variable region comprising the amino acid sequence of SEQ ID NO: 448 or a functional sequence variant thereof and a light chain variable region comprising the amino acid sequence of SEQ ID NO: 449 or a functional sequence variant thereof; or (xxiii) a heavy chain variable region comprising the amino acid sequence of SEQ ID NO: 466 or a functional sequence variant thereof and a light chain variable region comprising the amino acid sequence of SEQ ID NO: 467 or a functional sequence variant thereof; or (xxiv) a heavy chain variable region comprising the amino acid sequence of SEQ ID NO: 484 or a functional sequence variant thereof and a light chain variable region comprising the amino acid sequence of SEQ ID NO: 485 or a functional sequence variant thereof; or (xxv) a heavy chain variable region comprising the amino acid sequence of SEQ ID NO: 502 or a functional sequence variant thereof and a light chain variable region comprising the amino acid sequence of SEQ ID NO: 503 or a functional sequence variant thereof; or (xxvi) a heavy chain variable region comprising the amino acid sequence of SEQ ID NO: 520 or a functional sequence variant thereof and a light chain variable region comprising the amino acid sequence of SEQ ID NO: 521 or a functional sequence variant thereof.

More preferably the second variable (V2) domain of a RIFIN/N-terminal semi-conserved domain of a RIFIN is/are able to bind to an antibody having a heavy chain variable region comprising the amino acid sequence of SEQ ID NO: 340 or a functional sequence variant thereof and a light chain variable region comprising the amino acid sequence of SEQ ID NO: 341 or a functional sequence variant thereof.

In particular, the pharmaceutical composition according to the present invention comprises a polypeptide, which comprises or consists of a second variable (V2) domain and/or an N-terminal semi-conserved domain of a RIFIN, which is/are able to bind to a LAIR-1 fragment as defined herein. Preferably, the pharmaceutical composition according to the present invention comprises a polypeptide, which comprises or consists of a second variable (V2) domain of a RIFIN, which is able to bind to a LAIR-1 fragment as defined herein.

A “RIFIN” as used herein refers to a protein of the RIFIN family (repetitive interspersed family proteins). In addition to proteins, which are classified as RIFINs, the skilled person may easily determine whether any (unknown) protein is a RIFIN by use of appropriate computer programs, for example “RSpred”, which is freely accessible under http://www.bioinfo.ifm.liu.se/ and described by Joannin N. et al., 2011: RSpred, a set of Hidden Markov Models to detect and classify the RIFIN and STEVOR proteins of Plasmodium falciparum. BMC genomics 12:119.

A RIFIN is a Plasmodium falciparum variant surface antigen. “Plasmodium falciparum variant surface antigens” include—without being limited thereto—PfEMP1 (P. falciparum erythrocyte membrane protein 1), RIFIN (repetitive interspersed family proteins), STEVOR (sub-telomeric variable open reading frame proteins) and SURFIN (surface-associated interspersed gene family proteins).

The function of RIFINs remains largely unknown, however, RIFINs were initially linked with rosetting and described as strain-specific, antigenically distinct, P. falciparum-derived polypeptides termed as rosettins (Helmby et al., 1993, Infect Immun. 61(1):284-8). Rifgenes have a two-exon structure with first exon coding for a predicted signal peptide and the second for a protein that is highly variable but contains stretches of relative amino acid conservation and conserved cysteine residues. RIFINs have deduced molecular masses between 27 and 45 kDa and carry a semi-conserved domain and cysteine-rich regions at the N-terminus, while the C-terminal half is highly polymorphic.

RIFINS are described as small polypeptides comprising in the direction from N- to C-terminus:
(1) a putative signal peptide (SP),
(2) a first variable domain (V1),
(3) a plasmodium export element (PEXEL),
(4) an N-terminal semi-conserved domain (C1, also referred to as “constant region 1”),
(5) a hydrophobic patch, which is proposed to be a transmembrane domain (TM1),
(6) a second variable domain, also known as hypervariable domain (V2),
(7) a (second) transmembrane domain (TM2), and
(8) a C-terminal conserved domain (C2)
   as described for example by Joannin N. et al., 2008, BMC genomics 9:19 (FIG. 1 and corresponding description) and by Templeton T. J., 2009, Molecular & Biochemical Parasitology 166: 109-116. By using this literature, the skilled person can easily assign the different protein domains of a RIFIN to any RIFIN. Moreover, the skilled person is also aware of databases for protein domain prediction, for example “NCBI conserved domain search” (www.ncbi.nlm.nih.gov/Structure/cdd/wrpsb.cgi), “SMART” (smartembl-heidelberg.de/), InterPro protein (www.ebi.ac.uk/interpro/), “PredictProtein” (https://www.predictprotein.org/), and the like.

The second variable (V2) domain (also known as “hypervariable domain”; (6)) comprises approximately 170 polymorphic residues and is predicted to be exposed on the cell surface (i.e. extracellular localization). A role of the second variable (V2) domain (hypervariable domain; (6)) in antigenic variation was suggested. However, the actual orientation of RIFINs within membrane is still debatable, since only the C-terminal transmembrane domain (7) is widely accepted as transmembrane domain, whereas the more N-terminal “hydrophobic patch” (5) was initially suggested to be a second transmembrane domain, which is, however, under discussion (for review see Templeton T.)., 2009, Molecular & Biochemical Parasitology 166: 109-116, in particular FIG. 3 suggesting different models). Depending on whether the hydrophobic patch (5) indeed constitutes a second transmembrane domain the N-terminus of the RIFINS including the N-terminal semi-conserved domain ((4); C1, also referred to as “constant region 1”) is located either intracellularly or extracellularly (cf. Templeton T.)., 2009, Molecular & Biochemical Parasitology 166: 109-116, in particular FIG. 3 suggesting different models).

Binding to a second variable (V2) domain of a RIFIN, binding to an N-terminal semi-conserved domain of a RIFIN and/or binding to a RIFIN, preferably to RIFIN PF3D7_1400600 and/or to RIFIN PF3D7_1040300, may be easily determined. For example, 1) a RIFIN may be expressed on the surface of cell of mammalian cells (293 Expi) used for transfection and they are then stained with the protein in question, e.g. with the (exemplary) antibodies and/or the (“exon”-)fusion proteins as described herein; or 2) a RIFIN may be expressed as fusion protein in mammalian cells (293 Expi) and they are then tested if they bind to the protein in question, e.g. to the (exemplary) antibodies and/or the (“exon”-)fusion proteins as described herein by ELISA.

Methods for testing proteins, in particular (monoclonal and/or polyclonal) antibodies, for their binding affinities are well known in the art. One possibility among others is to characterize the binding affinity of an antibody by means of a sandwich ELISA by using the target peptide as well as negative controls (e.g. the same peptide with L-amino acids only). The ELISA limit can—without being limited thereto—be calculated on blank replicates as follows:

ELISA limit=average (negative control)+(3×standard deviation of negative control).

If the sample value is less or equal to the ELISA limit the tested antibody may be considered to have no affinity to the target peptide. If the sample value exceeds the ELISA limit the tested antibody may be considered to exhibit affinity to the target peptide. Moreover, the higher the sample value, the stronger is the affinity of the tested antibody for the target.

Preferably, the polypeptide comprised by the pharmaceutical composition according to the present invention comprises or consists of a second variable (V2) domain of a RIFIN, which is able to bind to a LAIR-1 fragment as described above. More preferably, the polypeptide comprised by the pharmaceutical composition according to the present invention comprises or consists of a second variable (V2) domain of a RIFIN as described herein, but does not comprise an N-terminal semi-conserved domain of a RIFIN as described herein. Alternatively, it is also preferred that the polypeptide comprised by the pharmaceutical composition according to the present invention comprises (i) a second variable (V2) domain of a RIFIN as described herein and (ii) an N-terminal semi-conserved domain of a RIFIN as described herein.

In the following, the second variable (V2) domain of a RIFIN, which is comprised by the polypeptide (which is, in turn, comprised by the pharmaceutical composition according to the present invention), is described in more detail.

Preferably, the second variable (V2) domain of a RIFIN is the second variable (V2) domain of an A-type RIFIN. RIFINs are grouped into A-type RIFINs (also referred to as A-RIFINs) and B-type RIFINs (also referred to as B-RIFINs), whereby A-type RIFINs have an N-terminal semi-conserved domain (4), which is 25 amino acids longer than that of B-type RIFINs Qoannin N. et al., 2008, BMC genomics 9:19). In the context of the present invention a polypeptide comprising or consisting of the second variable (V2) domain of an A-type RIFIN is preferred.

Preferably, the second variable (V2) domain of a RIFIN comprises or consists of an amino acid sequence according to SEQ ID NO: 625:

HXTXXXXXAXXXDXE

wherein X is any amino acid.

More preferably, the second variable (V2) domain of a RIFIN comprises or consists of an amino acid sequence according to SEQ ID NO: 626:

HYTXXXXXAXXIDTE

wherein X is any amino acid.

Preferably, the second variable (V2) domain of a RIFIN, which may or may not comprise an amino acid sequence according to SEQ ID NO: 625 or 626, comprises or consists of an amino acid sequence according to SEQ ID NO: 627:

IXXXRXXLXXXXXXXXXMV

wherein X is any amino acid.

More preferably, the second variable (V2) domain of a RIFIN, which may or may not comprise an amino acid sequence according to SEQ ID NO: 625 or 626, comprises or consists of an amino acid sequence according to SEQ ID NO: 628:

ICXXRXXLGXXXKXGXXMV

wherein X is any amino acid.

It is also preferred that the second variable (V2) domain of a RIFIN comprises or consists of an amino acid sequence according to SEQ ID NO: 629:

HXTXXXXXAXXXDXEXXXXXXXXXXXXXXIXXXRXXLXXXXXXXXXMV

wherein X is any amino acid.

More preferably, the second variable (V2) domain of a RIFIN comprises or consists of an amino acid sequence according to SEQ ID NO: 630:

HYTXXXXXAXXIDTEXXXXCXXXXXXXXXICXXRXXLGXXXKXGXXMV

wherein X is any amino acid.

Preferably, the second variable (V2) domain of a RIFIN, which may or may not comprise an amino acid sequence according to SEQ ID NO: 625, 626, 627 and/or 628, preferably according to SEQ ID NO: 629 or 630, comprises or consists of an amino acid sequence according to SEQ ID NO: 631:

KXXXXXSXXXXXHXT

wherein X is any amino acid.

More preferably, the second variable (V2) domain of a RIFIN, which may or may not comprise an amino acid sequence according to SEQ ID NO: 625, 626, 627 and/or 628, preferably according to SEQ ID NO: 629 or 630, comprises or consists of an amino acid sequence according to SEQ ID NO: 632:

LKXXXXXSFXXXXHYT

wherein X is any amino acid.

Preferably, the second variable (V2) domain of a RIFIN, which may or may not comprise an amino acid sequence according to SEQ ID NO: 625, 626, 627, 628, 631 and/or 632, preferably according to SEQ ID NO: 629 or 630, comprises or consists of an amino acid sequence according to SEQ ID NO: 633:

MVXQXXXTXXXXXXXXKXXXXXE

wherein X is any amino acid.

More preferably, the second variable (V2) domain of a RIFIN, which may or may not comprise an amino acid sequence according to SEQ ID NO: 625, 626, 627, 628, 631 and/or 632, preferably according to SEQ ID NO: 629 or 630, comprises or consists of an amino acid sequence according to SEQ ID NO: 634:

MVXQKXAITXXXXXXXXKXXXXAEA

wherein X is any amino acid.

It is more preferred that the second variable (V2) domain of a RIFIN comprises or consists of an amino acid sequence according to SEQ ID NO: 635:

KXXXXXSXXXXXHXTXXXXXAXXXDXEXXXXXXXXXXXXXXIXXXRXXLX
XXXXXXXXMVXQXXXTXXXXXXXXKXXXXXE

wherein X is any amino acid.

Even more preferably, the second variable (V2) domain of a RIFIN comprises or consists of an amino acid sequence according to SEQ ID NO: 636:

LKXXXXXSFXXXXHYTXXXXXAXXIDTEXXXXCXXXXXXXXXICXXRXXL
GXXXKXGXXMVXQKXAITXXXXXXXXKXXXXAEA

wherein X is any amino acid.

Most preferably, the second variable (V2) domain of a RIFIN comprises or consists of an amino acid sequence according to SEQ ID NO: 637:

IXXLXXXAWKXXALXXAXXXAXKAGXAAGXXAGXXXGXXXXIXXXXXXXX
XXXLKXXXXXSFXXXXHYTXXXXXAXXIDTEXXXXCXXXXXXXXXICXXR
XXLGXXXKXGXXMVXQKXAITXXXXXXXXKXXXXAEAXXXXXAXXXXAX
XXXXXTXAIXXXXXXXXT

wherein X is any amino acid.

In a particular preferred embodiment, the second variable (V2) domain of a RIFIN comprises or consists of an amino acid sequence according to SEQ ID NO: 638 or 639 (shown below) or according to a functional sequence variant thereof as described herein (which has a sequence identity of at least 70%, preferably at least 80%, more preferably at least 90%, even more preferably at least 95% and particularly preferably at least 99% to a reference sequence—which is here SEQ ID NO: 638 or 639).

SEQ ID NO: 638:
IAALAVNAWKTTALKNAIAAAQKAGDAAGKIAGESKGVETIIGILEQYYS
IYELKGTPLKSFFATTHYTDISNIATVIDTELNTSCGLNSLANQAICGLR
TKLGLVAKPGQVMVTQKEAITKMITNVVHKSEITAEAAKTEVAATKTAAA
IKMNTEAIEAATTPYYT
(second variable (V2) domain of RIFIN
PF3D7_1400600)
SEQ ID NO: 639:
IGQLGLDAWKAAALVTAKELAEKAGAAAGLKAGDIHGMKIVIEGLKALKV
DTLKSGIFNSFVNNSHYTEVTGLAIAIDTEMNEVCSATYIGIHPICVVRE
KLGVIPKAGGTMVKQKDAITNVLKQALEKATQSAEALSETTAEDVAAKLT
AQKTGAINTIFMSNQT
(second variable (V2) domain of RIFIN
PF3D7_1040300).

In the following, the N-terminal semi-conserved domain of a RIFIN, which is optionally comprised by the polypeptide (which is, in turn, comprised by the pharmaceutical composition according to the present invention), is described in more detail.

The polypeptide comprised by the pharmaceutical composition according to the present invention may (also) comprise an N-terminal semi-conserved domain of a RIFIN. Such an N-terminal semi-conserved domain of a RIFIN may or may not be able to bind to a LAIR-1 fragment as described herein. Preferably, the polypeptide comprises (i) a second variable (V2) domain of a RIFIN, which is able to bind to a LAIR-1 fragment as described herein, and (ii) an N-terminal semi-conserved domain of a RIFIN, which is not able to bind to a LAIR-1 fragment as described herein.

However, in another preferred embodiment, the polypeptide comprises an N-terminal semi-conserved domain of a RIFIN, which is able to bind to a LAIR-1 fragment as described herein. Such a polypeptide may or may not further comprise a second variable (V2) domain of a RIFIN as described herein, preferably, the polypeptide does not comprise a second variable (V2) domain of a RIFIN as described herein.

Preferably, the N-terminal semi-conserved domain of a RIFIN is the N-terminal semi-conserved domain of an A-type RIFIN. RIFINs are grouped into A-type RIFINs (also referred to as A-RIFINs) and B-type RIFINs (also referred to as B-RIFINs), whereby A-type RIFINs have an N-terminal semi-conserved domain (4), which is 25 amino acids longer than that of B-type RIFINs (Joannin N. et al., 2008, BMC genomics 9:19). In the context of the present invention a polypeptide comprising or consisting of the N-terminal semi-conserved domain of an A-type RIFIN (which is about 25 amino acids longer than that of a B-type RIFIN) is preferred.

Preferably, the polypeptide comprised by the pharmaceutical composition according to the present invention comprises an N-terminal semi-conserved domain of a RIFIN, which comprises an amino acid sequence according to SEQ ID NO: 530:

CXXYXXXXXDXDXXMKXVMXXFXXXTXQRFHEYDEXXXXXRXXCKXXCDK
EIQKIILKDXXEKEXXXKXXXLXTDXXXXXIPTCXCEKSXXDKXEKXXXX
CXXXLXXXXXXXXXXXX

wherein X may be any amino acid.

More preferably, the polypeptide comprised by the pharmaceutical composition according to the present invention comprises an N-terminal semi-conserved domain of a RIFIN, which comprises an amino acid sequence according to SEQ ID NO: 531:

CXXYXXTXXDSDXXMKXVMXXFXXXTXQRFHEYDEXXXXXRXXCKXXCDK
EIQKIILKDXXEKEXXXKXXXLXTDXXXXXIPTCXCEKSXXDKXEKXXXX
CXXXLXXXXXXXXXXXX

wherein X may be any amino acid.

Even more preferably, the polypeptide comprised by the pharmaceutical composition according to the present invention comprises an N-terminal semi-conserved domain of a RIFIN, which comprises an amino acid sequence according to SEQ ID NO: 532:

CELYSPTNYDSDPEMKRVMQQFXXXTXQRFHEYDEXXXXXRXXCKXXCDK
EIQKIILKDXXEKEXXXKXXXLXTDXXXXXIPTCXCEKSXXDKXEKXXXX
CXXXLXXXXXXXXXXXX

wherein X may be any amino acid.

Particularly preferably, the polypeptide comprised by the pharmaceutical composition according to the present invention comprises an N-terminal semi-conserved domain of a RIFIN, which comprises an amino acid sequence according to SEQ ID NO: 533:

CELYSPTNYDSDPEMKRVMQQFXDRTTQRFHEYDEXXXXXRXXCKXQCDK
EIQKIILKDXXEKEXXXKXXTLXTDIXXXXIPTCVCEKSLADKXEKXCLX
CXXXLGGXVXXXXGXLG

wherein X may be any amino acid.

Most preferably, the polypeptide comprised by the pharmaceutical composition according to the present invention comprises an N-terminal semi-conserved domain of a RIFIN, which comprises an amino acid sequence according to SEQ ID NO: 534 or 535 or a functional sequence variant thereof, preferably an amino acid sequence according to SEQ ID NO: 534 or a functional sequence variant thereof.

SEQ ID NO: 534
CELYSPTNYDSDPEMKRVMQQFVDRTTQRFHEYDESLQSKRKQCKDQCDK
EIQKIILKDKIEKEFTEKLSTLQTDITTKDIPTCVCEKSLADKMEKVCLK
CAQNLGGIVAPSTGVLG
(N-terminal semi-conserved domain of
RIFIN PF3D7_1400600)
SEQ ID NO: 535
CELYSPTNYDSDPEMKRVMQQFHDRTTQRFHEYDERMKTTRQECKEQCDK
EIQKIILKDRLEKELMDKFATLHTDIQSDAIPTCVCEKSLADKTEKFCLN
CGVQLGGGVLQASGLLG
(N-terminal semi-conserved domain of
RIFIN PF3D7_1040300)

Preferably, the polypeptide comprised by the pharmaceutical composition according to the present invention is a recombinant polypeptide. A “recombinant polypeptide” is a polypeptide, which is not naturally occurring, in particular a polypeptide which is prepared, expressed, created or isolated by recombinant means.

Preferably, the polypeptide comprised by the pharmaceutical composition according to the present invention comprises a RIFIN as described above, preferably the polypeptide is a RIFIN as described above.

In the context of the present invention it is also preferred that the polypeptide comprised by the pharmaceutical composition according to the present invention comprises a truncated RIFIN, preferably the polypeptide is a truncated RIFIN. A truncated RIFIN is a RIFIN as described herein, which is truncated at the C-terminus, at the N-terminus or at both, C_{—} and N-terminus.

Preferably, a truncated RIFIN is truncated at the C-terminus. Preferably a truncated RIFIN lacks one or more of the following protein domains: putative signal peptide (SP), first variable domain (V1), a plasmodium export element (PEXEL), N-terminal semi-conserved domain (C1, also referred to as “constant region 1”), hydrophobic patch (proposed to be a transmembrane domain (TM1)), second variable domain (also known as hypervariable domain (V2)), (second) transmembrane domain (TM2), and/or C-terminal conserved domain (C2). More preferably, a truncated RIFIN lacks the C-terminal conserved domain (C2). Even more preferably, a truncated RIFIN lacks the (second) transmembrane domain (TM2), and the adjacent C-terminal conserved domain (C2). Particularly preferably, a truncated RIFIN lacks the hydrophobic patch (proposed to be a transmembrane domain (TM1)), the second variable domain (also known as hypervariable domain (V2)), the (second) transmembrane domain (TM2), and the C-terminal conserved domain (C2).

More preferably, a truncated RIFIN is truncated at the N-terminus. Preferably a truncated RIFIN lacks one or more of the following protein domains: putative signal peptide (SP), first variable domain (V1), a plasmodium export element (PEXEL), N-terminal semi-conserved domain (C1, also referred to as “constant region 1”), hydrophobic patch (proposed to be a transmembrane domain (TM1)), second variable domain (also known as hypervariable domain (V2)), (second) transmembrane domain (TM2), and/or C-terminal conserved domain (C2). Preferably, a truncated RIFIN lacks the N-terminal putative signal peptide (SP). More preferably, a truncated RIFIN lacks the N-terminal putative signal peptide (SP) and the first variable domain (V1). Even more preferably, a truncated RIFIN lacks the N-terminal putative signal peptide (SP), the first variable domain (V1) and the plasmodium export element (PEXEL). Most preferably, a truncated RIFIN lacks the N-terminal putative signal peptide (SP), the first variable domain (V1), the plasmodium export element (PEXEL) and the N-terminal semi-conserved domain (C1, also referred to as “constant region 1”). Particularly preferably, a truncated RIFIN lacks the N-terminal putative signal peptide (SP), the first variable domain (V1), the plasmodium export element (PEXEL), the N-terminal semi-conserved domain (C1, also referred to as “constant region 1”) and the hydrophobic patch (proposed to be a transmembrane domain (TM1)).

It is also preferred that a truncated RIFIN is truncated at the N-terminus and at the C-terminus. In this case, the preferred embodiments for N-terminal and C-terminal truncations as described above are preferably combined. For example, a truncated RIFIN lacks the N-terminal putative signal peptide (SP) and the C-terminal conserved domain (C2). Preferably, a truncated RIFIN lacks the N-terminal putative signal peptide (SP), the first variable domain (V1) and the C-terminal conserved domain (C2). It is also preferred that a truncated RIFIN lacks the N-terminal putative signal peptide (SP), the (second) transmembrane domain (TM2), and the adjacent C-terminal conserved domain (C2). More preferably, a truncated RIFIN lacks the N-terminal putative signal peptide (SP), the first variable domain (V1), the plasmodium export element (PEXEL), the (second) transmembrane domain (TM2), and the adjacent C-terminal conserved domain (C2). Even more preferably, a truncated RIFIN lacks the N-terminal putative signal peptide (SP), the first variable domain (V1), the plasmodium export element (PEXEL), the N-terminal semi-conserved domain (C1, also referred to as “constant region 1”), the (second) transmembrane domain (TM2), and the adjacent C-terminal conserved domain (C2). Most preferably, a truncated RIFIN lacks the N-terminal putative signal peptide (SP), the first variable domain (V1), the plasmodium export element (PEXEL), the N-terminal semi-conserved domain (C1, also referred to as “constant region 1”), the hydrophobic patch (proposed to be a transmembrane domain (TM1)), the (second) transmembrane domain (TM2), and the adjacent C-terminal conserved domain (C2).

Preferably, the polypeptide comprised by the pharmaceutical composition according to the present invention comprises an A-type RIFIN as described above, preferably the polypeptide is an A-type RIFIN as described above.

More preferably, the polypeptide comprised by the pharmaceutical composition according to the present invention comprises an amino acid sequence according to SEQ ID NO: 538 (PF3D7_1040300) or according to SEQ ID NO: 536 (PF3D7_1400600) or a functional sequence variant thereof, preferably the polypeptide comprised by the pharmaceutical composition according to the present invention consists of an amino acid sequence according to SEQ ID NO: 538 (PF3D7_1040300) or according to SEQ ID NO: 536 (PF3D7_1400600) or a functional sequence variant thereof. Even more preferably, the polypeptide comprised by the pharmaceutical composition according to the present invention comprises an amino acid sequence according to SEQ ID NO: 536 (PF3D7_1400600) or a functional sequence variant thereof, preferably the polypeptide comprised by the pharmaceutical composition according to the present invention consists of an amino acid sequence according to SEQ ID NO: 536 (PF3D7_1400600) or a functional sequence variant thereof.

The amino acid sequences, as well as exemplary nucleic acid sequences encoding them, of RIFINs PF3D7_1040300 and PF3D7_1400600 are shown below in Table 3.

TABLE 3
Amino acid sequences and nucleic acid sequences of
RIFINs PF3D7_1400600 and PF3D7_1040300.

| SEQ ID | | |
|---|---|---|
| NO | Description | Sequence |
| 536 | PF3D7_1400600 | MKDHYINILLFALPLNILVYNQRNYYITPRHTETNRSLCE |
| | aa | CELYSPTNYDSDPEMKRVMQQFVDRTTQRFHEYDESLQSK |
| | | RKQCKDQCDKEIQKIILKDKIEKEFTEKLSTLQTDITTKD |
| | | IPTCVCEKSLADKMEKVCLKCAQNLGGIVAPSTGVLGEIA |
| | | ALAVNAWKTTALKNAIAAAQKAGDAAGKIAGESKGVETII |
| | | GILEQYYSIYELKGTPLKSFFATTHYTDISNIATVTDTEL |
| | | NTSCGLNSLANQAICGLRTKLGLVAKPGQVMVTQKEAITK |
| | | MITNVVHKSEITAEAAKTEVAATKTAAAIKMNTEAIEAAT |
| | | TPYYTPIIASIVAIVVIVLIMVIIYLILRYRRKKKMKKKL |
| | | QYIKLLN* |
| 537 | PF3D7_1400600 | ATGAAAGACCATTATATTAATATATTATTGTTTGCTCTTC |
| | nucl | CATTAAATATATTGGTATATAATCAAAGGAACTATTACAT |
| | | TACACCACGTCATACAGAAACCAACAGATCTTTATGTGAA |
| | | TGTGAATTATATTCACCTACGAACTATGATAGTGATCCCG |
| | | AAATGAAAAGGGTAATGCAACAATTTGTGGATCGTACAAC |
| | | ACAACGATTTCACGAATATGATGAAAGTTTGCAAAGTAAA |
| | | CGAAAGCAATGCAAAGATCAATGCGATAAAGAAATCCAAA |
| | | AAATTATATTAAAAGATAAAATCGAAAAGGAATTTACAGA |
| | | AAAATTATCAACATTACAAACAGATATAACGACTAAAGAC |
| | | ATACCCACCTGTGTTTGCGAAAAATCCTTGGCGGACAAAA |
| | | TGGAAAAAGTATGCTTGAAATGTGCACAAAATTTGGGAGG |
| | | TATTGTTGCACCCTCTACAGGAGTATTAGGCGAAATTGCT |
| | | GCACTTGCTGTAAATGCCTGGAAAACTACGGCACTTAAGA |
| | | ACGCTATTGCGGCAGCTCAAAAAGCAGGTGATGCGGCCGG |
| | | TAAAATTGCGGGGGAATCCAAGGGTGTTGAAACAATTATT |
| | | GGAATATTAGAACAATATTACTCTATATATGAGTTAAAAG |
| | | GAACACCATTGAAATCCTTTTTTGCTACAACGCATTATAC |
| | | TGATATCTCAAATATTGCTACTGTTATTGATACGGAATTG |
| | | AATACGTCTTGTGGGTTGAATTCCTTAGCTAATCAGGCTA |
| | | TTTGCGGTCTTCGTACGAAATTAGGTCTTGTTGCAAAACC |
| | | TGGTCAAGTTATGGTTACACAGAAAGAAGCTATAACAAAG |
| | | ATGATAACCAACGTTGTTCATAAATCTGAAATTACTGCTG |
| | | AAGCTGCAAAGACTGAGGTGGCTGCAACTAAAACAGCAGC |
| | | AGCTATAAAGATGAACACAGAAGCTATAGAAGCTGCAACT |
| | | ACTCCTTACTATACTCCTATAATAGCATCCATCGTTGCAA |
| | | TAGTGGTCATAGTTTTAATTATGGTGATAATTTATTTGAT |
| | | TTTACGTTATCGAAGAAAAAAAAAAATGAAGAAAAAACTC |
| | | CAATATATAAAATTATTAAATTAA |
| 538 | PF3D7_1040300 | MKFNYTNIILFSLSLNILLLSSRVYNKRNHKSIILHTSNE |
| | aa | NPIKTHRSLCECELYSPTNYDSDPEMKRVMQQFHDRTTQR |
| | | FHEYDERMKTTRQECKEQCDKEIQKIILKDRLEKELMDKF |
| | | ATLHTDIQSDAIPTCVCEKSLADKTEKFCLNCGVQLGGGV |
| | | LQASGLLGGIGQLGLDAWKAAALVTAKELAEKAGAAAGLK |
| | | AGDIHGMKIVIEGLKALKVDTLKSGIFNSFVNNSHYTEVT |
| | | GLAIAIDTEMNEVCSATYIGIHPICVVREKLGVIPKAGGT |
| | | MVKQKDAITNVLKQALEKATQSAEALSETTAEDVAAKLTA |
| | | QKTGAINTIFMSNQTAIIASIVAIVVIVLIMVIIYLILRY |
| | | RRKKKMKKKLQYIKLLEE |
| 539 | PF3D7_1040300 | ATGAAGTTCAATTACACTAATATAATATTATTTTCCCTTT |
| | nucl | CATTAAATATATTGTTATTATCATCACGGGTATACAATAA |
| | | AAGGAATCATAAAAGCATTATACTTCATACATCAAACGAA |
| | | AACCCAATAAAAACACATAGATCATTATGCGAATGCGAAT |
| | | TATATTCACCTACGAACTATGATAGTGATCCCGAAATGAA |
| | | AAGGGTAATGCAACAATTTCATGATCGTACAACACAACGA |
| | | TTTCACGAATACGACGAAAGGATGAAAACTACACGCCAAG |
| | | AATGTAAAGAACAATGCGATAAAGAAATACAAAAAATTAT |
| | | TTTAAAAGACAGATTAGAAAAAGAATTAATGGACAAATTT |
| | | GCCACACTACACACAGATATACAAAGTGATGCTATTCCAA |
| | | CATGTGTTTGCGAAAAGTCGTTAGCAGATAAAACAGAAAA |
| | | ATTTTGTCTGAACTGTGGGGTGCAACTAGGAGGTGGTGTG |
| | | TTGCAAGCTTCGGGTTTATTAGGAGGAATTGGTCAACTTG |
| | | GGCTAGATGCATGGAAAGCAGCCGCGTTGGTAACTGCTAA |
| | | GGAACTTGCCGAAAAAGCCGGTGCTGCAGCAGGTCTTAAA |
| | | GCAGGTGATATCCATGGTATGAAAATAGTTATTGAAGGAT |
| | | TAAAAGCATTGAAAGTAGATACATTAAAATCTGGAATATT |
| | | TAATTCCTTTGTTAATAACAGCCATTATACTGAAGTCACA |
| | | GGGCTTGCTATTGCTATTGATACTGAAATGAATGAGGTGT |
| | | GTTCAGCGACGTATATTGGTATTCATCCTATCTGCGTTGT |
| | | TCGTGAGAAATTAGGTGTAATACCAAAGGCTGGTGGAACA |
| | | ATGGTTAAACAGAAAGATGCTATAACAAATGTGTTAAAGC |
| | | AAGCTCTTGAAAAAGCTACACAAAGTGCTGAAGCACTTTC |
| | | TGAGACTACTGCTGAAGACGTTGCTGCTAAACTCACAGCT |
| | | CAAAAGACGGGTGCGATAAATACTATATTTATGAGTAATC |
| | | AGACTGCTATTATTGCTTCCATCGTTGCAATAGTAGTTAT |
| | | AGTTTTAATTATGGTGATAATATATTTAATTTTACGTTAT |
| | | CGACGAAAAAAAAAAATGAAGAAAAAACTCCAATATATCA |
| | | AATTATTAGAAGAATAG |

Optionally, the pharmaceutical composition according to the present invention may also comprise one or more additional pharmaceutically active components and/or one or more pharmaceutically inactive components.

Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions according to the present invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, like Synagis™ and Herceptin™, for reconstitution with sterile water containing a preservative). The pharmaceutical composition may be prepared for topical administration e.g., as an ointment, cream or powder. The pharmaceutical composition may be prepared for oral administration e.g., as a tablet or capsule, as a spray, or as a syrup (optionally flavored). The pharmaceutical composition may be prepared for pulmonary administration e.g., as an inhaler, using a fine powder or a spray. The pharmaceutical composition may be prepared as a suppository or pessary. The pharmaceutical composition may be prepared for nasal, aural or ocular administration e.g., as drops. The pharmaceutical composition may be in kit form, designed such that a combined composition is reconstituted just prior to administration to a subject. For example, a lyophilized polypeptide can be provided in kit form with sterile water or a sterile buffer.

It is preferred that the active ingredient in the composition is the polypeptide comprised by the pharmaceutical composition as described herein. As such, it may be susceptible to degradation in the gastrointestinal tract. Thus, if the composition is to be administered by a route using the gastrointestinal tract, the composition may contain agents which protect the polypeptide comprised by the pharmaceutical composition as described herein from degradation but which release the polypeptide once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH in particular between 5.5 and 8.5, for example between 6 and 8, for example about 7. The pH may be maintained by the use of a buffer. The pharmaceutical composition may be sterile and/or pyrogen free. The pharmaceutical composition may be isotonic with respect to humans. The pharmaceutical composition of the invention may be supplied in hermetically-sealed containers.

Within the scope of the invention are compositions present in several forms for different administration methods; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the polypeptide may be in dry form, for reconstitution before use with an appropriate sterile liquid. A vehicle is typically understood to be a material that is suitable for storing, transporting, and/or administering a compound, such as a pharmaceutically active compound, in particular the polypeptide as described herein. For example, the vehicle may be a physiologically acceptable liquid, which is suitable for storing, transporting, and/or administering a pharmaceutically active compound, in particular the antibodies according to the present invention. Once formulated, the pharmaceutical composition according to the present invention may be administered directly to the subject. In one embodiment the pharmaceutical composition according to the present invention is adapted for administration to mammalian, e.g., human subjects.

The pharmaceutical composition according to the present invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical composition according to the present invention. Preferably, the pharmaceutical composition according to the present invention may be prepared for oral administration, e.g. as tablets, capsules and the like, for topical administration, or as injectable, e.g. as liquid solutions or suspensions. Solid forms suitable for solution in, or suspension in, liquid vehicles prior to injection may also be prepared.

For injection, e.g. intravenous, cutaneous or subcutaneous injection, or injection at the site of affliction, the active ingredient will preferably be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection.

Preferably, preservatives, stabilizers, buffers, antioxidants and/or other additives may be included in the pharmaceutical composition according to the present invention, as required.

Whether it is a polypeptide, a nucleic acid molecule, or a cell according to the present invention that is to be given to an individual by administering the pharmaceutical composition according to the present invention, administration is preferably in a “prophylactically effective amount” (of the polypeptide, the nucleic acid molecule, or the cell according to the present invention) or a “therapeutically effective amount” (of the polypeptide, the nucleic acid molecule, or the cell according to the present invention) (as the case may be), this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The pharmaceutical composition according to the present invention may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the polypeptide as defined above, is combined with emulsifying and suspending agents. If desired, certain sweetening, flavoring or coloring agents may also be added.

The inventive pharmaceutical composition may also be administered topically. For topical applications, the pharmaceutical composition according to the present invention may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition according to the present invention may be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule, whereby in the context of the present invention a multiple dose schedule is preferred.

For example, the pharmaceutical composition according to the present invention may be administered daily, e.g. once or several times per day, e.g. once, twice, three times or four times per day, preferably once or twice per day, more preferable once per day, for 1, 2, 3, 4, 5, 6, 7, 8, 9, 10, 11, 12, 13, 14, 15, 16, 17, 18, 19, 20 or 21 or more days, e.g. daily for 1, 2, 3, 4, 5, 6 months. Preferably, the pharmaceutical composition according to the present invention may be administered weekly, e.g. once or twice, preferably once per week, for 1, 2, 3, 4, 5, 6, 7, 8, 9, 10, 11, 12, 13, 14, 15, 16, 17, 18, 19, 20 or 21 or more weeks, e.g. weekly 1, 2, 3, 4, 5, 6, 7, 8, 9, 10, 11, or 12 months or weekly for 2, 3, 4, or 5 years.

In particular, it is preferred that for a single dose, e.g. a daily, weekly or monthly dose, preferably for a weekly dose, the amount of the polypeptide in the pharmaceutical composition according to the present invention, does not exceed 150 mg, preferably does not exceed 100 mg, more preferably does not exceed 50 mg, even more preferably does not exceed 20 mg, and particularly preferably does not exceed 10 mg. This amount of polypeptide preferably refers to a single dose as described above, which is for example administered daily, weekly etc. as described above. Such a low amount of the polypeptide comprised by the pharmaceutical composition as described herein could be produced and formulated in a stable form (e.g., in a lyophilized formulation, where for instance previous studies have shown that monoclonal antibodies preserved by lyophilization are stable for 33 months at 40° C. and 5 months at 50° C.) and at an affordable cost.

Pharmaceutical compositions typically include an effective amount of one or more polypeptides as described herein, in particular polypeptides comprising or consisting of a second variable (V2) domain and/or an N-terminal semi-conserved domain of a RIFIN as described herein, i.e. an amount that is sufficient to treat, ameliorate, attenuate or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose will generally be from about 0.005 to about 100 mg/kg, preferably from about 0.0075 to about 50 mg/kg, more preferably from about 0.01 to about 10 mg/kg, even more preferably from about 0.02 to about 5 mg/kg, and particularly preferably from about 0.03 to about 1 mg/kg of the polypeptide (e.g. amount of the polypeptide in the pharmaceutical composition) in relation to the bodyweight (e.g., in kg) of the individual to which it is administered.

Preferably, the pharmaceutical composition according to the present invention may include two or more (e.g., 2, 3, 4, 5 etc.) polypeptides as described herein, in particular polypeptides comprising or consisting of a second variable (V2) domain and/or an N-terminal semi-conserved domain of a RIFIN as described herein, to provide an additive or synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

It is also preferred that the pharmaceutical composition according to the present invention may comprise one or more (e.g., 2, 3, etc.) antibodies according the invention and one or more (e.g., 2, 3, etc.) additional antibodies, preferably against malaria, more preferably against P. falciparum, even more preferably against a variant surface antigen of P. falciparum, and particularly preferably against a P. falciparum RIFIN. Further, the administration of a polypeptide as described herein, in particular a polypeptide comprising or consisting of a second variable (V2) domain and/or an N-terminal semi-conserved domain of a RIFIN as described herein, together with antibodies specific to other antigens, are within the scope of the invention. The polypeptide as described herein, in particular a polypeptide comprising or consisting of a second variable (V2) domain and/or an N-terminal semi-conserved domain of a RIFIN as described herein, can be administered either combined/simultaneously or at separate times from antibodies specific to other cytokines or, more generally, to other antigens.

In one embodiment, a composition of the invention may include polypeptides as described herein, in particular polypeptides comprising or consisting of a second variable (V2) domain and/or an N-terminal semi-conserved domain of a RIFIN as described herein, wherein the polypeptides may make up at least 50% by weight (e.g., 60%, 70%, 75%, 80%, 85%, 90%, 95%, 97%, 98%, 99% or more) of the total protein in the pharmaceutical composition. In such a pharmaceutical composition, the polypeptides as described herein, in particular polypeptides comprising or consisting of a second variable (V2) domain and/or an N-terminal semi-conserved domain of a RIFIN as described herein, are preferably in purified form.

The present invention also provides a method of preparing a pharmaceutical composition comprising the steps of: (i) preparing a polypeptide as described herein; and (ii) admixing the optionally purified polypeptide with one or more pharmaceutically-acceptable carriers.

The pharmaceutical composition according to the present invention may include an antimicrobial, particularly if packaged in a multiple dose format. They may comprise detergent e.g., a Tween (polysorbate), such as Tween 80. Detergents are generally present at low levels e.g., less than 0.01%. The pharmaceutical composition according to the present invention may also include a sodium salt (e.g., sodium chloride) to give tonicity. For example, a concentration of 10±2 mg/ml NaCl is typical.

Further, the pharmaceutical composition according to the present invention may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilisation may be adjusted to between 5 and 8, or between 5.5 and 7, or around 6.1 prior to lyophilisation.

The pharmaceutical composition according to the present invention may also comprise one or more immunoregulatory agents. One or more of the immunoregulatory agents may include an adjuvant.

Preferably, the pharmaceutical composition according to the present invention as described herein is a vaccine. The term “vaccine” as used herein is typically understood to be a prophylactic or therapeutic material providing at least one antigen, preferably an immunogen. The antigen or immunogen may be derived from any material that is suitable for vaccination.

In the context of the present invention, the antigen/immunogen is the polypeptide as described herein, in particular the polypeptide comprising or consisting of a second variable (V2) domain and/or an N-terminal semi-conserved domain of a RIFIN as described herein. Thus, the antigen or immunogen is derived from a RIFIN. The antigen or immunogen stimulates the body's adaptive immune system to provide an adaptive immune response. In particular, an “antigen” or an “immunogen” refers typically to a substance which may be recognized by the immune system, preferably by the adaptive immune system, and which is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells.

Thus, the present invention also provides a vaccine, which comprises the polypeptide as defined above, in particular the polypeptide comprising or consisting of a second variable (V2) domain and/or an N-terminal semi-conserved domain of a RIFIN as described above, and optionally one or more pharmaceutically active components. The term “pharmaceutically active component” refers to any compound or composition which, when administered to a human or animal induces a desired pharmacologic, immunogenic, and/or physiologic effect by local and/or systemic action. In one embodiment, the inventive vaccine composition may comprise optionally an inactive carrier (vaccine excipient), such as e.g. aluminium salts, egg protein, formaldehyde, monosodium glutamate, or e.g. carbohydrates, including, but not limited to, sorbitol, mannitol, starch, sucrose, dextran, glutamate or glucose, or e.g. proteins, including, but not limited to, dried milk, serum albumin, casein.

Preferably, the vaccine according to the invention comprises one or more adjuvants selected from the group comprising mineral salts, surface-active agents, microparticles, cytokines, hormones, antigen constructs, polyanions, polyacrylics, or water-in-oil emulsions. Accordingly, the inventive vaccine may comprise one or more, e.g. two, three, four or more adjuvants in addition to the polypeptide comprising or consisting of a second variable (V2) domain and/or an N-terminal semi-conserved domain of a RIFIN as described above. The term “adjuvant,” as used herein, refers to compounds which, when administered to an individual, such as e.g. a human, or tested in vitro, increase the immune response to an antigen, such as the polypeptide comprising or consisting of a second variable (V2) domain and/or an N-terminal semi-conserved domain of a RIFIN as described above, in the individual or test system to which said antigen is administered. The use of an adjuvant typically enhances the immune response of the individual to the antigen (e.g. the polypeptide as described herein, in particular a polypeptide comprising or consisting of a second variable (V2) domain and/or an N-terminal semi-conserved domain of a RIFIN as described herein) by rendereing the antigen more strongly immunogenic. The adjuvant effect may also enable the use of a lower the dose of antigen necessary to achieve an immune response in said individual, e.g. a lower dose of the inventive vaccine may be required to achieve the desired immune response.

More specifically, the inventive vaccine may comprise one or more adjuvants selected from the group comprising mineral salts, surface-active agents, microparticles, cytokines, hormones, antigen constructs, polyanions, polyacrylics, or water-in-oil emulsions. Accordingly, the inventive vaccine composition may comprise one more adjuvants, e.g. one, two, three, four, five, six, seven, eight, nine, or ten or more adjuvants. For example the inventive vaccine may comprise one, two, three, four, five, six, seven, eight, nine, or ten or more adjuvants selected from aluminum (“Alum”), aluminum hydroxide, aluminum phosphate, calcium phosphate, nonionic block polymer surfactants, virosomes, Saponin (QS-21), meningococcal outer membrane proteins (Proteosomes), immune stimulating complexes (ISCOMs), Cochleates Dimethyl dioctadecyl ammonium bromide (DDA), Avridine (CP20,961), vitamin A, vitamin E, cell wall skeleton of Mycobacterium phlei (Detox®), muramyl dipeptides and tripeptides, Threonyl MDP (SAF-1), Butyl-ester MDP (Murabutide®), Dipalmitoyl phosphatidylethanolamine MTP, Monophosphoryl lipid A, Klebsiella pneumonia glycoprotein, Bordetella pertussis, Bacillus Calmette-Guérin, Vibrio cholerae and Escherichia coli heat labile enterotoxin, Trehalose dimycolate, CpG oligodeoxynucleotides, Interleukin-2, Interferon-γ, Interferon-β, granulocyte-macrophage colony stimulating factor, dehydroepiandrosterone, Flt3 ligand, 1,25-dihydroxy vitamin D3, Interleukin-1, Interleukin-6, Interleukin-12, human growth hormone, 2-microglobulin, lymphotactin, Polyanions, e.g. Dextran, double-stranded polynucleotides, polyacrylics, e.g. polymethylmethacrylate, acrylic acid crosslinked with allyl sucrose (Carbopol 934P), or e.g N-acetyl-glucosamine-3yl-acetyl-L-alanyl-D-isoglutamine (CGP-11637), gamma inulin+aluminum hydroxide (Algammulin), human dendritic cells, lysophosphatidyl glycerol, stearyl tyrosine, tripalmitoyl pentapeptide, Carbopol 974P NF polymer, water-in-oil emulsions, mineral oil (Freund's incomplete), vegetable oil (peanut oil), squalene and squalane, oil-in-water emulsions, Squalene+Tween-80+Span 85 (MF59), or e.g. liposomes, or e.g. biodegradable polymer microspheres, lactide and glycolide, polyphosphazenes, beta-glucan, or e.g. proteinoids. A list of typically used vaccine adjuvants may also be found in e.g. “Vaccine Adjuvants”, edited by D. T. O'Hogan, Humana Press 2000. The adjuvant comprised in the inventive vaccine composition may also include e.g. a synthetic derivative of lipid A, some of which are TLR-4 agonists, and include, but are not limited to: 0M174 (2-deoxy-6-o-[2-deoxy-2-[(R)-3-dodecanoyloxytetra-decanoylamino]-4-o-phosphono-D-D-glucopyranosyl]-2-[(R)-3-hydroxy-tetradecanoylamino]-p-D-glucopyranosyldihydrogen-phosphate), (WO 95/14026) OM-294-DP (3S, 9R)-3˜[(R)-dodecanoyloxytetradecanoylam, [(R)-3-hydroxytetradecanoylamino] decan-1,10-diol, 1,10-bis(dihydrogenophosphate) (WO 99/64301 and WO 00/0462) OM 197 MP-Ac DP(3S-,9R)-3-D(R)-dodecanoyl-oxytetradecanoylamino]-4-oxo-5-aza-9-[(R)-3-hydroxytetra-decanoylamino]decan-1,10-diol,1-dihydrogenophosphate-10-(6-aminohexanoate) (WO 01/46127). For example the inventive vaccine may comprise only one of the above adjuvants, or e.g. two of the above adjuvants, e.g. combination adjuvants such as e.g. Alum and MPL, or Oil-in-water emulsion and MPL and QS-21, or liposomes and MPL and QS21.

It is particularly preferred that the vaccine according to the invention comprises an adjuvant selected from the group comprising Alum, Ribi (Monophosphoryl lipid A, MPL), or MF59. Accordingly, the inventive vaccine composition may comprise Alum, or Ribi (Monophosphoryl lipid A, MPL), or MF59, or e.g. Alum and Ribi, or e.g. Alum and MF59, or e.g. Ribi and MF59.

The inventive vaccine may be formulated as a liquid formulation, or alternatively and as a preferred embodiment as a lyophilized formulation. The term “liquid formulation” as used for the inventive vaccine refers to a water-based formulation, in particular, a formulation that is an aqueous solution. The liquid composition may e.g. further comprise ethanol, or e.g. non-ionic detergents, or e.g. anti-oxidants, such as oxygen scavengers to prevent oxidation of the inventive vaccine, e.g. vitamin E, or e.g. vitamin C. The water for use with the inventive liquid vaccine may e.g. be USP-grade water for injection. The inventive liquid vaccine formulation may for example also consist of, or comprise an emulsion. An emulsion comprises a liquid suspended in another liquid, typically with the aid of an emulsifier. The inventive liquid vaccine may also e.g. be a microemulsion, which is a thermodynamically stable solution that is clear upon visual inspection.

Preferably, the inventive vaccine may be provided as a lyophilized formulation. The term “lyophilized formulation” as used with the inventive vaccine means a freeze-dried formulation prepared by the processes known in the art, such as e.g. those provided in “Cryopreservation and Freeze-Drying Protocols” (2007), J G Day, G N Stacey (eds)., Springer, ISBN 978-1-58829-377-0, and comprising as essential ingredient the polypeptide as described herein, in particular a polypeptide comprising or consisting of a second variable (V2) domain and/or an N-terminal semi-conserved domain of a RIFIN as described herein.

More specifically, the inventive vaccine may comprise a buffer selected from the group of phosphate buffer, Na-acetate buffer, Tris buffer, MOPS buffer, preferably the buffer is a phosphate buffer. Accordingly, the inventive vaccine composition may comprise a phosphate buffer, or a Na-acetate buffer, or a Tris buffer, or a MOPS buffer, preferably the inventive vaccine comprises a phosphate buffer. For example, the inventive vaccine composition may comprise a a Na-acetate buffer in a concentration of about 0.1 mM to about 500 mM, or of about 1 mM to about 250 mM, or of about 10 mM to about 125 mM, or of about 25 mM to about 100 mM, or of about 50 mM to about 75 mM, or of about 60 mM to about 70 mM, or of about 7.5 mM, 10 mM, 12.5 mM, 15 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM to about 125 mM, 130 mM, 135 mM, 137 mM, 140 mM, 145 mM, 150 mM, 155 mM, 160 mM, 165 mM, 170 mM, 175 mM, 180 mM, 185 mM, 190 mM, 195 mM, 200 mM, or e.g. about 1 mM, 2 mM, 3 mM, 4 mM, 5 mM, 7.5 mM, 10 mM, 12.5 mM, 15 mM, 17.5 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM, 125 mM, 150 mM, 200 mM, 250 mM, or about 500 mM. The inventive vaccine composition may also comprise a Tris buffer (tris(hydroxymethyl)aminomethane), in the above concentrations, or e.g. a 3-(N-morpholino)propanesulfonic acid) (MPOS) buffer in the above concentrations, or e.g. a (4-(2-hydroxyethyl)-1-piperazineethanesulfonic acid) (HEPES) buffer in the above concentrations, or e.g. a 2-(N-morpholino)ethanesulfonic acid (MES) buffer in the above concentrations, or e.g. a N-cyclohexyl-3-aminopropanesulfonic acid (CAPS) buffer in the above concentrations. According to a preferred embodiment, the inventive vaccine comprises a phosphate buffer. Accordingly, the total phosphate concentrations for the buffer may be from about 5 mM to about 500 mM, or from about 7.5 mM, 10 mM, 12.5 mM, 15 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM to about 125 mM, 130 mM, 135 mM, 137 mM, 140 mM, 145 mM, 150 mM, 155 mM, 160 mM, 165 mM, 170 mM, 175 mM, 180 mM, 185 mM, 190 mM, 195 mM, 200 mM, or e.g. 7.5 mM, 10 mM, 12.5 mM, 15 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 137 mM, 140 mM, 145 mM, 150 mM, 155 mM, 160 mM, 165 mM, 170 mM, 175 mM, 180 mM, 185 mM, 190 mM, 195 mM, 200 mM, 225 mM, 250 mM, 300 mM, 325 mM, 350 mM, 400 mM, 450 mM, or 500 mM. For example, the inventive vaccine composition may also comprise PBS as phosphate buffer, which comprises 137 mM NaCl, 2.7 mM KCl, 10 mM Na₂HPO₄ and 1.8 mM KH₂PO₄, or e.g. NaCl in a concentration of about 158 mM.

More specifically, the inventive vaccine is buffered by the buffer at a pH range of about pH 7-9, preferably of about pH 7.5 to about pH 8.8, or of about pH 7.8 to about pH 8.6, or of about pH 8.0 to about pH 8.4. Accordingly, the inventive vaccine is buffered by a buffer as disclosed above, e.g. by a Tris buffer, MOPS buffer, Na-acetate buffer, or phosphate buffer in concentrations as disclosed above. For example the inventive vaccine may be buffered at a pH range of about pH 7-9, e.g. of about pH 7.0, pH 7.1, pH 7.2, pH 7.3, pH 7.4, pH 7.5, pH 7.6, pH 7.7, pH 7.8, pH 7.9, pH 8.0 to about pH 8.4, pH 8.5, pH 8.6, pH 8.7, pH 8.8, pH 8.9, pH 9.0, or e.g. of about pH 7.8 to about pH 8.6, e.g. of about pH 7.8, pH 7.9, pH 8.0, pH 8.1, pH 8.2 to about pH 8.4, pH 8.5, pH 8.6, or at a pH range of about pH 8.0 to about pH 8.4, e.g. at about pH 8.0, pH 8.1, pH 8.2, pH 8.3, or pH 8.4. The pH of the buffer system as used above may be calculated according to any method known in the art, such as e.g. the Henderson-Haselbalch equation (pH=pKa+log₁₀([A⁻]/[HA]))

Moreover, the vaccine according to the invention may also comprise a preservative. The term “preservative” as used in the present invention shall mean any compound that when added to the inventive vaccine prolongs the time the inventive vaccine may be stored prior to use. Preservatives included with the inventive vaccine may include e.g. albumin, phenols, glycine, Thimerosal, benzalkonium chloride, polyaminopropyl biguanide, phenoxyethanol, merthiolate, gentamicin, neomycin, nystatin, amphotericin B, tetracycline, penicillin, streptomycin, polymyxin B, and any combination thereof. Accordingly, the inventive vaccine composition may comprise any of the above compounds in a concentration of about 0.001% (w/v)/(w/w) to about 5% (w/v)/(w/w), or of about 0.02% (w/v)/(w/w), 0.03% (w/v)/(w/w), 0.04% (w/v)/(w/w), 0.05% (w/v)/(w/w), 0.06% (w/v)/(w/w), 0.07% (w/v)/(w/w), 0.08% (w/v)/(w/w), 0.09% (w/v)/(w/w), 0.1% (w/v)/(w/w) to about 0.2% (w/v)/(w/w), 0.25% (w/v)/(w/w), 0.3% (w/v)/(w/w), 0.4% (w/v)/(w/w), 0.5% (w/v)/(w/w), 0.6% (w/v)/(w/w), 0.7% (w/v)/(w/w), 0.8% (w/v)/(w/w), 0.9% (w/v)/(w/w), 1.0% (w/v)/(w/w), 1.25% (w/v)/(w/w), 1.5% (w/v)/(w/w), 2.0% (w/v)/(w/w), 2.25% (w/v)/(w/w), 2.5% (w/v)/(w/w), 3% (w/v)/(w/w), 3.5% (w/v)/(w/w), 4% (w/v)/(w/w), 4.5% (w/v)/(w/w), 5% (w/v)/(w/w).

In a preferred embodiment, the inventive vaccine as disclosed above is for use in the vaccination of humans. The term “vaccination” as used in the context of the inventive vaccine refers to the administration of antigenic material, such as e.g. the inventive vaccine, to stimulate an individual's immune system to develop an adaptive immune response to a pathogen, such as P. falciparum in order to prevent, or reduce the risk of infection. Accordingly, the inventive vaccine will be administered to a human in a dose suitable to induce a sufficient immune response, e.g. an immune response that comprises T- and B-cell memory and neutralizing antibodies to provide protective immunity against P. falciparum, preferably against more than one strain of P. falciparum.

### Medical Treatments and Uses

In a further aspect, the present invention provides the use of the pharmaceutical composition, in particular the vaccine, according to the present invention in prevention and/or treatment of malaria, preferably of P. falciparum-induced malaria.

Malaria is caused by Plasmodium parasites. The parasites are spread to people through the bites of infected Anopheles mosquitoes, called “malaria vectors”, which bite mainly between dusk and dawn. There are four parasite species that cause malaria in humans Plasmodium falciparum, Plasmodium vivax, Plasmodium malariae, and Plasmodium ovale. Plasmodium falciparum and Plasmodium vivax are the most common causes of malaria. Plasmodium falciparum is the most deadly.

Within the scope of the invention are several forms and routes of administration of the polypeptide as described herein, the nucleic acid, the vector, the cell, or the pharmaceutical composition as described herein. This applies also in the context of the use of the polypeptide, the nucleic acid, the vector, the cell as described herein, in particular regarding preferred forms and routes of administration.

In a further aspect, the present invention provides the use of the pharmaceutical composition, in particular the vaccine, according to the present invention in diagnosis of malaria, preferably of P. falciparum-induced malaria.

Methods of diagnosis may include contacting a polypeptide as defined above, in particular the polypeptide comprising or consisting of a second variable (V2) domain and/or an N-terminal semi-conserved domain of a RIFIN as described above, with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, preferably serum.

In the context of the present invention, diagnosis of malaria is preferably done by contacting a polypeptide as defined above, in particular the polypeptide comprising or consisting of a second variable (V2) domain and/or an N-terminal semi-conserved domain of a RIFIN as described above, with a sample, which is preferably isolated, e.g. from a patient. The sample is preferably an (isolated) sample comprising erythrocytes, more preferably a blood sample.

The methods of diagnosis may also include the detection of an antigen/protein complex, e.g. an antigen/antibody complex, in particular following the contacting of a polypeptide with a sample. Such a detection step is typically performed at the bench, i.e. without any contact to the human or animal body. Examples of detection methods include e.g. ELISA (enzyme-linked immunosorbent assay).

Diagnosis of malaria, e.g. in a blood sample, is important for example (i) for a subject, which may potentially suffer from malaria, and (ii) for blood transfusions to avoid transmission of malaria by infected blood transfusions. In particular in this context the polypeptide as defined above, in particular the polypeptide comprising or consisting of a second variable (V2) domain and/or an N-terminal semi-conserved domain of a RIFIN as described above, may be very useful to determine whether a blood sample is malaria-free.

The present invention also provides the use of the pharmaceutical composition, in particular the vaccine, according to the present invention in determining whether a subject has antibodies against P. falciparum.

This may also include contacting a polypeptide as defined above, in particular the polypeptide comprising or consisting of a second variable (V2) domain and/or an N-terminal semi-conserved domain of a RIFIN as described above, with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, preferably serum.

Determining whether a subject has antibodies against P. falciparum is preferably done by contacting a polypeptide as defined above, in particular the polypeptide comprising or consisting of a second variable (V2) domain and/or an N-terminal semi-conserved domain of a RIFIN as described above, with a sample, which is preferably isolated, e.g. from a patient. The sample is preferably an (isolated) sample comprising erythrocytes, more preferably a blood sample.

This methods may also include the detection of an antigen/protein complex, e.g. an antigen/antibody complex, in particular following the contacting of a polypeptide with a sample. Such a detection step is typically performed at the bench, i.e. without any contact to the human or animal body. Examples of detection methods include e.g. ELISA (enzyme-linked immunosorbent assay).

The present invention also provides a method for treating a subject, comprising the step of administering to the subject the pharmaceutical composition, in particular the vaccine, according to the present invention. The present invention also provides a method of preventing and/or treating malaria in a subject, wherein the method comprises administering to a subject in need thereof the pharmaceutical composition, in particular the vaccine, according to the present invention in a therapeutically effective amount as described herein. The present invention also provides a method of vaccinating a subject, wherein the method comprises administering to a subject the pharmaceutical composition, in particular the vaccine, according to the present invention in a therapeutically effective amount as described herein.

In some embodiments the subject may be a human. One way of checking efficacy of therapeutic treatment involves monitoring disease symptoms after administration of the composition of the invention. Treatment can be a single dose schedule or a multiple close schedule.

Polypeptide for Use in Prevention and/or Treatment of Malaria

In a further aspect, the present invention also provides an isolated polypeptide comprising or consisting of a second variable (V2) domain of a RIFIN and/or an N-terminal semi-conserved domain of a RIFIN for use in prevention and/or treatment of malaria, preferably of P. falciparum-malaria. Preferably, the isolated polypeptide for use in prevention and/or treatment of malaria, preferably of P. falciparum-malaria comprises or consists of a second variable (V2) domain of a RIFIN.

Malaria is caused by Plasmodium parasites. The parasites are spread to people through the bites of infected Anopheles mosquitoes, called “malaria vectors”, which bite mainly between dusk and dawn. There are four parasite species that cause malaria in humans Plasmodium falciparum, Plasmodium vivax, Plasmodium malariae, and Plasmodium ovale. Plasmodium falciparum and Plasmodium vivax are the most common causes of malaria. Plasmodium falciparum is the most deadly.

Within the scope of the invention are several forms and routes of administration of the polypeptide as described herein.

Thereby, the “polypeptide comprising or consisting of a second variable (V2) domain of a RIFIN and/or an N-terminal semi-conserved domain of a RIFIN” is a polypeptide as described above in the context of the pharmaceutical composition comprising such a peptide. Accordingly, preferred embodiments of a polypeptide as described above comprised by the pharmaceutical composition according to the present invention apply accordingly to the isolated polypeptide comprising or consisting of a second variable (V2) domain of a RIFIN and/or an N-terminal semi-conserved domain of a RIFIN for use in prevention and/or treatment of malaria, preferably of P. falciparum-malaria.

In the following, preferred embodiments are briefly summarized, whereby in these briefly summarized aspects the same detailed description and more preferred embodiments apply to the isolated polypeptide comprising or consisting of a second variable (V2) domain of a RIFIN and/or an N-terminal semi-conserved domain of a RIFIN for use in prevention and/or treatment of malaria, preferably of P. falciparum-malaria as described for the polypeptide comprised by the pharmaceutical composition according to the present invention.

Thus, the present invention also provides an isolated polypeptide comprising or consisting of a second variable (V2) domain of a RIFIN and/or an N-terminal semi-conserved domain of a RIFIN for use in prevention and/or treatment of malaria, preferably of P. falciparum-malaria, wherein the polypeptide comprising or consisting of a second variable (V2) domain of a RIFIN and/or the N-terminal semi-conserved domain of a RIFIN is able to bind to a LAIR-1 fragment, wherein the LAIR-1 fragment has an amino acid sequence according to SEQ ID NO: 1:

XXLPRPXXSXXXXXXXXLGSXXTXVCRGPXGXXTFRLXXXXXXX₁YX₂XX
EXVXXX₃XPXXSEARFRXXSVXXGXXGXXRCXYYXX₄X₅XWSXXSXXXXX
XVK

wherein
X is any amino acid or no amino acid;
X₁ is T, L, G, I, R, K or no amino acid; however, if X₂ is N, X₃ is A, X₄ is P and X₅ is P, then X₁ is L, G, I, R, K or no amino acid;
X₂ is N, S or T; however, if X₁ is T, X₃ is A, X₄ is P and X₅ is P, then X₂ is S or T;
X₃ is A, T, P, or V; however, if X₁ is T, X₂ is N, X₄ is P and X₅ is P, then X₃ is T, P, or V;
X₄ is P, S, A, or D; however, if X₁ is T, X₂ is N, X₃ is A and X₅ is P, then X₄ is S, A, or D; and
X₅ is P, R, or S; however, if X₁ is T, X₂ is N, X₃ is A and X₄ is P, then X₅ is R or S;
   and wherein the LAIR-1 fragment has at least 70% amino acid sequence identity to amino acids 24 to 121 of native human LAIR-1 (SEQ ID NO: 10).

Preferably, the polypeptide for use in prevention and/or treatment of malaria, preferably of P. falciparum-malaria, according to the present invention is able to bind to a LAIR-1 fragment, wherein the LAIR-1 fragment has an amino acid sequence according to any of SEQ ID NOs 12, 14, 16, 18, 20, 22, 24, 26, 28, 30, 32, 34, 36, 38, 40, 42, 44, 46, 48, 50, 52 and 54 or to a functional sequence variant thereof, more preferably the LAIR-1 fragment has an amino acid sequence according to any of SEQ ID NO: 28, 34, 42, 46, 50 and 52 or is a functional sequence variant thereof.

Preferably, the polypeptide for use in prevention and/or treatment of malaria, preferably of P. falciparum-malaria, according to the present invention is able to bind to a LAIR-1 fragment, wherein the LAIR-1 fragment has an amino acid sequence according to SEQ ID NO: 34 or a functional sequence variant thereof.

Preferably, the polypeptide for use in prevention and/or treatment of malaria, preferably of P. falciparum-malaria, according to the present invention is able to bind to an antibody having a heavy chain variable region comprising the amino acid sequence of SEQ ID NO: 340 or a functional sequence variant thereof and a light chain variable region comprising the amino acid sequence of SEQ ID NO: 341 or a functional sequence variant thereof.

Preferably, the polypeptide for use in prevention and/or treatment of malaria, preferably of R falciparum-malaria, according to the present invention comprises a second variable (V2) domain of a RIFIN, which is preferably able to bind to a LAIR-1 fragment as described herein. More preferably, the polypeptide for use in prevention and/or treatment of malaria, preferably of P. falciparum-mal aria, according to the present invention comprises a second variable (V2) domain of a RIFIN, which is preferably able to bind to a LAIR-1 fragment as described herein, but does not comprise an N-terminal semi-conserved domain of a RIFIN as described herein. Even more preferably, the polypeptide for use in prevention and/or treatment of malaria, preferably of P. falciparum-malaria, according to the present invention comprises (i) a second variable (V2) domain of a RIFIN, which is preferably able to bind to a LAIR-1 fragment as described herein, and (ii) an N-terminal semi-conserved domain of a RIFIN, which is preferably not able to bind to a LAIR-1 fragment as described herein.

Preferably, the second variable (V2) domain of a RIFIN comprised by the polypeptide for use in prevention and/or treatment of malaria, preferably of P. falciparum-malaria, according to the present invention, is the second variable (V2) domain of an A-type RIFIN.

Preferably, the second variable (V2) domain of a RIFIN comprised by the polypeptide for use in prevention and/or treatment of malaria, preferably of P falciparum-malaria, according to the present invention, comprises an amino acid sequence according to SEQ ID NO: 625:

HXTXXXXXAXXXDXE

wherein X is any amino acid.

More preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 626:

HYTXXXXXAXXIDTE

wherein X is any amino acid.

Preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 627:

IXXXRXXLXXXXXXXXXMV

wherein X is any amino acid.

More preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 628:

ICXXRXXLGXXXKXGXXMV

wherein X is any amino acid.

Preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 629:

HXTXXXXXAXXXDXEXXXXXXXXXXXXXXIXXXRXXLXXXXXXXXXMV

wherein X is any amino acid.

More preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 630:

HYTXXXXXAXXIDTEXXXXCXXXXXXXXXICXXRXXLGXXXKXGXXMV

wherein X is any amino acid.

Preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 631:

KXXXXXSXXXXXHXT

wherein X is any amino acid.

More preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 632:

LKXXXXXSFXXXXHYT

wherein X is any amino acid.

Preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 633:

MVXQXXXTXXXXXXXXKXXXXXE

wherein X is any amino acid.

More preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 634:

MVXQKXAITXXXXXXXXKXXXXAEA

wherein X is any amino acid.

Preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 635:

KXXXXXSXXXXXHXTXXXXXAXXXDXEXXXXXXXXXXXXXXIXXXRXXLX
XXXXXXXXMVXQXXXTXXXXXXXXKXXXXXE

wherein X is any amino acid.

More preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 636:

LKXXXXXSFXXXXHYTXXXXXAXXIDTEXXXXCXXXXXXXXXICXXRXXL
GXXXKXGXXMVXQKXAITXXXXXXXXKXXXXAEA

wherein X is any amino acid.

Even more preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 637:

IXXLXXXAWKXXALXXAXXXAXKAGXAAGXXAGXXXGXXXXIXXXXXXXX
XXXLKXXXXXSFXXXXHYTXXXXXAXXIDTEXXXXCXXXXXXXXXICXXR
XXLGXXXKXGXXMVXQKXAITXXXXXXXXKXXXXAEAXXXXXAXXXXAXX
XXXXTXAIXXXXXXXXT

wherein X is any amino acid.

Particularly preferably, the second variable (V2) domain of a RIFIN comprised by the polypeptide for use in prevention and/or treatment of malaria, preferably of P. falciparum-malaria, according to the present invention, comprises an amino acid sequence according to SEQ ID NO: 638 or 639 or a functional sequence variant thereof.

The polypeptide for use in prevention and/or treatment of malaria, preferably of P. falciparum-malaria, according to the present invention may also comprise an N-terminal semi-conserved domain of a RIFIN, which is preferably able to bind to a LAIR-1 fragment as described herein. Such a polypeptide comprising an N-terminal semi-conserved domain of a RIFIN may or may not comprise a second variable (V2) domain of a RIFIN as described herein.

Preferably, the polypeptide for use in prevention and/or treatment of malaria, preferably of P. falciparum-malaria, according to the present invention comprises the N-terminal semi-conserved domain of an A-type RIFIN.

Preferably, the polypeptide for use in prevention and/or treatment of malaria, preferably of P. falciparum-malaria, according to the present invention comprises the N-terminal semi-conserved domain of a RIFIN, wherein the RIFIN comprises an amino acid sequence according to SEQ ID NO: 530:

CXXYXXXXXDXDXXMKXVMXXFXXXTXQRFHEYDEXXXXXRXXCKXXCDK
EIQKIILKDXXEKEXXXKXXXLXTDXXXXXIPTCXCEKSXXDKXEKXXXX
CXXXLXXXXXXXXXXXX

wherein X is any amino acid.

More preferably, the polypeptide for use in prevention and/or treatment of malaria, preferably of P. falciparum-malaria, according to the present invention comprises the N-terminal semi-conserved domain of a RIFIN, wherein the RIFIN comprises an amino acid sequence according to SEQ ID NO: 531

CXXYXXTXXDSDXXMKXVMXXFXXXTXQRFHEYDEXXXXXRXXCKXXCDK
EIQKIILKDXXEKEXXXKXXXLXTDXXXXXIPTCXCEKSXXDKXEKXXXX
CXXXLXXXXXXXXXXXX

wherein X is any amino acid.

Even more preferably, the polypeptide for use in prevention and/or treatment of malaria, preferably of P. falciparum-malaria, according to the present invention comprises the N-terminal semi-conserved domain of a RIFIN, wherein the RIFIN comprises an amino acid sequence according to SEQ ID NO: 532:

CELYSPTNYDSDPEMKRVNIQQFXXXTXQRFHEYDEXXXXXRXXCKXXCD
KEIQKIILKDXXEKEXXXKXXXLXTDXXXXXIPTCXCEKSXXDKXEKXXX
XCXXXLXXXXXXXXXXXX

wherein X is any amino acid.

Particularly preferably, the polypeptide for use in prevention and/or treatment of malaria, preferably of P. falciparum-malaria, according to the present invention comprises the N-terminal semi-conserved domain of a RIFIN, wherein the RIFIN comprises an amino acid sequence according to SEQ ID NO: 533:

CELYSPTNYDSDPEMKRVMQQFXDRTTQRFHEYDEXXXXXRXXCKXQCDK
EIQKIILKDXXEKEXXXKXXTLXTDIXXXXIPTCVCEKSLADKXEKXCLX
CXXXLGGXVXXXXGXLG

wherein X is any amino acid.

Preferably, the polypeptide for use in prevention and/or treatment of malaria, preferably of P. falciparum-malaria, according to the present invention comprises an amino acid sequence according to SEQ ID NO: 534 or 535 or a functional sequence variant thereof.

Preferably, the polypeptide for use in prevention and/or treatment of malaria, preferably of P. falciparum-malaria, according to the present invention is a recombinant polypeptide.

Preferably, the polypeptide for use in prevention and/or treatment of malaria, preferably of P. falciparum-malaria, according to the present invention comprises a RIFIN, preferably the polypeptide is a RI FIN.

Preferably, the polypeptide for use in prevention and/or treatment of malaria, preferably of P. falciparum-malaria, according to the present invention comprises a truncated RIFIN, preferably the polypeptide is a truncated RIFIN.

Preferably, the polypeptide for use in prevention and/or treatment of malaria, preferably of P. falciparum-malaria, according to the present invention comprises an A-type RIFIN, preferably the polypeptide is an A-type RI FI N.

Preferably, the polypeptide for use in prevention and/or treatment of malaria, preferably of P. falciparum-malaria, according to the present invention comprises an amino acid sequence according to SEQ ID NO: 538 (PF3D7_1040300) or according to SEQ ID NO: 536 (PF3D7_1400600) or a functional sequence variant thereof.

### Nucleic Acid Molecule According to the Present Invention

In another aspect, the present invention provides a nucleic acid molecule encoding a polypeptide according to the present invention as described herein for use in prevention and/or treatment of malaria, preferably of P. falciparum-malaria.

A nucleic acid molecule is a molecule comprising, preferably consisting of nucleic acid components. The term nucleic acid molecule preferably refers to DNA or RNA molecules. In particular, it is used synonymous with the term “polynucleotide”. Preferably, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

Preferably, the nucleic acid molecule encoding a polypeptide according to the present invention as described herein comprises or consists of a nucleic acid sequence according to SEQ ID NO: 540 or 541 or of a functional sequence variant thereof, more preferably the nucleic acid molecule encoding a polypeptide according to the present invention as described herein comprises or consists of a nucleic acid sequence according to SEQ ID NO: 540 or of a functional sequence variant thereof.

SEQ ID NO: 540

TGTGAATTATATTCACCTACGAACTATGATAGTGATCCCGAAATGAAAAG
GGTAATGCAACAATTTGTGGATCGTACAACACAACGATTTCACGAATATG
ATGAAAGTTTGCAAAGTAAACGAAAGCAATGCAAAGATCAATGCGATAAA
GAAATCCAAAAAATTATATTAAAAGATAAAATCGAAAAGGAATTTACAGA
AAAATTATCAACATTACAAACAGATATAACGACTAAAGACATACCCACCT
GTGTTTGCGAAAAATCCTTGGCGGACAAAATGGAAAAAGTATGCTTGAAA
TGTGCACAAAATTTGGGAGGTATTGTTGCACCCTCTACAGGAGTATTA
GGC
(PF3D71400600 N-terminal semi-conserved domain)

SEQ ID NO: 541

TGCGAATGCGAATTATATTCACCTACGAACTATGATAGTGATCCCGAAAT
GAAAAGGGTAATGCAACAATTTCATGATCGTACAACACAACGATTTCACG
AATACGACGAAAGGATGAAAACTACACGCCAAGAATGTAAAGAACAATGC
GATAAAGAAATACAAAAAATTATTTTAAAAGACAGATTAGAAAAAGAATT
AATGGACAAATTTGCCACACTACACACAGATATACAAAGTGATGCTATTC
CAACATGTGTTTGCGAAAAGTCGTTAGCAGATAAAACAGAAAAATTTTGT
CTGAACTGTGGGGTGCAACTAGGAGGTGGTGTGTTGCAAGCTTCGGGTTT
ATTAGGA
(PF3D71040300 N-terminal semi-conserved domain)

Preferably, the nucleic acid molecule encoding a polypeptide according to the present invention as described herein comprises or consists of a nucleic acid sequence according to SEQ ID NO: 537 or 539 or of a functional sequence variant thereof, more preferably the nucleic acid molecule encoding a polypeptide according to the present invention as described herein comprises or consists of a nucleic acid sequence according to SEQ ID NO: 537 or of a functional sequence variant thereof. SEQ ID NO: 537 and 539 encode full-length RIFINs PF3D71400600 and PF3D71040300, respectively (cf. Table 3).

Preferably, the nucleic acid molecule as described herein may be used for the manufacture of a medicament for prevention and/or treatment of malaria, preferably of P. falciparum-malaria. In particular, the nucleic acid molecule as described herein may be used for the expression of a polypeptide as described herein, in particular a polypeptide comprising or consisting of a second variable (V2) domain of a RIFIN and/or an N-terminal semi-conserved domain of a RIFIN. For such an expression a vector and a cell may be used as described in the following.

### Vector According to the Present Invention

In another aspect, the present invention provides a vector comprising the nucleic acid molecule according to the present invention, for example a nucleic acid molecule as described above. Such a vector according to the present invention is preferably a storage vector, an expression vector, a cloning vector, or a transfer vector, more preferably an expression vector or a cloning vector, and even more preferably an expression vector.

The term “vector” refers to a nucleic acid molecule, preferably to an artificial nucleic acid molecule, i.e. a nucleic acid molecule which does not occur in nature. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired polypeptide comprising or consisting of a second variable (V2) domain of a RIFIN and/or an N-terminal semi-conserved domain of a RIFIN as described herein. An expression vector may be used for production of expression products such as RNA, e.g. mRNA, or peptides, polypeptides or proteins. For example, an expression vector may comprise sequences needed for transcription of a sequence stretch of the vector, such as a promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. Preferably, a vector is a DNA molecule. For example, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector.

### Cell According to the Present Invention

In another aspect, the present invention provides a cell expressing the polypeptide as described herein, in particular the polypeptide comprising or consisting of a second variable (V2) domain of a RIFIN and/or an N-terminal semi-conserved domain of a RIFIN, or comprising the vector according to the present invention.

Thus, cells transformed with a vector according to the present invention are also included within the scope of the invention. Examples of such cells include but are not limited to, eukaryotic cells, e.g., yeast cells, animal cells or plant cells. In one embodiment the cells are mammalian, e.g., human, CHO, HEK293T, PER.C6, NS0, myeloma or hybridoma cells.

In particular, the cell may be transfected with a vector according to the present invention, preferably with an expression vector. The term “transfection” refers to the introduction of nucleic acid molecules, such as DNA or RNA (e.g. mRNA) molecules, into cells, preferably into eukaryotic cells. In the context of the present invention, the term “transfection” encompasses any method known to the skilled person for introducing nucleic acid molecules into cells, preferably into eukaryotic cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. Preferably, the introduction is non-viral.

In a further aspect, the polypeptide as described herein, in particular the polypeptide comprising or consisting of a second variable (V2) domain of a RIFIN and/or an N-terminal semi-conserved domain of a RIFIN, the nucleic acid molecule according to the present invention, the vector according to the present invention and/or the cell according to the present invention may be used in diagnosis of malaria, preferably of P. falciparum-malaria as described herein.

Moreover, the polypeptide as described herein, in particular the polypeptide comprising or consisting of a second variable (V2) domain of a RIFIN and/or an N-terminal semi-conserved domain of a RIFIN, the nucleic acid molecule according to the present invention, the vector according to the present invention and/or the cell according to the present invention may be used in the identification of antibodies binding to infected erythrocytes, preferably of antibodies broadly binding to erythrocytes infected with more than one P. falciparum strain. To this end, the skilled person may assess binding of an antibody to a second variable (V2) domain of a RIFIN and/or binding to an N-terminal semi-conserved domain of a RIFIN and/or binding to a RIFIN, preferably to RIFIN PF3D7_1400600 and/or to RIFIN PF3D7_1040300. As described above, the polypeptide as described herein, in particular the polypeptide comprising or consisting of a second variable (V2) domain of a RIFIN and/or an N-terminal semi-conserved domain of a RIFIN and/or the RIFIN may be expressed as fusion protein in mammalian cells and they may be then tested whether they bind to an antibody in question as described herein.

The present invention is not to be limited in scope by the specific embodiments described herein. Indeed, various modifications of the invention in addition to those described herein will become apparent to those skilled in the art from the description and accompanying figures. Such modifications fall within the scope of the appended claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated by reference in their entirety. In the case of conflict, the present specification, including definitions, will control.

The following Figures, Sequences and Examples are intended to illustrate the invention further. They are not intended to limit the subject matter of the invention thereto.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 an example of staining of P. falciparum-infected erythrocytes by a broadly cross-reactive antibody (MGD21). IEs are stained with SYBR Green I dye (DNA) to discriminate them from uninfected erythrocytes used as control. The graph shows that MGD21 specifically binds only to IEs.

FIG. 2 shows an alignment of selected monoclonal antibodies of Example 1 (antibodies MGD21, MGD39, MGD47 and MGD55 in FIG. 2A and antibodies MGC1, MGC7, MGC37 and MGC29 in FIG. 2B) to an amino acid sequence encoded by the corresponding fragment of genomic LAIR-1 sequence (exon+intron).

FIG. 3 shows a scheme of the different antibody variants constructed in Example 3. The different elements of the 10 antibody constructs are compared to MGD21 and FI499 (unrelated antibody). MGD21 binds to erythrocytes infected with 9/9 primary P. falciparum isolates and carries the LAIR-1 exon+intron insertion. FI499 is an IgG antibody that binds influenza hemagglutinin and uses different V, D and I elements. Dα and Dβ indicate two putative D elements. GGGGS is an artificial linker. VH4-4, JH6, VK1-8 and JK5 and LAIR-1 intron and exon were also tested in the germline form (GL). For LAIR-1 the genomic sequence (ENSG00000167613) was used. In the right column, it is indicated whether the antibody (construct) binds to IEs as tested in Example 4.

FIG. 4 shows the results of Example 4 indicating that the mutated LAIR-1 exon is the only element required for mAb MGD21 binding to P. falciparum-infected erythrocytes. The antibodies were quantitated and tested for their capacity to stain IEs. “Con1” refers to “FI499_DexinDJ”, “Con2” refers to “FI499VJ_DexinD”, “Con3” refers to “MGD21_exin_longGS”, “Con4” refers to “MGD21_exin_shortGS”, “Con5” refers to “MGD21_NOexin”, “Con6” refers to “MGD21_NOin”, “Con7” refers to “MGD21_NOVD”, “Con8” refers to “MGD21GL_exinWT”, “Con9” refers to “MGD21_wholeGL”.

FIG. 5 shows a scheme of the different fusion proteins produced in Example 5. M1, M2, M3 and M4 are four different mouse IgG2b fusion proteins comprising the mutated LAIR-1 fragment according to the present invention, while H1 and H2 are two different human IgG1 fusion proteins comprising the mutated LAIR-1 fragment according to the present invention. M1 and H1 share the same variable region. M4 and H2 share the same variable region. “D_{α}” and “D_{β}” refer to the expression products of a first fragment and of a second fragment, different from the first fragment, of the same or different D (Diversity) gene segment element of a heavy chain variable region of an IgG-type antibody. “JH6” refers to the expression product of a J (Joining) gene segment element of a heavy chain variable region of an IgG-type antibody. “Exon” refers to the mutated LAIR-1 fragment. “Intron” and “Intron_{α}” refer to further LAIR-1 elements (expression products from one LAIR-1 intron fragment, whereby “Intron_{α}” is a fragment of “Intron”). “Hinge”, “CH2”, and “CH3” form together the constant region provided by the plasmid.

FIG. 6 shows for Example 6 that the mutated LAIR-1 fragment expressed as a fusion protein (cf. Example 5) binds to IEs. The four fusion proteins expressed in the mouse IgG2b fusion-protein vector were quantitated and tested for their capacity to stain IE.

FIG. 7 shows for Example 7 that fusion proteins comprising the mutated LAIR-1 fragment efficiently opsonize P. falciparum-infected erythrocytes. Parasites were stained with DAPI and mixed with a titration of antibodies and fusion proteins, followed by incubation with monocytes at 37° C. for 1 hour. Monocytes were stained with anti-CD14-APC and MFI of DAPI (A) and the of DAPI-positive monocytes (B) were calculated in CD14-positive populations. “DexinDJ” and “exon” are two fusion proteins expressed in the human IgG1 vector (cf. Example 5, also referred to as H1 and H2). FI499 is an unrelated antibody used as control. FIG. 7C shows agglutinates of 3D7-MGD21⁺ or 11019-MGD21⁺ IEs formed by MGD21 or MGC34. Scale bar, 25 μm.

FIG. 8 shows for Example 7 that antibodies MGD21, MG47, MGD55, MGC28 and MGC34 efficiently opsonize P. falciparum-infected erythrocytes. The IEs were stained with 4′,6-diamidino-2-phenylindole (DAPI), which was quantified in monocytes as a measure of phagocytosis. (A) Opsonic phagocytosis of 3D7-MGD2⁺ IEs by monocytes (n=3 for MGD21, MGD21 LALA and BKC3, n=2 for others). (B) Opsonic phagocytosis of 11019-MGD21⁺ IEs by monocytes (n=2).

FIG. 9 shows for Example 8 an alignment of the mutated LAIR-1 exon of the human monoclonal antibodies of Example 1 with amino acids 24 to 121 of native human LAIR-1 (SEQ ID NO: 14). Positions T67, N69, A77, P106 and P107 are shown in frames.

FIG. 10 shows for Example 8 the mutated LAIR-1 fragment modeled on the structure of the LAIR-1 extracellular domain. The LAIR-1 structure is shown as cartoon (left) and as surface (right). The five positions, at which a mutation may occur in the mutated LAIR-1 fragment as compared to the native LAIR-1 structure are highlighted in black.

FIG. 11 shows for Example 9 that the LAIR-1 fragment expressed as a fusion protein and carrying different combinations of mutations at positions T67, N69, A77, P106 and P107 binds to IEs while the same LAIR-1 fragment with no mutations does not bind to IEs. “LAIR1 ex” is the fusion protein carrying the LAIR1 fragment corresponding to the genomic sequence (Gene: LAIR1 ENSG00000167613). “LAIR1 ex+X” are the fusion protein carrying the LAIR1 fragment corresponding to the genomic sequence with one or more mutations (only mutated residues [L,S1,T,S2,R] are indicated according to the 5 most preferred mutations respectively: T67L, N69S, A77T, P106S, and P107R, whereby “L” refers to T67L, “S1” refers to N69S, “T” refers to A77T, “S2” refers to P106S and “R” refers to P107R. For instance “LAIR1ex+L” carries the mutation T67L and “LAIR1ex+S2” carries the mutation P106S.

FIG. 12 shows for Example 10 that the different mutations in the LAIR-1 fragment expressed as a fusion protein and carrying different combinations of mutations at positions T67, N69, A77, P106 and P107 (cf. Example 9) influence binding to collagen. The fusion proteins are the same shown in FIG. 10 (cf. Example 9).

FIG. 13 depicts a western blot showing MGD21 binding to erythrocyte ghosts and MGD21 immunoprecipitates (IP) prepared from 3D7-MGD21⁺ and 3D7-MGD21⁻ IEs (representative of n=2 independent experiments). Controls include uninfected erythrocytes (uEs) and immunoprecipitates with an irrelevant antibody (BKC3). Specific bands are marked with asterisks. Anti-human IgG was used as the secondary antibody, resulting in detection of antibodies used for immunoprecipitation alongside antigens of interest. Numbers on right indicate kDa.

FIG. 14 shows a Volcano plot from LC-MS analysis of MGD21 immunoprecipitates prepared from 3D7-MGD21⁺ IEs versus from 3D7-MGD21⁻ IEs (from n=4 independent experiments). Statistical significance was evaluated by Welch tests (P<0.01 for PF3D7_1400600).

FIG. 15 shows a heat map from LC-MS analysis showing RIFIN expression levels (calculated as intensity-based absolute quantification (iBAQ) scores) in erythrocyte ghosts prepared from 3D7-MGD21⁺ and 3D7-MGD21⁻ IEs (two experiments shown). Boxes with crosses indicate that expression levels are below the detection limit.

FIG. 16 shows the percentage of IEs (representative of n=2 independent experiments) stained by the antibodies. BKC3 is a negative control antibody.

FIG. 17 shows for Example 11 a western blot (A) showing MGD21 binding to immunoprecipitates (IP) prepared from 9605-MGD21⁻ and 9605-MGD21⁺ IEs (representative of n=2 independent experiments). Specific bands are marked with an asterisk. Anti-human IgG was used as the secondary antibody, resulting in detection of antibodies used for immunoprecipitation alongside antigens of interest. FIG. 16B shows percentage of 9605-MGD21⁻ and 9605-MGD21⁺ IEs recognized by representative MGC and MGD antibodies (representative of n=2 independent experiments).

FIG. 18 shows (A) the percentage of transfectecl CHO cells (n=1) stained by the antibodies. BKC3 is a negative control antibody. FIG. 17B shows MGD21 and BKC3 staining of CHO cells transfected with a specific (PF3D7_1400600) or an irrelevant (PF3D7_0100200) RIFIN (representative of n=5 independent experiments).

FIG. 19 shows binding of MGD21 (left) or of an Fc fusion protein containing the LAIR1 domain of MGD21 (right) to CHO cells transfected with RIFINs (PF3D7_1400600 and PF3D7_0100200), a RIFIN chimaera containing the constant region of PF3D7_0100200 and the variable region of PF3D7_1400600 (PF3D7_0100200c_1400600 v), or the inverse chimaera (PF3D7_1400600c_0100200 v) (n=1).

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Isolation of Human Monoclonal Antibodies that Broadly React with P. falciparum-Infected Erythrocytes (IEs)

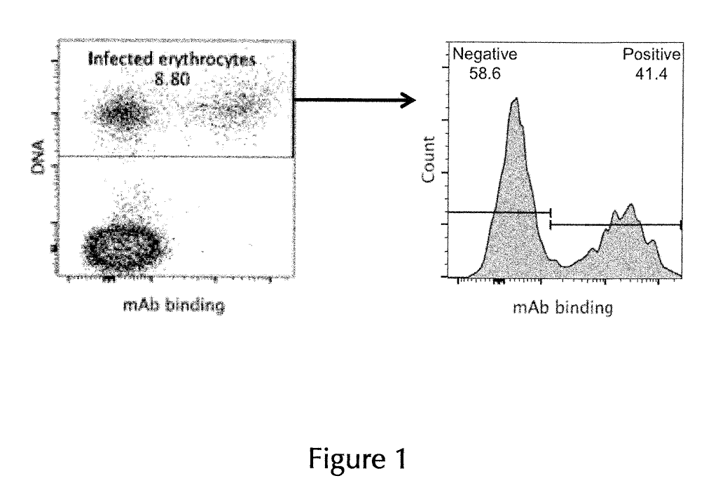

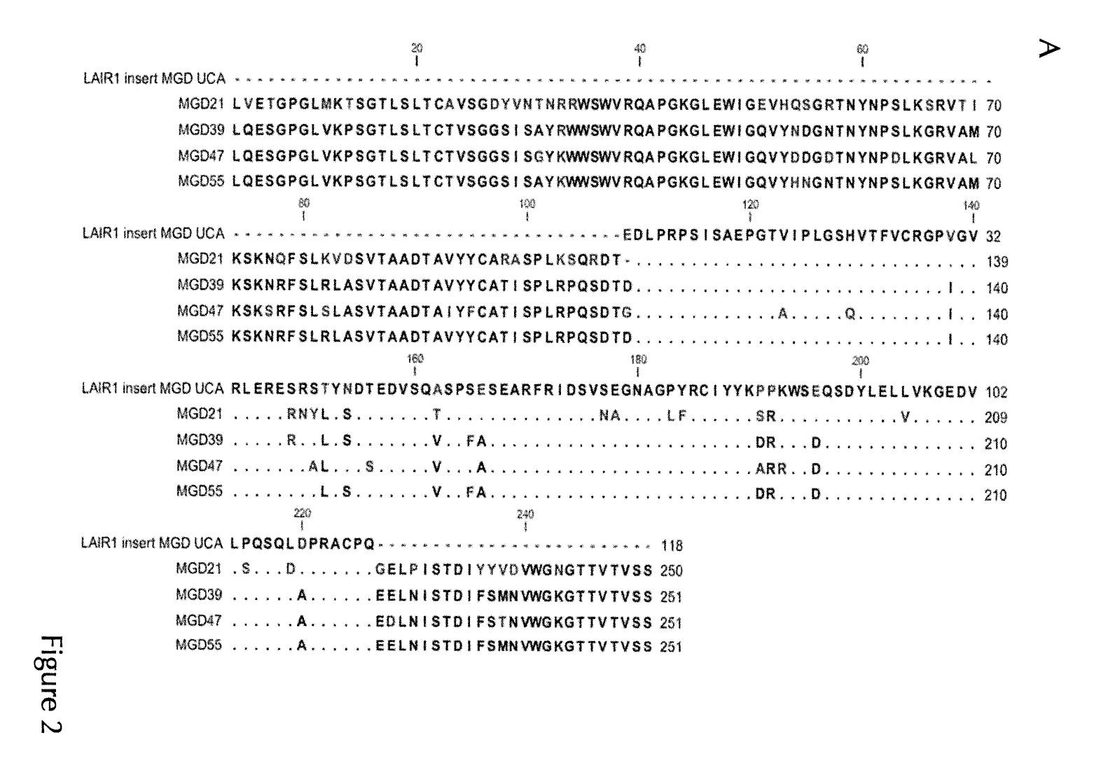

Two African donors (identified as donor C and D) were selected for their high levels of serum antibodies capable of cross-agglutinating erythrocytes infected with different field isolates of P. falciparum. Memory B cells were isolated and immortalized as described by Traggiai, E., et al. An efficient method to make human monoclonal antibodies from memory B cells: potent neutralization of SARS coronavirus. Nat. Med. 10, 871-875 (2004) to isolate monoclonal antibodies. Briefly, memory B cells were isolated from cryopreserved PBMCs using anti-FITC microbeads following staining of PBMCs with CD22-FITC, and were immortalized with Epstein-Barr virus and CpG in multiple wells. After 14 days culture supernatants were screened using a high throughput flow cytometer for their capacity to stain infected erythrocytes (IEs): IEs are stained with SYBR Green I dye (DNA) to discriminate them from uninfected erythrocytes used as control. Supernatants are added on top of IEs and binding of specific antibodies is detected using a secondary-anti-human IgG (Fc-specific) antibody. Positive cultures were expanded and the VH and VL genes from individual clones were sequenced. Several antibodies showed a broad reactivity with the different isolates, while others were specific for a single isolate. The reactivity of the panel of antibodies isolated from donor C and donor D with erythrocytes infected with 8 different field isolates of P. falciparum (9106, 9605, 11019, 9215, 9775, 10975, 10936 and 11014) is shown below in Table 7. An example of IE staining is shown in FIG. 1.

Table 4 shows the panel of antibodies isolated from donor C and donor D (“MGC1”-“MGD56”; Table 2) and their reactivity with erythrocytes infected with 8 different field isolates of P. falciparum (9106, 9605, 11019, 9215, 9775, 10975, 10936 and 11014). The numbers indicate the % of IEs that stained positive for the different antibodies. nd=not detectable.

| | | % parasite recognition | | | | | | | |
|---|---|---|---|---|---|---|---|---|---|
| | | 9106 | 9605 | 11019 | 9215 | 9775 | 10975 | 10936 | 11014 |
| Donor C | MGC1 | 6.7 | 19.5 | 32.7 | 14.9 | 5.7 | 1.4 | 2.0 | 3.4 |
| | MGC2 | 5.6 | 22.4 | 11.9 | 28.8 | 2.9 | 2.6 | 2.8 | 1.5 |
| | MGC4 | 6.7 | 22.2 | 31.1 | 21.7 | 6.1 | 6.7 | 2.3 | 3.6 |
| | MGC5 | 6.6 | 20.3 | 37.6 | 26.4 | 6.0 | 3.8 | 2.1 | 2.9 |
| | MGC7 | 6.9 | 22.8 | 13.8 | 19.3 | 4.6 | 0.7 | 1.7 | 2.9 |
| | MGC17 | 1.3 | 6.8 | 7.1 | 16.7 | 2.5 | 1.5 | 2.4 | 1.6 |
| | MGC26 | 8.5 | 21.1 | 50.8 | 9.5 | 3.4 | 7.3 | 3.6 | 5.4 |
| | MGC28 | 7.5 | 20.9 | 30.0 | 10.8 | 9.8 | 12.3 | 3.0 | 2.8 |
| | MGC29 | 6.7 | 21.8 | 48.8 | 26.9 | 8.2 | 10.5 | 3.9 | 3.7 |
| | MGC32 | 7.8 | 22.9 | 38.1 | 13.1 | 7.9 | 3.2 | 2.9 | 4.5 |
| | MGC33 | 7.5 | 22.3 | 23.5 | 11.5 | 9.7 | 11.5 | 3.6 | 2.9 |
| | MGC34 | 6.8 | 23.7 | 34.1 | 27.1 | 17.5 | 15.2 | 11.3 | 11.4 |
| | MGC35 | 6.5 | 15.9 | 3.2 | 19.5 | 7.2 | 7.4 | 2.5 | 3.6 |
| | MGC36 | 6.9 | 17.9 | 17.9 | 12.4 | 6.2 | 8.6 | 2.7 | 4.6 |
| | MGC37 | 7.2 | 22.2 | 51.8 | 9.9 | 4.0 | 7.5 | 4.1 | 5.8 |
| Donor D | MGD21 | 3.9 | 24.2 | 41.4 | 47.4 | 11.4 | 6.5 | 6.9 | 9.0 |
| | MGD23 | 5.7 | 14.7 | 7.8 | 11.4 | 7.3 | 3.4 | 4.3 | 6.3 |
| | MGD30 | 4.2 | 7.4 | 4.4 | 9.0 | 5.6 | 6.5 | 2.6 | 3.4 |
| | MGD33 | 4.3 | 12.3 | 9.6 | 15.5 | 8.5 | 14.2 | 6.1 | 7.0 |
| | MGD34 | 5.0 | 28.4 | 46.6 | 35.7 | 16.0 | 11.2 | 8.1 | 13.0 |
| | MGD35 | 6.1 | 3.6 | 6.3 | nd | nd | nd | nd | nd |
| | MGD39 | 13.7 | 31.7 | 43.0 | 37.4 | 15.0 | 14.1 | 10.5 | 11.5 |
| | MGD41 | 3.8 | 17.2 | 6.8 | 14.7 | 8.6 | 7.3 | 6.1 | 6.6 |
| | MGD47 | 10.7 | 28.7 | 24.6 | 22.3 | 14.4 | 11.2 | 11.3 | 10.2 |
| | MGD55 | 14.3 | 37.2 | 33.1 | 38.8 | 19.4 | 15.6 | 13.3 | 14.7 |
| | MGD56 | 3.3 | 17.3 | 4.3 | 12.0 | 6.6 | 6.7 | 2.4 | 9.5 |
| | | <2% | 2-5% | 5-10% | 10-20% | 20-40% | >40% | | |

### Example 2: The Human Monoclonal Antibodies that Broadly React with P. falciparum-Infected Erythrocytes are Characterized by a Large HCDR3 Containing a Mutated LAIR-1 Exon

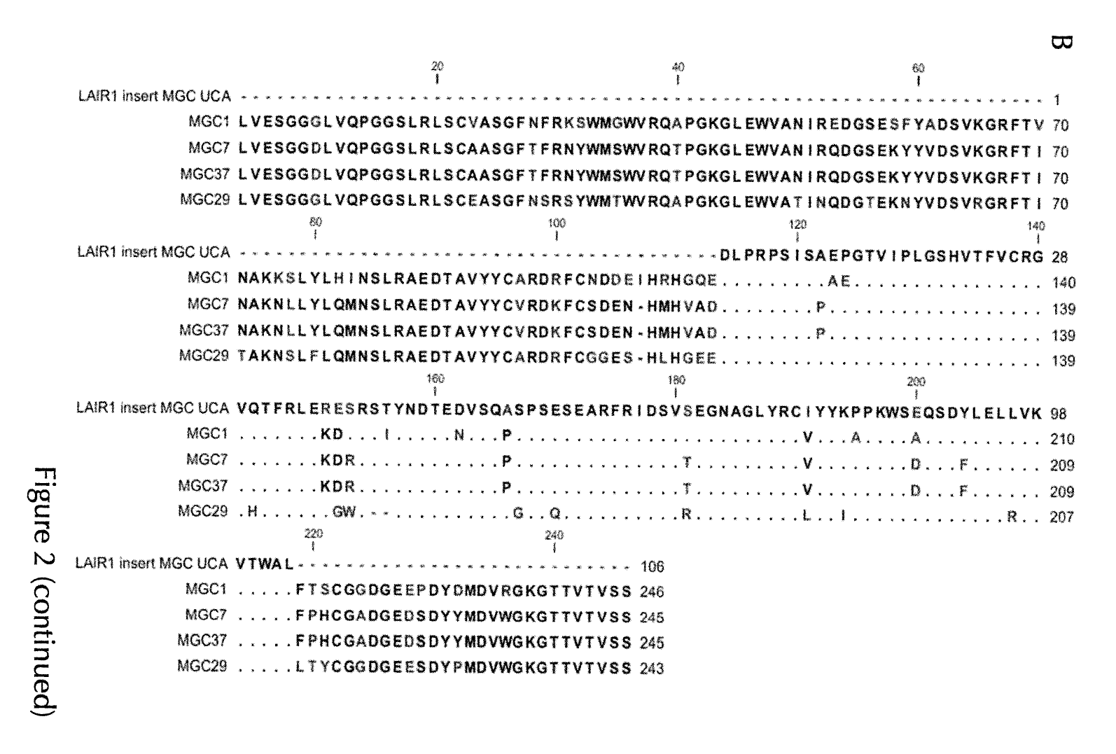

The VH and VL sequences of all of the IE-specific human mAbs of Example 1 were aligned and the V, D and J elements identified using the IMGT database. Surprisingly, all the broadly reactive mAbs isolated from both donors were characterized by an extraordinary long CDRH3 ranging from 120 to 130 amino acids, i.e. broadly reactive antibodies had an insert of more than 100 amino acids between the V and DJ segments, whereas narrowly reactive antibodies showed classical VD) organization of the heavy (H) chain gene. The middle and main part of this CDR3 was found to be highly homologous (92% to 98%) to the third exon plus a intronic sequence of LAIR-1, a gene encoding an inhibitory receptor specific for collagen which is present on chromosome 19. The aminoacidic alignment of these unusual heavy chain variable regions (VH) is shown with reference to the genomic elements (exon and intron) of the LAIR-1 gene (NCBI Reference Sequence: NC_018930.2) in FIG. 2 (cf. FIG. 2: alignment of the complete variable regions of selected antibodies to the genomic LAIR1 portion corresponding to the inserts. LAIR1 gene: ENSG00000167613). In addition, the LAIR-1 exon/intron insert was associated with VH4-4 and JH6 in donor D, and with VH3-7 and JH16 in donor C. All the antibodies carried several mutations both in the VD) elements and in the LAIR-1 insert. In both donors, the length and composition of VH and VL and the pattern of mutations define sister clones carrying different levels of mutations (Table 5).

Table 5 below shows the VH and VL gene usage of antibodies.

| | | Heavy chain | | Light chain | |
|---|---|---|---|---|---|
| | | VH | JH | VL | JL |
| Donor C | MGC4 | IGHV3-7 | IGHJ6 | IGLV7-43 | IGLJ3 |
| | MGC5 | IGHV3-7 | IGHJ6 | IGLV7-43 | IGLJ3 |
| | MGC8 | IGHV3-7 | IGHJ6 | IGLV7-43 | IGLJ3 |
| | MGC29 | IGHV3-7 | IGHJ6 | IGLV7-43 | IGLJ3 |
| | MGC33 | IGHV3-7 | IGHJ6 | IGLV7-43 | IGLJ3 |
| | MGC34 | IGHV3-7 | IGHJ6 | IGLV7-43 | IGLJ3 |
| | MGC35 | IGHV3-7 | IGHJ6 | IGLV7-43 | IGLJ3 |
| | MGC36 | IGHV3-7 | IGHJ6 | IGLV7-43 | IGLJ3 |
| | MGC2 | IGHV3-7 | IGHJ6 | IGKV1-5 | IGKJ2 |
| | MGC26 | IGHV3-7 | IGHJ6 | IGKV1-5 | IGKJ2 |
| | MGC37 | IGHV3-7 | IGHJ6 | IGKV1-5 | IGKJ2 |
| | MGC1 | IGHV3-7 | IGHJ6 | IGKV4-1 | IGKJ2 |
| | MGC17 | IGHV3-7 | IGHJ6 | IGKV4-1 | IGKJ2 |
| | MGC32 | IGHV3-7 | IGHJ6 | IGKV4-1 | IGKJ2 |
| | MGC7 | IGHV3-7 | IGHJ6 | IGKV1-12 | IGKJ4 |
| Donor D | MGD21 | IGHV4-4 | IGHJ6 | IGKV1-8 | IGKJ5 |
| | MGD23 | IGHV4-4 | IGHJ6 | IGKV1-8 | IGKJ5 |
| | MGD30 | IGHV4-4 | IGHJ6 | IGKV1-8 | IGKJ5 |
| | MGD33 | IGHV4-4 | IGHJ6 | IGKV1-8 | IGKJ5 |
| | MGD34 | IGHV4-4 | IGHJ6 | IGKV1-8 | IGKJ5 |
| | MGD35 | IGHV4-4 | IGHJ6 | IGKV1-8 | IGKJ5 |
| | MGD39 | IGHV4-4 | IGHJ6 | IGKV1-8 | IGKJ5 |
| | MGD41 | IGHV4-4 | IGHJ6 | IGKV1-8 | IGKJ5 |
| | MGD47 | IGHV4-4 | IGHJ6 | IGKV1-8 | IGKJ5 |
| | MGD55 | IGHV4-4 | IGHJ6 | IGKV1-8 | IGKJ5 |
| | MGD56 | IGHV4-4 | IGHJ6 | IGKV1-8 | IGKJ5 |

### Example 3: Construction of Antibody Variants of MGD21

Of the antibodies described in Example 1 and Example 2 one broadly binding antibody, namely MGD21, was selected. MGD21 (SEQ ID NOs: 326-343) is a monoclonal antibody that binds to erythrocytes infected with 8/8 primary P. falciparum isolates and carries the LAIR-1 exon+intron insertion (a part of the intron, intron_{α}, is shared with MGC antibodies, while the second part, intron_{β}, is shared only with MGD antibodies). To understand which elements are required for binding to IEs, variants of the MGD21 mAb were produced, in which single elements (V, D, J and LAIR-1 exon and intron insertions) were either deleted or substituted with corresponding elements taken from an irrelevant antibody (FI499 reactive to influenza virus hemagglutinin, HA). In addition, variants were produced, in which somatic mutations were reverted to the germline configuration. In particular, mutations in the LAIR-1 exon+intron insertion were reverted to the corresponding original genomic sequence of LAIR-1 gene (NCBI Reference Sequence: NC_018930.2).

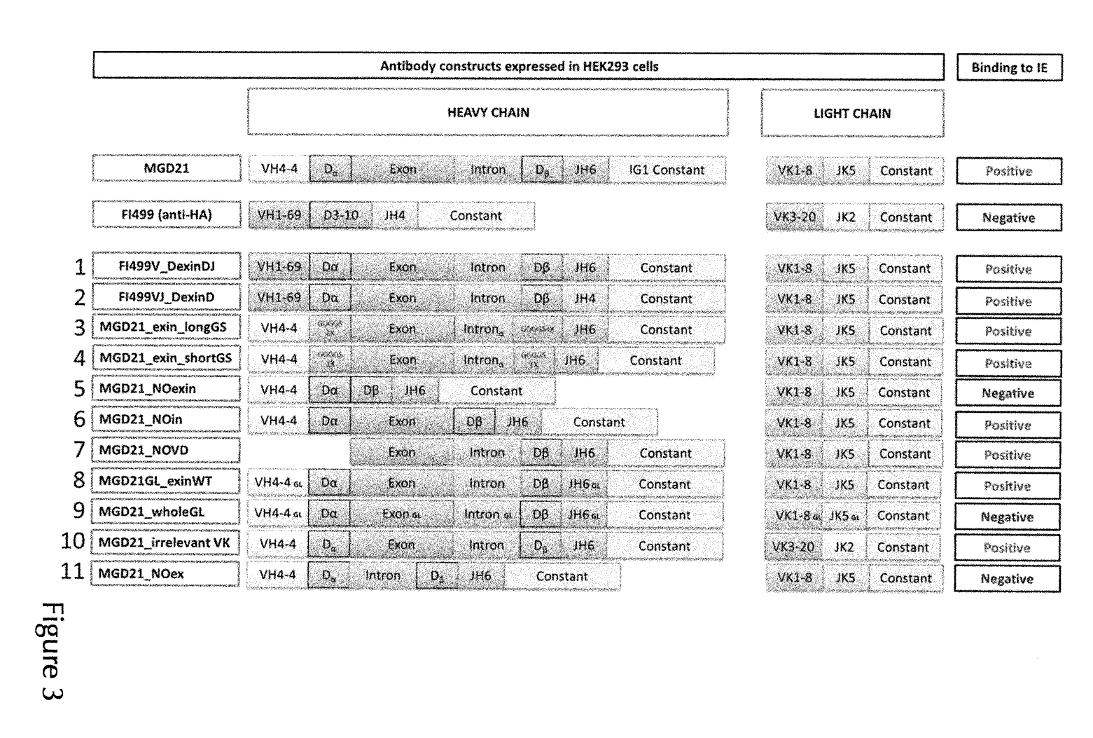

The following variants were produced, which are shown schematically in FIG. 3 (all the constructs have the same full complete constant region as antibody MGD21 as described herein and differ only in the heavy chain, while the light chain is not modified; the construct are finally expressed as monoclonal antibodies (H+L chain)):
1. “FI499V_DexinDJ” is formed by (in this order from N- to C-terminus): the expression product of a V (variable) gene segment of a heavy chain variable region of FI499 (“VH1-69”), the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
2. “FI499VJ_DexinD” is formed by (in this order from N- to C-terminus): the expression product of a V (variable) gene segment of a heavy chain variable region of FI499 (“VH1-69”), the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of FI499 (“JH4”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
3. “MGD21_exin_longGS” is formed by (in this order from N- to C-terminus): the expression product of a V (variable) gene segment of a heavy chain variable region of MGD21 (“VH4-4”); the expression product of a 10-amino-acid linker (“GGGGS 2×”); the mutated LAIR-1 fragment (“Exon”); the expression product of a LAIR-1 intron fragment (“Intron_{α}”); the expression product of a 20-amino-acid linker (“GGGGS 4×”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“V10-8”) and the expression product of a J (Joining) gene segment element of a light chain variable region of MGD21 (“JK5”); the expression product of a C (constant) gene segment of a light chain constant region.
4. “MGD21_exin_shortGS” is formed by (in this order from N- to C-terminus): the expression product of a V (variable) gene segment of a heavy chain variable region of MGD21 (“VH4-4”); the expression product of a 5-amino-acid linker (“GGGGS 1×”); the mutated LAIR-1 fragment (“Exon”); the expression product of a LAIR-1 intron fragment (“Intron_{α}”); the expression product of a 5-amino-acid linker (“GGGGS 1×”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a (Joining) gene segment element of a light chain variable region of MGD21 (“JK5”); the expression product of a C (constant) gene segment of a light chain constant region.
5. “MGD21_NOexin” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
6. “MGD21_NOin” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the mutated LAIR-1 fragment (“Exon”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
7. “MGD21_NOVD” is formed by (in this order from N- to C-terminus): the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
8. “MGD21GL_exinWT” is formed by (in this order from N- to C-terminus): the expression product of an unmutated V (variable) gene segment of a heavy chain variable region of MGD21 (“VH4-4 GL”); the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of an unmutated J (Joining) gene segment element of a heavy chain variable region of MGD21 (“JH6 GL”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
9. “MGD21_wholeGL” is formed by (in this order from N- to C-terminus): the expression product of an unmutated V (variable) gene segment of a heavy chain variable region of MGD21 (“VH4-4 GL”); the expression product of a first D (Diversity) gene segment element of a heavy chain variable region of MGD21 (“D_{α}”); the unmutated LAIR-1 fragment (“Exon GL”); the expression product of a unmutaed LAIR-1 intron fragment (“Intron GL”); the expression product of a second D (Diversity) gene segment element of a heavy chain variable region of MGD21 (“D_{β}”); the expression product of a J (Joining) gene segment element of a unmutated heavy chain variable region of MGD21 (“JH6 GL”); the expression product of a C (constant) gene segment of a heavy chain constant region (IgG1 isotype); and on a separate chain: the expression product of an unmutated V (variable) gene segment of a light chain variable region of MGD21 (“VK1-8 GL”) and the expression product of an unmutated J (Joining) gene segment element of a light chain variable region of MGD21 (“JK5 GL”); the expression product of a C (constant) gene segment of a light chain constant region.
10. “MGD21_irrelevant VK” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of FI499 (“VK3-20”) and the expression product of a J (Joining) gene segment element of a light chain variable region of FI499 (“JK2”); the expression product of a C (constant) gene segment of a light chain constant region.
11. “MGD21_NOex” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.

Table 6 below provides amino acid and nucleic acid sequences of the heavy chain variable regions of the constructs described above (Example 3).

TABLE 6
Sequences and Seq IDs of constructs

| | | |
|---|---|---|
| SEQ ID | | |
| NO | Description | Sequence* |

Heavy chain variable regions

| | | |
|---|---|---|
| 542 | FI499V_DexinDJ | QVQPVQSGAEVKEPGSSVKVSCKTSGGLIRKSAVSWVRQAP |
| | aa | GQGLEWMGGISALFNTKDYAEKFQGRLTITADESTATAYMEL |
| | | SSLTSEDTAIYYCATASPLKSQRDTDLPRPSISAEPGTVIPLGSHV |
| | | TFVCRGPVGVQTFRLERERNYLYSDTEDVSQTSPSESEARFRID |
| | | SVNAGNAGLFRCIYYKSRKWSEQSDYLELVVKGEDVTWALSQ |
| | | SQDDPRACPQGELPISTDIYYVDVWGNGTTVTVSS |
| 543 | FI499V_DexinDJ | CAGGTGCAGCCCGTCCAGTCTGGAGCAGAGGTGAAGGA |
| | nucl | ACCTGGCAGCTCCGTGAAGGTCTCTTGCAAAACAAGTGG |
| | | CGGGCTGATCCGCAAAAGTGCCGTGTCATGGGTCCGACA |
| | | GGCTCCTGGACAGGGACTGGAATGGATGGGAGGCATCA |
| | | GCGCACTGTTCAACACTAAGGACTACGCCGAAAAATTTCA |
| | | GGGCCGGCTGACTATTACCGCCGATGAGAGTACAGCCAC |
| | | TGCTTATATGGAACTGTCTAGTCTGACCAGCGAGGACACA |
| | | GCTATCTACTATTGCGCAACCGCCTCACCACTGAAGTCCC |
| | | AGAGAGACACCGACCTGCCAAGACCTTCCATCTCTGCAG |
| | | AACCTGGCACAGTGATTCCACTGGGGTCCCACGTGACTTT |
| | | CGTCTGTAGGGGACCAGTGGGCGTCCAGACCTTTCGCCT |
| | | GGAGCGGGAAAGAAATTACCTGTATTCCGACACTGAGGA |
| | | CGTGAGCCAGACCAGTCCCTCAGAGAGCGAAGCTAGGTT |
| | | CCGCATCGATTCCGTGAACGCTGGGAATGCAGGACTGTT |
| | | TAGATGCATCTACTATAAGTCTAGGAAATGGAGCGAGCA |
| | | GTCCGACTACCTGGAACTGGTGGTCAAAGGGGAGGATG |
| | | TGACATGGGCTCTGTCCCAGTCTCAGGACGATCCAAGAG |
| | | CATGTCCCCAGGGCGAGCTGCCCATCTCTACTGACATCTA |
| | | CTATGTGGATGTCTGGGGCAACGGGACCACAGTGACCGT |
| | | CTCAAGC |
| 544 | FI499VJ_DexinD | QVQPVQSGAEVKEPGSSVKVSCKTSGGLIRKSAVSWVRQAP |
| | aa | GQGLEWMGGISALFNTKDYAEKFQGRLTITADESTATAYMEL |
| | | SSLTSEDTAIYYCATASPLKSQRDTDLPRPSISAEPGTVIPLGSHV |
| | | TFVCRGPVGVQTFRLERERNYLYSDTEDVSQTSPSESEARFRID |
| | | SVNAGNAGLFRCIYYKSRKWSEQSDYLELVVKGEDVTWALSQ |
| | | SQDDPRACPQGELPISTDIFDYWGQGTLVTVSS |
| 545 | FI499VJ_DexinD | CAGGTGCAGCCCGTCCAGTCTGGAGCAGAGGTGAAGGA |
| | nucl | ACCTGGCAGCTCCGTGAAGGTCTCTTGCAAAACAAGTGG |
| | | CGGGCTGATCCGCAAAAGTGCCGTGTCATGGGTCCGACA |
| | | GGCTCCTGGACAGGGACTGGAATGGATGGGAGGCATCA |
| | | GCGCACTGTTCAACACTAAGGACTACGCCGAAAAATTTCA |
| | | GGGCCGGCTGACCATTACAGCCGATGAGAGTACTGCCAC |
| | | CGCTTATATGGAACTGTCTAGTCTGACCAGCGAGGACAC |
| | | AGCTATCTACTATTGCGCAACCGCCTCACCACTGAAGTCC |
| | | CAGAGAGACACCGACCTGCCAAGACCTTCCATCTCTGCA |
| | | GAACCTGGCACAGTGATTCCACTGGGGTCCCACGTGACT |
| | | TTCGTCTGTAGGGGACCAGTGGGCGTCCAGACCTTTCGC |
| | | CTGGAGCGGGAAAGAAATTACCTGTATTCCGACACTGAG |
| | | GACGTGAGCCAGACCAGTCCCTCAGAGAGCGAAGCTAG |
| | | GTTCCGCATCGATTCCGTGAACGCTGGGAATGCAGGACT |
| | | GTTTAGATGCATCTACTATAAGTCTAGGAAATGGAGCGAG |
| | | CAGTCCGACTACCTGGAACTGGTGGTCAAAGGGGAGGA |
| | | TGTGACTTGGGCTCTGTCCCAGTCTCAGGACGATCCAAG |
| | | AGCATGTCCCCAGGGCGAGCTGCCCATCTCTACCGACAT |
| | | TTTCGATTATTGGGGCCAGGGGACACTGGTGACTGTCTC |
| | | AAGC |
| 546 | MGD21_exin_longGS | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARGGGGS |
| | | GGGGSDLPRPSISAEPGTVIPLGSHVTFVCRGPVGVQTFRLER |
| | | ERNYLYSDTEDVSQTSPSESEARFRIDSVNAGNAGLFRCIYYKS |
| | | RKWSEQSDYLELVVKGEDVTWALGGGGS GGGGS GGGGS |
| | | GGGGSYYVDVWGNGTTVTVSS |
| 547 | MGD21_exin_longGS | GAAGTGCAGCTGGTGGAAACCGGCCCTGGACTGATGAA |
| | nucl | GACCTCTGGCACACTGAGTCTGACATGCGCTGTGAGTGG |
| | | GGACTACGTCAACACTAATCGGAGATGGTCTTGGGTGCG |
| | | ACAGGCACCAGGAAAAGGACTGGAGTGGATCGGGGAA |
| | | GTGCACCAGAGCGGAAGGACCAACTATAATCCTAGCCTG |
| | | AAGTCCCGCGTGACAATTTCAGTCGATAAGAGCAAAAAC |
| | | CAGTTCTCCCTGAAAGTGGACTCTGTCACTGCCGCTGATA |
| | | CCGCAGTGTACTATTGTGCCAGAGGCGGGGGAGGCTCT |
| | | GGGGGAGGCGGGAGTGACCTGCCCAGGCCTAGCATCTC |
| | | CGCTGAACCAGGGACTGTGATTCCCCTGGGATCTCACGT |
| | | GACCTTCGTCTGCAGAGGCCCTGTGGGGGTCCAGACATT |
| | | TCGCCTGGAGCGGGAAAGAAACTACCTGTATTCTGACAC |
| | | CGAGGATGTGAGTCAGACATCTCCCAGTGAGTCAGAAGC |
| | | AAGGTTCCGCATCGATTCCGTCAACGCCGGAAATGCTGG |
| | | CCTGTTTCGATGTATCTACTATAAGAGCCGGAAATGGAGC |
| | | GAGCAGTCCGACTACCTGGAACTGGTGGTCAAGGGCGA |
| | | GGATGTGACCTGGGCCCTGGGCGGGGGAGGCTCTGGG |
| | | GGAGGCGGGAGTGGAGGCGGGGGATCAGGTGGAGGC |
| | | GGGTCGTACTATGTGGACGTGTGGGGCAACGGGACCAC |
| | | AGTGACCGTCAGCTCC |
| 548 | MGD21_exin_short | EVQLVETGPGLMKTSGTLSLTCAVSGDYVNTNRRWSWVRQ |
| | GS aa | APGKGLEWIGEVHQSGRTNYNPSLKSRVTISVDKSKNQFSLKV |
| | | DSVTAADTAVYYCARGGGGS |
| | | DLPRPSISAEPGTVIPLGSHVTFVCRGPVGVQTFRLERERNYLY |
| | | SDTEDVSQTSPSESEARFRIDSVNAGNAGLFRCIYYKSRKWSE |
| | | QSDYLELVVKGEDVTWALGGGGS YYVDVWGNGTTVTVSS |
| 549 | MGD21_exin_short | GAAGTGCAGCTGGTGGAAACCGGCCCTGGACTGATGAA |
| | GS nucl | GACCTCTGGCACACTGAGTCTGACATGCGCTGTGAGTGG |
| | | GGACTACGTCAACACTAATCGGAGATGGTCTTGGGTGCG |
| | | ACAGGCACCAGGAAAAGGACTGGAGTGGATCGGGGAA |
| | | GTGCACCAGAGCGGAAGGACCAACTATAATCCTAGCCTG |
| | | AAGTCCCGCGTGACAATTTCAGTCGATAAGAGCAAAAAC |
| | | CAGTTCTCCCTGAAAGTGGACTCTGTCACTGCCGCTGATA |
| | | CCGCAGTGTACTATTGTGCCAGAGGGGGAGGCGGGAGT |
| | | GACCTGCCCAGGCCTAGCATCTCCGCTGAACCAGGGACT |
| | | GTGATTCCCCTGGGATCTCACGTGACCTTCGTCTGCAGAG |
| | | GCCCTGTGGGGGTCCAGACATTTCGCCTGGAGCGGGAA |
| | | AGAAACTACCTGTATTCTGACACCGAGGATGTGAGTCAG |
| | | ACATCTCCCAGTGAGTCAGAAGCAAGGTTCCGCATCGATT |
| | | CCGTCAACGCCGGAAATGCTGGCCTGTTTCGATGTATCTA |
| | | CTATAAGAGCCGGAAATGGAGCGAGCAGTCCGACTACCT |
| | | GGAACTGGTGGTCAAGGGCGAGGATGTGACCTGGGCCC |
| | | TGGGAGGCGGGGGATCATACTATGTGGACGTGTGGGGC |
| | | AACGGGACCACAGTGACCGTCAGCTCC |
| 550 | MGD21_NOexin | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARASPLKSQRDTGELPISTDIYYVDVWGN |
| | | GTTVTVSS |
| 551 | MGD21_NOexin | GAAGTGCAGCTGGTGGAAACCGGCCCTGGACTGATGAA |
| | nucl | GACTTCAGGAACCCTGAGCCTGACTTGTGCCGTGAGCGG |
| | | CGACTACGTCAACACCAATCGGAGATGGAGTTGGGTGCG |
| | | GCAGGCACCAGGAAAAGGCCTGGAGTGGATCGGCGAA |
| | | GTGCACCAGTCTGGGCGAACAAACTATAATCCCTCTCTGA |
| | | AGAGTAGAGTGACTATTTCCGTGGACAAGTCTAAAAACCA |
| | | GTTCAGCCTGAAAGTGGACTCCGTCACAGCCGCTGATAC |
| | | TGCCGTGTACTATTGTGCAAGGGCCAGTCCCCTGAAGTC |
| | | ACAGCGCGATACCGGGGAGCTGCCTATCAGCACAGACAT |
| | | CTACTATGTGGATGTCTGGGGGAATGGAACCACAGTGAC |
| | | AGTCAGCTCC |
| 552 | MGD21_NOin | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARASPLKSQRDTDLPRPSISAEPGTVIPLGS |
| | | HVTFVCRGPVGVQTFRLERERNYLYSDTEDVSQTSPSESEARF |
| | | RIDSVNAGNAGLFRCIYYKSRKWSEQSDYLELVVKGELPISTDI |
| | | YYVDVWGNGTTVTVSS |
| 553 | MGD21_NOin | GAGGTGCAGCTGGTCGAAACCGGCCCAGGGCTGATGAA |
| | nucl | GACTTCCGGAACCCTGTCTCTGACATGCGCCGTGTCCGG |
| | | GGACTACGTCAACACTAATCGGAGATGGTCTTGGGTGAG |
| | | GCAGGCTCCTGGAAAAGGCCTGGAGTGGATCGGGGAAG |
| | | TGCACCAGTCCGGACGGACCAACTATAATCCATCTCTGAA |
| | | GAGTAGAGTGACAATTAGTGTCGATAAGTCAAAAAACCA |
| | | GTTCTCTCTGAAAGTGGACAGTGTCACAGCCGCTGATACT |
| | | GCAGTGTACTATTGTGCAAGAGCAAGCCCCCTGAAGTCC |
| | | CAGAGAGACACCGACCTGCCCAGGCCTTCTATCAGTGCT |
| | | GAACCAGGCACTGTGATTCCCCTGGGGTCTCATGTGACC |
| | | TTCGTCTGTAGAGGCCCCGTGGGAGTCCAGACTTTCGC |
| | | CTGGAGAGGGAACGCAATTACCTGTATTCAGACACCGAG |
| | | GATGTGAGCCAGACATCACCTAGCGAGTCCGAAGCCCGA |
| | | TTCCGGATCGACAGTGTGAACGCTGGAAATGCAGGCCTG |
| | | TTTCGCTGTATCTACTATAAGAGCCGAAAATGGTCAGAGC |
| | | AGAGCGATTACCTGGAACTGGTGGTCAAAGGCGAGCTG |
| | | CCTATCAGCACTGACATCTACTATGTGGATGTCTGGGGGA |
| | | ACGGAACCACAGTGACCGTCAGCTCC |
| 554 | MGD21_NOVD | DLPRPSISAEPGTVIPLGSHVTFVCRGPVGVQTFRLERERNYLY |
| | aa | SDTEDVSQTSPSESEARFRIDSVNAGNAGLFRCIYYKSRKWSE |
| | | QSDYLELVVKGEDVTWALSQSQDDPRACPQGELPISTDIYYV |
| | | DVWGNGTTVTVSS |
| 555 | MGD21_NOVD | GACCTGCCACGACCATCTATTTCCGCCGAACCTGGGACT |
| | nucl | GTCATTCCTCTGGGGAGCCACGTCACATTTGTCTGCCGG |
| | | GGACCTGTCGGGGTGCAGACTTTCCGGCTGGAGCGGGA |
| | | AAGAAACTACCTGTATTCTGACACCGAAGATGTGAGTCAG |
| | | ACAAGCCCATCCGAGTCTGAAGCTAGGTTCCGCATCGAC |
| | | TCCGTCAACGCCGGCAATGCTGGGCTGTTTCGATGCATCT |
| | | ACTATAAGAGCAGAAAATGGAGCGAGCAGTCCGACTACC |
| | | TGGAACTGGTGGTCAAGGGAGAGGATGTCACCTGGGCA |
| | | CTGAGTCAGTCACAGGACGATCCCCGGGCCTGTCCTCAG |
| | | GGCGAGCTGCCCATCAGCACTGATATCTACTATGTGGAT |
| | | GTCTGGGGGAATGGCACTACTGTGACCGTCTCAAGC |
| 556 | MGD21GL_exin | QVQLQESGPGLVKPSGTLSLTCAVSGGSISSSNWWSWVRQP |
| | WT aa | PGKGLEWIGEIYHSGSTNYNPSLKSRVTISVDKSKNQFSLKLSS |
| | | VTAADTAVYYCARASPLKSQRDTDLPRPSISAEPGTVIPLGSHV |
| | | TFVCRGPVGVQTFRLERERNYLYSDTEDVSQTSPSESEARFRID |
| | | SVNAGNAGLFRCIYYKSRKWSEQSDYLELVVKGEDVTWALSQ |
| | | SQDDPRACPQGELPISTDIYYMDVWGKGTTVTVSS |
| 557 | MGD21GL_exin | CAGGTCCAGCTGCAGGAAAGCGGCCCAGGACTGGTGAA |
| | WT nucl | GCCTAGCGGAACACTGAGTCTGACTTGTGCCGTGAGCGG |
| | | AGGGAGCATCAGCTCCTCTAACTGGTGGTCTTGGGTGAG |
| | | GCAGCCCCCTGGCAAGGGACTGGAGTGGATCGGCGAAA |
| | | TCTACCACAGCGGGTCCACCAACTATAATCCTTCACTGAA |
| | | GAGCCGCGTGACAATCAGTGTGGACAAGTCAAAAAATCA |
| | | GTTCAGCCTGAAACTGAGTTCAGTGACCGCCGCTGATAC |
| | | AGCAGTCTACTATTGCGCACGGGCCAGCCCACTGAAATC |
| | | CCAGCGAGACACTGATCTGCCACGGCCCTCTATCAGTGC |
| | | TGAACCCGGAACAGTGATTCCTCTGGGCTCCCATGTGACT |
| | | TTCGTCTGTCGCGGACCAGTGGGCGTCCAGACCTTTCGA |
| | | CTGGAGCGGGAAAGAAACTACCTGTATTCTGACACTGAG |
| | | GATGTGAGTCAGACCTCACCCAGCGAGTCCGAAGCCAG |
| | | GTTCCGCATCGACAGCGTCAACGCTGGGAATGCAGGACT |
| | | GTTTAGATGCATCTACTATAAGTCCAGGAAATGGTCCGAG |
| | | CAGTCTGACTACCTGGAACTGGTGGTCAAGGGGGAGGA |
| | | TGTGACATGGGCCCTGTCTCAGAGTCAGGACGATCCTAG |
| | | AGCTTGTCCACAGGGCGAGCTGCCCATTTCAACCGATATC |
| | | TATTACATGGATGTCTGGGGCAAGGGCACCACCGTGACC |
| | | GTGAGCAGC |
| 558 | MGD21_wholeGL | QVQLQESGPGLVKPSGTLSLTCAVSGGSISSSNWWSWVRQP |
| | aa | PGKGLEWIGEIYHSGSTNYNPSLKSRVTISVDKSKNQFSLKLSS |
| | | VTAADTAVYYCARASPLKSQRDTEDLPRPSISAEPGTVIPLGSH |
| | | VTFVCRGPVGVQTFRLERESRSTYNDTEDVSQASPSESEARFRI |
| | | DSVSEGNAGPYRCIYYKPPKWSEQSDYLELLVKGEDVTWALP |
| | | QSQLDPRACPQGELPISTDIYYMDVWGKGTTVTVSS |
| 559 | MGD21_wholeGL | CAGGTGCAGCTGCAGGAAAGCGGACCAGGCCTGGTCAA |
| | nucl | GCCCTCAGGCACTCTGAGCCTGACCTGCGCTGTGAGTGG |
| | | CGGGTCAATCAGCTCCTCTAATTGGTGGTCCTGGGTGAG |
| | | GCAGCCCCCTGGGAAAGGACTGGAGTGGATCGGCGAAA |
| | | TCTACCACTCTGGGAGTACAAACTATAATCCCAGCCTGAA |
| | | GTCCCGCGTGACTATTTCCGTGGACAAGTCTAAAAATCAG |
| | | TTCAGCCTGAAACTGAGTTCAGTGACAGCCGCTGATACTG |
| | | CAGTCTACTATTGCGCACGAGCCAGTCCTCTGAAGTCCCA |
| | | GCGGGACACTGAGGACCTGCCTAGACCATCAATCAGCGC |
| | | CGAGCCTGGAACTGTGATTCCACTGGGCTCTCATGTGAC |
| | | CTTCGTCTGTAGAGGACCAGTGGGAGTCCAGACCTTCCG |
| | | GCTGGAGAGAGAATCCCGATCTACCTACAACGACACAGA |
| | | AGATGTGAGCCAGGCTAGTCCATCAGAGAGCGAAGCAC |
| | | GGTTTAGAATCGACTCCGTGTCTGAGGGGAATGCCGGAC |
| | | CCTACAGATGCATCTACTATAAGCCACCCAAATGGTCTGA |
| | | GCAGAGTGACTATCTGGAACTGCTGGTGAAAGGAGAGG |
| | | ATGTCACCTGGGCACTGCCTCAGTCTCAGCTGGACCCCA |
| | | GAGCTTGTCCTCAGGGAGAGCTGCCTATCAGCACCGACA |
| | | TCTACTATATGGACGTGTGGGGCAAAGGGACCACAGTGA |
| | | CAGTCAGCTCCGCGTCGACTTCGCA |
| 560 | MGD21_NOex | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARASPLKSQRDTGEDVTWALSQSQDDPR |
| | | ACPQGELPISTDIYYVDVWGNGTTVTVSS |
| 561 | MGD21_NOex | GAAGTGCAGCTGGTGGAAACCGGCCCTGGACTGATGAA |
| | nucl | GACTTCCGGAACCCTGTCTCTGACTTGCGCCGTGTCTGGC |
| | | GACTACGTCAACACCAATCGGAGATGGAGCTGGGTGCG |
| | | GCAGGCTCCAGGAAAAGGCCTGGAGTGGATCGGCGAAG |
| | | TGCACCAGTCCGGGCGAACAAACTATAATCCCTCACTGAA |
| | | GAGCAGAGTGACTATTAGTGTCGATAAGTCAAAAAACCA |
| | | GTTCTCTCTGAAAGTGGACAGTGTCACAGCCGCTGATACT |
| | | GCCGTGTACTATTGCGCAAGGGCCAGCCCTCTGAAGTCC |
| | | CAGAGAGACACCGGGGAGGATGTGACATGGGCTCTGTC |
| | | TCAGAGTCAGGACGATCCCCGGGCATGTCCTCAGGGCG |
| | | AACTGCCAATCAGCACCGACATCTACTATGTGGATGTCTG |
| | | GGGGAATGGAACCACAGTGACAGTCAGCTCC |

### Example 4: Identification of the Mutated LAIR-1 Exon as the Only Element Required for MGD21 mAb Binding to P. falciparum-Infected Erythrocytes (IEs)

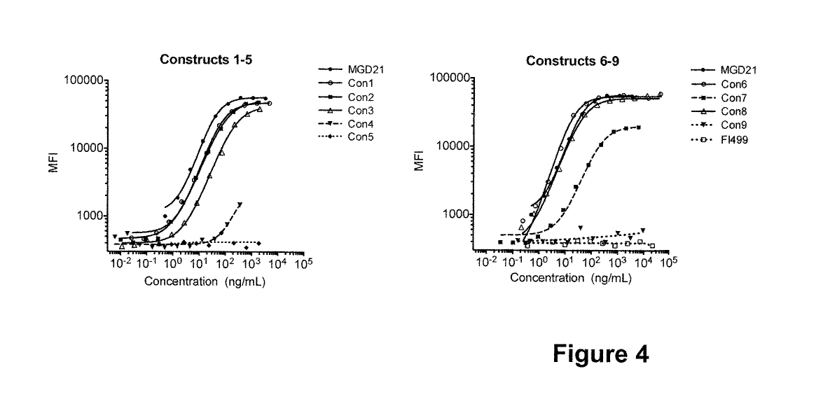

The 10 antibody variants constructed in Example 3 as well as the antibody MGD21 (cf. Examples 1 and 2) and the antibody FI499 (control: irrelevant antibody reactive to influenza virus hemagglutinin, HA) were expressed in HEK 293 cells and tested for their capacity to stain IEs as described in Example 1. Briefly, IEs are stained with SYBR Green I dye (DNA) to discriminate them from uninfected erythrocytes used as control. The antibody variants are added on top of IEs and binding of specific antibodies to IEs is detected using a secondary-anti-human IgG (Fc-specific) antibody. The binding data are shown in FIG. 4. Most constructs show binding to IEs, with the exception of those constructs wherein the exon is either not present or is in the original genomic form (Con5/“MGD21_NOexin”, Con9/“MGD21_wholeGL” and Con11 “MGD21_NOex”). The results indicate that the only element required for binding to IE is the mutated LAIR-1 exon.

### Example 5: Construction of Ig Fusion Proteins Comprising the Mutated LAIR-1 Fragment

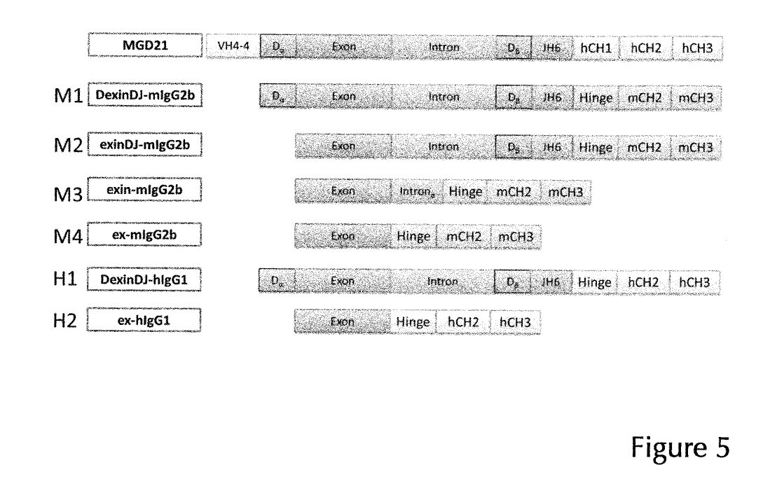

To investigate whether the mutated LAIR-1 exon alone is sufficient to bind to IEs, six different Ig fusion proteins comprising the mutated LAIR-1 fragment were constructed by inserting:
(a) the mutated LAIR-1 exon, preferably according to SEQ ID NO: 34 or a functional sequence variant thereof;
(b) optionally, one or more further elements (intron segments) of LAIR-1, preferably corresponding to such elements of the antibody MGD21 as shown in FIG. 5 and;
(c) optionally, one or more different elements of a heavy chain variable region of an IgG-type antibody, preferably of the antibody MGD21,
   into a plasmid designed for expression of mouse IgG2b fusion proteins (pINFUSE-mIgG2b-Fc2 by Invivogen) or human IgG1 fusion proteins (pINFUSE-hIgG1-Fc2 by Invivogen). Preferred sequences for the constant regions (hinge region and CH2 and CH3 domains) of mouse IgG2b fusion proteins comprise or consist of a sequence according to SEQ ID NO: 562 (amino acid) or SEQ ID NO: 563 (nucleic acid), or functional sequence variants thereof. Preferred sequences for the constant regions (hinge region and CH2 and CH3 domains) of human IgG1 fusion proteins comprise or consist of a sequence according to SEQ ID NO: 564 (amino acid) or SEQ ID NO: 565 (nucleic acid), or functional sequence variants thereof. Preferably, the mutated LAIR-1 fragment (“Exon”) in the following Ig fusion proteins comprises or consists of an amino acid sequence according to SEQ ID NO: 34 or a functional sequence variant thereof.

The different fusion proteins are shown schematically in FIG. 5 in comparison to the antibody MGD21 and described in the following:
1. M1 (also referred to as “DexinDJ-mIgG2b”) is formed by (in this order from N- to C-terminus): the expression product of a first D (Diversity) gene segment element of a heavy chain variable region of an IgG-type antibody, preferably of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of an IgG-type antibody, preferably of MGD21 (“D_{β}”); the expression product of a J (Joining) gene segment element of a heavy chain variable region of an IgG-type antibody, preferably of MGD21 (“JH6”); followed by a hinge region and CH2 and CH3 domains from mouse IgG2b.

An exemplary variable region of such an M1 fusion protein, which is particularly preferred, comprises or consists of an amino acid sequence according to SEQ ID NO: 566 or according to a functional sequence variant thereof, which may preferably be encoded by a nucleic acid sequence according to SEQ ID NO: 567 or by a functional sequence variant thereof. More preferably, a complete M1 fusion protein comprises or consists of an amino acid sequence according to SEQ ID NO: 568 or according to a functional sequence variant thereof, which may preferably be encoded by a nucleic acid sequence according to SEQ ID NO: 569 or by a functional sequence variant thereof.
2. M2 (also referred to as “exinDJ-mIgG2b”) is formed by (in this order from N- to C-terminus): the mutated LAIR-1 fragment (“Exon”); the expression product of a LAIR-1 intron fragment (“Intron”); the expression product of a second D (Diversity) gene segment element of a heavy chain variable region of an IgG-type antibody, preferably of MGD21 (“D_{β}”); the expression product of a J (Joining) gene segment element of a heavy chain variable region of an IgG-type antibody, preferably of MGD21 (“JH6”); followed by a hinge region and CH2 and CH3 domains from mouse IgG2b.
   An exemplary variable region of such an M2 fusion protein, which is particularly preferred, comprises or consists of an amino acid sequence according to SEQ ID NO: 572 or according to a functional sequence variant thereof, which may preferably be encoded by a nucleic acid sequence according to SEQ ID NO: 573 or by a functional sequence variant thereof. More preferably, a complete M2 fusion protein comprises or consists of an amino acid sequence according to SEQ ID NO: 574 or according to a functional sequence variant thereof, which may preferably be encoded by a nucleic acid sequence according to SEQ ID NO: 575 or by a functional sequence variant thereof.
3. M3 (also referred to as “exin-mIgG2b”) is formed by (in this order from N- to C-terminus): the mutated LAIR-1 fragment (“Exon”); the expression product of a partial LAIR-1 intron fragment (“Intron_{α}”); followed by a hinge region and CH2 and CH3 domains from mouse IgG2b.
   An exemplary variable region of such an M3 fusion protein, which is particularly preferred, comprises or consists of an amino acid sequence according to SEQ ID NO: 576 or according to a functional sequence variant thereof, which may preferably be encoded by a nucleic acid sequence according to SEQ ID NO: 577 or by a functional sequence variant thereof. More preferably, a complete M3 fusion protein comprises or consists of an amino acid sequence according to SEQ ID NO: 578 or according to a functional sequence variant thereof, which may preferably be encoded by a nucleic acid sequence according to SEQ ID NO: 579 or by a functional sequence variant thereof.
4. M4 (also referred to as “ex-mIgG2b”) is formed by (in this order from N- to C-terminus): the mutated LAIR-1 fragment (“Exon”); followed by a hinge region and CH2 and CH3 domains from mouse IgG2b.
   An exemplary variable region of such an M4 fusion protein, which is particularly preferred, comprises or consists of an amino acid sequence according to SEQ ID NO: 580 or according to a functional sequence variant thereof, which may preferably be encoded by a nucleic acid sequence according to SEQ ID NO: 581 or by a functional sequence variant thereof. More preferably, a complete M4 fusion protein comprises or consists of an amino acid sequence according to SEQ ID NO: 582 or according to a functional sequence variant thereof, which may preferably be encoded by a nucleic acid sequence according to SEQ ID NO: 583 or by a functional sequence variant thereof.
5. H1 (also referred to as “DexinDJ-hIgG1”) is formed by (in this order from N- to C-terminus): the expression product of a first D (Diversity) gene segment element of a heavy chain variable region of an IgG-type antibody, preferably of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of an IgG-type antibody, preferably of MGD21 (“D_{β}”); the expression product of a J (Joining) gene segment element of a heavy chain variable region of an IgG-type antibody, preferably of MGD21 (“JH6”); followed by a hinge region and CH2 and CH3 domains from human IgG1.

An exemplary variable region of such an H1 fusion protein, which is particularly preferred, comprises or consists of an amino acid sequence according to SEQ ID NO: 566 or according to a functional sequence variant thereof, which may preferably be encoded by a nucleic acid sequence according to SEQ ID NO: 567 or by a functional sequence variant thereof. More preferably, a complete H1 fusion protein comprises or consists of an amino acid sequence according to SEQ ID NO: 570 or according to a functional sequence variant thereof, which may preferably be encoded by a nucleic acid sequence according to SEQ ID NO: 571 or by a functional sequence variant thereof.
6. H2 (also referred to as “ex-hIgG1”) is formed by (in this order from N- to C-terminus): the mutated LAIR-1 fragment (“Exon”); followed by a hinge region and CH2 and CH3 domains from human IgG1.
   An exemplary variable region of such an H2 fusion protein, which is particularly preferred, comprises or consists of an amino acid sequence according to SEQ ID NO: 580 or according to a functional sequence variant thereof, which may preferably be encoded by a nucleic acid sequence according to SEQ ID NO: 581 or by a functional sequence variant thereof. More preferably, a complete H2 fusion protein comprises or consists of an amino acid sequence according to SEQ ID NO: 584 or according to a functional sequence variant thereof, which may preferably be encoded by a nucleic acid sequence according to SEQ ID NO: 585 or by a functional sequence variant thereof.

Table 7 below shows the amino acid and nucleotide sequences of the antibody constructs of Example 5, whereby the constant chain sequences are identical for the mouse IgG2b-antibody constructs M1, M2, M3, and M4 (“mIgG2b”) and for the human IgG1-antibody constructs H1 and H2 (“hIgG1”).

TABLE 7
Sequences and Seq IDs of Ig fusion proteins

| | | |
|---|---|---|
| SEQ ID | | |
| NO | Description | Sequence* |

Constant chains

| | | |
|---|---|---|
| 562 | mIgG2b aa | AMVRSPSGPISTINPCPPCKECHKCPAPNLEGGPSVFIFPPNIKD |
| | | VLMISLTPKVTCVVVDVSEDDPDVQISWFVNNVEVHTAQTQ |
| | | THREDYNSTIRVVSTLPIQHQDWMSGKEFKCKVNNKDLPSPIE |
| | | RTISKIKGLVRAPQVYILPPPAEQLSRKDVSLTCLVVGFNPGDIS |
| | | VEWTSNGHTEENYKDTAPVLDSDGSYFIYSKLNMKTSKWEKT |
| | | DSFSCNVRHEGLKNYYLKKTISRSPGK |
| 563 | mIgG2b nucl | GCCATGGTTAGATCTCCCAGCGGGCCCATTTCAACAATCA |
| | | ACCCCTGTCCTCCATGCAAGGAGTGTCACAAATGCCCAG |
| | | CTCCTAACCTCGAGGGTGGACCATCCGTCTTCATCTTCCC |
| | | TCCAAATATCAAGGATGTACTCATGATCTCCCTGACACCC |
| | | AAGGTCACGTGTGTGGTGGTGGATGTGAGCGAGGATGA |
| | | CCCAGACGTCCAGATCAGCTGGTTTGTGAACAACGTGGA |
| | | AGTACACACAGCTCAGACACAAACCCATAGAGAGGATTA |
| | | CAACAGTACTATCCGGGTGGTCAGCACCCTCCCCATCCA |
| | | GCACCAGGACTGGATGAGTGGCAAGGAGTTCAAATGCA |
| | | AGGTCAACAACAAAGACCTCCCATCACCCATCGAGAGAA |
| | | CCATCTCAAAAATTAAAGGGCTAGTCAGAGCTCCACAAGT |
| | | ATACATCTTGCCGCCACCAGCAGAGCAGTTGTCCAGGAA |
| | | AGATGTCAGTCTCACTTGCCTGGTCGTGGGCTTCAACCCT |
| | | GGAGACATCAGTGTGGAGTGGACCAGCAATGGGCATAC |
| | | AGAGGAGAACTACAAGGACACCGCACCAGTCCTGGACTC |
| | | TGACGGTTCTTACTTCATATATAGCAAGCTCAATATGAAAA |
| | | CAAGCAAGTGGGAGAAAACAGATTCCTTCTCATGCAACG |
| | | TGAGACACGAGGGTCTGAAAAATTACTACCTGAAGAAGA |
| | | CCATCTCCCGGTCTCCGGGTAAA |
| 564 | hIgG1 aa | AMVRSDKTHTCPPCPAPELLGGPSVFLFPPKPKDTLMISRTPEV |
| | | TCVVVDVSHEDPEVKFNWYVDGVEVHNAKTKPREEQYNSTY |
| | | RWSVLTVLHQDWLNGKEYKCKVSNKALPAPIEKTISKAKGQP |
| | | REPQVYTLPPSRDELTKNQVSLTCLVKGFYPSDIAVEWESNGQ |
| | | PENNYKTTPPVLDSDGSFFLYSKLTVDKSRWQQGNVFSCSVM |
| | | HEALHNHYTQKSLSLSPGK |
| 565 | hIgG1 nucl | GCCATGGTTAGATCTGACAAAACTCACACATGCCCACCGT |
| | | GCCCAGCACCTGAACTCCTGGGGGGACCGTCAGTCTTCC |
| | | TCTTCCCCCCAAAACCCAAGGACACCCTCATGATCTCCCG |
| | | GACCCCTGAGGTCACATGCGTGGTGGTGGACGTGAGCC |
| | | ACGAAGACCCTGAGGTCAAGTTCAACTGGTACGTGGACG |
| | | GCGTGGAGGTGCATAATGCCAAGACAAAGCCGCGGGAG |
| | | GAGCAGTACAACAGCACGTACCGTGTGGTCAGCGTCCTC |
| | | ACCGTCCTGCACCAGGACTGGCTGAATGGCAAGGAGTAC |
| | | AAGTGCAAGGTCTCCAACAAAGCCCTCCCAGCCCCCATC |
| | | GAGAAAACCATCTCCAAAGCCAAAGGGCAGCCCCGAGA |
| | | ACCACAGGTGTACACCCTGCCCCCATCCCGGGATGAGCT |
| | | GACCAAGAACCAGGTCAGCCTGACCTGCCTGGTCAAAGG |
| | | CTTCTATCCCAGCGACATCGCCGTGGAGTGGGAGAGCAA |
| | | TGGGCAGCCGGAGAACAACTACAAGACCACGCCTCCCGT |
| | | GCTGGACTCCGACGGCTCCTTCTTCCTCTACAGCAAGCTC |
| | | ACCGTGGACAAGAGCAGGTGGCAGCAGGGGAACGTCTT |
| | | CTCATGCTCCGTGATGCATGAGGCTCTGCACAACCACTAC |
| | | ACGCAGAAGAGCCTCTCCCTGTCTCCGGGTAAA |

MGD21-DexinDJ-mIgG2b

| | | |
|---|---|---|
| 566 | DexinDJ variable | ASPLKSQRDTDLPRPSISAEPGTVIPLGSHVTFVCRGPVGVQTF |
| | part aa | RLERERNYLYSDTEDVSQTSPSESEARFRIDSVNAGNAGLFRCI |
| | | YYKSRKWSEQSDYLELVVKGEDVTWALSQSQDDPRACPQGE |
| | | LPISTDIYYVDVWGNGTTVTVSS |
| 567 | DexinDJ variable | gcgtctccactcaaatctcagagggacaccgatctgcccagaccctccatctcggctg |
| | part nucl | agccgggcaccgtgatccccctggggagccatgtgactttcgtgtgccggggcccggt |
| | | tggggttcaaacattccgcctggagagggagaggaattatttatacagtgatactgaaga |
| | | tgtgtctcaaactagtccatctgagtcggaggccagattccgcattgactcagtaaatgc |
| | | aggcaatgccgggctttttcgctgcatctattacaagtcccgtaaatggtctgagcagag |
| | | tgactacctggagctggtggtgaaaggtgaggacgtcacctgggccctgtcccagtctc |
| | | aagacgaccctcgagcttgtccccagggggagctccccataagtaccgatatttacta |
| | | cgtggacgtctggggcaacgggaccacggtcaccgtctcctca |
| 568 | DexinDJ-mIgG2b | ASPLKSQRDTDLPRPSISAEPGTVIPLGSHVTFVCRGPVGVQTF |
| | complete | RLERERNYLYSDTEDVSQTSPSESEARFRIDSVNAGNAGLFRCI |
| | sequence aa | YYKSRKWSEQSDYLELVVKGEDVTWALSQSQDDPRACPQGE |
| | | LPISTDIYYVDVWGNGTTVTVSSAMVRSPSGPISTINPCPPCKE |
| | | CHKCPAPNLEGGPSVFIFPPNIKDVLMISLTPKVTCVVVDVSED |
| | | DPDVQISWFVNNVEVHTAQTQTHREDYNSTIRVVSTLPIQH |
| | | QDWMSGKEFKCKVNNKDLPSPIERTISKIKGLVRAPQVYILPPP |
| | | AEQLSRKDVSLTCLVVGFNPGDISVEWTSNGHTEENYKDTAP |
| | | VLDSDCSYFIYSKLNMKTSKWEKTDSFSCNVRHEGLKNYYLKK |
| | | TISRSPGK |
| 569 | DexinDJ-mIgG2b | gcgtctccactcaaatctcagagggacaccgatctgcccagaccctccatctcggctg |
| | complete | agccgggcaccgtgatccccctggggagccatgtgactttcgtgtgccggggcccggt |
| | sequence nucl | tggggttcaaacattccgcctggagagggagaggaattatttatacagtgatactgaaga |
| | | tgtgtctcaaactagtccatctgagtcggaggccagattccgcattgactcagtaaatgc |
| | | aggcaatgccgggctttttcgctgcatctattacaagtcccgtaaatggtctgagcagag |
| | | tgactacctggagctggtggtgaaaggtgaggacgtcacctgggccctgtcccagtctc |
| | | aagacgaccctcgagcttgtccccagggggagctccccataagtaccgatatttacta |
| | | cgtggacgtctggggcaacgggaccacggtcaccgtctcctcaGCCATGGTTA |
| | | GATCTCCCAGCGGGCCCATTTCAACAATCAACCCCTGTCC |
| | | TCCATGCAAGGAGTGTCACAAATGCCCAGCTCCTAACCTC |
| | | GAGGGTGGACCATCCGTCTTCATCTTCCCTCCAAATATCA |
| | | AGGATGTACTCATGATCTCCCTGACACCCAAGGTCACGTG |
| | | TGTGGTGGTGGATGTGAGCGAGGATGACCCAGACGTCC |
| | | AGATCAGCTGGTTTGTGAACAACGTGGAAGTACACACAG |
| | | CTCAGACACAAACCCATAGAGAGGATTACAACAGTACTAT |
| | | CCGGGTGGTCAGCACCCTCCCCATCCAGCACCAGGACTG |
| | | GATGAGTGGCAAGGAGTTCAAATGCAAGGTCAACAACAA |
| | | AGACCTCCCATCACCCATCGAGAGAACCATCTCAAAAATT |
| | | AAAGGGCTAGTCAGAGCTCCACAAGTATACATCTTGCCG |
| | | CCACCAGCAGAGCAGTTGTCCAGGAAAGATGTCAGTCTC |
| | | ACTTGCCTGGTCGTGGGCTTCAACCCTGGAGACATCAGT |
| | | GTGGAGTGGACCAGCAATGGGCATACAGAGGAGAACTA |
| | | CAAGGACACCGCACCAGTCCTGGACTCTGACGGTTCTTA |
| | | CTTCATATATAGCAAGCTCAATATGAAAACAAGCAAGTGG |
| | | GAGAAAACAGATTCCTTCTCATGCAACGTGAGACACGAG |
| | | GGTCTGAAAAATTACTACCTGAAGAAGACCATCTCCCGGT |
| | | CTCCGGGTAAA |
| 570 | DexinDJ-hIgG1 | ASPLKSQRDTDLPRPSISAEPGTVIPLGSHVTFVCRGPVGVQTF |
| | complete | RLERERNYLYSDTEDVSQTSPSESEARFRIDSVNAGNAGLFRCI |
| | sequence aa | YYKSRKWSEQSDYLELVVKGEDVTWALSQSQDDPRACPQGE |
| | | LPISTDIYYVDVWGNGTTVTVSSAMVRSDKTHTCPPCPAPELL |
| | | GGPSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSRDELTKNQV |
| | | SLTCLVKGFYPSDIAVEWESNGQPENNYKTTPPVLDSDGSFFL |
| | | YSKLTVDKSRWQQGNVFSCSVMHEALHNHYTQKSLSLSPGK |
| 571 | DexinDJ-hIgG1 | gcgtctccactcaaatctcagagggacaccgatctgcccagaccctccatctcggctg |
| | complete | agccgggcaccgtgatccccctggggagccatgtgactttcgtgtgccggggcccggt |
| | sequence nucl | tggggttcaaacattccgcctggagagggagaggaattatttatacagtgatactgaaga |
| | | tgtgtctcaaactagtccatctgagtcggaggccagattccgcattgactcagtaaatgc |
| | | aggcaatgccgggctttttcgctgcatctattacaagtcccgtaaatggtctgagcagag |
| | | tgactacctggagctggtggtgaaaggtgaggacgtcacctgggccctgtcccagtctc |
| | | aagacgaccctcgagcttgtccccagggggagctccccataagtaccgatatttacta |
| | | cgtggacgtctggggcaacgggaccacggtcaccgtctcctcaGCCATGGTTA |
| | | GATCTGACAAAACTCACACATGCCCACCGTGCCCAGCAC |
| | | CTGAACTCCTGGGGGGACCGTCAGTCTTCCTCTTCCCCCC |
| | | AAAACCCAAGGACACCCTCATGATCTCCCGGACCCCTGA |
| | | GGTCACATGCGTGGTGGTGGACGTGAGCCACGAAGACC |
| | | CTGAGGTCAAGTTCAACTGGTACGTGGACGGCGTGGAG |
| | | GTGCATAATGCCAAGACAAAGCCGCGGGAGGAGCAGTA |
| | | CAACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCCCAGCCCCCATCGAGAAAA |
| | | CCATCTCCAAAGCCAAAGGGCAGCCCCGAGAACCACAG |
| | | GTGTACACCCTGCCCCCATCCCGGGATGAGCTGACCAAG |
| | | AACCAGGTCAGCCTGACCTGCCTGGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGAC |
| | | TCCGACGGCTCCTTCTTCCTCTACAGCAAGCTCACCGTGG |
| | | ACAAGAGCAGGTGGCAGCAGGGGAACGTCTTCTCATGCT |
| | | CCGTGATGCATGAGGCTCTGCACAACCACTACACGCAGA |
| | | AGAGCCTCTCCCTGTCTCCGGGTAAA |

MGD21-exinDJ-mIgG2b

| | | |
|---|---|---|
| 572 | exinDJ variable | DLPRPSISAEPGTVIPLGSHVTFVCRGPVGVQTFRLERERNYLY |
| | part aa | SDTEDVSQTSPSESEARFRIDSVNAGNAGLFRCIYYKSRKWSE |
| | | QSDYLELVVKGEDVTWALSQSQDDPRACPQGELPISTDIYYV |
| | | DVWGNGTTVTVSS |
| 573 | exinDJ variable | gatctgcccagaccctccatctcggctgagccgggcaccgtgatccccctggggagc |
| | part nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctgtcccagtctcaagacgaccctcgagcttgtccccaggg |
| | | ggagctccccataagtaccgatatttactacgtggacgtctggggcaacgggaccacg |
| | | gtcaccgtctcctca |
| 574 | exinDJ-mIgG2b | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKGEDVTWALSQSQDDPRACPQGELPISTDIYYV |
| | | DVWGNGTTVTVSSAMVRSDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 575 | exinDJ-mIgG2b | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctgtcccagtctcaagacgaccctcgagcttgtccccaggg |
| | | ggagctccccataagtaccgatatttactacgtggacgtctggggcaacgggaccacg |
| | | gtcaccgtctcctcaGCCATGGTTAGATCTGACAAAACTCACACA |
| | | TGCCCACCGTGCCCAGCACCTGAACTCCTGGGGGGACC |
| | | GTCAGTCTTCCTCTTCCCCCCAAAACCCAAGGACACCCTC |
| | | ATGATCTCCCGGACCCCTGAGGTCACATGCGTGGTGGTG |
| | | GACGTGAGCCACGAAGACCCTGAGGTCAAGTTCAACTGG |
| | | TACGTGGACGGCGTGGAGGTGCATAATGCCAAGACAAA |
| | | GCCGCGGGAGGAGCAGTACAACAGCACGTACCGTGTGG |
| | | TCAGCGTCCTCACCGTCCTGCACCAGGACTGGCTGAATG |
| | | GCAAGGAGTACAAGTGCAAGGTCTCCAACAAAGCCCTCC |
| | | CAGCCCCCATCGAGAAAACCATCTCCAAAGCCAAAGGGC |
| | | AGCCCCGAGAACCACAGGTGTACACCCTGCCCCCATCCC |
| | | GGGATGAGCTGACCAAGAACCAGGTCAGCCTGACCTGC |
| | | CTGGTCAAAGGCTTCTATCCCAGCGACATCGCCGTGGAG |
| | | TGGGAGAGCAATGGGCAGCCGGAGAACAACTACAAGAC |
| | | CACGCCTCCCGTGCTGGACTCCGACGGCTCCTTCTTCCTC |
| | | TACAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTAAA |

MGD21-exin-mIgG2b

| | | |
|---|---|---|
| 576 | exin variable part | DLPRPSISAEPGTVIPLGSHVTFVCRGPVGVQTFRLERERNYLY |
| | aa | SDTEDVSQTSPSESEARFRIDSVNAGNAGLFRCIYYKSRKWSE |
| | | QSDYLELVVKGEDVTWAL |
| 577 | exin variable part | gatctgcccagaccctccatctcggctgagccgggcaccgtgatccccctggggagc |
| | nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctg |
| 578 | exin-mIgG2b | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKGEDVTWALAMVRSDKTHTCPPCPAPELLGGPS |
| | | VFLFPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGK |
| 579 | exin-mIgG2b | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctgGCCATGGTTAGATCTGACAAAACTCA |
| | | CACATGCCCACCGTGCCCAGCACCTGAACTCCTGGGGGG |
| | | ACCGTCAGTCTTCCTCTTCCCCCCAAAACCCAAGGACACC |
| | | CTCATGATCTCCCGGACCCCTGAGGTCACATGCGTGGTG |
| | | GTGGACGTGAGCCACGAAGACCCTGAGGTCAAGTTCAAC |
| | | TGGTACGTGGACGGCGTGGAGGTGCATAATGCCAAGAC |
| | | AAAGCCGCGGGAGGAGCAGTACAACAGCACGTACCGTG |
| | | TGGTCAGCGTCCTCACCGTCCTGCACCAGGACTGGCTG |
| | | AATGGCAAGGAGTACAAGTGCAAGGTCTCCAACAAAGCCC |
| | | TCCCAGCCCCCATCGAGAAAACCATCTCCAAAGCCAAAG |
| | | GGCAGCCCCGAGAACCACAGGTGTACACCCTGCCCCCAT |
| | | CCCGGGATGAGCTGACCAAGAACCAGGTCAGCCTGACCT |
| | | GCCTGGTCAAAGGCTTCTATCCCAGCGACATCGCCGTGG |
| | | AGTGGGAGAGCAATGGGCAGCCGGAGAACAACTACAAG |
| | | ACCACGCCTCCCGTGCTGGACTCCGACGGCTCCTTCTTCC |
| | | TCTACAGCAAGCTCACCGTGGACAAGAGCAGGTGGCAG |
| | | CAGGGGAACGTCTTCTCATGCTCCGTGATGCATGAGGCT |
| | | CTGCACAACCACTACACGCAGAAGAGCCTCTCCCTGTCTC |
| | | CGGGTAAA |

MGD21-ex-mIgG2b

| | | |
|---|---|---|
| 580 | exon variable | DLPRPSISAEPCTVIPLGSHVTFVCRCPVGVQTFRLERERNYLYSDTEDVSQTS |
| | part aa | PSESEARFRIDSVNAGNAGLFRCIYYKSRKWSEQSDYLELVVK |
| 581 | exon variable | gatctgcccagaccctccatctcggctgagccgggcaccgtgatccccctggggagc |
| | part nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaa |
| 582 | ex-mIgG2b | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKAMVRSPSGPISTINPCPPCKECHKCPAPNLEGG |
| | | PSVFIFPPNIKDVLMISLTPKVTCVVVDVSEDDPDVQISWFVN |
| | | NVEVHTAQTQTHREDYNSTIRVVSTLPIQHQDWMSGKEFKC |
| | | KVNNKDLPSPIERTISKIKGLVRAPQVYILPPPAEQLSRKDVSLT |
| | | CLVVGFNPGDISVEWTSNCHTEENYKDTAPVLDSDGSYFIYS |
| | | KLNMKTSKWEKTDSFSCNVRHEGLKNYYLKKTISRSPGK |
| 583 | ex-mIgG2b | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaGCC |
| | | ATGGTTAGATCTCCCAGCGGGCCCATTTCAACAATCAACC |
| | | CCTGTCCTCCATGCAAGGAGTGTCACAAATGCCCAGCTCC |
| | | TAACCTCGAGGGTGGACCATCCGTCTTCATCTTCCCTCCA |
| | | AATATCAAGGATGTACTCATGATCTCCCTGACACCCAAGG |
| | | TCACGTGTGTGGTGGTGGATGTGAGCGAGGATGACCCA |
| | | GACGTCCAGATCAGCTGGTTTGTGAACAACGTGGAAGTA |
| | | CACACAGCTCAGACACAAACCCATAGAGAGGATTACAAC |
| | | AGTACTATCCGGGTGGTCAGCACCCTCCCCATCCAGCAC |
| | | CAGGACTGGATGAGTGGCAAGGAGTTCAAATGCAAGGT |
| | | CAACAACAAAGACCTCCCATCACCCATCGAGAGAACCATC |
| | | TCAAAAATTAAAGGGCTAGTCAGAGCTCCACAAGTATACA |
| | | TCTTGCCGCCACCAGCAGAGCAGTTGTCCAGGAAAGATG |
| | | TCAGTCTCACTTGCCTGGTCGTGGGCTTCAACCCTGGAGA |
| | | CATCAGTGTGGAGTGGACCAGCAATGGGCATACAGAGG |
| | | AGAACTACAAGGACACCGCACCAGTCCTGGACTCTGACG |
| | | GTTCTTACTTCATATATAGCAAGCTCAATATGAAAACAAGC |
| | | AAGTGGGAGAAAACAGATTCCTTCTCATGCAACGTGAGA |
| | | CACGAGGGTCTGAAAAATTACTACCTGAAGAAGACCATCT |
| | | CCCGGTCTCCGGGTAAA |
| 584 | ex-hIgG1 | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKAMVRSDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPAP |
| | | IEKTISKAKGQPREPQVYTLPPSRDELTKNQVSLTCLVKCFYPS |
| | | DIAVEWESNGQPENNYKTTPPVLDSDGSFFLYSKLTVDKSRW |
| | | QQGNVFSCSVMHEALHNHYTQKSLSLSPGK |
| 585 | ex-hIgG1 | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaGCC |
| | | ATGGTTAGATCTGACAAAACTCACACATGCCCACCGTGCC |
| | | CAGCACCTGAACTCCTGGGGGGACCGTCAGTCTTCCTCTT |
| | | CCCCCCAAAACCCAAGGACACCCTCATGATCTCCCGGAC |
| | | CCCTGAGGTCACATGCGTGGTGGTGGACGTGAGCCACG |
| | | AAGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCCCAGCCCCCATCGA |
| | | GAAAACCATCTCCAAAGCCAAAGGGCAGCCCCGAGAACC |
| | | ACAGGTGTACACCCTGCCCCCATCCCGGGATGAGCTGAC |
| | | CAAGAACCAGGTCAGCCTGACCTGCCTGGTCAAAGGCTT |
| | | CTATCCCAGCGACATCGCCGTGGAGTGGGAGAGCAATG |
| | | GGCAGCCGGAGAACAACTACAAGACCACGCCTCCCGTG |
| | | CTGGACTCCGACGGCTCCTTCTTCCTCTACAGCAAGCTCA |
| | | CCGTGGACAAGAGCAGGTGGCAGCAGGGGAACGTCTTC |
| | | TCATGCTCCGTGATGCATGAGGCTCTGCACAACCACTACA |
| | | CGCAGAAGAGCCTCTCCCTGTCTCCGGGTAAA |

### Example 6: Ig Fusion Proteins Comprising the Mutated LAIR-1 Fragment Bind to IEs

The four exemplary mouse IgG2b fusion proteins constructed in Example 5 (i.e. one of each type: M1, M2, M3, and M4), which were consisting of amino acid sequences as outlined for the “complete fusion protein”, respectively, were used to investigate whether the mutated LAIR-1 fragment is sufficient to bind to infected erythrocytes (IEs). To this end, HEK 293 cells were transfected with the fusion proteins only and supernatants were collected and tested for binding to IEs as described in Example 1. Briefly, IEs are stained with SYBR Green I dye (DNA) to discriminate them from uninfected erythrocytes used as control. The surnatants are added on top of IEs and binding of fusion proteins to IEs is detected using a secondary-anti-human or anti-mouse IgG (Fc-specific) antibody.

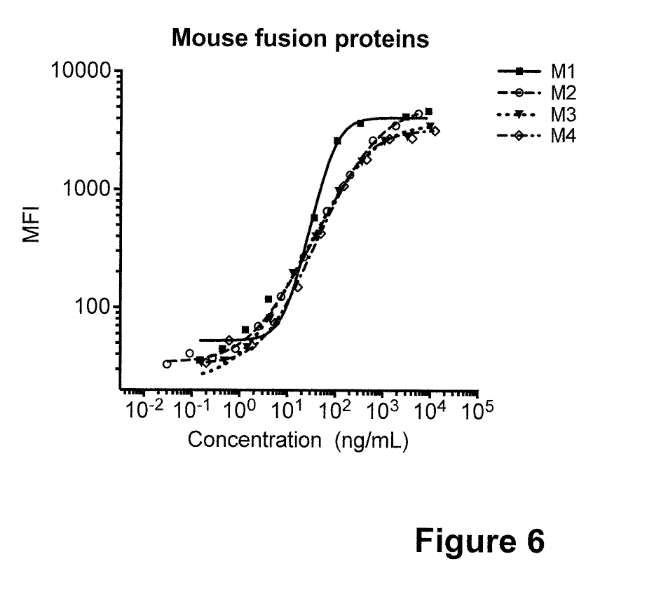

All fusion proteins were found to bind to infected erythrocytes (FIG. 6). These results identify the mutated LAIR-1 fragment as a unique domain that binds to malaria-infected erythrocytes.

### Example 7: Antibodies and Ig Fusion Proteins Efficiently Opsonize and Agglutinate P. falciparum-Infected Erythrocytes

To investigate the potential therapeutic impact of selected broadly reactive antibodies of Example 1 and of the Ig fusion proteins constructed in Example 5, i.e. whether these antibodies/fusion proteins could opsonize infected erythrocytes and thus mediate their phagocytosis and destruction by mononuclear phagocytes, their capacity to opsonize infected erythrocytes was measured.

To this end, P. falciparum (3D7) were stained with DAPI and mixed with different concentrations of the two exemplary human IgG1 fusion proteins constructed in Example 5 (i.e. one of each type: H1 and H2), which were consisting of amino acid sequences as outlined for the “complete fusion protein”, respectively. Thereafter, they were incubated with human monocytes at 37° C. for 1 hour.

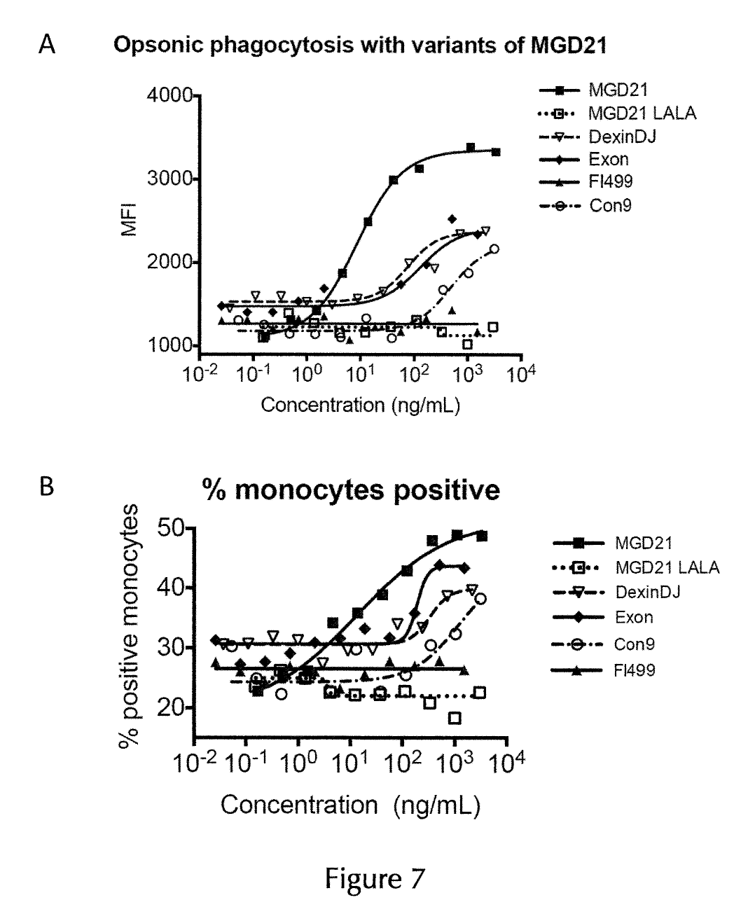

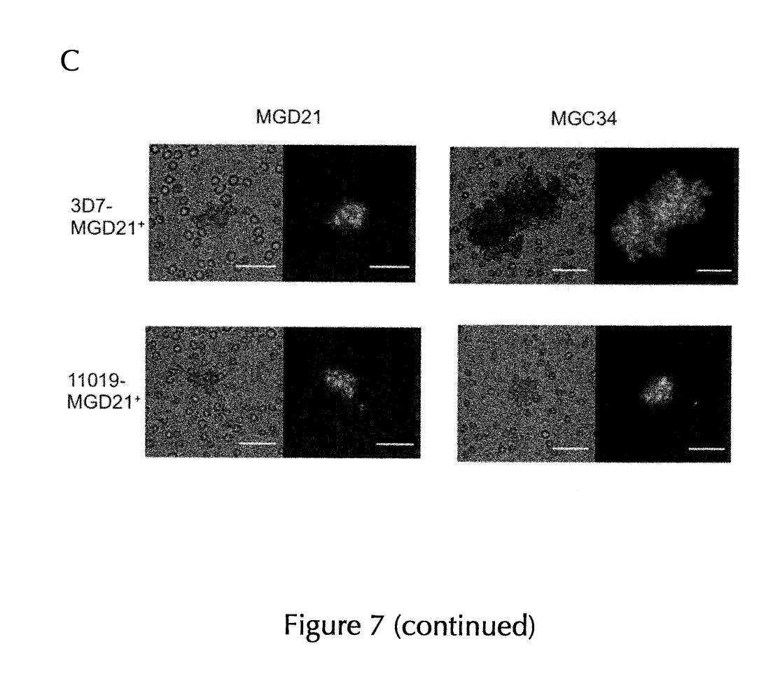

Thereafter, monocytes were stained with anti-CD14-APC to measure the fraction of monocytes that contained parasites. The results are shown in FIG. 7 with FIG. 7A showing the MFI (mean fluorecnce intensity) of DAPI and FIG. 7B showing the percentage of DAPI-positive monocytes calculated in CD14-positive populations.

The results demonstrate that low concentrations of the two exemplary human IgG1 fusion proteins constructed in Example 5 can efficiently opsonize infected erythrocytes. These findings indicate that the Ig fusion proteins constructed in Example 5 can potently mediate phagocytosis and destruction of infected erythrocytes in vivo.

Finally, it was tested whether the antibodies MGD21 and MGC34 were able to agglutinate erythrocytes infected with P. falciparum 3D7 or the Kenyan P. falciparum isolate 11019. As shown in FIG. 7C MGD21, as well as MGC34, could agglutinate erythrocytes infected with 3D7 or the Kenyan isolate 11019.

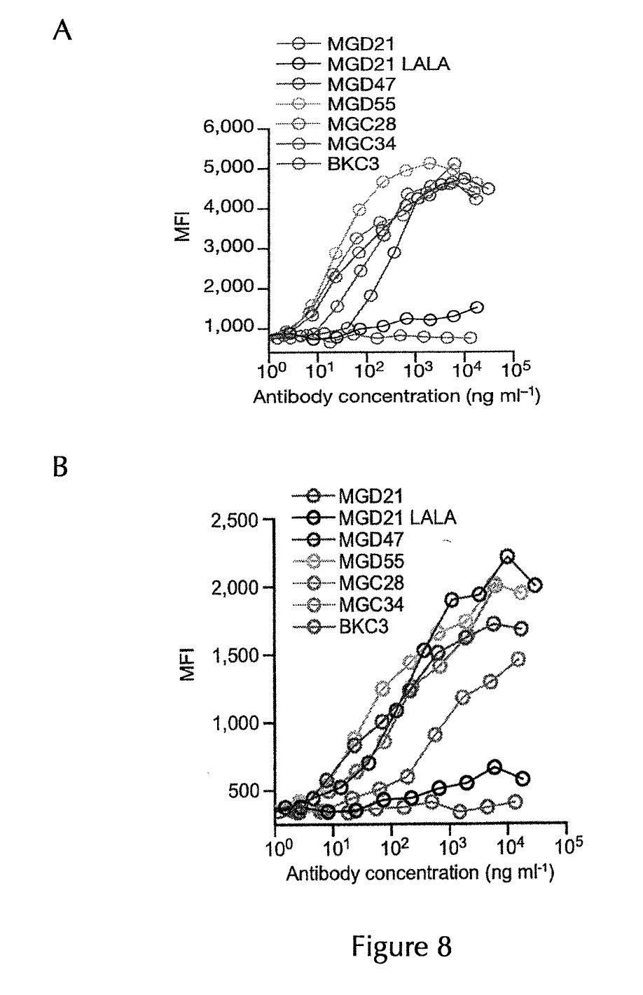

Next, P. falciparum (3D7 or 11019) were stained with DAPI and mixed with different concentrations of the five broadly reactive antibodies described in Table 2 and Example 1 (i.e. one of each type: MGD21, MGD47, MGD55, MGC28 and MGC34). BKC3 was used as control. Thereafter, they were incubated with human monocytes at 37° C. for 1 hour and, then, monocytes were stained with anti-CD14-APC to measure the fraction of monocytes that contained parasites. The results are shown in FIG. 8 with FIG. 8A showing the MFI (mean fluorecnce intensity) of DAPI in 3D7 and FIG. 8B showing the MFI (mean fluorecnce intensity) of DAPI in 11019-MGD21⁺ IEs. Results show that low concentrations of all five antibodies tested constructed (MGD21, MGD47, MGD55, MGC28 and MGC34; cf. Table 2) can efficiently opsonize infected erythrocytes, whereas MGD21LALA and BKC3 controls show no effect. These findings indicate that the broadly reactive antibodies can potently mediate phagocytosis and destruction of infected erythrocytes in vivo.

### Example 8: A Model of the Mutated LAIR-1 Fragment: Somatic Mutations in the LAIR1 Fragment are Critical Both for Binding IE and Losing Binding to Collagen

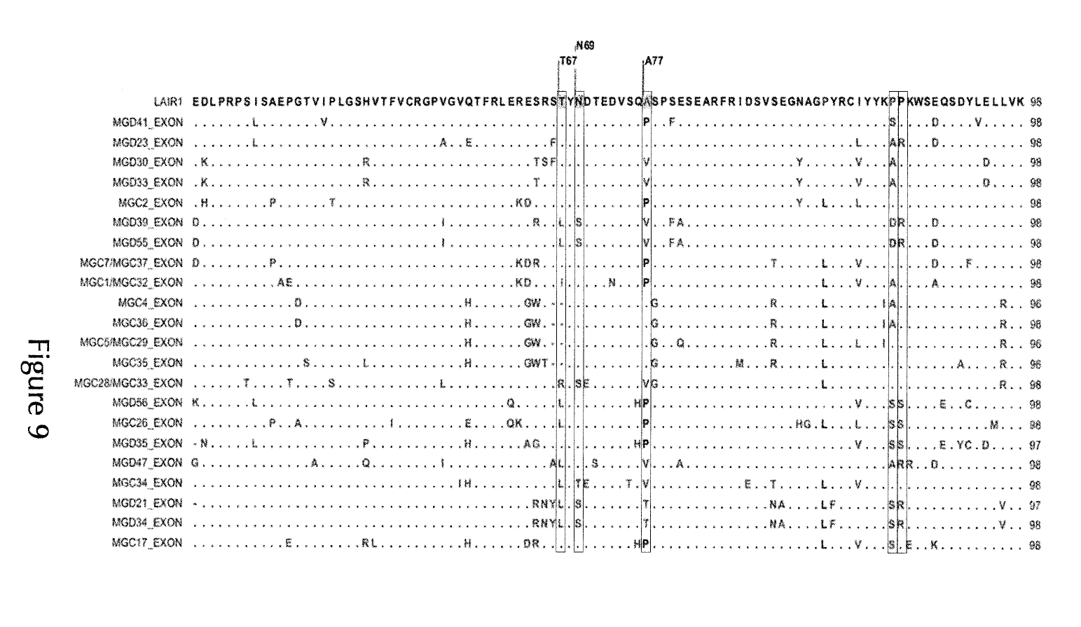

The mutated LAIR-1 fragment of the antibodies of Example 1 has a sequence homology ranging from 84% to 96% with the amino acids 24 to 121 of native human LAIR-1 (SEQ ID NO: 14; for example: MGD53_exon=96%; MGC2_exon=91%; MGD21_exon=86%; MGD35_exon=84%). FIG. 9 shows an alignment of the mutated LAIR-1 exon of the human monoclonal antibodies of Example 1 (cf. SEQ ID NOs 61, 63, 65, 67, 69, 71, 73, 75, 77, 79, 81, 83, 85, 87, 89, 91, 93, 95, 97, 99, 101 and 103—Table 1) with amino acids 24 to 121 of native human LAIR-1 (SEQ ID NO: 14).

From the human monoclonal antibodies of Example 1 those antibodies were selected, which most strongly bind to the most of the IEs infected with different P. falciparum strains (“broadest” binding to IEs). These were MGD21, MGD34, MGD39, MGD47, and MGD55 (cf. Table 7 of Example 1). An alignment of the amino acid sequences of the LAIR-1 exon fragment of these antibodies, i.e. amino acid sequences according to SEQ ID NOs: 83, 91, 95, 99 and 101 with an exemplary genomic LAIR-1 sequence, revealed five mutated residues, which are crucial to increase the affinity and the breadth of binding to P. falciparum-IE. The same five mutated residues were also found to be important for losing binding to collagen that is the natural ligand of the native LAIR-1 receptor (see Example 9). The five crucial positions are T67, N69, A77, P106 and P107 and are shown in frames in FIG. 9.

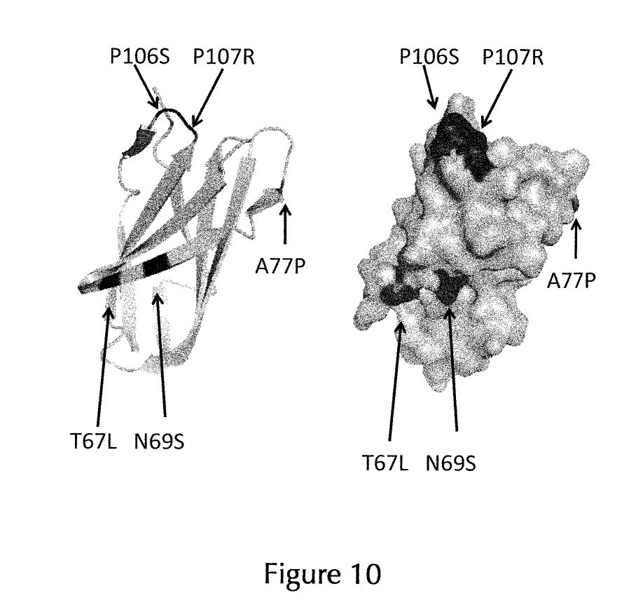

The mutated LAIR-1 fragment according to the present invention was modelled based on a crystal structure of native LAIR-1 extracellular domain (residues: 24 to 121) (FIG. 10; for the crystal structure of native LAIR-1 see MMDB ID: 78950, PDB ID: 3KGR). According to the crystal structure of LAIR-1, at least one of the following five residues must be mutated to lose collagen binding and to gain binding to infected erythrocytes (positions are defined in respect to the amino acid sequence of native human LAIR-1):

### T67, N69, A77, P106, and P107 (FIG. 10).

Preferred mutations are shown below in Table 8, with T67L, N69S, A77T, P106S, and P107R being the most preferred mutations for each of the five positions.

TABLE 8
preferred mutations for each of the five positions
in the mutated LAIR-1 fragment.

| Position | Mutation |
|---|---|
| T67 | T67L, T67G, T67I, T67R, T67K |
| N69 | N69S, N69T |
| A77 | A77T, A77P, A77V |
| P106 | P106S, P106A, P106D |
| P107 | P107R, P107S |

### Example 9: Identification of Mutations of LAIR1 Fragment that are Crucial for Binding to P. falciparum-IE

To identify which of the five mutations are crucial for binding to IEs, fusion proteins comprising the LAIR-1 fragment, which was either unmutated (SEQ ID NO: 14) or carrying one or more of the following five mutations: T67L (“L”); N69S (“S1”); A77T (“T”); P106S (“S2”); and P107R (“R”), were produced. The principal structure of these fusion proteins (i.e. except for the mutated LAIR-1 fragment) is identical to that of “H2” of Example 5 as described above (also referred to as “ex-hIgG1”). While in the construct “H2” of Example 5 (also referred to as “ex-hIgG1”) the mutated LAIR-1 exon of the antibody MGD21 was used (SEQ ID NO: 83), the present constructs are instead based on the native human LAIR-1 fragment (amino acids 24-121; SEQ ID NO: 14) and differ from that (i.e. from SEQ ID NO: 14) only in one or more of the following five mutations: T67L (“L”); N69S (“S1”); A77T (“T”); P106S (“S2”); and P107R (“R”).

Table 9 shows SEQ ID and sequences of the different fusion proteins.

TABLE 9
Sequences and Seq ID NOs of the LAIR-1 Ig fusion protein
constructs of Example 9, whereby only the sequences of the
(mutated) LAIR-1 fragment are shown. Mutations in comparison
to native human LAIR-1 (SEQ ID NO: 14) are shown underlined
in the amino acid sequence.

| SEQ ID | | |
|---|---|---|
| NO | Description | Sequence* |
| 10 | LAIR1ex aa | EDLPRPSISAEPGTVIPLGSHVTFVCRGPVGVQTFRLERESRSTY |
| | | NDTEDVSQASPSESEARFRIDSVSEGNAGPYRCIYYKPPKWSE |
| | | QSDYLELLVK |
| 586 | LAIR1ex nucl | GAGGACCTGCCAAGACCCAGCATCTCCGCAGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCCAGCGAGTCCGAAGCTCGGTTCAGAAT |
| | | CGACTCTGTCAGTGAAGGAAATGCCGGCCCTTACAGATG |
| | | CATCTACTATAAGCCCCCTAAATGGTCAGAGCAGAGCGAT |
| | | TATCTGGAACTGCTGGTGAAG |
| 587 | LAIR1ex + L aa | EDLPRPSISAEPGTVIPLGSHVTFVCRGPVGVQTFRLERESRSLY |
| | | NDTEDVSQASPSESEARFRIDSVSEGNAGPYRCIYYKPPKWSE |
| | | QSDYLELLVK |
| 588 | LAIR1ex + L nucl | GAGGACCTGCCAAGACCCAGCATCTCCGCAGAACCTGG |
| | | GACCGTGATTCCACTGGGCTCCCACGTGACATTCGTCTGC |
| | | AGAGGCCCCGTGGGAGTCCAGACTTTTAGGCTGGAGCG |
| | | CGAATCTCGAAGTCTGTACAACGACACAGAGGACGTGAG |
| | | CCAGGCCTCACCAAGCGAGTCCGAAGCTCGGTTCAGAAT |
| | | CGACTCTGTCAGTGAAGGAAATGCCGGCCCTTACAGATG |
| | | CATCTACTATAAGCCCCCTAAATGGTCAGAGCAGAGCGAT |
| | | TATCTGGAACTGCTGGTGAAG |
| 589 | LAIR1ex + LR aa | EDLPRPSISAEPGTVIPLGSHVTFVCRGPVGVQTFRLERESRSLY |
| | | NDTEDVSQASPSESEARFRIDSVSEGNAGPYRCIYYKPRKWSE |
| | | QSDYLELLVK |
| 590 | LAIR1ex + LR nucl | GAGGACCTGCCCCGCCCTAGCATCTCCGCAGAACCAGG |
| | | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACAACGACACCGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGCCAAGAAAATGGTCAGAGCAGAGCGA |
| | | TTATCTGGAACTGCTGGTGAAG |
| 591 | LAIR1ex + LS1 aa | EDLPRPSISAEPGTVIPLGSHVTFVCRGPVGVQTFRLERESRSLY |
| | | SDTEDVSQASPSESEARFRIDSVSEGNAGPYRCIYYKPPKWSEQ |
| | | SDYLELLVK |
| 592 | LAIR1ex + LSI | GAGGACCTGCCAAGACCCAGCATCTCCGCAGAACCTGG |
| | nucl | GACCGTGATTCCACTGGGCTCCCACGTGACATTCGTCTGC |
| | | AGAGGCCCCGTGGGAGTCCAGACTTTTAGGCTGGAGCG |
| | | CGAATCTCGAAGTCTGTACTCCGACACAGAGGACGTGAG |
| | | CCAGGCCTCACCAAGCGAGTCCGAAGCTCGGTTCAGAAT |
| | | CGACTCTGTCAGTGAAGGAAACGCCGGCCCTTACAGATG |
| | | CATCTACTATAAGCCCCCTAAATGGTCAGAGCAGAGCGAT |
| | | TATCTGGAACTGCTGGTGAAG |
| 593 | LAIR1ex + LS1R | EDLPRPSISAEPGTVIPLGSHVTFVCRGPVGVQTFRLERESRSLY |
| | aa | SDTEDVSQASPSESEARFRIDSVSEGNAGPYRCIYYKPRKWSEQ |
| | | SDYLELLVK |
| 594 | LAIR1ex + LS1R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACTCCGACACCGAGGACGTGAG |
| | | CCAGGCCTCACCTAGCGAGTCCGAAGCTCGGTTCAGAAT |
| | | CGACTCTGTCAGTGAAGGAAACGCCGGCCCTTACAGATG |
| | | CATCTACTATAAGCCAAGAAAATGGTCAGAGCAGAGCGA |
| | | TTATCTGGAACTGCTGGTGAAG |
| 595 | LAIR1ex + LS1S2R | EDLPRPSISAEPGTVIPLGSHVTFVCRGPVGVQTFRLERESRSLY |
| | aa | SDTEDVSQASPSESEARFRIDSVSEGNAGPYRCIYYKSRKWSEQ |
| | | SDYLELLVK |
| 596 | LAIR1ex + LS1S2R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACTCCGACACCGAGGACGTGAG |
| | | CCAGGCCTCACCTAGCGAGTCCGAAGCTCGGTTCAGAAT |
| | | CGACTCTGTCAGTGAAGGAAACGCCGGCCCATACAGATG |
| | | CATCTACTATAAGAGCAGAAAATGGTCAGAGCAGAGCGA |
| | | TTATCTGGAACTGCTGGTGAAG |
| 597 | LAIR1ex + LS1T aa | EDLPRPSISAEPGTVIPLGSHVTFVCRGPVGVQTFRLERESRSLY |
| | | SDTEDVSQTSPSESEARFRIDSVSEGNAGPYRCIYYKPPKWSEQ |
| | | SDYLELLVK |
| 598 | LAIR1ex + LS1T | GAGGACCTGCCAAGACCCAGCATCTCCGCCGAACCTGG |
| | nucl | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTCTGTACTCCGACACCGAGGACGTGAG |
| | | CCAGACATCACCCAGCGAGTCCGAAGCCCGGTTCAGAAT |
| | | CGACTCTGTCAGTGAAGGAAACGCTGGCCCTTACAGATG |
| | | CATCTACTATAAGCCCCCTAAATGGTCAGAGCAGAGCGAT |
| | | TATCTGGAACTGCTGGTGAAG |
| 599 | LAIR1ex + LS1TR | EDLPRPSISAEPGTVIPLGSHVTFVCRGPVGVQTFRLERESRSLY |
| | aa | SDTEDVSQTSPSESEARFRIDSVSEGNAGPYRCIYYKPRKWSEQ |
| | | SDYLELLVK |
| 600 | LAIR1ex + LS1TR | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTCTGTACTCCGACACCGAGGACGTGAGC |
| | | CAGACATCACCTAGCGAGTCCGAAGCCCGGTTCAGAATC |
| | | GACTCTGTCAGTGAAGGAAACGCTGGCCCTTACAGATGC |
| | | ATCTACTATAAGCCAAGAAAATGGTCAGAGCAGAGCGAT |
| | | TATCTGGAACTGCTGGTGAAG |
| 601 | LAIR1ex + LS1TS2R | EDLPRPSISAEPGTVIPLGSHVTFVCRGPVGVQTFRLERESRSLY |
| | aa | SDTEDVSQTSPSESEARFRIDSVSEGNAGPYRCIYYKSRKWSEQ |
| | | SDYLELLVK |
| 602 | LAIR1ex + LS1TS2R | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTCTGTACTCCGACACCGAGGACGTGAGC |
| | | CAGACATCACCTAGCGAGTCCGAAGCCCGGTTCAGAATC |
| | | GACTCTGTCAGTGAAGGAAACGCTGGCCCATACAGATGC |
| | | ATCTACTATAAGAGCAGAAAATGGTCAGAGCAGAGCGAT |
| | | TATCTGGAACTGCTGGTGAAG |
| 603 | LAIR1ex + LS2R | EDLPRPSISAEPGTVIPLGSHVTFVCRGPVGVQTFRLERESRSLY |
| | aa | NDTEDVSQASPSESEARFRIDSVSEGNAGPYROYYKSRKWSE |
| | | QSDYLELLVK |
| 604 | LAIR1ex + LS2R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACAACGACACCGAGGACGTGAG |
| | | CCAGGCCTCACCTAGCGAGTCCGAAGCTCGGTTCAGAAT |
| | | CGACTCTGTCAGTGAAGGAAATGCCGGCCCATACAGATG |
| | | CATCTACTATAAGTCTAGAAAATGGTCAGAGCAGAGCGAT |
| | | TATCTGGAACTGCTGGTGAAG |
| 605 | LAIR1ex + LT aa | EDLPRPSISAEPGTVIPLGSHVTFVCRGPVGVQTFRLERESRSLY |
| | | NDTEDVSQTSPSESEARFRIDSVSEGNAGPYRCIYYKPPKWSE |
| | | QSDYLELLVK |
| 606 | LAIR1ex + LT nucl | GAGGACCTGCCAAGACCCAGCATCTCCGCCGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTCTGTACAACGACACCGAGGACGTGAG |
| | | CCAGACATCACCCAGCGAGTCCGAAGCCCGGTTCAGAAT |
| | | CGACTCTGTCAGTGAAGGAAATGCTGGCCCTTACAGATG |
| | | CATCTACTATAAGCCCCCTAAATGGTCAGAGCAGAGCGAT |
| | | TATCTGGAACTGCTGGTGAAG |
| 607 | LAIR1ex + R aa | EDLPRPSISAEPGTVIPLGSHVTFVCRGPVGVQTFRLERESRSTY |
| | | NDTEDVSQASPSESEARFRIDSVSEGNAGPYRCIYYKPRKWSE |
| | | QSDYLELLVK |
| 608 | LAIR1ex + R nucl | GAGGACCTGCCCCGCCCTAGCATCTCCGCAGAACCAGG |
| | | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGCCAAGAAAATGGTCAGAGCAGAGCGA |
| | | TTATCTGGAACTGCTGGTGAAG |
| 609 | LAIR1ex + S1 aa | EDLPRPSISAEPGTVIPLGSHVTFVCRGPVGVQTFRLERESRSTY |
| | | SDTEDVSQASPSESEARFRIDSVSEGNAGPYRCIYYKPPKWSEQ |
| | | SDYLELLVK |
| 610 | LAIR1ex + S1 nucl | GAGGACCTGCCAAGACCCAGCATCTCCGCAGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCCAGCGAGTCCGAAGCTCGGTTCAGAAT |
| | | CGACTCTGTCAGTGAAGGAAACGCCGGCCCTTACAGATG |
| | | CATCTACTATAAGCCCCCTAAATGGTCAGAGCAGAGCGAT |
| | | TATCTGGAACTGCTGGTGAAG |
| 611 | LAIR1ex + S1R aa | EDLPRPSISAEPGTVIPLGSHVTFVCRGPVGVQTFRLERESRSTY |
| | | SDTEDVSQASPSESEARFRIDSVSEGNAGPYRCIYYKPRKWSEQ |
| | | SDYLELLVK |
| 612 | LAIR1ex + S1R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCTAGCGAGTCCGAAGCTCGGTTCAGAAT |
| | | CGACTCTGTCAGTGAAGGAAACGCCGGCCCTTACAGATG |
| | | CATCTACTATAAGCCAAGAAAATGGTCAGAGCAGAGCGA |
| | | TTATCTGGAACTGCTGGTGAAG |
| 613 | LAIR1ex + S1S2R | EDLPRPSISAEPGTVIPLGSHVTFVCRGPVGVQTFRLERESRSTY |
| | aa | SDTEDVSQASPSESEARFRIDSVSEGNAGPYRCIYYKSRKWSEQ |
| | | SDYLELLVK |
| 614 | LAIR1 ex + S1S2R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCTAGCGAGTCCGAAGCTCGGTTCAGAAT |
| | | CGACTCTGTCAGTGAAGGAAACGCCGGCCCATACAGATG |
| | | CATCTACTATAAGAGCAGAAAATGGTCAGAGCAGAGCGA |
| | | TTATCTGGAACTGCTGGTGAAG |
| 615 | LAIR1ex + S1T aa | EDLPRPSISAEPGTVIPLGSHVTFVCRGPVGVQTFRLERESRSTY |
| | | SDTEDVSQTSPSESEARFRIDSVSEGNAGPYRCIYYKPPKWSEQ |
| | | SDYLELLVK |
| 616 | LAIR1ex + S1T | GAGGACCTGCCAAGACCCAGCATCTCCGCCGAACCTGG |
| | nucl | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGACCTCACCCAGCGAGTCCGAAGCCCGGTTCAGAAT |
| | | CGACTCTGTCAGTGAAGGAAACGCTGGCCCTTACAGATG |
| | | CATCTACTATAAGCCCCCTAAATGGTCAGAGCAGAGCGAT |
| | | TATCTGGAACTGCTGGTGAAG |
| 617 | LAIR1ex + S2 aa | EDLPRPSISAEPGTVIPLGSHVTFVCRGPVGVQTFRLERESRSTY |
| | | NDTEDVSQASPSESEARFRIDSVSEGNAGPYRCIYYKSPKWSE |
| | | QSDYLELLVK |
| 618 | LAIR1ex + S2 nucl | GAGGACCTGCCCAGACCTAGCATCTCCGCAGAACCAGG |
| | | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGTCTCCAAAATGGTCAGAGCAGAGCGAT |
| | | TATCTGGAACTGCTGGTGAAG |
| 619 | LAIR1ex + S2R aa | EDLPRPSISAEPGTVIPLGSHVTFVCRGPVGVQTFRLERESRSTY |
| | | NDTEDVSQASPSESEARFRIDSVSEGNAGPYRCIYYKSRKWSE |
| | | QSDYLELLVK |
| 620 | LAIR1ex + S2R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCATACAGATG |
| | | CATCTACTATAAGTCTAGAAAATGGTCAGAGCAGAGCGAT |
| | | TATCTGGAACTGCTGGTGAAG |
| 621 | LAIRIex + T aa | EDLPRPSISAEPGTVIPLGSHVTFVCRGPVGVQTFRLERESRSTY |
| | | NDTEDVSQTSPSESEARFRIDSVSEGNAGPYRCIYYKPPKWSE |
| | | QSDYLELLVK |
| 622 | LAIR1ex + T nucl | GAGGACCTGCCAAGACCCAGCATCTCCGCCGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGACCTCACCCAGCGAGTCCGAAGCCCGGTTCAGAAT |
| | | CGACTCTGTCAGTGAAGGAAATGCTGGCCCTTACAGATG |
| | | CATCTACTATAAGCCCCCTAAATGGTCAGAGCAGAGCGAT |
| | | TATCTGGAACTGCTGGTGAAG |
| 623 | LAIR1ex + TS2R | EDLPRPSISAEPGTVIPLGSHVTFVCRGPVGVQTFRLERESRSTY |
| | aa | NDTEDVSQTSPSESEARFRIDSVSEGNAGPYRCIYYKSRKWSE |
| | | QSDYLELLVK |
| 624 | LAIR1 ex + TS2R | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTACCTACAACGACACAGAGGACGTGAGC |
| | | CAGACCTCACCTAGCGAGTCCGAAGCCCGGTTCAGAATC |
| | | GACTCTGTCAGTGAAGGAAATGCTGGCCCATACAGATGC |
| | | ATCTACTATAAGTCTAGAAAATGGTCAGAGCAGAGCGATT |
| | | ATCTGGAACTGCTGGTGAAG |

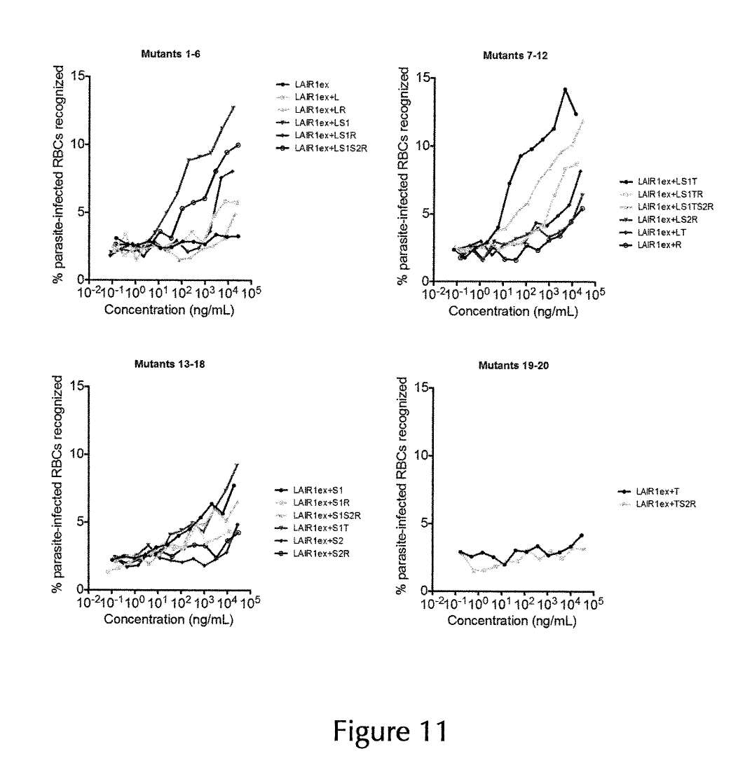

The 20 fusion proteins were expressed in HEK293 cells and the binding to P. falciparum was assessed by staining IEs, as described in Example 1. The results are shown in FIG. 11. These results show that native human LAIR-1 (“LAIR1 ex”) does not bind to IEs and that at least one of the mutations T67L (“L”); N69S (“S1”); A77T (“T”); P106S (“S2”) and P107R (“R”) is necessary for gaining binding to IEs.

### Example 10: Influence of the Mutations of LAIR1 Fragment on Binding to Collagen

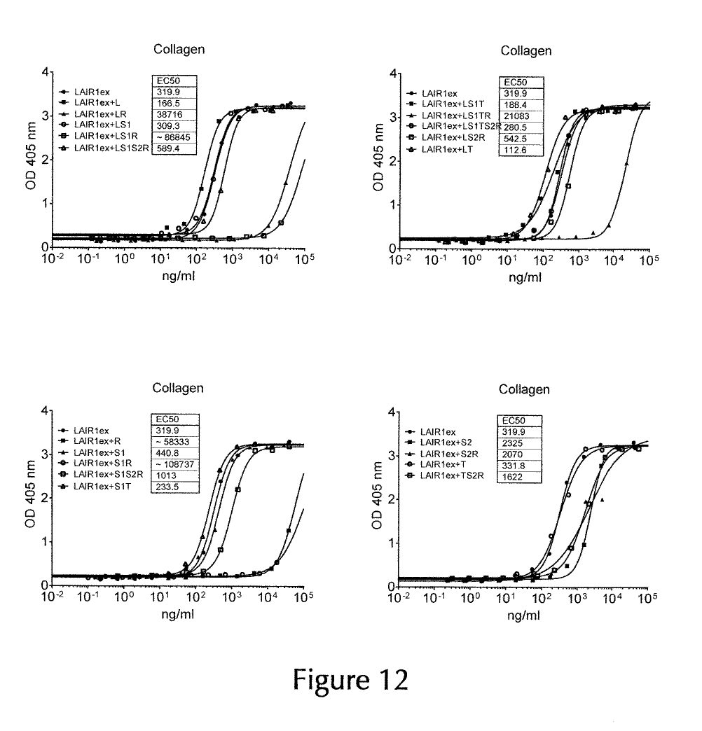

Native human LAIR-1 is well-known to bind collagen, in particular via its extracellular domain (T. Harma C. Brondijk, Talitha de Ruiter, Joost Ballering, Hans Wienk, Robert Jan Lebbink, Hugo van Ingen, Rolf Boelens, Richard W. Farndale, Linde Meyaard, and Eric G. Huizinga (2010): Crystal structure and collagen-binding site of immune inhibitory receptor LAIR-1: unexpected implications for collagen binding by platelet receptor GPVI. Blood 115:7). To identify whether the five mutations influence binding to collagen, the 20 fusion proteins of Example 9 were expressed in HEK293 cells and the binding to collagen was assessed by ELISA. Briefly ELISA plates were coated with Collagen type 1, blocked with PBS 1% BSA, followed by incubation with supernatants and a secondary-anti-human (Fc-specific) antibody for detection. The results are shown in FIG. 12. These results show that in particular mutation P107R appears to deteriorate binding to collagen (FIG. 12).

### Example 11: Identification of the P. falciparum Antigen(s) Recognized by MGD21

To identify the antigen(s) recognized by the LAIR1-containing antibodies, stable P. falciparum 3D7 lines, which were enriched (3D7-MGD21⁺) or depleted (3D7-MGD21⁻) of MGD21 reactivity were generated.

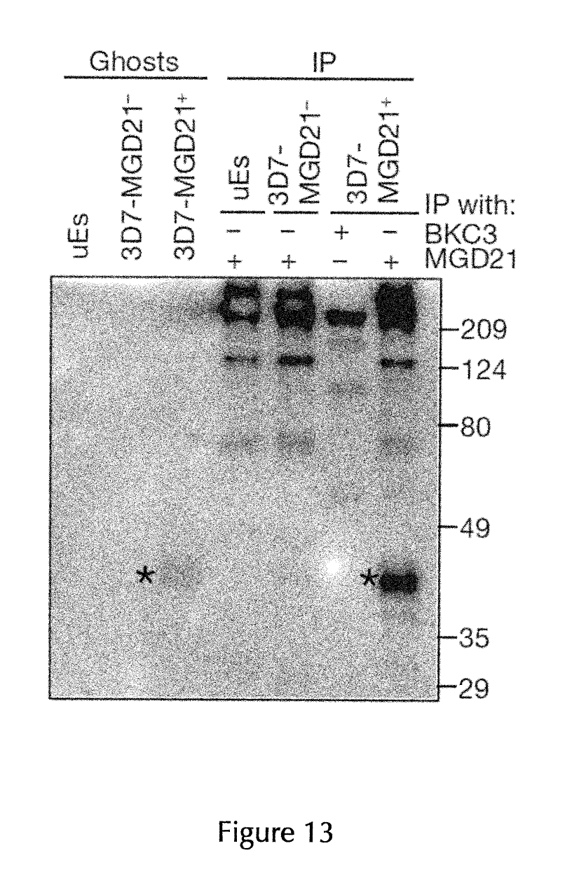

To investigate MGD21 binding to erythrocyte ghosts and MGD21 immunoprecipitates (IP) prepared from 3D7-MGD21⁺ and 3D7-MGD21⁻ IEs, a western blot was performed. Controls included uninfected erythrocytes (uEs) and immunoprecipitates with an irrelevant antibody (BKC3). Anti-human IgG was used as the secondary antibody, resulting in detection of antibodies used for immunoprecipitation alongside antigens of interest. As shown in FIG. 13, western blot analysis revealed two specific MGD21-reactive bands of 40-45 kilodaltons (kDa) in erythrocyte ghosts and in MGD21 immunoprecipitates prepared from 3D7-MGD21⁺ IEs.

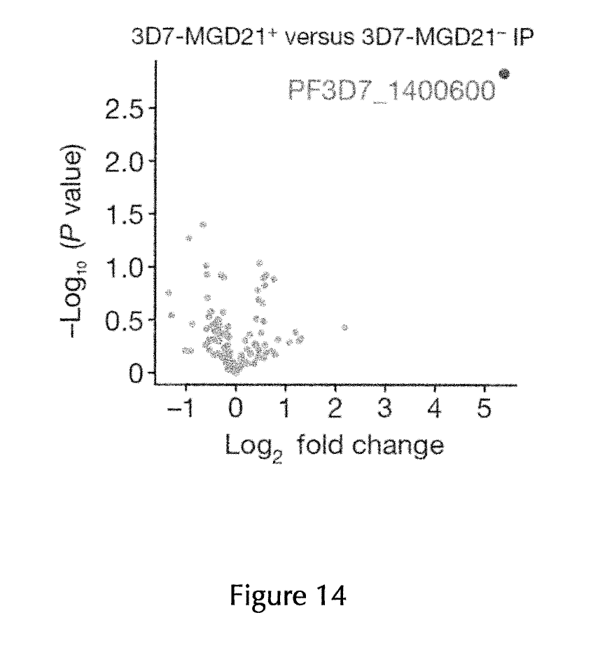

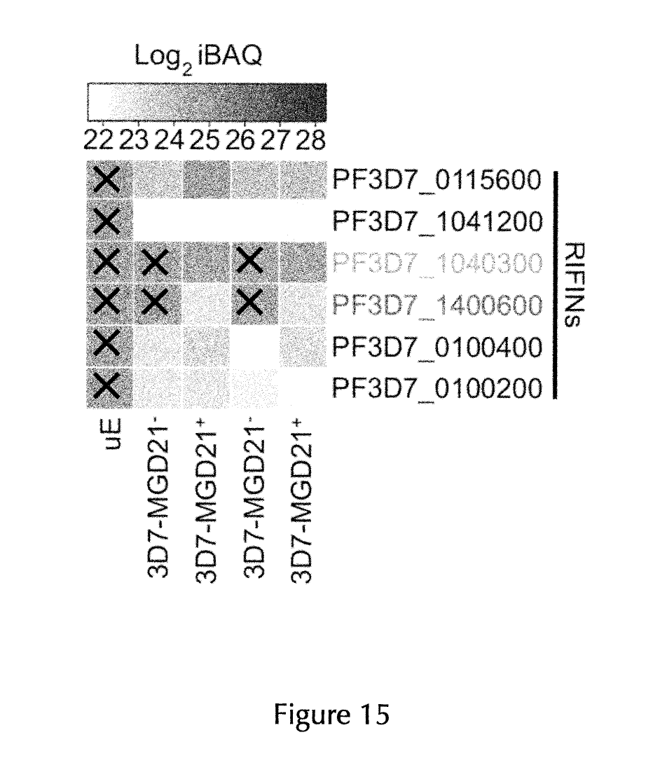

Next, analysis of the MGD21 immunoprecipitates by liquid chromatography coupled with mass spectrometry (LC-MS) was performed. As shown in FIG. 14, this experiment revealed that a member of the A-type RIFIN family (PF3D7_1400600 was significantly enriched in 3D7-MGD21⁺ immunoprecipitates as compared to 3D7-MGD21⁻ immunoprecipitates (log₂ fold change >2; P<0.01). Moreover, RIFIN expression levels in erythrocyte ghosts prepared from 3D7-MGD21⁺ and 3D7-MGD21⁻ IEs revealed that PF3D7_1400600 and a second A-type RIFIN (PF3D7_1040300) were also present in 3D7-MGD21⁺ but not in 3D7-MGD21⁻ ghosts in the absence of immunoprecipitation (FIG. 15). In contrast, four other RIFINs, including one recently characterized for its capacity to induce rosetting (PF3D7_0100400), were detected in similar amounts in both 3D7-MGD21⁺ and 3D7-MGD21⁻ ghosts (FIG. 15).

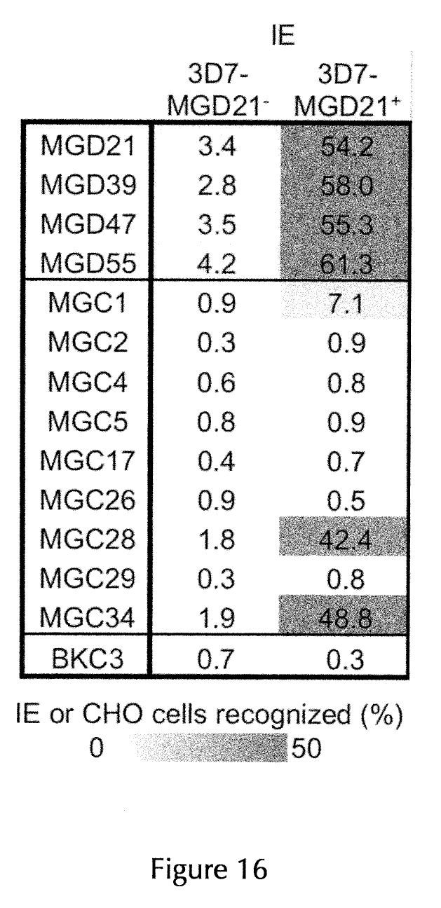

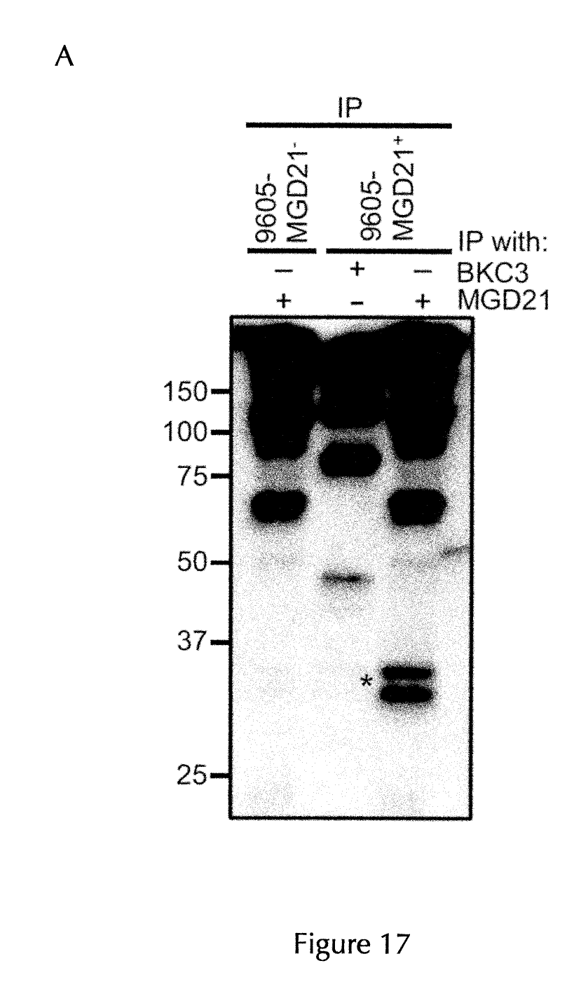

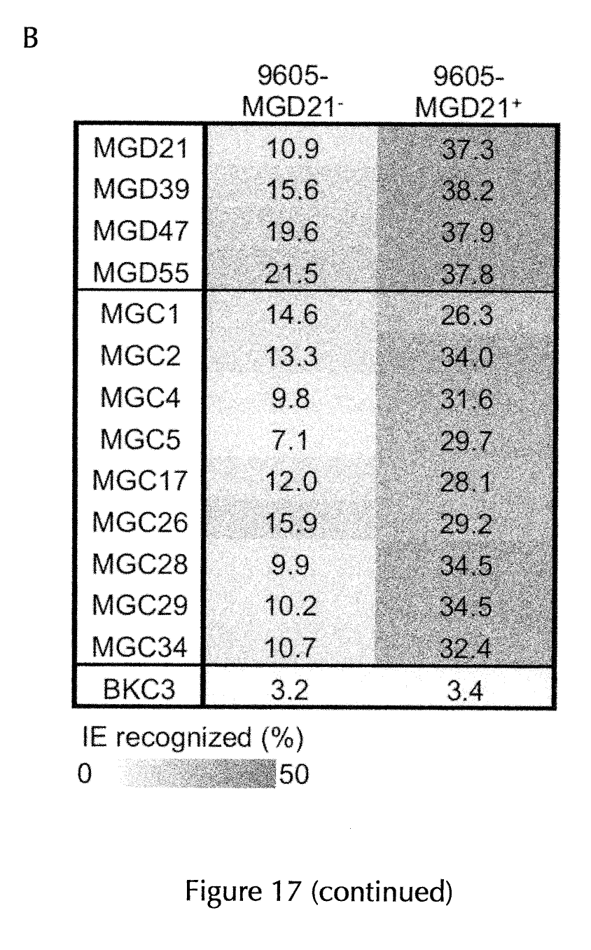

In the next step, recognition of 3D7-MGD21⁺ IEs and 3D7-MGD21⁻ IEs by other broadly reactive antibodies from donors C (MGC1, MGC2, MGC4, MGC5, MGC17, MGC26, MGC28, MGC29, MGC34) and D (MGD21, MGD39, MGD47, MGD55) were investigated. BKC3 was used as negative control antibody. As shown in FIG. 16, this experiment revealed that enrichment for 3D7-MGD21⁺ IEs greatly increased recognition by all the other broadly reactive antibodies from donor D tested and, notably, by two broadly reactive antibodies from donor C. These results suggest that these antibodies recognize the same antigens. Similar results were also obtained with the Kenyan isolate 9605 (FIG. 17A-B).

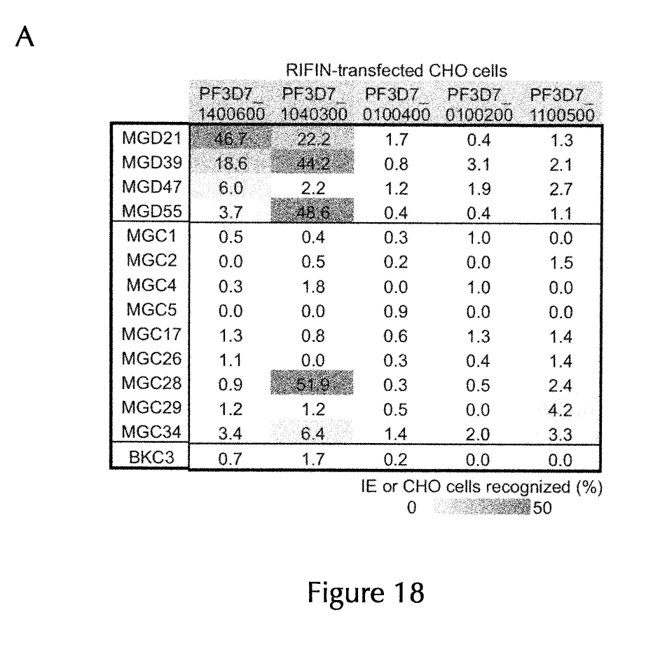

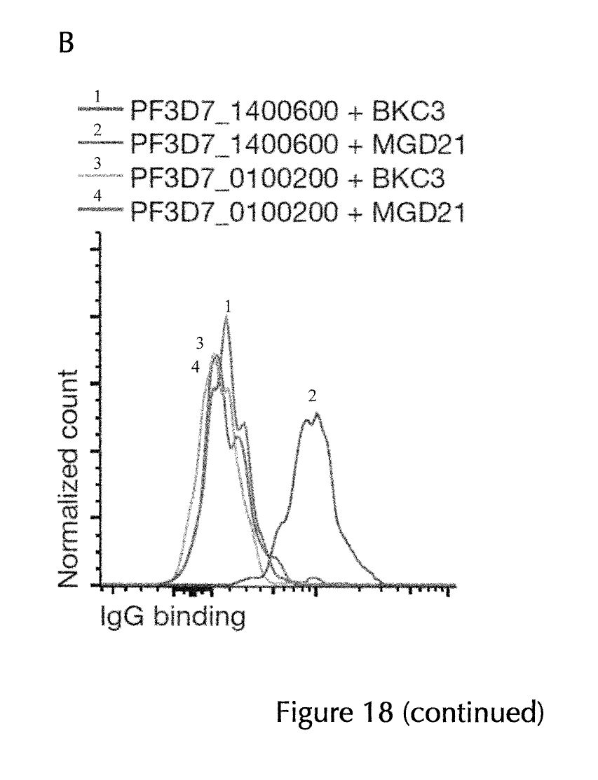

The binding of the LAIR1-containing antibodies to specific RIFINs was determined by use of CHO cells transfected with PF3D7_1400600 and PF3D7_1040300, PF3D7_0100400, PF3D7_0100200 and PF3D7_1100500. As shown in FIG. 18A, this experiment confirmed the finding that MGD21 stained CHO cells transfected with the candidate antigens PF3D7_1400600 and PF3D7_1040300, but not with irrelevant RIFINs that were similarly expressed (PF3D7_0100400 and PF3D7_0100200) or not detected (PF3D7_1100500) in 3D7-MGD21⁺ and 3D7-MGD21⁻ ghosts. FIG. 18B shows MGD21 and BKC3 staining of CHO cells transfected with a specific (PF3D7_1400600) or an irrelevant (PF3D7_0100200) RIFIN, confirming that the specificity of the binding of MGD21 to the specific RIFIN PF3D7_1400600.

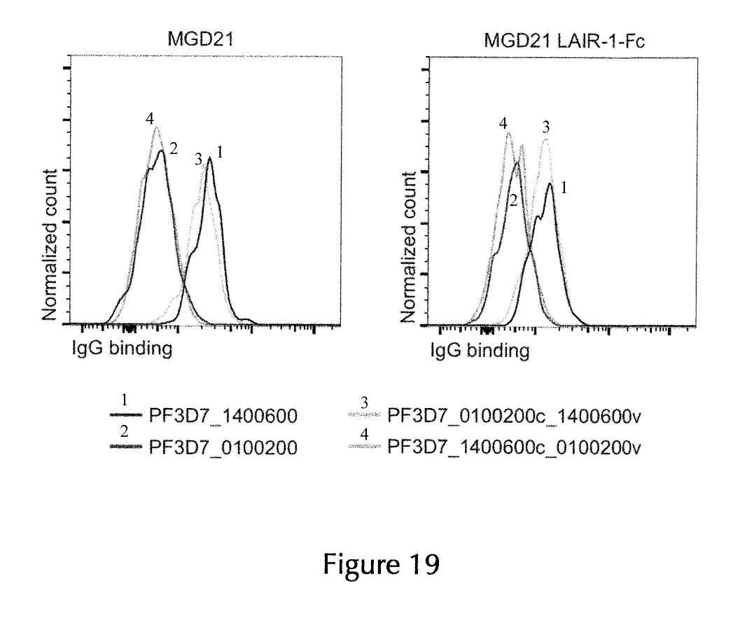

Furthermore, CHO cells were transfected with a specific (PF3D7_1400600) or an irrelevant (PF3D7_0100200) RIFIN as well as with a RIFIN chimaera containing the constant region of PF3D7_0100200 and the variable region of PF3D7_1400600 and a RIFIN chimaera containing the constant region of PF3D7_1400600 and the variable region of PF3D7_0100200. MGD21 and an Fc fusion protein containing the MGD21 LAIR1 domain stained only those CHO cells, which were transfected with the specific RIFIN PF3D7_1400600 or with the RIFIN chimaera containing the constant region of PF3D7_0100200 and the variable region of PF3D7_1400600, but not cells transfected with the inverse chimaera. Results are shown in FIG. 19, indicating that MGD21 binds to the variable region.

Collectively, the results obtained in Example 11 indicate that the LAIR1-containing antibodies recognize specific members of the RIFIN family in different P. falciparum isolates.

In particular, these results identify RIFIN PF3D7_1400600 (amino acid sequence according to SEQ ID NO: 536, nucleotide sequence according to SEQ ID NO: 537) as one major target of the mutated LAIR-1 fragment in P. falciparum and RIFIN PF3D7_1040300 (amino acid sequence according to SEQ ID NO: 538, nucleotide sequence according to SEQ ID NO: 539) as another target of the mutated LAIR-1 fragment in P. falciparum.

Since RIFINs are highly polymorphic in different strains and the mutated LAIR-1 fragment according to the present invention binds to erythrocytes infected by different P. falciparum strains, it is anticipated that the mutated LAIR-1 fragment according to the present invention will recognize additional RIFINs.

1.-86. (canceled)

87. A pharmaceutical composition comprising a polypeptide comprising a second variable (V2) domain and/or an N-terminal semi-conserved domain of a RIFIN, which is/are able to bind to a LAIR-1 fragment, wherein the LAIR-1 fragment has an amino acid sequence according to SEQ ID NO: 1:
XXLPRPXXSXXXXXXXXLGSXXTXVCRGPXGXXTFRLXXXXXXX₁YX₂XX
EXVXXX₃XPXXSEARFRXXSVXXGXXGXXRCXYYXX₄X₅XWSXXSXXXXX
XVK
wherein
X is any amino acid or no amino acid;
X₁ is T, L, G, I, R, K or no amino acid; however, if X₂ is N, X₃ is A, X₄ is P and X₅ is P, then X₁ is L, G, I, R, K or no amino acid;
X₂ is N, S or T; however, if X₁ is T, X₃ is A, X₄ is P and X₅ is P, then X₂ is S or T;
X₃ is A, T, P, or V; however, if X₁ is T, X₂ is N, X₄ is P and X₅ is P, then X₃ is T, P, or V;
X₄ is P, S, A, or D; however, if X₁ is T, X₂ is N, X₃ is A and X₅ is P, then X₄ is S, A, or D; and
X₅ is P, R, or S; however, if X₁ is T, X₂ is N, X₃ is A and X₄ is P, then X₅ is R, or S;
and wherein the LAIR-1 fragment has at least 70% amino acid sequence identity to amino acids 24 to 121 of native human LAIR-1 (SEQ ID NO: 10).

88. The pharmaceutical composition according to claim 87, wherein the polypeptide comprises a second variable (V2) domain of a RIFIN, which is able to bind to a LAIR-1 fragment as defined in claim 87.

89. The pharmaceutical composition according to claim 88, wherein the polypeptide does not comprise an N-terminal semi-conserved domain of a RIFIN as defined in claim 87.

90. The pharmaceutical composition according to claim 88, wherein the second variable (V2) domain of a RIFIN comprises an amino acid sequence according to SEQ ID NO: 625:
HXTXXXXXAXXXDXE
wherein X is any amino acid.

91. The pharmaceutical composition according to claim 88, wherein the second variable (V2) domain of a RIFIN comprises an amino acid sequence according to SEQ ID NO: 627:
IXXXRXXLXXXXXXXXXMV
wherein X is any amino acid.

92. The pharmaceutical composition according to claim 88, wherein the second variable (V2) domain of a RIFIN comprises an amino acid sequence according to SEQ ID NO: 638 or 639 or a functional sequence variant thereof.

93. The pharmaceutical composition according to claim 87, wherein the polypeptide comprises an N-terminal semi-conserved domain of a RIFIN, which is able to bind to a LAIR-1 fragment as defined in claim 87.

94. The pharmaceutical composition according to claim 93, wherein the polypeptide does not comprise a second variable (V2) domain of a RIFIN as defined in claim 87.

95. The pharmaceutical composition according to claim 93, wherein the N-terminal semi-conserved domain of a RIFIN comprises an amino acid sequence according to SEQ ID NO: 534 or 535 or a functional sequence variant thereof.

96. The pharmaceutical composition according to claim 87, wherein the polypeptide comprises a truncated RIFIN.

97. The pharmaceutical composition according to claim 87, wherein the polypeptide comprises an amino acid sequence according to SEQ ID NO: 538 (PF3D7_1040300) or according to SEQ ID NO: 536 (PF3D7_1400600) or a functional sequence variant thereof.

98. A method of preventing and/or treating malaria in a subject, wherein the method comprises administering to a subject in need thereof the pharmaceutical composition according to claim 87 in a therapeutically effective amount.

99. A method of preventing and/or treating malaria, wherein the method comprises administering to a subject an isolated polypeptide comprising a second variable (V2) domain and/or an N-terminal semi-conserved domain of a RIFIN, wherein the polypeptide comprising the second variable (V2) domain and/or the N-terminal semi-conserved domain of a RIFIN is able to bind to a LAIR-1 fragment, wherein the LAIR-1 fragment has an amino acid sequence according to SEQ ID NO: 1:
XXLPRPXXSXXXXXXXXLGSXXTXVCRGPXGXXTFRLXXXXXXX₁YX₂XX
EXVXXX₃XPXXSEARFRXXSVXXGXXGXXRCXYYXX₄X₅XWSXXSXXXXX
XVK
wherein
X is any amino acid or no amino acid;
X₁ is T, L, G, I, R, K or no amino acid; however, if X₂ is N, X₃ is A, X₄ is P and X₅ is P, then X₁ is L, G, I, R, K or no amino acid;
X₂ is N, S or T; however, if X₁ is T, X₃ is A, X₄ is P and X₅ is P, then X₂ is S or T;
X₃ is A, T, P, or V; however, if X₁ is T, X₂ is N, X₄ is P and X₅ is P, then X₃ is T, P, or V;
X₄ is P, S, A, or D; however, if X₁ is T, X₂ is N, X₃ is A and X₅ is P, then X₄ is S, A, or D; and
X₅ is P, R, or S; however, if X₁ is T, X₂ is N, X₃ is A and X₄ is P, then X₅ is R or S;
and wherein the LAIR-1 fragment has at least 70% amino acid sequence identity to amino acids 24 to 121 of native human LAIR-1 (SEQ ID NO: 10).

100. The method according to claim 99, wherein the polypeptide comprises a second variable (V2) domain of a RIFIN, which is able to bind to a LAIR-1 fragment as defined in claim 87.

101. The method according to claim 100, wherein the polypeptide does not comprise an N-terminal semi-conserved domain of a RIFIN as defined in claim 87.

102. The method according to claim 100, wherein the second variable (V2) domain of a RIFIN comprises an amino acid sequence according to SEQ ID NO: 625:
HXTXXXXXAXXXDXE
wherein X is any amino acid.

103. The method according to claim 100, wherein the second variable (V2) domain of a RIFIN comprises an amino acid sequence according to SEQ ID NO: 627:
IXXXRXXLXXXXXXXXXMV
wherein X is any amino acid.

104. The method according to claim 100, wherein the second variable (V2) domain of a RIFIN comprises an amino acid sequence according to SEQ ID NO: 638 or 639 or a functional sequence variant thereof.

105. The method according to claim 99, wherein the polypeptide comprises an N-terminal semi-conserved domain of a RIFIN, which is able to bind to a LAIR-1 fragment as defined in claim 87.

106. The method according to claim 105, wherein the polypeptide does not comprise a second variable (V2) domain of a RIFIN as defined in claim 87.

107. The method according to claim 105, wherein the polypeptide comprises an amino acid sequence according to SEQ ID NO: 534 or 535 or a functional sequence variant thereof.

108. The method according to claim 99, wherein the polypeptide comprises a truncated RIFIN.

109. The method according to claim 99, wherein the polypeptide comprises an amino acid sequence according to SEQ ID NO: 538 (PF3D7_1040300) or according to SEQ ID NO: 536 (PF3D7_1400600) or a functional sequence variant thereof.

110. A method of preventing and/or treating malaria, in a subject, wherein the method comprises administering to a subject a nucleic acid molecule encoding a polypeptide as defined in claim 99.

111. The method according to claim 110, wherein the nucleic acid molecule comprises a nucleic acid sequence according to SEQ ID NO: 540 or 541 or a functional sequence variant thereof.

112. A vector comprising a nucleic acid molecule as defined in claim 110.

113. A cell comprising a nucleic acid molecule as defined in claim 110.

114. A method for diagnosing malaria in a subject, the method comprising the use of:
(a) a polypeptide as defined in claim 99,
(b) a nucleic acid molecule encoding the polypeptide of (a),
(c) a vector comprising the nucleic acid molecule of (b), or
(d) a cell comprising the nucleic acid molecule of (b) or the vector of (c).

115. A method for identification of antibodies binding to infected erythrocytes, the method comprising the use of:
(e) a polypeptide as defined in claim 99,
(f) a nucleic acid molecule encoding the polypeptide of (a),
(g) a vector comprising the nucleic acid molecule of (b), or
(h) a cell comprising the nucleic acid molecule of (b) or the vector of (c).


SEQUENCE LISTING (639 sequences)

SEQ ID NO: 1 - protein, 98 aa, Artificial Sequence
  LAIR-1 fragment
  X (1)..(2): X is any amino acid or no amino acid (deletion mutation)
  X (7)..(8): X is any amino acid or no amino acid (deletion mutation)
  X (10)..(17): X is any amino acid or no amino acid (deletion mutation)
  X (21)..(22): X is any amino acid or no amino acid (deletion mutation)
  X (24)..(24): X is any amino acid or no amino acid (deletion mutation)
  X (30)..(30): X is any amino acid or no amino acid (deletion mutation)
  X (32)..(33): X is any amino acid or no amino acid (deletion mutation)
  X (38)..(43): X is any amino acid or no amino acid (deletion mutation)
  X (44)..(44): X is T, L, G, I, R, K or no amino acid; however, if X (Pos. 46) is N, X (Pos. 54) is A, X (Pos. 83) is P and X (Pos.84) is P, then X (Pos. 44) is L, G, I, R, K or no amino acid
  X (46)..(46): X is N, S or T; however, if X (Pos. 44) is T, X (Pos. 54) is A, X (Pos. 83) is P and X (Pos. 84) is P, then X (Pos. 46) is S or T
  X (47)..(48): X is any amino acid or no amino acid (deletion mutation)
  X (50)..(50): X is any amino acid or no amino acid (deletion mutation)
  X (52)..(53): X is any amino acid or no amino acid (deletion mutation)
  X (54)..(54): X is A, T, P, or V; however, if X (Pos. 44) is T, X (Pos. 46) is N, X (Pos. 83) is P and X (Pos. 84) is P, then X (Pos. 54) is T, P, or V
  X (55)..(55): X is any amino acid or no amino acid (deletion mutation)
  X (57)..(58): X is any amino acid or no amino acid (deletion mutation)
  X (65)..(66): X is any amino acid or no amino acid (deletion mutation)
  X (69)..(70): X is any amino acid or no amino acid (deletion mutation)
  X (72)..(73): X is any amino acid or no amino acid (deletion mutation)
  X (75)..(76): X is any amino acid or no amino acid (deletion mutation)
  X (79)..(79): X is any amino acid or no amino acid (deletion mutation)
  X (82)..(82): X is any amino acid or no amino acid (deletion mutation)
  X (83)..(83): X4 is P, S, A, or D; however, if X (Pos. 44) is T, X (Pos. 46) is N, X (Pos. 54) is A and X (Pos. 84) is P, then X (Pos. 83) is S, A, or D
  X (84)..(84): X is P, R, or S; however, if X (Pos. 44) is T, X (Pos. 46) is N, X (Pos. 54) is A and X (Pos. 83) is P, then X (Pos. 84) is R or S
  X (85)..(85): X is any amino acid or no amino acid (deletion mutation)
  X (88)..(89): X is any amino acid or no amino acid (deletion mutation)
  X (91)..(96): X is any amino acid or no amino acid (deletion mutation)
XXLPRPXXSXXXXXXXXLGSXXTXVCRGPXGXXTFRLXXXXXXXYXXXEXVXXXXPXXSEARFRXXSVXXGXXGXXRCXYYXXXXWSXXSXXXXXXVK

SEQ ID NO: 2 - protein, 287 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 3 - DNA, 864 bp, Homo sapiens
atgtctccccaccccaccgccctcctgggcctagtgctctgcctggcccagaccatccacacgcaggaggaagatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaagaaacctctggaggcccggactccccggacacagagcccggctcctcagctggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacactga

SEQ ID NO: 4 - protein, 270 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 5 - DNA, 813 bp, Homo sapiens
atgtctccccaccccaccgccctcctgggcctagtgctctgcctggcccagaccatccacacgcaggaggaagatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaaggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacactga

SEQ ID NO: 6 - protein, 269 aa, Homo sapiens
MSPHPTALLGLVLCLAQTIHTQEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 7 - DNA, 810 bp, Homo sapiens
atgtctccccaccccaccgccctcctgggcctagtgctctgcctggcccagaccatccacacgcaggaggatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaaggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacactga

SEQ ID NO: 8 - protein, 209 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQR

SEQ ID NO: 9 - DNA, 630 bp, Homo sapiens
atgtctccccaccccaccgccctcctgggcctagtgctctgcctggcccagaccatccacacgcaggaggaagatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaagaaacctctggaggcccggactccccggacacagagcccggctcctcagctggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggtga

SEQ ID NO: 10 - protein, 98 aa, Homo sapiens
EDLPRPSISAEPGTVIPLGSHVTFVCRGPVGVQTFRLERESRSTYNDTEDVSQASPSESEARFRIDSVSEGNAGPYRCIYYKPPKWSEQSDYLELLVK

SEQ ID NO: 11 - protein, 98 aa, Artificial Sequence; identical to SEQ ID NO: 1
  LAIR-1 fragment
  X (1)..(2): X is any amino acid or no amino acid (deletion)
  X (7)..(8): X is any amino acid or no amino acid (deletion)
  X (10)..(17): X is any amino acid or no amino acid (deletion)
  X (21)..(22): X is any amino acid or no amino acid (deletion)
  X (24)..(24): X is any amino acid or no amino acid (deletion)
  X (30)..(30): X is any amino acid or no amino acid (deletion)
  X (32)..(33): X is any amino acid or no amino acid (deletion)
  X (38)..(43): X is any amino acid or no amino acid (deletion)
  X (44)..(44): X is T or L; however, if X (Pos. 46) is N, X (Pos. 54) is A, X (Pos. 83) is P and X (Pos. 84) is P, then X (Pos. 44) is L
  X (46)..(46): X is N or S; however, if X (Pos. 44) is T, X (Pos. 54) is A, X (Pos. 83) is P and X (Pos. 84) is P, then X (Pos. 46) is S
  X (47)..(48): X is any amino acid or no amino acid (deletion)
  X (50)..(50): X is any amino acid or no amino acid (deletion)
  X (52)..(53): X is any amino acid or no amino acid (deletion)
  X (54)..(54): X is A or T; however, if X (Pos. 44) is T, X (Pos. 46) is N, X (Pos. 83) is P and X(Pos. 84) is P, then X (Pos. 54) is T
  X (55)..(55): X is any amino acid or no amino acid (deletion)
  X (57)..(58): X is any amino acid or no amino acid (deletion)
  X (65)..(66): X is any amino acid or no amino acid (deletion)
  X (69)..(70): X is any amino acid or no amino acid (deletion)
  X (72)..(73): X is any amino acid or no amino acid (deletion)
  X (75)..(76): X is any amino acid or no amino acid (deletion)
  X (79)..(79): X is any amino acid or no amino acid (deletion)
  X (82)..(82): X is any amino acid or no amino acid (deletion)
  X (83)..(83): X is P or S; however, if X (Pos. 44) is T, X (Pos. 46) is N, X (Pos. 54) is A and X (Pos. 84) is P, then X (Pos. 83) is S
  X (84)..(84): X is P or R; however, if X (Pos. 44) is T, X (Pos. 46) is N, X (Pos. 54) is A and X (Pos. 83) is P, then X (Pos. 84) is R
  X (85)..(85): X is any amino acid or no amino acid (deletion)
  X (88)..(89): X is any amino acid or no amino acid (deletion)
  X (91)..(96): X is any amino acid or no amino acid (deletion)

SEQ ID NO: 12 - protein, 98 aa, Artificial Sequence
  MGC1/MGC32_EXON aa
EDLPRPSISAAEGTVIPLGSHVTFVCRGPVGVQTFRLEKDSRSIYNDTENVSQPSPSESEARFRIDSVSEGNAGLYRCVYYKAPKWSAQSDYLELLVK

SEQ ID NO: 13 - DNA, 294 bp, Artificial Sequence
  MGC1/MGC32_EXON nucl
gaagatctgcccagaccctccatctcggctgccgaaggcaccgtgatccccctggggagccatgtgactttcgtgtgccggggcccggttggggttcaaacattccgcctggagaaggacagtagatccatatacaatgatactgaaaatgtgtctcaacctagtccatctgagtcagaggccagatttcgcattgactcagtgagtgaaggaaatgccggactttatcggtgcgtctattataaggcccctaaatggtctgcgcagagtgattacctggagctgctggtgaaa

SEQ ID NO: 14 - protein, 98 aa, Artificial Sequence
  MGC2_EXON aa
EHLPRPSISPEPGTVITLGSHVTFVCRGPVGVQTFRLEKDSRSTYNDTEDVSQPSPSESEARFRIDSVSEGYAGLYRCLYYKPPKWSEQSDYLELLVK

SEQ ID NO: 15 - DNA, 294 bp, Artificial Sequence
  MGC2_EXON nucl
gagcatctgcccagaccctccatctcgcctgagccaggcaccgtgatcaccctggggagccatgtgactttcgtgtgccggggcccggttggggttcaaacattccgcctggagaaggacagtagatccacatacaatgatactgaagatgtgtctcaacctagtccatctgagtcagaggccagattccgcattgactcagtaagtgaaggatatgccgggctttatcgctgcctctattataagccccctaaatggtctgagcagagtgactacctggagctgctggtgaaa

SEQ ID NO: 16 - protein, 96 aa, Artificial Sequence
  MGC4_EXON aa
EDLPRPSISAEPDTVIPLGSHVTFVCRGPVGVHTFRLERGWRYNDTEDVSQAGPSESEARFRIDSVREGNAGLYRCIYYIAPKWSEQSDYLELRVK

SEQ ID NO: 17 - DNA, 288 bp, Artificial Sequence
  MGC4_EXON nucl
gaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaa

SEQ ID NO: 18 - protein, 96 aa, Artificial Sequence
  MGC5/MGC29_EXON aa
EDLPRPSISAEPGTVIPLGSHVTFVCRGPVGVHTFRLERGWRYNDTEDVSQAGPSQSEARFRIDSVREGNAGLYRCLYYIPPKWSEQSDYLELRVK

SEQ ID NO: 19 - DNA, 288 bp, Artificial Sequence
  MGC5/MGC29_EXON nucl
gaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaa

SEQ ID NO: 20 - protein, 98 aa, Artificial Sequence
  MGC7/MGC37_EXON aa
DDLPRPSISPEPGTVIPLGSHVTFVCRGPVGVQTFRLEKDRRSTYNDTEDVSQPSPSESEARFRIDSVTEGNAGLYRCVYYKPPKWSDQSDFLELLVK

SEQ ID NO: 21 - DNA, 294 bp, Artificial Sequence
  MGC7/MGC37_EXON nucl
gatgatctgcccagaccctctatctcgcctgagccaggcaccgtgatccccctggggagccatgtgactttcgtgtgtcggggcccggttggggttcaaacattccgcctggagaaggacagaagatccacatacaatgatactgaagatgtgtctcaacctagtccatctgagtcagaggccagattccgcattgactcagtaactgaaggaaatgccgggctttatcgctgcgtctattataagccccctaaatggtctgaccagagtgacttcctggagttgctggtgaag

SEQ ID NO: 22 - protein, 98 aa, Artificial Sequence
  MGC17_EXON aa
EDLPRPSISAEEGTVIPLGSRLTFVCRGPVGVHTFRLERDRRSTYNDTEDVSHPSPSESEARFRIDSVSEGNAGLYRCVYYKSPEWSKQSDYLELLVK

SEQ ID NO: 23 - DNA, 294 bp, Artificial Sequence
  MGC17_EXON nucl
gaagatctgcccagaccctccatctcggctgaggaaggcaccgtgattcccctggggagccgtctgactttcgtgtgccggggcccggttggggttcacacattccgcctggagagggaccgtagatccacatacaatgatactgaagatgtgtctcaccctagtccatctgagtctgaggccagatttcgcattgactcagtgagtgaaggaaatgccgggctttatcgctgcgtctattataagtcccctgaatggtctaagcagagtgattacctggagctgctggtgaaa

SEQ ID NO: 24 - protein, 98 aa, Artificial Sequence
  MGC26_EXON aa
EDLPRPSISPEPATVIPLGSHVTIVCRGPVGVETFRLQKESRSLYNDTEDVSQPSPSESEARFRIDSVSEGHGGLYRCLYYKSSKWSEQSDYLEMLVK

SEQ ID NO: 25 - DNA, 294 bp, Artificial Sequence
  MGC26_EXON nucl
gaagatctgcccagaccctccatctcgccggagccagccaccgtgatccccctggggagccatgtgactatcgtgtgccggggcccggttggggttgaaacattccgcctgcagaaggagagtagatccctgtacaatgacactgaagatgtgtctcaacctagtccatctgagtcagaggccagattccgcattgactcagtaagtgaagggcatggcgggctttatcgctgcctctattataagtcttctaaatggtctgagcagagtgactacctggagatgctggtgaaa

SEQ ID NO: 26 - protein, 98 aa, Artificial Sequence
  MGC28/MGC33_EXON aa
EDLPRPTISAETGTVISLGSHVTFVCRGPLGVQTFRLERESRSRYSETEDVSQVGPSESEARFRIDSVSEGNAGLYRCIYYKPPKWSEQSDYLELRVK

SEQ ID NO: 27 - DNA, 294 bp, Artificial Sequence
  MGC28/MGC33_EXON nucl
gaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaa

SEQ ID NO: 28 - protein, 98 aa, Artificial Sequence
  MGC34_EXON aa
EDLPRPSISAEPGTVIPLGSHVTFVCRGPVGIHTFRLERESRSLYTETEDVTQVSPSESEARFRIESVTEGNAGLYRCVYYKPPKWSEQSDYLELLVK

SEQ ID NO: 29 - DNA, 294 bp, Artificial Sequence
  MGC34_EXON nucl
gaagatctgcccagaccctccatctcggctgagccaggcaccgtgatccccctggggagtcatgtgaccttcgtgtgccggggcccggttgggattcacacattccgcctggagagggagagtagatccctatacactgaaactgaagatgtgactcaagtaagtccttctgagtcagaggccagattccgcattgagtcagtaactgaaggaaatgccgggctttatcgctgcgtctattataagccccctaaatggtctgagcagagtgactacctggagctgctggtgaaa

SEQ ID NO: 30 - protein, 96 aa, Artificial Sequence
  MGC35_EXON aa
EDLPRPSISAEPGSVIPLGSLVTFVCRGPVGVHTFRLERGWTYNDTEDVSQAGPSESEARFRMDSVREGNAGLYRCIYYKPPKWSEQSAYLELRVK

SEQ ID NO: 31 - DNA, 288 bp, Artificial Sequence
  MGC35_EXON nucl
gaagatctgcccagaccctccatctcggctgagccaggctccgtgatccccctggggagccttgtgactttcgtgtgccggggcccggttggggttcacacattccgcctcgagagggggtggacatacaacgacactgaagatgtgtctcaagctggtccatctgagtcagaggccagattccgcatggactcggtaagggaaggaaatgccgggctttatcgatgcatctattacaaaccccctaaatggtctgagcagagtgcctacctggaactgcgggtgaaa

SEQ ID NO: 32 - protein, 97 aa, Artificial Sequence
  MGC36_EXON aa
EEDLPRPSISAEPDTVIPLGSHVTFVCRGPVGVHTFRLERGWRYNDTEDVSQAGPSESEARFRIDSVREGNAGLYRCIYYIAPKWSEQSDYLELRVK

SEQ ID NO: 33 - DNA, 291 bp, Artificial Sequence
  MGC36_EXON nucl
gaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaa

SEQ ID NO: 34 - protein, 97 aa, Artificial Sequence
  MGD21_EXON aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVK

SEQ ID NO: 35 - DNA, 291 bp, Artificial Sequence
  MGD21_EXON nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaa

SEQ ID NO: 36 - protein, 98 aa, Artificial Sequence
  MGD23_EXON aa
EDLPRPSLSAEPGTVIPLGSHVTFVCRGPAGVETFRLERESRFTYNDTEDVSQASPSESEARFRIDSVSEGNAGPYRCLYYKARKWSDQSDYLELLVK

SEQ ID NO: 37 - DNA, 294 bp, Artificial Sequence
  MGD23_EXON nucl
gaagatctgcccagaccctccctctcggctgaaccaggcaccgtgatccccctggggagtcacgtgactttcgtgtgccggggcccggctggggtcgaaacattccgcctggagagggagagtagattcacttacaacgatactgaagatgtgtctcaagcgagtccatctgagtcagaggccagattccgcattgactcagtaagtgaaggaaatgccgggccttatcgctgcctctattataaggcccgtaaatggtctgaccagagtgactacttggaattgctggtgaag

SEQ ID NO: 38 - protein, 98 aa, Artificial Sequence
  MGD30_EXON aa
EKLPRPSISAEPGTVIPLGSRVTFVCRGPVGVQTFRLERETSFTYNDTEDVSQVSPSESEARFRIDSVSEGYAGPYRCVYYKAPKWSEQSDYLDLLVK

SEQ ID NO: 39 - DNA, 294 bp, Artificial Sequence
  MGD30_EXON nucl
gaaaaactgcccagaccctccatctcggctgagccgggcaccgtgatccccctggggagccgtgtgactttcgtgtgccggggcccggttggggttcaaacattccgcctagagagggagactagctttacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaa

SEQ ID NO: 40 - protein, 98 aa, Artificial Sequence
  MGD33_EXON aa
EKLPRPSISAEPGTVIPLGSRVTFVCRGPVGVQTFRLERETRSTYNDTEDVSQVSPSESEARFRIDSVSEGYAGPYRCVYYKAPKWSEQSDYLDLLVK

SEQ ID NO: 41 - DNA, 294 bp, Artificial Sequence
  MGD33_EXON nucl
gaaaaactgcccagaccctccatctcggctgagccgggcaccgtgatccccctggggagccgtgtgactttcgtgtgccggggcccggttggggttcaaacattccgcctagagagggagactagatctacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaa

SEQ ID NO: 42 - protein, 98 aa, Artificial Sequence
  MGD34_EXON aa
EDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVK

SEQ ID NO: 43 - DNA, 294 bp, Artificial Sequence
  MGD34_EXON nucl
gaagatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaa

SEQ ID NO: 44 - protein, 97 aa, Artificial Sequence
  MGD35_EXON aa
NLPRPSLSAEPGTVIPLGSPVTFVCRGPVGVHTFRLERAGRSTYNDTEDVSHPSPSESEARFRIDSVSEGNAGPYRCVYYKSSKWSEESYCLDLLVK

SEQ ID NO: 45 - DNA, 291 bp, Artificial Sequence
  MGD35_EXON nucl
aatttgcccagaccctccctctcggcggagccaggcaccgtgatccccctggggagccctgtgactttcgtgtgccggggcccggttggggttcacacattccgcctggagagggcgggtagatccacatacaatgatactgaagatgtgtctcatcctagtccatctgagtcagaggccagattccgcattgactcagtgagtgagggaaatgccgggccttatcgctgcgtctattataagtcctctaaatggtccgaggagagttactgcctggacctgctggtcaaa

SEQ ID NO: 46 - protein, 98 aa, Artificial Sequence
  MGD39_EXON aa
DDLPRPSISAEPGTVIPLGSHVTFVCRGPIGVQTFRLERERRSLYSDTEDVSQVSPFASEARFRIDSVSEGNAGPYRCIYYKDRKWSDQSDYLELLVK

SEQ ID NO: 47 - DNA, 294 bp, Artificial Sequence
  MGD39_EXON nucl
gacgatctgcccagaccctccatctcggctgagccaggcaccgtgatccccctggggagccatgtgaccttcgtgtgccggggcccaattggggttcaaacattccgcctggagagggagagaagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaa

SEQ ID NO: 48 - protein, 98 aa, Artificial Sequence
  MGD41_EXON aa
EDLPRPSLSAEPGTVVPLGSHVTFVCRGPVGVQTFRLERESRSTYNDTEDVSQPSPFESEARFRIDSVSEGNAGPYRCIYYKSPKWSDQSDYVELLVK

SEQ ID NO: 49 - DNA, 294 bp, Artificial Sequence
  MGD41_EXON nucl
gaagatctgcccagaccctccctctcggctgagccaggcaccgtggtccccctggggagccatgtgactttcgtgtgccggggcccggttggggttcaaacattccgcctggagagggagagcagatccacatacaatgatactgaagatgtgtctcaacctagtccatttgagtcagaggccagatttcgcattgactcagtaagtgaaggaaatgccgggccttatcgctgcatctattataagtcccctaaatggtctgaccagagtgactacgtggagttgctggtgaaa

SEQ ID NO: 50 - protein, 98 aa, Artificial Sequence
  MGD47_EXON aa
GDLPRPSISAEPGTAIPLGSQVTFVCRGPIGVQTFRLERESRALYNDSEDVSQVSPSASEARFRIDSVSEGNAGPYRCIYYKARRWSDQSDYLELLVK

SEQ ID NO: 51 - DNA, 294 bp, Artificial Sequence
  MGD47_EXON nucl
ggagatctgcccagaccctccatctcggctgagccaggcaccgcgatccccctggggagccaagtgactttcgtgtgccggggcccaattggggttcaaacattccgcctggagagggagagtcgcgccttatataatgattctgaagatgtgtctcaagttagtccatctgcgtcagaggccagattccgcattgactcagtaagtgaaggcaatgccgggccttatcgctgtatctattataaggcccgcagatggtctgaccagagtgactatttggagttgttggtgaaa

SEQ ID NO: 52 - protein, 98 aa, Artificial Sequence
  MGD55_EXON aa
DDLPRPSISAEPGTVIPLGSHVTFVCRGPIGVQTFRLERESRSLYSDTEDVSQVSPFASEARFRIDSVSEGNAGPYRCIYYKDRKWSDQSDYLELLVK

SEQ ID NO: 53 - DNA, 294 bp, Artificial Sequence
  MGD55_EXON nucl
gacgatctgcccagaccctccatctcggctgagccaggcaccgtgatccccctggggagccatgtgactttcgtgtgccggggcccaattggggttcaaacattccgcctggagagggagagtagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaa

SEQ ID NO: 54 - protein, 98 aa, Artificial Sequence
  MGD56_EXON aa
KDLPRPSLSAEPGTVIPLGSHVTFVCRGPVGVQTFRLQRESRSLYNDTEDVSHPSPSESEARFRIDSVSEGNAGPYRCVYYKSSKWSEESDCLELLVK

SEQ ID NO: 55 - DNA, 294 bp, Artificial Sequence
  MGD56_EXON nucl
aaagatttgcccagaccctccctctcggctgagccaggcaccgtgatccccctggggagtcatgtgactttcgtgtgccggggcccggttggggttcagacattccgcctgcagagggagagtagatccctttacaatgatactgaagatgtgtctcatcctagtccatctgagtcagaggccagattccgcattgactcagtgagtgagggaaatgccgggccttatcgctgcgtctattataagtcctctaaatggtccgaggagagtgactgcctggagctgctggtcaaa

SEQ ID NO: 56 - protein, 8 aa, Artificial Sequence
  MGC1 CDRH1 aa
GFNFRKSW

SEQ ID NO: 57 - protein, 8 aa, Artificial Sequence
  MGC1 CDRH2 aa
IREDGSES

SEQ ID NO: 58 - protein, 139 aa, Artificial Sequence
  MGC1 CDRH3 aa
ARDRFCNDDEIHRHGQEDLPRPSISAAEGTVIPLGSHVTFVCRGPVGVQTFRLEKDSRSIYNDTENVSQPSPSESEARFRIDSVSEGNAGLYRCVYYKAPKWSAQSDYLELLVKGQEVTWALFTSCGGDGEEPDYDMDV

SEQ ID NO: 59 - protein, 12 aa, Artificial Sequence
  MGC1 CDRL1 aa
QSVLYRSKNKNY

SEQ ID NO: 60 - skipped

SEQ ID NO: 61 - protein, 15 aa, Artificial Sequence
  MGC1 CDRL2 long aa
YYCLQYYITPYTFGQ

SEQ ID NO: 62 - protein, 9 aa, Artificial Sequence
  MGC1 CDRL3 aa
LQYYITPYT

SEQ ID NO: 63 - DNA, 24 bp, Artificial Sequence
  MGC1 CDRH1 nuc
gggttcaactttagaaagtcttgg

SEQ ID NO: 64 - DNA, 24 bp, Artificial Sequence
  MGC1 CDRH2 nuc
ataagagaagatggaagtgagagt

SEQ ID NO: 65 - DNA, 417 bp, Artificial Sequence
  MGC1 CDRH3 nuc
gcgagagatagattctgcaatgatgatgagattcacagacacggacaagaagatctgcccagaccctccatctcggctgccgaaggcaccgtgatccccctggggagccatgtgactttcgtgtgccggggcccggttggggttcaaacattccgcctggagaaggacagtagatccatatacaatgatactgaaaatgtgtctcaacctagtccatctgagtcagaggccagatttcgcattgactcagtgagtgaaggaaatgccggactttatcggtgcgtctattataaggcccctaaatggtctgcgcagagtgattacctggagctgctggtgaaaggtcaggaagtcacctgggccctgtttacctcctgtggtggtgatggagaggaacccgactacgacatggacgtc

SEQ ID NO: 66 - DNA, 36 bp, Artificial Sequence
  MGC1 CDRL1 nuc
cagagtgttttatacaggtccaagaataagaactac

SEQ ID NO: 67 - skipped

SEQ ID NO: 68 - DNA, 27 bp, Artificial Sequence
  MGC1 CDRL2 long nuc
ctcatttactcgacatctactcgggcg

SEQ ID NO: 69 - DNA, 27 bp, Artificial Sequence
  MGC1 CDRL3 nuc
ctgcaatattatattactccctacact

SEQ ID NO: 70 - protein, 246 aa, Artificial Sequence
  MGC1 heavy chain aa
EVQLVESGGGLVQPGGSLRLSCVASGFNFRKSWMGWVRQAPGKGLEWVANIREDGSESFYADSVKGRFTVSRDNAKKSLYLHINSLRAEDTAVYYCARDRFCNDDEIHRHGQEDLPRPSISAAEGTVIPLGSHVTFVCRGPVGVQTFRLEKDSRSIYNDTENVSQPSPSESEARFRIDSVSEGNAGLYRCVYYKAPKWSAQSDYLELLVKGQEVTWALFTSCGGDGEEPDYDMDVRGKGTTVTVSS

SEQ ID NO: 71 - protein, 113 aa, Artificial Sequence
  MGC1 light chain aa
DIVMTQSPDSLAVSLGERATINCKSSQSVLYRSKNKNYLAWFQQKPGQPPKVLIYSTSTRASGVPDRFTGSGSGTDFTLTISSLQAEDVAVYYCLQYYITPYTFGQGTKLEIK

SEQ ID NO: 72 - DNA, 738 bp, Artificial Sequence
  MGC1 heavy chain nuc
gaggtgcagctggtggagtctgggggaggcttggtccagccgggggggtccctgagactctcctgtgtagcctctgggttcaactttagaaagtcttggatgggttgggtccgccaggctccagggaaggggctggagtgggtggcaaacataagagaagatggaagtgagagtttctatgcggactctgtgaagggccgcttcaccgtctccagagacaacgccaagaaatcactgtatctccatatcaacagcctgagagccgaggacacggctgtctattactgtgcgagagatagattctgcaatgatgatgagattcacagacacggacaagaagatctgcccagaccctccatctcggctgccgaaggcaccgtgatccccctggggagccatgtgactttcgtgtgccggggcccggttggggttcaaacattccgcctggagaaggacagtagatccatatacaatgatactgaaaatgtgtctcaacctagtccatctgagtcagaggccagatttcgcattgactcagtgagtgaaggaaatgccggactttatcggtgcgtctattataaggcccctaaatggtctgcgcagagtgattacctggagctgctggtgaaaggtcaggaagtcacctgggccctgtttacctcctgtggtggtgatggagaggaacccgactacgacatggacgtccggggcaaagggaccacggtcaccgtctcctca

SEQ ID NO: 73 - DNA, 339 bp, Artificial Sequence
  MGC1 light chain nuc
gacatcgtgatgacccagtctccagactccctggctgtgtctctgggcgagagggccaccatcaactgcaagtccagtcagagtgttttatacaggtccaagaataagaactacttagcttggttccagcagaaaccaggacagcctcctaaggtgctcatttactcgacatctactcgggcgtccggggtccctgaccgattcactggcagcgggtctgggacagatttcactctcaccatcagcagcctgcaggctgaagatgtggcagtttattactgtctgcaatattatattactccctacacttttggccaggggaccaagttggagatcaaa

SEQ ID NO: 74 - protein, 8 aa, Artificial Sequence
  MGC2 CDRH1 aa
GFTFSNFW

SEQ ID NO: 75 - protein, 8 aa, Artificial Sequence
  MGC2 CDRH2 aa
IKEDGSEK

SEQ ID NO: 76 - protein, 136 aa, Artificial Sequence
  MGC2 CDRH3 aa
VRERFCSNHIHKEEHLPRPSISPEPGTVITLGSHVTFVCRGPVGVQTFRLEKDSRSTYNDTEDVSQPSPSESEARFRIDSVSEGYAGLYRCLYYKPPKWSEQSDYLELLVKGDDVTWALYPSCGGDGEASDYNMDV

SEQ ID NO: 77 - protein, 6 aa, Artificial Sequence
  MGC2 CDRL1 aa
QRFSGW

SEQ ID NO: 78 - skipped

SEQ ID NO: 79 - protein, 9 aa, Artificial Sequence
  MGC2 CDRL2 long aa
LIYKASPLA

SEQ ID NO: 80 - protein, 9 aa, Artificial Sequence
  MGC2 CDRL3 aa
QHYSNYSYT

SEQ ID NO: 81 - DNA, 24 bp, Artificial Sequence
  MGC2 CDRH1 nuc
ggattcacctttagtaacttttgg

SEQ ID NO: 82 - DNA, 24 bp, Artificial Sequence
  MGC2 CDRH2 nuc
ataaaggaagatggaagtgagaaa

SEQ ID NO: 83 - DNA, 408 bp, Artificial Sequence
  MGC2 CDRH3 nuc
gtgagagagagattctgcagtaatcatatccacaaagaagagcatctgcccagaccctccatctcgcctgagccaggcaccgtgatcaccctggggagccatgtgactttcgtgtgccggggcccggttggggttcaaacattccgcctggagaaggacagtagatccacatacaatgatactgaagatgtgtctcaacctagtccatctgagtcagaggccagattccgcattgactcagtaagtgaaggatatgccgggctttatcgctgcctctattataagccccctaaatggtctgagcagagtgactacctggagctgctggtgaaaggtgacgacgtcacctgggccctgtacccctcttgtggtggtgatggagaggcttccgactacaacatggacgtc

SEQ ID NO: 84 - DNA, 18 bp, Artificial Sequence
  MGC2 CDRL1 nuc
cagcgttttagtggctgg

SEQ ID NO: 85 - skipped

SEQ ID NO: 86 - DNA, 27 bp, Artificial Sequence
  MGC2 CDRL2 long nuc
ctgatctataaggcgtctcctttagca

SEQ ID NO: 87 - DNA, 27 bp, Artificial Sequence
  MGC2 CDRL3 nuc
caacactacagtaattattcatatact

SEQ ID NO: 88 - protein, 243 aa, Artificial Sequence
  MGC2 heavy chain aa
EVQLVESGGGLVQPGGSLRLSCAASGFTFSNFWMGWVRQTPGKGLEWVANIKEDGSEKYYVDSVRGRFTISRDSAKNSLYLQMNSLRAEDTAVYYCVRERFCSNHIHKEEHLPRPSISPEPGTVITLGSHVTFVCRGPVGVQTFRLEKDSRSTYNDTEDVSQPSPSESEARFRIDSVSEGYAGLYRCLYYKPPKWSEQSDYLELLVKGDDVTWALYPSCGGDGEASDYNMDVWGKGTTVTVSS

SEQ ID NO: 89 - protein, 107 aa, Artificial Sequence
  MGC2 light chain aa
DIQMTQSPSTLSASVGDRVTISCRASQRFSGWLAWYQQKPGKAPNLLIYKASPLAGGGPSRFSGSGSGTDFTLTISSLQPDDSATYYCQHYSNYSYTFGQGTKLEIR

SEQ ID NO: 90 - DNA, 729 bp, Artificial Sequence
  MGC2 heavy chain nuc
gaggtgcagctggtggagtctgggggaggcttggtccagcctggggggtccctgagactctcctgtgcagcctctggattcacctttagtaacttttggatgggttgggtccgccagactccagggaaggggctggagtgggtggccaatataaaggaagatggaagtgagaaatactatgtggactctgtgaggggccgattcaccatctccagagacagcgccaagaactcactttatctgcagatgaacagcctgagagccgaggacacggctgtctattattgtgtgagagagagattctgcagtaatcatatccacaaagaagagcatctgcccagaccctccatctcgcctgagccaggcaccgtgatcaccctggggagccatgtgactttcgtgtgccggggcccggttggggttcaaacattccgcctggagaaggacagtagatccacatacaatgatactgaagatgtgtctcaacctagtccatctgagtcagaggccagattccgcattgactcagtaagtgaaggatatgccgggctttatcgctgcctctattataagccccctaaatggtctgagcagagtgactacctggagctgctggtgaaaggtgacgacgtcacctgggccctgtacccctcttgtggtggtgatggagaggcttccgactacaacatggacgtctggggcaaagggaccacggtcaccgtctcctca

SEQ ID NO: 91 - DNA, 321 bp, Artificial Sequence
  MGC2 light chain nuc
gacatccagatgacccagtctccttccaccctgtctgcatctgtgggagacagagtcaccatctcttgccgggccagtcagcgttttagtggctggttggcctggtatcagcagaaaccagggaaagcccctaacctcctgatctataaggcgtctcctttagcaggtgggggcccatcaaggttcagcggcagtggatctgggacagacttcactctcaccatcagcagcctgcagcctgatgattctgcaacttattactgccaacactacagtaattattcatatacttttggccaggggaccaagctggagatcaga

SEQ ID NO: 92 - protein, 8 aa, Artificial Sequence
  MGC4 CDRH1 aa
GFNSRSYW

SEQ ID NO: 93 - protein, 8 aa, Artificial Sequence
  MGC4 CDRH2 aa
INQDGTEK

SEQ ID NO: 94 - protein, 136 aa, Artificial Sequence
  MGC4 CDRH3 aa
ARDRFCGGESHLHGEEDLPRPSISAEPDTVIPLGSHVTFVCRGPVGVHTFRLERGWRYNDTEDVSQAGPSESEARFRIDSVREGNAGLYRCIYYIAPKWSEQSDYLELRVKGGDVTWALLTYCGGDGEESDYPMDV

SEQ ID NO: 95 - protein, 9 aa, Artificial Sequence
  MGC4 CDRL1 aa
TGPVTSAYY

SEQ ID NO: 96 - skipped

SEQ ID NO: 97 - protein, 9 aa, Artificial Sequence
  MGC4 CDRL2 long aa
LIYSINKKH

SEQ ID NO: 98 - protein, 11 aa, Artificial Sequence
  MGC4 CDRL3 aa
LLSCGGAQPWV

SEQ ID NO: 99 - DNA, 24 bp, Artificial Sequence
  MGC4 CDRH1 nuc
ggattcaactctcgtagttattgg

SEQ ID NO: 100 - DNA, 24 bp, Artificial Sequence
  MGC4 CDRH2 nuc
ataaatcaagatgggactgagaaa

SEQ ID NO: 101 - DNA, 408 bp, Artificial Sequence
  MGC4 CDRH3 nuc
gcgagagacagattctgtggtggtgagagtcacttgcacggagaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaaggtggggacgtcacctgggccctgttaacgtactgtggcggtgatggagaggaatccgactaccccatggacgtc

SEQ ID NO: 102 - DNA, 27 bp, Artificial Sequence
  MGC4 CDRL1 nuc
actggacctgtcaccagtgcttactat

SEQ ID NO: 103 - skipped

SEQ ID NO: 104 - DNA, 27 bp, Artificial Sequence
  MGC4 CDRL2 long nuc
cttatttatagtataaacaaaaaacac

SEQ ID NO: 105 - DNA, 33 bp, Artificial Sequence
  MGC4 CDRL3 nuc
ctgctctcctgtggtggtgctcagccttgggtg

SEQ ID NO: 106 - protein, 243 aa, Artificial Sequence
  MGC4 heavy chain aa
EVQLVESGGGLVQPGGSLRLSCEGSGFNSRSYWMTWVRQAPGKGLEWVASINQDGTEKNYVDSVKGRFTISRDSAKNSLYLQMSSLRADDTAVYYCARDRFCGGESHLHGEEDLPRPSISAEPDTVIPLGSHVTFVCRGPVGVHTFRLERGWRYNDTEDVSQAGPSESEARFRIDSVREGNAGLYRCIYYIAPKWSEQSDYLELRVKGGDVTWALLTYCGGDGEESDYPMDVWGKGTTVTVSS

SEQ ID NO: 107 - protein, 111 aa, Artificial Sequence
  MGC4 light chain aa
QTVVTQEPSLTVSPGGTVTLTCASSTGPVTSAYYPNWFQQKPGQAPRSLIYSINKKHSWTPARFSGSLLGGKAALTLSGVQPEDEADYYCLLSCGGAQPWVFGGGTKLTVQ

SEQ ID NO: 108 - DNA, 729 bp, Artificial Sequence
  MGC4 heavy chain nuc
gaggtgcagctggtggagtctgggggaggcttggtacagcctggggggtccctgagactctcctgtgaaggctctggattcaactctcgtagttattggatgacctgggtccgccaggctccagggaaggggctggagtgggtggccagtataaatcaagatgggactgagaaaaattatgtggactctgtgaagggccggttcaccatctccagagactccgccaagaactcactgtatctgcaaatgagcagcctgagagccgacgacacggctgtatattactgtgcgagagacagattctgtggtggtgagagtcacttgcacggagaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaaggtggggacgtcacctgggccctgttaacgtactgtggcggtgatggagaggaatccgactaccccatggacgtctggggcaaagggaccacggtcaccgtctcctca

SEQ ID NO: 109 - DNA, 334 bp, Artificial Sequence
  MGC4 light chain nuc
cagactgtggttactcaggagccctcactgactgtgtccccaggagggacagtcactctcacctgtgcttccagcactggacctgtcaccagtgcttactatccaaactggttccagcagaagcctggacaagcacccaggtctcttatttatagtataaacaaaaaacactcctggacccctgcccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaag

SEQ ID NO: 110 - protein, 8 aa, Artificial Sequence
  MGC5 CDRH1 aa
GFNSRSYW

SEQ ID NO: 111 - protein, 8 aa, Artificial Sequence
  MGC5 CDRH2 aa
INQDGTEK

SEQ ID NO: 112 - protein, 136 aa, Artificial Sequence
  MGC5 CDRH3 aa
ARDRFCGGESHLHGEEDLPRPSISAEPGTVIPLGSHVTFVCRGPVGVHTFRLERGWRYNDTEDVSQAGPSQSEARFRIDSVREGNAGLYRCLYYIPPKWSEQSDYLELRVKGGDVTWALLTYCGGDGEESDYPMDV

SEQ ID NO: 113 - protein, 9 aa, Artificial Sequence
  MGC5 CDRL1 aa
TGPVTSAYY

SEQ ID NO: 114 - skipped

SEQ ID NO: 115 - protein, 9 aa, Artificial Sequence
  MGC5 CDRL2 long aa
LIYNINKKH

SEQ ID NO: 116 - protein, 11 aa, Artificial Sequence
  MGC5 CDRL3 aa
LLSCGGAQPWV

SEQ ID NO: 117 - DNA, 24 bp, Artificial Sequence
  MGC5 CDRH1 nuc
ggattcaactctcgtagttattgg

SEQ ID NO: 118 - DNA, 24 bp, Artificial Sequence
  MGC5 CDRH2 nuc
ataaatcaagatggaactgagaaa

SEQ ID NO: 119 - DNA, 408 bp, Artificial Sequence
  MGC5 CDRH3 nuc
gcgagagacagattctgtggtggtgagagtcacttgcacggagaagaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaaggtggggacgtcacctgggccctgttaacgtactgtggtggtgatggagaggaatccgactaccccatggacgtc

SEQ ID NO: 120 - DNA, 27 bp, Artificial Sequence
  MGC5 CDRL1 nuc
actggacctgtcaccagtgcttactat

SEQ ID NO: 121 - skipped

SEQ ID NO: 122 - DNA, 27 bp, Artificial Sequence
  MGC5 CDRL2 long nuc
cttatttataatataaacaaaaaacac

SEQ ID NO: 123 - DNA, 33 bp, Artificial Sequence; identical to SEQ ID NO: 105
  MGC5 CDRL3 nuc

SEQ ID NO: 124 - protein, 243 aa, Artificial Sequence
  MGC5 heavy chain aa
EVQLVESGGGLVQPGGSLRLSCEASGFNSRSYWMTWVRQAPGKGLEWVATINQDGTEKNYVDSVRGRFTISRDTAKNSLFLQMNSLRAEDTAVYYCARDRFCGGESHLHGEEDLPRPSISAEPGTVIPLGSHVTFVCRGPVGVHTFRLERGWRYNDTEDVSQAGPSQSEARFRIDSVREGNAGLYRCLYYIPPKWSEQSDYLELRVKGGDVTWALLTYCGGDGEESDYPMDVWGKGTTVTVSS

SEQ ID NO: 125 - protein, 111 aa, Artificial Sequence
  MGC5 light chain aa
QTVVTQEPSLTVSPGGTVTLTCASNTGPVTSAYYPNWFQQKPGQAPRSLIYNINKKHSWTPARFSGSLLGGKAALTLSGVQPEDEADYYCLLSCGGAQPWVFGGGTKLTVQ

SEQ ID NO: 126 - DNA, 729 bp, Artificial Sequence
  MGC5 heavy chain nuc
gaggtgcagctggtggagtctgggggaggcttggtccagcctggggggtcactgagactctcctgtgaagcctctggattcaactctcgtagttattggatgacctgggtccgccaggctccagggaaggggctggagtgggtggccactataaatcaagatggaactgagaaaaattatgtggactctgtgaggggccggttcaccatctccagagacaccgccaagaactcactgtttctgcaaatgaacagcctgagagccgaggacacggctgtatattactgcgcgagagacagattctgtggtggtgagagtcacttgcacggagaagaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaaggtggggacgtcacctgggccctgttaacgtactgtggtggtgatggagaggaatccgactaccccatggacgtctggggcaaagggaccacggtcaccgtctcctca

SEQ ID NO: 127 - DNA, 333 bp, Artificial Sequence
  MGC5 light chain nuc
cagactgtggtgactcaggagccctcactgactgtgtccccaggagggacagtcactctcacctgtgcttccaacactggacctgtcaccagtgcttactatccaaactggttccagcagaagcctggacaagcacccaggtctcttatttataatataaacaaaaaacactcctggacccctgcccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaa

SEQ ID NO: 128 - protein, 8 aa, Artificial Sequence
  MGC7 CDRH1 aa
GFTFRNYW

SEQ ID NO: 129 - protein, 8 aa, Artificial Sequence
  MGC7 CDRH2 aa
IRQDGSEK

SEQ ID NO: 130 - protein, 138 aa, Artificial Sequence
  MGC7 CDRH3 aa
VRDKFCSDENHMHVADDLPRPSISPEPGTVIPLGSHVTFVCRGPVGVQTFRLEKDRRSTYNDTEDVSQPSPSESEARFRIDSVTEGNAGLYRCVYYKPPKWSDQSDFLELLVKGEDVTWALFPHCGADGEDSDYYMDV

SEQ ID NO: 131 - protein, 6 aa, Artificial Sequence
  MGC7 CDRL1 aa
QGLSTW

SEQ ID NO: 132 - skipped

SEQ ID NO: 133 - protein, 9 aa, Artificial Sequence
  MGC7 CDRL2 long aa
LIYAASSLQ

SEQ ID NO: 134 - protein, 9 aa, Artificial Sequence
  MGC7 CDRL3 aa
QQANSFPLT

SEQ ID NO: 135 - DNA, 24 bp, Artificial Sequence
  MGC7 CDRH1 nuc
ggattcaccttcagaaattattgg

SEQ ID NO: 136 - DNA, 24 bp, Artificial Sequence
  MGC7 CDRH2 nuc
ataaggcaagatggaagtgagaag

SEQ ID NO: 137 - DNA, 414 bp, Artificial Sequence
  MGC7 CDRH3 nuc
gtgagagataaattctgcagtgatgagaatcacatgcacgtagcagatgatctgcccagaccctctatctcgcctgagccaggcaccgtgatccccctggggagccatgtgactttcgtgtgtcggggcccggttggggttcaaacattccgcctggagaaggacagaagatccacatacaatgatactgaagatgtgtctcaacctagtccatctgagtcagaggccagattccgcattgactcagtaactgaaggaaatgccgggctttatcgctgcgtctattataagccccctaaatggtctgaccagagtgacttcctggagttgctggtgaagggtgaggacgtcacctgggccctgttcccccattgtggtgctgatggagaggactccgactactacatggacgtc

SEQ ID NO: 138 - DNA, 18 bp, Artificial Sequence
  MGC7 CDRL1 nuc
cagggtcttagtacctgg

SEQ ID NO: 139 - skipped

SEQ ID NO: 140 - DNA, 45 bp, Artificial Sequence
  MGC7 CDRL2 long nuc
tattattgtcaacaggctaacagtttccctctcactttcggcgga

SEQ ID NO: 141 - DNA, 27 bp, Artificial Sequence
  MGC7 CDRL3 nuc
caacaggctaacagtttccctctcact

SEQ ID NO: 142 - protein, 245 aa, Artificial Sequence
  MGC7 heavy chain aa
EVQLVESGGDLVQPGGSLRLSCAASGFTFRNYWMSWVRQTPGKGLEWVANIRQDGSEKYYVDSVKGRFTISRDNAKNLLYLQMNSLRAEDTAVYYCVRDKFCSDENHMHVADDLPRPSISPEPGTVIPLGSHVTFVCRGPVGVQTFRLEKDRRSTYNDTEDVSQPSPSESEARFRIDSVTEGNAGLYRCVYYKPPKWSDQSDFLELLVKGEDVTWALFPHCGADGEDSDYYMDVWGKGTTVTVSS

SEQ ID NO: 143 - protein, 107 aa, Artificial Sequence
  MGC7 light chain aa
DIQMTQSPSSVSASVGDRVTITCRASQGLSTWLAWYQQKPGKAPKILIYAASSLQSGVPSRFSGSGSGTDFTLTISSLQPEDFATYYCQQANSFPLTFGGGTKVEIK

SEQ ID NO: 144 - DNA, 735 bp, Artificial Sequence
  MGC7 heavy chain nuc
gaggtgcagctggtggagtctgggggagacttggtccagcctggggggtccctgagactctcctgtgcagcctctggattcaccttcagaaattattggatgagttgggtccgccagactccagggaagggactggagtgggtggccaacataaggcaagatggaagtgagaagtattatgtggactctgtgaagggccgattcaccatctccagagacaacgccaagaacttattatatctacaaatgaacagcctgagagccgaggacacggctgtgtattactgtgtgagagataaattctgcagtgatgagaatcacatgcacgtagcagatgatctgcccagaccctctatctcgcctgagccaggcaccgtgatccccctggggagccatgtgactttcgtgtgtcggggcccggttggggttcaaacattccgcctggagaaggacagaagatccacatacaatgatactgaagatgtgtctcaacctagtccatctgagtcagaggccagattccgcattgactcagtaactgaaggaaatgccgggctttatcgctgcgtctattataagccccctaaatggtctgaccagagtgacttcctggagttgctggtgaagggtgaggacgtcacctgggccctgttcccccattgtggtgctgatggagaggactccgactactacatggacgtctggggcaaagggaccacggtcaccgtctcctca

SEQ ID NO: 145 - DNA, 321 bp, Artificial Sequence
  MGC7 light chain nuc
gacatccagatgacccagtctccatcttccgtgtctgcatctgtaggagacagagtcaccatcacttgtcgggcgagtcagggtcttagtacctggttagcctggtatcagcagaaaccagggaaagcccctaagatcctgatctatgctgcatccagtttgcaaagtggggtcccatcaaggttcagcggcagtggatctgggacagatttcactctcaccatcagcagcctgcagcctgaagattttgcaacttattattgtcaacaggctaacagtttccctctcactttcggcggagggaccaaggtggagatcaaa

SEQ ID NO: 146 - protein, 8 aa, Artificial Sequence
  MGC17 CDRH1 aa
GFNFRKSW

SEQ ID NO: 147 - protein, 8 aa, Artificial Sequence
  MGC17 CDRH2 aa
IREDGSKA

SEQ ID NO: 148 - protein, 139 aa, Artificial Sequence
  MGC17 CDRH3 aa
ARDRFCSDDEDHSHGAEDLPRPSISAEEGTVIPLGSRLTFVCRGPVGVHTFRLERDRRSTYNDTEDVSHPSPSESEARFRIDSVSEGNAGLYRCVYYKSPEWSKQSDYLELLVKGQEVTWALFTSCGGDGEVPDYDMDV

SEQ ID NO: 149 - protein, 12 aa, Artificial Sequence
  MGC17 CDRL1 aa
QSVLYRSKNKKY

SEQ ID NO: 150 - skipped

SEQ ID NO: 151 - protein, 9 aa, Artificial Sequence
  MGC17 CDRL2 long aa
LIYWTSTRA

SEQ ID NO: 152 - protein, 9 aa, Artificial Sequence
  MGC17 CDRL3 aa
QQYFIFPYT

SEQ ID NO: 153 - DNA, 24 bp, Artificial Sequence
  MGC17 CDRH1 nuc
gggttcaattttagaaagtcttgg

SEQ ID NO: 154 - DNA, 24 bp, Artificial Sequence
  MGC17 CDRH2 nuc
ataagagaagatggaagtaaggca

SEQ ID NO: 155 - DNA, 417 bp, Artificial Sequence
  MGC17 CDRH3 nuc
gcgagagatagattctgcagtgatgatgaggatcacagccacggagcagaagatctgcccagaccctccatctcggctgaggaaggcaccgtgattcccctggggagccgtctgactttcgtgtgccggggcccggttggggttcacacattccgcctggagagggaccgtagatccacatacaatgatactgaagatgtgtctcaccctagtccatctgagtctgaggccagatttcgcattgactcagtgagtgaaggaaatgccgggctttatcgctgcgtctattataagtcccctgaatggtctaagcagagtgattacctggagctgctggtgaaaggtcaggaagtcacctgggccctgtttacttcttgtggtggtgatggagaggtacccgactacgacatggacgtc

SEQ ID NO: 156 - DNA, 36 bp, Artificial Sequence
  MGC17 CDRL1 nuc
cagagtgttttatacaggtccaagaataagaaatat

SEQ ID NO: 157 - skipped

SEQ ID NO: 158 - DNA, 27 bp, Artificial Sequence
  MGC17 CDRL2 long nuc
ctcatttactggacatctactcgggcg

SEQ ID NO: 159 - DNA, 27 bp, Artificial Sequence
  MGC17 CDRL3 nuc
cagcagtattttatttttccgtacact

SEQ ID NO: 160 - protein, 246 aa, Artificial Sequence
  MGC17 heavy chain aa
EVQLVESGGGLVQPGGSLKLSCVASGFNFRKSWMSWVRQAPGKGLEWVANIREDGSKAYYVDSVKGRFTVSRDNAKNSLYLQINSLRADDTAVYYCARDRFCSDDEDHSHGAEDLPRPSISAEEGTVIPLGSRLTFVCRGPVGVHTFRLERDRRSTYNDTEDVSHPSPSESEARFRIDSVSEGNAGLYRCVYYKSPEWSKQSDYLELLVKGQEVTWALFTSCGGDGEVPDYDMDVRGKGTTVTVSS

SEQ ID NO: 161 - protein, 113 aa, Artificial Sequence
  MGC17 light chain aa
DIVMTQSPDSLAVSLGERATINCKSSQSVLYRSKNKKYLAWFQQRPGQPPKVLIYWTSTRASGVPDRFSGSGSGTDFTLTISSLQADDVAVYYCQQYFIFPYTFGQGTKLEIR

SEQ ID NO: 162 - DNA, 738 bp, Artificial Sequence
  MGC17 heavy chain nuc
gaggtgcagctggtggagtcggggggaggcttggtccagcctggggggtccctgaaactgtcctgtgtagcctctgggttcaattttagaaagtcttggatgagttgggtccgccaggctccagggaaggggctggagtgggtggcaaacataagagaagatggaagtaaggcatactatgtggactctgtcaagggccgattcaccgtctccagagacaacgccaagaactcgctgtatctgcagatcaacagcctgagagccgacgacacggctgtctattactgtgcgagagatagattctgcagtgatgatgaggatcacagccacggagcagaagatctgcccagaccctccatctcggctgaggaaggcaccgtgattcccctggggagccgtctgactttcgtgtgccggggcccggttggggttcacacattccgcctggagagggaccgtagatccacatacaatgatactgaagatgtgtctcaccctagtccatctgagtctgaggccagatttcgcattgactcagtgagtgaaggaaatgccgggctttatcgctgcgtctattataagtcccctgaatggtctaagcagagtgattacctggagctgctggtgaaaggtcaggaagtcacctgggccctgtttacttcttgtggtggtgatggagaggtacccgactacgacatggacgtccggggcaaagggaccacggtcaccgtctcttca

SEQ ID NO: 163 - DNA, 339 bp, Artificial Sequence
  MGC17 light chain nuc
gacatcgtgatgacccaatctcctgactccctggctgtgtctctgggcgagagggccaccatcaactgcaagtccagccagagtgttttatacaggtccaagaataagaaatatttagcttggttccagcagagaccaggacagcctcctaaggttctcatttactggacatctactcgggcgtccggggtccctgaccgattcagtggcagcgggtctgggacagatttcactctcaccatcagcagcctgcaggctgatgatgtggcagtttattattgtcagcagtattttatttttccgtacacttttggccaggggaccaagttggagatcaga

SEQ ID NO: 164 - protein, 8 aa, Artificial Sequence
  MGC26 CDRH1 aa
GFTFSTYW

SEQ ID NO: 165 - protein, 8 aa, Artificial Sequence
  MGC26 CDRH2 aa
IKQDGTER

SEQ ID NO: 166 - protein, 136 aa, Artificial Sequence
  MGC26 CDRH3 aa
VRDRFCRDHMHIEEDLPRPSISPEPATVIPLGSHVTIVCRGPVGVETFRLQKESRSLYNDTEDVSQPSPSESEARFRIDSVSEGHGGLYRCLYYKSSKWSEQSDYLEMLVKGEDVTWALFPYCGGDGEESDYYMDV

SEQ ID NO: 167 - protein, 6 aa, Artificial Sequence
  MGC26 CDRL1 aa
QRLSRS

SEQ ID NO: 168 - skipped

SEQ ID NO: 169 - protein, 9 aa, Artificial Sequence
  MGC26 CDRL2 long aa
LIYKASPLE

SEQ ID NO: 170 - protein, 9 aa, Artificial Sequence
  MGC26 CDRL3 aa
QQYSNYSYS

SEQ ID NO: 171 - DNA, 24 bp, Artificial Sequence
  MGC26 CDRH1 nuc
ggattcacctttagtacttattgg

SEQ ID NO: 172 - protein, 24 aa, Artificial Sequence
  MGC26 CDRH2 nuc
ATAAAGCAAGATGGAACTGAGAGA

SEQ ID NO: 173 - DNA, 408 bp, Artificial Sequence
  MGC26 CDRH3 nuc
gtgagagacagattctgcagagatcacatgcacatagaagaagatctgcccagaccctccatctcgccggagccagccaccgtgatccccctggggagccatgtgactatcgtgtgccggggcccggttggggttgaaacattccgcctgcagaaggagagtagatccctgtacaatgacactgaagatgtgtctcaacctagtccatctgagtcagaggccagattccgcattgactcagtaagtgaagggcatggcgggctttatcgctgcctctattataagtcttctaaatggtctgagcagagtgactacctggagatgctggtgaaaggtgaggacgtcacctgggccctgttcccctattgtggtggtgatggagaggaatccgactactacatggacgtc

SEQ ID NO: 174 - DNA, 18 bp, Artificial Sequence
  MGC26 CDRL1 nuc
cagcgtcttagtcgctcg

SEQ ID NO: 175 - skipped

SEQ ID NO: 176 - DNA, 27 bp, Artificial Sequence
  MGC26 CDRL2 long nuc
ctgatctataaggcgtctcctttagaa

SEQ ID NO: 177 - DNA, 27 bp, Artificial Sequence
  MGC26 CDRL3 nuc
caacaatacagtaattattcatatagt

SEQ ID NO: 178 - protein, 243 aa, Artificial Sequence
  MGC26 heavy chain aa
EVQLVDSGGGLVQPGGSLRLSCAASGFTFSTYWMTWVRQTPGKGLEWVASIKQDGTERYYVDSVKGRFIISRDNAKNSLYLQMHSLRAEDTAVYYCVRDRFCRDHMHIEEDLPRPSISPEPATVIPLGSHVTIVCRGPVGVETFRLQKESRSLYNDTEDVSQPSPSESEARFRIDSVSEGHGGLYRCLYYKSSKWSEQSDYLEMLVKGEDVTWALFPYCGGDGEESDYYMDVWGKGTTVTVSS

SEQ ID NO: 179 - protein, 107 aa, Artificial Sequence
  MGC26 light chain aa
DIQLTQSPSTLSASVGDRVTISCRASQRLSRSLAWYQQRPRKAPNLLIYKASPLEIGGPSRFTGSGSGTEFTLTISSLQPDDSATYYCQQYSNYSYSFGQGTKLEIR

SEQ ID NO: 180 - DNA, 729 bp, Artificial Sequence
  MGC26 heavy chain nuc
gaggtgcagctggtggattctgggggaggcttggtccagcctggggggtccctgagactctcctgtgcagcctctggattcacctttagtacttattggatgacctgggtccgccagactccagggaaggggctggagtgggtggccagcataaagcaagatggaactgagagatactatgtggactctgtgaagggccgattcattatctccagagacaacgccaagaactcactatatttgcaaatgcacagcctgagagccgaggacacggctgtgtattattgtgtgagagacagattctgcagagatcacatgcacatagaagaagatctgcccagaccctccatctcgccggagccagccaccgtgatccccctggggagccatgtgactatcgtgtgccggggcccggttggggttgaaacattccgcctgcagaaggagagtagatccctgtacaatgacactgaagatgtgtctcaacctagtccatctgagtcagaggccagattccgcattgactcagtaagtgaagggcatggcgggctttatcgctgcctctattataagtcttctaaatggtctgagcagagtgactacctggagatgctggtgaaaggtgaggacgtcacctgggccctgttcccctattgtggtggtgatggagaggaatccgactactacatggacgtctggggcaaagggaccacggtcaccgtctcctca

SEQ ID NO: 181 - DNA, 321 bp, Artificial Sequence
  MGC26 light chain nuc
gacatccagctgacccagtctccttccaccctgtctgcatctgtaggagacagagtcaccatctcttgccgggccagtcagcgtcttagtcgctcgttggcctggtatcagcagagaccacggaaagcccctaacctcctgatctataaggcgtctcctttagaaattgggggcccatcaaggttcaccggcagtggatctgggacagaattcactctcaccatcagcagcctgcagcctgatgattctgcaacttattactgccaacaatacagtaattattcatatagttttggccaggggaccaagctggagatcaga

SEQ ID NO: 182 - protein, 8 aa, Artificial Sequence
  MGC28 CDRH1 aa
GFTFSSYW

SEQ ID NO: 183 - protein, 8 aa, Artificial Sequence
  MGC28 CDRH2 aa
INQDGSER

SEQ ID NO: 184 - protein, 137 aa, Artificial Sequence
  MGC28 CDRH3 aa
ARQRFCSDGSLFHGEDLPRPTISAETGTVISLGSHVTFVCRGPLGVQTFRLERESRSRYSETEDVSQVGPSESEARFRIDSVSEGNAGLYRCIYYKPPKWSEQSDYLELRVKGEDVTWALLTYCGGDRDESDYYMDV

SEQ ID NO: 185 - protein, 9 aa, Artificial Sequence
  MGC28 CDRL1 aa
TGSVTSGSF

SEQ ID NO: 186 - skipped

SEQ ID NO: 187 - protein, 9 aa, Artificial Sequence
  MGC28 CDRL2 long aa
LIYSTTKKH

SEQ ID NO: 188 - protein, 11 aa, Artificial Sequence
  MGC28 CDRL3 aa
LLYCGGGQPWV

SEQ ID NO: 189 - DNA, 24 bp, Artificial Sequence
  MGC28 CDRH1 nuc
ggattcacgtttagttcttattgg

SEQ ID NO: 190 - DNA, 24 bp, Artificial Sequence
  MGC28 CDRH2 nuc
ataaaccaagatggaagtgagaga

SEQ ID NO: 191 - DNA, 411 bp, Artificial Sequence
  MGC28 CDRH3 nuc
gcgagacaaagattctgcagtgatgggagtctctttcacggagaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaaggtgaggacgtcacctgggccctgttaacctattgtggtggtgatagagacgaatccgactactacatggacgtc

SEQ ID NO: 192 - DNA, 27 bp, Artificial Sequence
  MGC28 CDRL1 nuc
actggatcagtcaccagtggttccttt

SEQ ID NO: 193 - skipped

SEQ ID NO: 194 - DNA, 27 bp, Artificial Sequence
  MGC28 CDRL2 long nuc
ctgatttatagtacaaccaaaaaacac

SEQ ID NO: 195 - DNA, 33 bp, Artificial Sequence
  MGC28 CDRL3 nuc
ctactctactgtggtggtggtcaaccttgggtg

SEQ ID NO: 196 - protein, 244 aa, Artificial Sequence
  MGC28 heavy chain aa
EVQLVESGGGLVQPGGSLRLSCEASGFTFSSYWMTWVRQAPGKGLEWVANINQDGSERYYVDSVKGRFTISRDTVKNSLYLQMNNLRAEDTAVYYCARQRFCSDGSLFHGEDLPRPTISAETGTVISLGSHVTFVCRGPLGVQTFRLERESRSRYSETEDVSQVGPSESEARFRIDSVSEGNAGLYRCIYYKPPKWSEQSDYLELRVKGEDVTWALLTYCGGDRDESDYYMDVWGKGTTVTVSS

SEQ ID NO: 197 - protein, 111 aa, Artificial Sequence
  MGC28 light chain aa
QTVVTQEPSLTVSPGGTVTLTCASSTGSVTSGSFPNWFQQTPGQAPRSLIYSTTKKHSWTPARFSGSLLGGKAALTVSDTQPEDEAEYYCLLYCGGGQPWVFGGGTKLTVL

SEQ ID NO: 198 - DNA, 732 bp, Artificial Sequence
  MGC28 heavy chain nuc
gaggtgcagctggtggagtctgggggaggcttggtccagccgggggggtccctgagactctcctgtgaagcctctggattcacgtttagttcttattggatgacctgggtccgccaggctccagggaaggggctggagtgggtggccaatataaaccaagatggaagtgagagatattatgtggactctgtgaagggccggttcaccatctccagagacaccgtcaagaactcactgtatttgcaaatgaacaacctgagagccgaggacacggctgtatattactgcgcgagacaaagattctgcagtgatgggagtctctttcacggagaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaaggtgaggacgtcacctgggccctgttaacctattgtggtggtgatagagacgaatccgactactacatggacgtctggggcaaagggaccacggtcaccgtctcctca

SEQ ID NO: 199 - DNA, 333 bp, Artificial Sequence
  MGC28 light chain nuc
cagactgtggtgactcaggagccctcactgactgtgtccccaggagggacagtcactctcacctgtgcttccagtactggatcagtcaccagtggttcctttccaaactggttccagcagacacctggacaagcacccaggtcactgatttatagtacaaccaaaaaacactcttggacccctgcccggttctcaggctctctccttgggggcaaagctgccctgacagtgtcagatacacagccggaggacgaggctgagtattactgcctactctactgtggtggtggtcaaccttgggtgttcggcggagggaccaagctgaccgtccta

SEQ ID NO: 200 - protein, 8 aa, Artificial Sequence
  MGC29 CDRH1 aa
GFNSRSYW

SEQ ID NO: 201 - protein, 8 aa, Artificial Sequence
  MGC29 CDRH2 aa
INQDGTEK

SEQ ID NO: 202 - protein, 136 aa, Artificial Sequence; identical to SEQ ID NO: 112
  MGC29 CDRH3 aa

SEQ ID NO: 203 - protein, 9 aa, Artificial Sequence
  MGC29 CDRL1 aa
TGPVTSAYY

SEQ ID NO: 204 - skipped

SEQ ID NO: 205 - protein, 9 aa, Artificial Sequence
  MGC29 CDRL2 long aa
LIYNINKKH

SEQ ID NO: 206 - protein, 11 aa, Artificial Sequence
  MGC29 CDRL3 aa
LLSCGGAQPWV

SEQ ID NO: 207 - DNA, 24 bp, Artificial Sequence
  MGC29 CDRH1 nuc
ggattcaactctcgtagttattgg

SEQ ID NO: 208 - DNA, 24 bp, Artificial Sequence
  MGC29 CDRH2 nuc
ataaatcaagatggaactgagaaa

SEQ ID NO: 209 - DNA, 408 bp, Artificial Sequence; identical to SEQ ID NO: 119
  MGC29 CDRH3 nuc

SEQ ID NO: 210 - DNA, 27 bp, Artificial Sequence
  MGC29 CDRL1 nuc
actggacctgtcaccagtgcttactat

SEQ ID NO: 211 - skipped

SEQ ID NO: 212 - DNA, 27 bp, Artificial Sequence
  MGC29 CDRL2 long nuc
cttatttataatataaacaaaaaacac

SEQ ID NO: 213 - DNA, 33 bp, Artificial Sequence; identical to SEQ ID NO: 105
  MGC29 CDRL3 nuc

SEQ ID NO: 214 - protein, 243 aa, Artificial Sequence; identical to SEQ ID NO: 124
  MGC29 heavy chain aa

SEQ ID NO: 215 - protein, 111 aa, Artificial Sequence; identical to SEQ ID NO: 125
  MGC29 light chain aa

SEQ ID NO: 216 - DNA, 729 bp, Artificial Sequence
  MGC29 heavy chain nuc
gaggtgcagctggtggagtctgggggaggcttggtccagcctggggggtccctgagactctcctgtgaagcctctggattcaactctcgtagttattggatgacctgggtccgccaggctccagggaaggggctggagtgggtggccactataaatcaagatggaactgagaaaaattatgtggactctgtgaggggccggttcaccatctccagagacaccgccaagaactcactgtttctgcaaatgaacagcctgagagccgaggacacggctgtatattactgcgcgagagacagattctgtggtggtgagagtcacttgcacggagaagaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaaggtggggacgtcacctgggccctgttaacgtactgtggtggtgatggagaggaatccgactaccccatggacgtctggggcaaagggaccacggtcaccgtctcctca

SEQ ID NO: 217 - DNA, 333 bp, Artificial Sequence; identical to SEQ ID NO: 127
  MGC29 light chain nuc

SEQ ID NO: 218 - protein, 8 aa, Artificial Sequence
  MGC32 CDRH1 aa
GFNFRKSW

SEQ ID NO: 219 - protein, 8 aa, Artificial Sequence
  MGC32 CDRH2 aa
IREDGSES

SEQ ID NO: 220 - protein, 139 aa, Artificial Sequence; identical to SEQ ID NO: 58
  MGC32 CDRH3 aa

SEQ ID NO: 221 - protein, 12 aa, Artificial Sequence
  MGC32 CDRL1 aa
QSVLYRSKNKNY

SEQ ID NO: 222 - skipped

SEQ ID NO: 223 - protein, 9 aa, Artificial Sequence
  MGC32 CDRL2 long aa
LIYSTSTRA

SEQ ID NO: 224 - protein, 9 aa, Artificial Sequence
  MGC32 CDRL3 aa
LQYYITPYT

SEQ ID NO: 225 - DNA, 24 bp, Artificial Sequence
  MGC32 CDRH1 nuc
gggttcaactttagaaagtcttgg

SEQ ID NO: 226 - DNA, 24 bp, Artificial Sequence
  MGC32 CDRH2 nuc
ataagagaagatggaagtgagagt

SEQ ID NO: 227 - DNA, 417 bp, Artificial Sequence; identical to SEQ ID NO: 65
  MGC32 CDRH3 nuc

SEQ ID NO: 228 - DNA, 36 bp, Artificial Sequence; identical to SEQ ID NO: 66
  MGC32 CDRL1 nuc

SEQ ID NO: 229 - skipped

SEQ ID NO: 230 - DNA, 27 bp, Artificial Sequence
  MGC32 CDRL2 long nuc
ctcatttactcgacatctactcgggcg

SEQ ID NO: 231 - DNA, 27 bp, Artificial Sequence
  MGC32 CDRL3 nuc
ctgcaatattatattactccctacact

SEQ ID NO: 232 - protein, 246 aa, Artificial Sequence; identical to SEQ ID NO: 70
  MGC32 heavy chain aa

SEQ ID NO: 233 - protein, 113 aa, Artificial Sequence
  MGC32 light chain aa
DILMTQSPDSLAVSLGERATINCKSSQSVLYRSKNKNYLAWFQQKPGQPPKVLIYSTSTRASGVPDRFTGSGSGTDFTLTISSLQAEDVAVYYCLQYYITPYTFGQGTKLEIK

SEQ ID NO: 234 - DNA, 738 bp, Artificial Sequence; identical to SEQ ID NO: 72
  MGC32 heavy chain nuc

SEQ ID NO: 235 - DNA, 339 bp, Artificial Sequence
  MGC32 light chain nuc
gacatcctcatgacccagtctccagactccctggctgtgtctctgggcgagagggccaccatcaactgcaagtccagtcagagtgttttatacaggtccaagaataagaactacttagcttggttccagcagaaaccaggacagcctcctaaggtgctcatttactcgacatctactcgggcgtccggggtccctgaccgattcactggcagcgggtctgggacagatttcactctcaccatcagcagcctgcaggctgaagatgtggcagtttattactgtctgcaatattatattactccctacacttttggccaggggaccaagttggagatcaaa

SEQ ID NO: 236 - protein, 8 aa, Artificial Sequence
  MGC33 CDRH1 aa
GFTFSSYW

SEQ ID NO: 237 - protein, 8 aa, Artificial Sequence
  MGC33 CDRH2 aa
INQDGSER

SEQ ID NO: 238 - protein, 137 aa, Artificial Sequence; identical to SEQ ID NO: 184
  MGC33 CDRH3 aa

SEQ ID NO: 239 - protein, 9 aa, Artificial Sequence
  MGC33 CDRL1 aa
TGSVTSGSF

SEQ ID NO: 240 - skipped

SEQ ID NO: 241 - protein, 9 aa, Artificial Sequence
  MGC33 CDRL2 long aa
LIYSTTKKH

SEQ ID NO: 242 - protein, 11 aa, Artificial Sequence
  MGC33 CDRL3 aa
LLYCGGGQPWV

SEQ ID NO: 243 - DNA, 24 bp, Artificial Sequence
  MGC33 CDRH1 nuc
ggattcacgtttagttcttattgg

SEQ ID NO: 244 - DNA, 24 bp, Artificial Sequence
  MGC33 CDRH2 nuc
ataaaccaagatggaagtgagaga

SEQ ID NO: 245 - DNA, 411 bp, Artificial Sequence; identical to SEQ ID NO: 191
  MGC33 CDRH3 nuc

SEQ ID NO: 246 - DNA, 27 bp, Artificial Sequence
  MGC33 CDRL1 nuc
actggatcagtcaccagtggttccttt

SEQ ID NO: 247 - skipped

SEQ ID NO: 248 - DNA, 27 bp, Artificial Sequence
  MGC33 CDRL2 long nuc
ctgatttatagtacaaccaaaaaacac

SEQ ID NO: 249 - DNA, 33 bp, Artificial Sequence; identical to SEQ ID NO: 195
  MGC33 CDRL3 nuc

SEQ ID NO: 250 - protein, 244 aa, Artificial Sequence
  MGC33 heavy chain aa
EVHLVESGGGLVQPGGSLRLSCEASGFTFSSYWMTWVRQAPGKGLEWVANINQDGSERYYVDSVKGRFTISRDTVKNSLYLQMNNLRAEDTAVYYCARQRFCSDGSLFHGEDLPRPTISAETGTVISLGSHVTFVCRGPLGVQTFRLERESRSRYSETEDVSQVGPSESEARFRIDSVSEGNAGLYRCIYYKPPKWSEQSDYLELRVKGEDVTWALLTYCGGDRDESDYYMDVWGKGTTVTVSS

SEQ ID NO: 251 - protein, 111 aa, Artificial Sequence
  MGC33 light chain aa
QTVVTQEPSLTVSPGGTVTLTCASSTGSVTSGSFPNWFQQTPGQAPRSLIYSTTKKHSWTPARFSGSLLGGKAALTVSDTQPEDEAEYYCLLYCGGGQPWVFGGGTKLTVQ

SEQ ID NO: 252 - DNA, 732 bp, Artificial Sequence
  MGC33 heavy chain nuc
gaggtgcacctggtggagtctgggggaggcttggtccagccgggggggtccctgagactctcctgtgaagcctctggattcacgtttagttcttattggatgacctgggtccgccaggctccagggaaggggctggagtgggtggccaatataaaccaagatggaagtgagagatattatgtggactctgtgaagggccggttcaccatctccagagacaccgtcaagaactcactgtatttgcaaatgaacaacctgagagccgaggacacggctgtatattactgcgcgagacaaagattctgcagtgatgggagtctctttcacggagaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaaggtgaggacgtcacctgggccctgttaacctattgtggtggtgatagagacgaatccgactactacatggacgtctggggcaaagggaccacggtcaccgtctcctca

SEQ ID NO: 253 - DNA, 333 bp, Artificial Sequence
  MGC33 light chain nuc
cagactgtggtgactcaggagccctcactgactgtgtccccaggagggacagtcactctcacctgtgcttccagtactggatcagtcaccagtggttcctttccaaactggttccagcagacacctggacaagcacccaggtcactgatttatagtacaaccaaaaaacactcttggacccctgcccggttctcaggctctctccttgggggcaaagctgccctgacagtgtcagatacacagccggaggacgaggctgagtattactgcctactctactgtggtggtggtcaaccttgggtgttcggcggagggaccaagctgaccgtccaa

SEQ ID NO: 254 - protein, 8 aa, Artificial Sequence
  MGC34 CDRH1 aa
GFTFSSYW

SEQ ID NO: 255 - protein, 8 aa, Artificial Sequence
  MGC34 CDRH2 aa
INQDGSQK

SEQ ID NO: 256 - protein, 138 aa, Artificial Sequence
  MGC34 CDRH3 aa
ARERLCTDDSHMHGEEDLPRPSISAEPGTVIPLGSHVTFVCRGPVGIHTFRLERESRSLYTETEDVTQVSPSESEARFRIESVTEGNAGLYRCVYYKPPKWSEQSDYLELLVKGEDVTRALFTHCGGDGKESDYHMDV

SEQ ID NO: 257 - protein, 9 aa, Artificial Sequence
  MGC34 CDRL1 aa
TGAVTSGYY

SEQ ID NO: 258 - skipped

SEQ ID NO: 259 - protein, 9 aa, Artificial Sequence
  MGC34 CDRL2 long aa
LIYSTSKTH

SEQ ID NO: 260 - protein, 11 aa, Artificial Sequence
  MGC34 CDRL3 aa
LLYYGGPQPWV

SEQ ID NO: 261 - DNA, 24 bp, Artificial Sequence
  MGC34 CDRH1 nuc
ggattcacctttagtagttattgg

SEQ ID NO: 262 - DNA, 24 bp, Artificial Sequence
  MGC34 CDRH2 nuc
ataaaccaagatggaagtcagaaa

SEQ ID NO: 263 - DNA, 414 bp, Artificial Sequence
  MGC34 CDRH3 nuc
gcgagagaaagattgtgcactgatgatagtcacatgcacggagaagaagatctgcccagaccctccatctcggctgagccaggcaccgtgatccccctggggagtcatgtgaccttcgtgtgccggggcccggttgggattcacacattccgcctggagagggagagtagatccctatacactgaaactgaagatgtgactcaagtaagtccttctgagtcagaggccagattccgcattgagtcagtaactgaaggaaatgccgggctttatcgctgcgtctattataagccccctaaatggtctgagcagagtgactacctggagctgctggtgaaaggtgaggacgtcacccgggccctgttcacccactgtggtggtgatggaaaggagtccgactaccacatggacgtc

SEQ ID NO: 264 - DNA, 27 bp, Artificial Sequence
  MGC34 CDRL1 nuc
actggagcagtcaccagtggttactat

SEQ ID NO: 265 - skipped

SEQ ID NO: 266 - DNA, 27 bp, Artificial Sequence
  MGC34 CDRL2 long nuc
ctgatttatagtacaagcaaaacacac

SEQ ID NO: 267 - DNA, 33 bp, Artificial Sequence
  MGC34 CDRL3 nuc
ctgctctattatggtggtcctcagccttgggtg

SEQ ID NO: 268 - protein, 245 aa, Artificial Sequence
  MGC34 heavy chain aa
EVQLVESGGGLVQPGGSLRLSCEASGFTFSSYWMSWVRQAPGKGLEWVANINQDGSQKDYVDSVKGRFTISRDTAKNSLYLQMNSLRAEDTAVYYCARERLCTDDSHMHGEEDLPRPSISAEPGTVIPLGSHVTFVCRGPVGIHTFRLERESRSLYTETEDVTQVSPSESEARFRIESVTEGNAGLYRCVYYKPPKWSEQSDYLELLVKGEDVTRALFTHCGGDGKESDYHMDVWGKGTTVTVSS

SEQ ID NO: 269 - protein, 111 aa, Artificial Sequence
  MGC34 light chain aa
QTVVTQEPSLTVSPGGTVTLTCASSTGAVTSGYYPSWFHQKPGQPVRALIYSTSKTHSWTPARFSGSLLGGKAALTLSNVQPEDEADYYCLLYYGGPQPWVFGGGTKLTVQ

SEQ ID NO: 270 - DNA, 735 bp, Artificial Sequence
  MGC34 heavy chain nuc
gaggtgcagctggtggagtctgggggaggcttggtccagcctggggggtcactgagactctcctgtgaagcctccggattcacctttagtagttattggatgagctgggtccgccaggctccagggaaggggctggagtgggtggccaatataaaccaagatggaagtcagaaagattatgtggattctgtgaagggccgattcaccatctccagagacaccgccaagaattcattatatctccaaatgaacagcctgagagccgaggacacggctgtttactactgtgcgagagaaagattgtgcactgatgatagtcacatgcacggagaagaagatctgcccagaccctccatctcggctgagccaggcaccgtgatccccctggggagtcatgtgaccttcgtgtgccggggcccggttgggattcacacattccgcctggagagggagagtagatccctatacactgaaactgaagatgtgactcaagtaagtccttctgagtcagaggccagattccgcattgagtcagtaactgaaggaaatgccgggctttatcgctgcgtctattataagccccctaaatggtctgagcagagtgactacctggagctgctggtgaaaggtgaggacgtcacccgggccctgttcacccactgtggtggtgatggaaaggagtccgactaccacatggacgtctggggcaaagggaccacggtcaccgtctcctca

SEQ ID NO: 271 - DNA, 333 bp, Artificial Sequence
  MGC34 light chain nuc
cagactgtggtgactcaggagccctcactgactgtgtccccaggagggacagtcactctcacctgtgcttctagcactggagcagtcaccagtggttactatccaagctggttccaccagaaacctggacaaccagtcagggcactgatttatagtacaagcaaaacacactcctggacccctgcccgcttctcaggctccctccttgggggcaaagctgccctgacactgtcaaatgtccagcctgaggacgaggctgactattactgcctgctctattatggtggtcctcagccttgggtgttcggcggagggaccaagctgaccgtccaa

SEQ ID NO: 272 - protein, 8 aa, Artificial Sequence
  MGC35 CDRH1 aa
GFNSRSYW

SEQ ID NO: 273 - protein, 8 aa, Artificial Sequence
  MGC35 CDRH2 aa
INQDATEK

SEQ ID NO: 274 - protein, 136 aa, Artificial Sequence
  MGC35 CDRH3 aa
ARDRFCGGESHLHGQEDLPRPSISAEPGSVIPLGSLVTFVCRGPVGVHTFRLERGWTYNDTEDVSQAGPSESEARFRMDSVREGNAGLYRCIYYKPPKWSEQSAYLELRVKGGDVTWALLTYCGGDGEESDYPMDV

SEQ ID NO: 275 - protein, 9 aa, Artificial Sequence
  MGC35 CDRL1 aa
TGPVTSAYY

SEQ ID NO: 276 - skipped

SEQ ID NO: 277 - protein, 9 aa, Artificial Sequence
  MGC35 CDRL2 long aa
LIYNINKKH

SEQ ID NO: 278 - protein, 11 aa, Artificial Sequence
  MGC35 CDRL3 aa
LLSCGGAQPWV

SEQ ID NO: 279 - DNA, 24 bp, Artificial Sequence
  MGC35 CDRH1 nuc
ggattcaactctcgtagttattgg

SEQ ID NO: 280 - DNA, 24 bp, Artificial Sequence
  MGC35 CDRH2 nuc
ataaatcaagatgcaactgagaaa

SEQ ID NO: 281 - DNA, 408 bp, Artificial Sequence
  MGC35 CDRH3 nuc
gcgagagacagattctgtggtggtgagagtcacttgcacggacaagaagatctgcccagaccctccatctcggctgagccaggctccgtgatccccctggggagccttgtgactttcgtgtgccggggcccggttggggttcacacattccgcctcgagagggggtggacatacaacgacactgaagatgtgtctcaagctggtccatctgagtcagaggccagattccgcatggactcggtaagggaaggaaatgccgggctttatcgatgcatctattacaaaccccctaaatggtctgagcagagtgcctacctggaactgcgggtgaaaggtggggacgtcacctgggccctgttaacgtactgtggtggtgatggagaggaatccgactaccccatggacgtc

SEQ ID NO: 282 - DNA, 27 bp, Artificial Sequence
  MGC35 CDRL1 nuc
actggacctgtcaccagtgcttactat

SEQ ID NO: 283 - skipped

SEQ ID NO: 284 - DNA, 27 bp, Artificial Sequence
  MGC35 CDRL2 long nuc
cttatttataatataaacaaaaaacac

SEQ ID NO: 285 - DNA, 33 bp, Artificial Sequence; identical to SEQ ID NO: 105
  MGC35 CDRL3 nuc

SEQ ID NO: 286 - protein, 243 aa, Artificial Sequence
  MGC35 heavy chain aa
EVQLVESGGGLVQPGGSLRLSCEASGFNSRSYWMTWVRQAPGKGLEWVASINQDATEKNYVDSVKGRFTISRDTAKNSLYLQMNSLRAEDTAVYYCARDRFCGGESHLHGQEDLPRPSISAEPGSVIPLGSLVTFVCRGPVGVHTFRLERGWTYNDTEDVSQAGPSESEARFRMDSVREGNAGLYRCIYYKPPKWSEQSAYLELRVKGGDVTWALLTYCGGDGEESDYPMDVWGKGTTVTVSS

SEQ ID NO: 287 - protein, 111 aa, Artificial Sequence
  MGC35 light chain aa
QTVVTQEPSLTVSPGGTVTLTCASSTGPVTSAYYPNWFQQKPGQAPRSLIYNINKKHSWTPDRFSGSLLGGKAALTLSGVQPEDEADYYCLLSCGGAQPWVFGGGTKLTVQ

SEQ ID NO: 288 - DNA, 729 bp, Artificial Sequence
  MGC35 heavy chain nuc
gaggtgcaactggtggagtctgggggaggcttggtccagcctggggggtccctgagactctcctgtgaagcctctggattcaactctcgtagttattggatgacctgggtccgccaggctccagggaaggggctggagtgggtggccagtataaatcaagatgcaactgagaaaaattatgtggactctgtgaagggccggttcaccatctccagagacaccgccaagaactcactgtatctgcaaatgaacagcctgagagccgaggacacggctgtatattactgcgcgagagacagattctgtggtggtgagagtcacttgcacggacaagaagatctgcccagaccctccatctcggctgagccaggctccgtgatccccctggggagccttgtgactttcgtgtgccggggcccggttggggttcacacattccgcctcgagagggggtggacatacaacgacactgaagatgtgtctcaagctggtccatctgagtcagaggccagattccgcatggactcggtaagggaaggaaatgccgggctttatcgatgcatctattacaaaccccctaaatggtctgagcagagtgcctacctggaactgcgggtgaaaggtggggacgtcacctgggccctgttaacgtactgtggtggtgatggagaggaatccgactaccccatggacgtctggggcaaagggaccacggtcaccgtctcctca

SEQ ID NO: 289 - DNA, 333 bp, Artificial Sequence
  MGC35 light chain nuc
cagactgtggtgactcaggagccctcactgactgtgtccccaggagggacagtcactctcacctgtgcttccagcactggacctgtcaccagtgcttactatccaaactggttccagcagaagcctggacaagcacccaggtctcttatttataatataaacaaaaaacactcctggacccctgaccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaa

SEQ ID NO: 290 - protein, 8 aa, Artificial Sequence
  MGC36 CDRH1 aa
GFNSRSYW

SEQ ID NO: 291 - protein, 8 aa, Artificial Sequence
  MGC36 CDRH2 aa
INQDGTEK

SEQ ID NO: 292 - protein, 136 aa, Artificial Sequence; identical to SEQ ID NO: 94
  MGC36 CDRH3 aa

SEQ ID NO: 293 - protein, 9 aa, Artificial Sequence
  MGC36 CDRL1 aa
TGPVTSAYY

SEQ ID NO: 294 - skipped

SEQ ID NO: 295 - protein, 9 aa, Artificial Sequence
  MGC36 CDRL2 long aa
LIYSINKKH

SEQ ID NO: 296 - protein, 11 aa, Artificial Sequence
  MGC36 CDRL3 aa
LLSCGGAQPWV

SEQ ID NO: 297 - DNA, 24 bp, Artificial Sequence
  MGC36 CDRH1 nuc
ggattcaactctcgtagttattgg

SEQ ID NO: 298 - DNA, 24 bp, Artificial Sequence
  MGC36 CDRH2 nuc
ataaatcaagatgggactgagaaa

SEQ ID NO: 299 - DNA, 408 bp, Artificial Sequence; identical to SEQ ID NO: 101
  MGC36 CDRH3 nuc

SEQ ID NO: 300 - DNA, 27 bp, Artificial Sequence
  MGC36 CDRL1 nuc
actggacctgtcaccagtgcttactat

SEQ ID NO: 301 - skipped

SEQ ID NO: 302 - DNA, 27 bp, Artificial Sequence
  MGC36 CDRL2 long nuc
cttatttatagtataaacaaaaaacac

SEQ ID NO: 303 - DNA, 33 bp, Artificial Sequence; identical to SEQ ID NO: 105
  MGC36 CDRL3 nuc

SEQ ID NO: 304 - protein, 243 aa, Artificial Sequence
  MGC36 heavy chain aa
EVVLVESGGGLVQPGGSLRLSCEASGFNSRSYWMTWVRQAPGKGLEWVASINQDGTEKNYVDSVKGRFTISRDSAKNSLYLQMSSLRADDTAVYYCARDRFCGGESHLHGEEDLPRPSISAEPDTVIPLGSHVTFVCRGPVGVHTFRLERGWRYNDTEDVSQAGPSESEARFRIDSVREGNAGLYRCIYYIAPKWSEQSDYLELRVKGGDVTWALLTYCGGDGEESDYPMDVWGKGTTVTVSS

SEQ ID NO: 305 - protein, 111 aa, Artificial Sequence; identical to SEQ ID NO: 107
  MGC36 light chain aa

SEQ ID NO: 306 - DNA, 729 bp, Artificial Sequence
  MGC36 heavy chain nuc
gaggtggtactggtggagtctgggggaggcttggtccagcctggggggtccctgagactctcctgtgaagcctctggattcaactctcgtagttattggatgacctgggtccgccaggctccagggaaggggctggagtgggtggccagtataaatcaagatgggactgagaaaaattatgtggactctgtgaagggccggttcaccatctccagagactccgccaagaactcactgtatctgcaaatgagcagcctgagagccgacgacacggctgtatattactgtgcgagagacagattctgtggtggtgagagtcacttgcacggagaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaaggtggggacgtcacctgggccctgttaacgtactgtggcggtgatggagaggaatccgactaccccatggacgtctggggcaaagggaccacggtcaccgtctcctca

SEQ ID NO: 307 - DNA, 333 bp, Artificial Sequence
  MGC36 light chain nuc
cagactgtggttactcaggagccctcactgactgtgtccccaggagggacagtcactctcacctgtgcttccagcactggacctgtcaccagtgcttactatccaaactggttccagcagaagcctggacaagcacccaggtctcttatttatagtataaacaaaaaacactcctggacccctgcccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaa

SEQ ID NO: 308 - protein, 8 aa, Artificial Sequence
  MGC37 CDRH1 aa
GFTFRNYW

SEQ ID NO: 309 - protein, 8 aa, Artificial Sequence
  MGC37 CDRH2 aa
IRQDGSEK

SEQ ID NO: 310 - protein, 138 aa, Artificial Sequence; identical to SEQ ID NO: 130
  MGC37 CDRH3 aa

SEQ ID NO: 311 - protein, 6 aa, Artificial Sequence
  MGC37 CDRL1 aa
QRLSRS

SEQ ID NO: 312 - skipped

SEQ ID NO: 313 - protein, 9 aa, Artificial Sequence
  MGC37 CDRL2 long aa
LIYKASPLE

SEQ ID NO: 314 - protein, 9 aa, Artificial Sequence
  MGC37 CDRL3 aa
QQYSNYSYS

SEQ ID NO: 315 - DNA, 24 bp, Artificial Sequence
  MGC37 CDRH1 nuc
ggattcaccttcagaaattattgg

SEQ ID NO: 316 - DNA, 24 bp, Artificial Sequence
  MGC37 CDRH2 nuc
ataaggcaagatggaagtgagaag

SEQ ID NO: 317 - DNA, 414 bp, Artificial Sequence; identical to SEQ ID NO: 137
  MGC37 CDRH3 nuc

SEQ ID NO: 318 - DNA, 18 bp, Artificial Sequence
  MGC37 CDRL1 nuc
cagcgtcttagtcgctcg

SEQ ID NO: 319 - skipped

SEQ ID NO: 320 - DNA, 27 bp, Artificial Sequence
  MGC37 CDRL2 long nuc
ctgatctataaggcgtctcctttagaa

SEQ ID NO: 321 - DNA, 27 bp, Artificial Sequence
  MGC37 CDRL3 nuc
caacaatacagtaattattcatatagt

SEQ ID NO: 322 - protein, 245 aa, Artificial Sequence; identical to SEQ ID NO: 142
  MGC37 heavy chain aa

SEQ ID NO: 323 - protein, 107 aa, Artificial Sequence; identical to SEQ ID NO: 179
  MGC37 light chain aa

SEQ ID NO: 324 - DNA, 735 bp, Artificial Sequence; identical to SEQ ID NO: 144
  MGC37 heavy chain nuc

SEQ ID NO: 325 - DNA, 321 bp, Artificial Sequence; identical to SEQ ID NO: 181
  MGC37 light chain nuc

SEQ ID NO: 326 - protein, 9 aa, Artificial Sequence
  MGD21 CDRH1 aa
GDYVNTNRR

SEQ ID NO: 327 - protein, 7 aa, Artificial Sequence
  MGD21 CDRH2 aa
VHQSGRT

SEQ ID NO: 328 - protein, 143 aa, Artificial Sequence
  MGD21 CDRH3 aa
ARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDV

SEQ ID NO: 329 - protein, 6 aa, Artificial Sequence
  MGD21 CDRL1 aa
QHINDS

SEQ ID NO: 330 - skipped

SEQ ID NO: 331 - protein, 9 aa, Artificial Sequence
  MGD21 CDRL2 long aa
LIYGASNLH

SEQ ID NO: 332 - protein, 9 aa, Artificial Sequence
  MGD21 CDRL3 aa
QQCNCFPPD

SEQ ID NO: 333 - DNA, 27 bp, Artificial Sequence
  MGD21 CDRH1 nuc
ggtgactacgtcaatactaataggagg

SEQ ID NO: 334 - DNA, 21 bp, Artificial Sequence
  MGD21 CDRH2 nuc
gttcatcaaagtgggagaacc

SEQ ID NO: 335 - DNA, 429 bp, Artificial Sequence
  MGD21 CDRH3 nuc
gcgagagcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtc

SEQ ID NO: 336 - DNA, 18 bp, Artificial Sequence
  MGD21 CDRL1 nuc
caacatattaatgattct

SEQ ID NO: 337 - skipped

SEQ ID NO: 338 - DNA, 27 bp, Artificial Sequence
  MGD21 CDRL2 long nuc
ctgatatatggtgcatccaatttgcac

SEQ ID NO: 339 - DNA, 27 bp, Artificial Sequence
  MGD21 CDRL3 nuc
caacagtgtaattgtttccctccggac

SEQ ID NO: 340 - protein, 250 aa, Artificial Sequence
  MGD21 heavy chain aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 341 - protein, 107 aa, Artificial Sequence
  MGD21 light chain aa
AIRMTQSPSSLSASPGDKVSITCRASQHINDSLAWFQQRPGKAPKLLIYGASNLHSGVPSRFSGTGSGTDFTLTITGLQSEDFATYFCQQCNCFPPDFGQGTRLEIK

SEQ ID NO: 342 - DNA, 750 bp, Artificial Sequence
  MGD21 heavy chain nuc
gaggtgcagctggtggagacgggcccaggactgatgaagacttcggggaccctgtccctcacgtgcgctgtgtctggtgactacgtcaatactaataggaggtggagttgggtccgccaggccccagggaagggcctggagtggattggagaggttcatcaaagtgggagaaccaattacaacccgtccctcaagagccgagtcaccatatcagtagacaagtctaagaatcagttctctctgaaggtggactctgtgaccgccgcggacacggccgtgtattactgtgcgagagcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctca

SEQ ID NO: 343 - DNA, 321 bp, Artificial Sequence
  MGD21 light chain nuc
gccatccggatgacccagtctccatcctcactctctgcatcaccaggggacaaagtcagcatcacttgtcgggcgagtcaacatattaatgattctttggcctggtttcaacaaaggccagggaaagccccaaaactcctgatatatggtgcatccaatttgcacagtggggtcccatcgaggttcagcggcactgggtcagggacagatttcactctcactatcaccggcctgcagtctgaagattttgcaacttatttctgtcaacagtgtaattgtttccctccggacttcggccaagggacacgactggagattaaa

SEQ ID NO: 344 - protein, 9 aa, Artificial Sequence
  MGD23 CDRH1 aa
GGSISSNKW

SEQ ID NO: 345 - protein, 7 aa, Artificial Sequence
  MGD23 CDRH2 aa
VYQTGIT

SEQ ID NO: 346 - protein, 144 aa, Artificial Sequence
  MGD23 CDRH3 aa
ATISQLRPQGDTEDLPRPSLSAEPGTVIPLGSHVTFVCRGPAGVETFRLERESRFTYNDTEDVSQASPSESEARFRIDSVSEGNAGPYRCLYYKARKWSDQSDYLELLVKGADVTWALPQSQLAPRACPQGELRISTDVFSMNV

SEQ ID NO: 347 - protein, 6 aa, Artificial Sequence
  MGD23 CDRL1 aa
QYVGNY

SEQ ID NO: 348 - skipped

SEQ ID NO: 349 - protein, 9 aa, Artificial Sequence
  MGD23 CDRL2 long aa
LIHGVSTLQ

SEQ ID NO: 350 - protein, 9 aa, Artificial Sequence
  MGD23 CDRL3 aa
QQYYTSPPD

SEQ ID NO: 351 - DNA, 27 bp, Artificial Sequence
  MGD23 CDRH1 nuc
ggtggctccattagtagtaataagtgg

SEQ ID NO: 352 - DNA, 21 bp, Artificial Sequence
  MGD23 CDRH2 nuc
gtgtatcagactggtattacc

SEQ ID NO: 353 - DNA, 432 bp, Artificial Sequence
  MGD23 CDRH3 nuc
gcgacaatttctcaactgaggccgcagggggacaccgaagatctgcccagaccctccctctcggctgaaccaggcaccgtgatccccctggggagtcacgtgactttcgtgtgccggggcccggctggggtcgaaacattccgcctggagagggagagtagattcacttacaacgatactgaagatgtgtctcaagcgagtccatctgagtcagaggccagattccgcattgactcagtaagtgaaggaaatgccgggccttatcgctgcctctattataaggcccgtaaatggtctgaccagagtgactacttggaattgctggtgaagggtgcggacgtcacctgggccctgccccagtctcagctcgcccctcgagcttgtccccagggagaactccgcattagtaccgatgttttctccatgaacgtc

SEQ ID NO: 354 - DNA, 18 bp, Artificial Sequence
  MGD23 CDRL1 nuc
caatatgttgggaattat

SEQ ID NO: 355 - skipped

SEQ ID NO: 356 - DNA, 27 bp, Artificial Sequence
  MGD23 CDRL2 long nuc
ctcattcacggtgtatccactttgcaa

SEQ ID NO: 357 - DNA, 27 bp, Artificial Sequence
  MGD23 CDRL3 nuc
cagcagtattatacttcccctccggac

SEQ ID NO: 358 - protein, 251 aa, Artificial Sequence
  MGD23 heavy chain aa
QVQLQESGPGLVKPSGTLSLTCSVSGGSISSNKWWSWVRQSPGKGLEWIGEVYQTGITNYNPSLKGRVTMSVDKSKNQFSLRLTSVTAADTAVYYCATISQLRPQGDTEDLPRPSLSAEPGTVIPLGSHVTFVCRGPAGVETFRLERESRFTYNDTEDVSQASPSESEARFRIDSVSEGNAGPYRCLYYKARKWSDQSDYLELLVKGADVTWALPQSQLAPRACPQGELRISTDVFSMNVWGNGTTVTVSS

SEQ ID NO: 359 - protein, 107 aa, Artificial Sequence
  MGD23 light chain aa
AVRVTQSPTSLSASTGDRVTITCRTSQYVGNYLDWYQQKPGKAPKLLIHGVSTLQNGVPSRFSGSASGTDFTLNITCLQSEDSATYYCQQYYTSPPDFGQGTRLEIK

SEQ ID NO: 360 - DNA, 753 bp, Artificial Sequence
  MGD23 heavy chain nuc
caggtgcagctgcaggagtcgggcccaggactggtgaagccttcgggaaccctgtccctcacctgcagtgtctctggtggctccattagtagtaataagtggtggagttgggtccgccagtccccagggaagggcctggagtggattggggaggtgtatcagactggtattaccaactacaacccgtccctcaagggtcgagtcaccatgtcagtggacaagtccaagaaccaattctccctgagactgacttctgtgaccgccgcggacacggccgtgtattactgtgcgacaatttctcaactgaggccgcagggggacaccgaagatctgcccagaccctccctctcggctgaaccaggcaccgtgatccccctggggagtcacgtgactttcgtgtgccggggcccggctggggtcgaaacattccgcctggagagggagagtagattcacttacaacgatactgaagatgtgtctcaagcgagtccatctgagtcagaggccagattccgcattgactcagtaagtgaaggaaatgccgggccttatcgctgcctctattataaggcccgtaaatggtctgaccagagtgactacttggaattgctggtgaagggtgcggacgtcacctgggccctgccccagtctcagctcgcccctcgagcttgtccccagggagaactccgcattagtaccgatgttttctccatgaacgtctggggcaacgggaccacggtcaccgtctcttca

SEQ ID NO: 361 - DNA, 321 bp, Artificial Sequence
  MGD23 light chain nuc
gccgtccgggtgacccagtctccaacctcactgtctgcatctacaggagacagagtcaccatcacttgtcggacgagtcaatatgttgggaattatttagattggtatcagcaaaaaccagggaaagcccctaaactcctcattcacggtgtatccactttgcaaaatggggtcccatcaaggttcagtggcagtgcctccgggacagacttcactctcaacatcacctgcctacagtctgaagattctgcaacttattactgtcagcagtattatacttcccctccggacttcggccaagggacacgcctggaaattaag

SEQ ID NO: 362 - protein, 9 aa, Artificial Sequence
  MGD30 CDRH1 aa
GGSITSSKW

SEQ ID NO: 363 - protein, 7 aa, Artificial Sequence
  MGD30 CDRH2 aa
IYHNGTT

SEQ ID NO: 364 - protein, 145 aa, Artificial Sequence
  MGD30 CDRH3 aa
ATASPFKSHHRTTEKLPRPSISAEPGTVIPLGSRVTFVCRGPVGVQTFRLERETSFTYNDTEDVSQVSPSESEARFRIDSVSEGYAGPYRCVYYKAPKWSEQSDYLDLLVKGEDVTWALTQPQLDPRACPQGDLRMSTDIYCMDV

SEQ ID NO: 365 - protein, 6 aa, Artificial Sequence
  MGD30 CDRL1 aa
QDISTF

SEQ ID NO: 366 - skipped

SEQ ID NO: 367 - protein, 9 aa, Artificial Sequence
  MGD30 CDRL2 long aa
LIFAASTLQ

SEQ ID NO: 368 - protein, 9 aa, Artificial Sequence
  MGD30 CDRL3 aa
QQYYCFPPD

SEQ ID NO: 369 - DNA, 27 bp, Artificial Sequence
  MGD30 CDRH1 nuc
ggtggctccatcaccagtagtaagtgg

SEQ ID NO: 370 - DNA, 21 bp, Artificial Sequence
  MGD30 CDRH2 nuc
atctatcataatgggaccacc

SEQ ID NO: 371 - DNA, 435 bp, Artificial Sequence
  MGD30 CDRH3 nuc
gcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagctttacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtc

SEQ ID NO: 372 - DNA, 18 bp, Artificial Sequence
  MGD30 CDRL1 nuc
caggatattagcactttt

SEQ ID NO: 373 - skipped

SEQ ID NO: 374 - DNA, 27 bp, Artificial Sequence
  MGD30 CDRL2 long nuc
ctaatctttgctgcatctactttacaa

SEQ ID NO: 375 - DNA, 27 bp, Artificial Sequence
  MGD30 CDRL3 nuc
caacagtattattgtttccctccggac

SEQ ID NO: 376 - protein, 252 aa, Artificial Sequence
  MGD30 heavy chain aa
QVQLQESGPGLVKPSETLSLSCAVTGGSITSSKWWTWVRQGPDKGLEWIGKIYHNGTTNYNPSLKSRVAMSVDKSRNQFSLRLTSVTAADTALYYCATASPFKSHHRTTEKLPRPSISAEPGTVIPLGSRVTFVCRGPVGVQTFRLERETSFTYNDTEDVSQVSPSESEARFRIDSVSEGYAGPYRCVYYKAPKWSEQSDYLDLLVKGEDVTWALTQPQLDPRACPQGDLRMSTDIYCMDVWGKGTTVTVSS

SEQ ID NO: 377 - protein, 107 aa, Artificial Sequence
  MGD30 light chain aa
AIRLTQSPSSLSASIGDRVTITCRASQDISTFLAWYQQESGKAPRLLIFAASTLQTGVPSRFSGSGSGTDFTLTISGLQSEDFATYYCQQYYCFPPDFGQGTRLDIK

SEQ ID NO: 378 - DNA, 756 bp, Artificial Sequence
  MGD30 heavy chain nuc
caggtgcagctgcaggagtcgggcccaggactggtgaagccttcagaaaccctgtccctctcctgcgctgtcactggtggctccatcaccagtagtaagtggtggacttgggtccgccagggcccagataaggggctggagtggattgggaaaatctatcataatgggaccaccaactacaatccgtccctcaagagtcgagtcgccatgtcggtggacaagtccaggaaccagttctccctgagactgacctccgtgaccgccgcggacacggccttgtattactgtgcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagctttacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtctggggcaaagggaccacggtcaccgtctcctca

SEQ ID NO: 379 - DNA, 321 bp, Artificial Sequence
  MGD30 light chain nuc
gccatccggttgacccaatctccatcctcactctctgcatctataggagacagagtcaccatcacttgtcgggcgagtcaggatattagcacttttttggcctggtatcaacaagagtcaggtaaagccccaaggctcctaatctttgctgcatctactttacaaactggggtcccttcaaggttcagcggcagtggatctgggacagatttcactctcaccatcagcggcctgcaatctgaagattttgcaacttattactgtcaacagtattattgtttccctccggacttcggccaagggacacgactggacattaaa

SEQ ID NO: 380 - protein, 9 aa, Artificial Sequence
  MGD33 CDRH1 aa
GGSITSSKW

SEQ ID NO: 381 - protein, 7 aa, Artificial Sequence
  MGD33 CDRH2 aa
IYHNGTT

SEQ ID NO: 382 - protein, 145 aa, Artificial Sequence
  MGD33 CDRH3 aa
ATASPFKSHHRTTEKLPRPSISAEPGTVIPLGSRVTFVCRGPVGVQTFRLERETRSTYNDTEDVSQVSPSESEARFRIDSVSEGYAGPYRCVYYKAPKWSEQSDYLDLLVKGEDVTWALTQPQLDPRACPQGDLRMSTDIYCMDV

SEQ ID NO: 383 - protein, 6 aa, Artificial Sequence
  MGD33 CDRL1 aa
QDISTY

SEQ ID NO: 384 - skipped

SEQ ID NO: 385 - protein, 9 aa, Artificial Sequence
  MGD33 CDRL2 long aa
LIFAASSLQ

SEQ ID NO: 386 - protein, 9 aa, Artificial Sequence
  MGD33 CDRL3 aa
QQYYCFPPD

SEQ ID NO: 387 - DNA, 27 bp, Artificial Sequence
  MGD33 CDRH1 nuc
ggtggctccatcaccagtagtaagtgg

SEQ ID NO: 388 - DNA, 21 bp, Artificial Sequence
  MGD33 CDRH2 nuc
atctatcataatgggaccacc

SEQ ID NO: 389 - DNA, 435 bp, Artificial Sequence
  MGD33 CDRH3 nuc
gcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagatctacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtc

SEQ ID NO: 390 - DNA, 18 bp, Artificial Sequence
  MGD33 CDRL1 nuc
caggatattagcacttat

SEQ ID NO: 391 - skipped

SEQ ID NO: 392 - DNA, 27 bp, Artificial Sequence
  MGD33 CDRL2 long nuc
ctaatctttgctgcatctagtttacaa

SEQ ID NO: 393 - DNA, 27 bp, Artificial Sequence
  MGD33 CDRL3 nuc
caacaatattattgtttccctccggac

SEQ ID NO: 394 - protein, 252 aa, Artificial Sequence
  MGD33 heavy chain aa
HVQLQESGPGLVKPSETLSLSCAVTGGSITSSKWWTWVRQGPDKGLEWIGKIYHNGTTNYNPSLKSRVAMSVDKSKNQFSLRLTSVTAADTAVYYCATASPFKSHHRTTEKLPRPSISAEPGTVIPLGSRVTFVCRGPVGVQTFRLERETRSTYNDTEDVSQVSPSESEARFRIDSVSEGYAGPYRCVYYKAPKWSEQSDYLDLLVKGEDVTWALTQPQLDPRACPQGDLRMSTDIYCMDVWGKGTTVTVSS

SEQ ID NO: 395 - protein, 107 aa, Artificial Sequence
  MGD33 light chain aa
AIRLTQSPSSLSASIGDRVTITCRASQDISTYLAWYQQESGKAPRLLIFAASSLQTGVPSRFSGSGSGTDFTLTISGLQSEDFATYYCQQYYCFPPDFGQGTRLDIK

SEQ ID NO: 396 - DNA, 756 bp, Artificial Sequence
  MGD33 heavy chain nuc
cacgtgcagctgcaggagtcgggcccaggactggtgaagccttcagaaaccctgtccctctcctgcgctgtcactggtggctccatcaccagtagtaagtggtggacttgggtccgccagggcccagataaggggctggagtggattgggaaaatctatcataatgggaccaccaactacaatccgtccctcaagagtcgagtcgccatgtcggtggacaagtccaagaaccagttctccctgagactgacctccgtgaccgccgcggacacggccgtgtattactgtgcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagatctacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtctggggcaaagggaccacggtcaccgtctcctca

SEQ ID NO: 397 - DNA, 321 bp, Artificial Sequence
  MGD33 light chain nuc
gccatccggttgacccaatctccatcctcactctctgcatctataggagacagagtcaccatcacttgtcgggcgagtcaggatattagcacttatttggcctggtatcaacaagagtcaggtaaagccccaaggctcctaatctttgctgcatctagtttacaaactggggtcccttcaaggttcagcggcagtggatctgggacagatttcactctcaccatcagcggcctgcagtctgaagattttgcaacttattactgtcaacaatattattgtttccctccggacttcggccaagggacacgactggacattaaa

SEQ ID NO: 398 - protein, 9 aa, Artificial Sequence
  MGD34 CDRH1 aa
GDYVNTNRR

SEQ ID NO: 399 - protein, 7 aa, Artificial Sequence
  MGD34 CDRH2 aa
VHQSGRT

SEQ ID NO: 400 - protein, 144 aa, Artificial Sequence
  MGD34 CDRH3 aa
ARASPLKSQRDTEDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQVDPRACPQGELPISTDIYYVDV

SEQ ID NO: 401 - protein, 6 aa, Artificial Sequence
  MGD34 CDRL1 aa
QHINDS

SEQ ID NO: 402 - skipped

SEQ ID NO: 403 - protein, 9 aa, Artificial Sequence
  MGD34 CDRL2 long aa
LIYGASNLH

SEQ ID NO: 404 - protein, 9 aa, Artificial Sequence
  MGD34 CDRL3 aa
QQCNCFPPD

SEQ ID NO: 405 - DNA, 27 bp, Artificial Sequence
  MGD34 CDRH1 nuc
ggtgactacgtcaatactaataggagg

SEQ ID NO: 406 - DNA, 21 bp, Artificial Sequence
  MGD34 CDRH2 nuc
gttcatcaaagtgggagaacc

SEQ ID NO: 407 - DNA, 432 bp, Artificial Sequence
  MGD34 CDRH3 nuc
gcgagagcgtctccactcaaatctcagagggacaccgaagatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagtcgaccctcgagcttgtccccagggggagctccccataagtaccgatatttactacgtggacgtc

SEQ ID NO: 408 - DNA, 18 bp, Artificial Sequence
  MGD34 CDRL1 nuc
caacatattaatgattct

SEQ ID NO: 409 - skipped

SEQ ID NO: 410 - DNA, 27 bp, Artificial Sequence
  MGD34 CDRL2 long nuc
ctgatatatggtgcatccaatttgcac

SEQ ID NO: 411 - DNA, 27 bp, Artificial Sequence
  MGD34 CDRL3 nuc
caacagtgtaattgtttccctccggac

SEQ ID NO: 412 - protein, 251 aa, Artificial Sequence [sequence omitted]

SEQ ID NO: 413 - protein, 107 aa, Artificial Sequence; identical to SEQ ID NO: 341
  MGD34 light chain aa

SEQ ID NO: 414 - DNA, 753 bp, Artificial Sequence
  MGD34 heavy chain nuc
gaggtgcagctggtggagtcgggcccaggactgatgaagacttcggggaccctgtccctcacgtgcgctgtgtctggtgactacgtcaatactaataggaggtggagttgggtccgccaggccccagggaagggcctggagtggattggagaggttcatcaaagtgggagaaccaattacaacccgtccctcaagagccgagtcaccatatcagtagacaagtctaagaatcagttctctctgaaggtggactctgtgaccgccgcggacacggccgtgtattactgtgcgagagcgtctccactcaaatctcagagggacaccgaagatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagtcgaccctcgagcttgtccccagggggagctccccataagtaccgatatttactacgtggacgtctggggcaacgggaccacgttcaccgtctcctca

SEQ ID NO: 415 - DNA, 321 bp, Artificial Sequence; identical to SEQ ID NO: 343
  MGD34 light chain nuc

SEQ ID NO: 416 - protein, 9 aa, Artificial Sequence
  MGD35 CDRH1 aa
GASISSINW

SEQ ID NO: 417 - protein, 7 aa, Artificial Sequence
  MGD35 CDRH2 aa
IHHNGST

SEQ ID NO: 418 - protein, 143 aa, Artificial Sequence
  MGD35 CDRH3 aa
ATASSLKSQRDTNLPRPSLSAEPGTVIPLGSPVTFVCRGPVGVHTFRLERAGRSTYNDTEDVSHPSPSESEARFRIDSVSEGNAGPYRCVYYKSSKWSEESYCLDLLVKTEDVTWARPQPQLDPRACPQGDLRISTDFYYMDV

SEQ ID NO: 419 - protein, 6 aa, Artificial Sequence
  MGD35 CDRL1 aa
QAIGTY

SEQ ID NO: 420 - skipped

SEQ ID NO: 421 - protein, 9 aa, Artificial Sequence
  MGD35 CDRL2 long aa
LIYNASTLQ

SEQ ID NO: 422 - protein, 9 aa, Artificial Sequence
  MGD35 CDRL3 aa
QHYYNYPPA

SEQ ID NO: 423 - DNA, 27 bp, Artificial Sequence
  MGD35 CDRH1 nuc
ggtgcctccatcagtagtattaattgg

SEQ ID NO: 424 - DNA, 21 bp, Artificial Sequence
  MGD35 CDRH2 nuc
atccatcataatgggagcacc

SEQ ID NO: 425 - DNA, 429 bp, Artificial Sequence
  MGD35 CDRH3 nuc
gcgactgcctcttcattgaagtctcagagggacaccaatttgcccagaccctccctctcggcggagccaggcaccgtgatccccctggggagccctgtgactttcgtgtgccggggcccggttggggttcacacattccgcctggagagggcgggtagatccacatacaatgatactgaagatgtgtctcatcctagtccatctgagtcagaggccagattccgcattgactcagtgagtgagggaaatgccgggccttatcgctgcgtctattataagtcctctaaatggtccgaggagagttactgcctggacctgctggtcaaaactgaggacgtcacgtgggcccggccccagcctcagctcgaccctcgagcttgtccccagggggacctccgcattagcaccgatttttactacatggacgtc

SEQ ID NO: 426 - DNA, 18 bp, Artificial Sequence
  MGD35 CDRL1 nuc
caggctattggcacttat

SEQ ID NO: 427 - skipped

SEQ ID NO: 428 - DNA, 27 bp, Artificial Sequence
  MGD35 CDRL2 long nuc
ctgatctataatgcttccactttgcaa

SEQ ID NO: 429 - DNA, 27 bp, Artificial Sequence
  MGD35 CDRL3 nuc
caacactattataattatcctccggcc

SEQ ID NO: 430 - protein, 250 aa, Artificial Sequence
  MGD35 heavy chain aa
QVQLQESGPGLVKPSGTLSLTCAVSGASISSINWWSWVRQTPEKGLEWIGQIHHNGSTNYNPSLKSRVAISVDKSKNQFSLKLTSFTAADTAVYYCATASSLKSQRDTNLPRPSLSAEPGTVIPLGSPVTFVCRGPVGVHTFRLERAGRSTYNDTEDVSHPSPSESEARFRIDSVSEGNAGPYRCVYYKSSKWSEESYCLDLLVKTEDVTWARPQPQLDPRACPQGDLRISTDFYYMDVWGKGTTVTVSS

SEQ ID NO: 431 - protein, 107 aa, Artificial Sequence
  MGD35 light chain aa
AIRMTQSPSSLSASTGDRVTITCRTSQAIGTYLAWYQQNPGKAPNLLIYNASTLQSGVPSRFSASGSGTDFTLTISGLQSDDFVTYFCQHYYNYPPAFGQGTRLEIQ

SEQ ID NO: 432 - DNA, 750 bp, Artificial Sequence
  MGD35 heavy chain nuc
caggtgcagctgcaggagtcgggcccaggactggtgaagccttcggggaccctgtccctcacctgcgctgtctctggtgcctccatcagtagtattaattggtggagttgggtccgtcagaccccagaaaaggggctggagtggattggacaaatccatcataatgggagcaccaactacaacccgtccctcaagagtcgggtcgccatatcagttgacaagtccaagaaccagttctccctgaagttgacttctttcaccgccgcggacacggccgtgtattattgtgcgactgcctcttcattgaagtctcagagggacaccaatttgcccagaccctccctctcggcggagccaggcaccgtgatccccctggggagccctgtgactttcgtgtgccggggcccggttggggttcacacattccgcctggagagggcgggtagatccacatacaatgatactgaagatgtgtctcatcctagtccatctgagtcagaggccagattccgcattgactcagtgagtgagggaaatgccgggccttatcgctgcgtctattataagtcctctaaatggtccgaggagagttactgcctggacctgctggtcaaaactgaggacgtcacgtgggcccggccccagcctcagctcgaccctcgagcttgtccccagggggacctccgcattagcaccgatttttactacatggacgtctggggcaaagggaccacggtcaccgtctcttca

SEQ ID NO: 433 - DNA, 321 bp, Artificial Sequence
  MGD35 light chain nuc
gccatccggatgacccagtctccatcctcactctctgcatctacgggagacagagtcaccatcacttgtcggacgagtcaggctattggcacttatttagcgtggtatcagcagaacccagggaaagcccctaacctcctgatctataatgcttccactttgcaaagtggggtcccatcaaggttcagcgccagtggctctgggacagatttcactctcaccatcagcggcctgcagtctgacgattttgtcacttatttctgccaacactattataattatcctccggccttcggccaagggacacgactggagattcaa

SEQ ID NO: 434 - protein, 9 aa, Artificial Sequence
  MGD39 CDRH1 aa
GGSISAYRW

SEQ ID NO: 435 - protein, 7 aa, Artificial Sequence
  MGD39 CDRH2 aa
VYNDGNT

SEQ ID NO: 436 - protein, 144 aa, Artificial Sequence
  MGD39 CDRH3 aa
ATISPLRPQSDTDDLPRPSISAEPGTVIPLGSHVTFVCRGPIGVQTFRLERERRSLYSDTEDVSQVSPFASEARFRIDSVSEGNAGPYRCIYYKDRKWSDQSDYLELLVKGEDVTWALPQSQLAPRACPQEELNISTDIFSMNV

SEQ ID NO: 437 - protein, 6 aa, Artificial Sequence
  MGD39 CDRL1 aa
HDVGNY

SEQ ID NO: 438 - skipped

SEQ ID NO: 439 - protein, 9 aa, Artificial Sequence
  MGD39 CDRL2 long aa
LIHGASTLQ

SEQ ID NO: 440 - protein, 9 aa, Artificial Sequence
  MGD39 CDRL3 aa
QQYYSSPPG

SEQ ID NO: 441 - DNA, 27 bp, Artificial Sequence
  MGD39 CDRH1 nuc
ggtggctccatcagtgcttataggtgg

SEQ ID NO: 442 - DNA, 21 bp, Artificial Sequence
  MGD39 CDRH2 nuc
gtctataatgatggcaatacc

SEQ ID NO: 443 - DNA, 432 bp, Artificial Sequence
  MGD39 CDRH3 nuc
gcgacaatttctccactgaggcctcagagtgacaccgacgatctgcccagaccctccatctcggctgagccaggcaccgtgatccccctggggagccatgtgaccttcgtgtgccggggcccaattggggttcaaacattccgcctggagagggagagaagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtc

SEQ ID NO: 444 - DNA, 18 bp, Artificial Sequence
  MGD39 CDRL1 nuc
catgatgttggtaattat

SEQ ID NO: 445 - skipped

SEQ ID NO: 446 - DNA, 27 bp, Artificial Sequence
  MGD39 CDRL2 long nuc
ctgatccacggtgcgtccactttgcaa

SEQ ID NO: 447 - DNA, 27 bp, Artificial Sequence
  MGD39 CDRL3 nuc
caacaatattacagttcccctccgggc

SEQ ID NO: 448 - protein, 251 aa, Artificial Sequence
  MGD39 heavy chain aa
QVRLQESGPGLVKPSGTLSLTCTVSGGSISAYRWWSWVRQAPGKGLEWIGQVYNDGNTNYNPSLKGRVAMSVDKSKNRFSLRLASVTAADTAVYYCATISPLRPQSDTDDLPRPSISAEPGTVIPLGSHVTFVCRGPIGVQTFRLERERRSLYSDTEDVSQVSPFASEARFRIDSVSEGNAGPYRCIYYKDRKWSDQSDYLELLVKGEDVTWALPQSQLAPRACPQEELNISTDIFSMNVWGKGTTVTVSS

SEQ ID NO: 449 - protein, 107 aa, Artificial Sequence
  MGD39 light chain aa
AIRMTQSPASLSASIGDRVTITCRTSHDVGNYLDWYQQKPGKAPKLLIHGASTLQTGVPSRFSGSGAGTDFTLNITCLQSGDFAMYYCQQYYSSPPGFGQGTRLEIK

SEQ ID NO: 450 - DNA, 753 bp, Artificial Sequence
  MGD39 heavy chain nuc
caggtgcggctgcaggagtcgggcccaggactggtgaagccttcggggaccctgtccctcacctgcactgtctctggtggctccatcagtgcttataggtggtggagttgggtccgccaggccccaggcaagggcctggagtggattggacaggtctataatgatggcaataccaactacaacccgtccctcaagggtcgagtcgccatgtcagtggacaagtccaagaatcgattttccctgagattagcgtctgtgaccgccgcggacacggccgtgtattactgtgcgacaatttctccactgaggcctcagagtgacaccgacgatctgcccagaccctccatctcggctgagccaggcaccgtgatccccctggggagccatgtgaccttcgtgtgccggggcccaattggggttcaaacattccgcctggagagggagagaagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtctggggcaaagggaccacggtcaccgtctcctca

SEQ ID NO: 451 - DNA, 321 bp, Artificial Sequence
  MGD39 light chain nuc
gccatccggatgacccagtctccagcgtctctgtctgcatctataggagacagagtcaccatcacttgtcggacgagtcatgatgttggtaattatttagattggtatcaacaaaaaccaggaaaagcccctaaactcctgatccacggtgcgtccactttgcaaactggggtcccatcacggttcagcggcagtggagccgggacagatttcactctcaacatcacctgcctgcagtctggagatttcgcaatgtattattgtcaacaatattacagttcccctccgggcttcggccaagggacacgactggagattaaa

SEQ ID NO: 452 - protein, 9 aa, Artificial Sequence
  MGD41 CDRH1 aa
GGSINTDKW

SEQ ID NO: 453 - protein, 7 aa, Artificial Sequence
  MGD41 CDRH2 aa
VLHTGST

SEQ ID NO: 454 - protein, 144 aa, Artificial Sequence
  MGD41 CDRH3 aa
ATISTLRPQRDIEDLPRPSLSAEPGTVVPLGSHVTFVCRGPVGVQTFRLERESRSTYNDTEDVSQPSPFESEARFRIDSVSEGNAGPYRCIYYKSPKWSDQSDYVELLVKGEDVTWAPPQSQLAPRACPQGELRTSTDIFSMNV

SEQ ID NO: 455 - protein, 6 aa, Artificial Sequence
  MGD41 CDRL1 aa
QDIGNY

SEQ ID NO: 456 - skipped

SEQ ID NO: 457 - protein, 9 aa, Artificial Sequence
  MGD41 CDRL2 long aa
LIHGASTLL

SEQ ID NO: 458 - protein, 9 aa, Artificial Sequence
  MGD41 CDRL3 aa
LQYYSSPPA

SEQ ID NO: 459 - DNA, 27 bp, Artificial Sequence
  MGD41 CDRH1 nuc
ggtggctccatcaacactgataagtgg

SEQ ID NO: 460 - DNA, 21 bp, Artificial Sequence
  MGD41 CDRH2 nuc
gtccttcatactgggagcacc

SEQ ID NO: 461 - DNA, 432 bp, Artificial Sequence
  MGD41 CDRH3 nuc
gcgactatttctacattgaggcctcagcgggacatcgaagatctgcccagaccctccctctcggctgagccaggcaccgtggtccccctggggagccatgtgactttcgtgtgccggggcccggttggggttcaaacattccgcctggagagggagagcagatccacatacaatgatactgaagatgtgtctcaacctagtccatttgagtcagaggccagatttcgcattgactcagtaagtgaaggaaatgccgggccttatcgctgcatctattataagtcccctaaatggtctgaccagagtgactacgtggagttgctggtgaaaggtgaggacgtcacctgggccccgccccagtctcagctcgcccctcgagcttgtccccagggagaactccgcactagcaccgatattttctccatgaacgtc

SEQ ID NO: 462 - DNA, 18 bp, Artificial Sequence
  MGD41 CDRL1 nuc
caggatattggtaattac

SEQ ID NO: 463 - skipped

SEQ ID NO: 464 - DNA, 27 bp, Artificial Sequence
  MGD41 CDRL2 long nuc
ctgatccatggtgcatccactttgctg

SEQ ID NO: 465 - DNA, 27 bp, Artificial Sequence
  MGD41 CDRL3 nuc
ctacaatattacagttcccctccggcc

SEQ ID NO: 466 - protein, 251 aa, Artificial Sequence
  MGD41 heavy chain aa
EVQLVESGPGLVKPSGTLSVTCTISGGSINTDKWWTWVRQPPGKGLEWVGEVLHTGSTNYNPSLRGRVTISVDKSKNQFSLRLSSVTAADTAVYYCATISTLRPQRDIEDLPRPSLSAEPGTVVPLGSHVTFVCRGPVGVQTFRLERESRSTYNDTEDVSQPSPFESEARFRIDSVSEGNAGPYRCIYYKSPKWSDQSDYVELLVKGEDVTWAPPQSQLAPRACPQGELRTSTDIFSMNVWGKGTTVTVSS

SEQ ID NO: 467 - protein, 107 aa, Artificial Sequence
  MGD41 light chain aa
AIRMTQSPSSLSAFTGDRVTISCRASQDIGNYLDWYHQKPGRAPKLLIHGASTLLTGVPSRFSGSGSGTDFTLNITCLQSGDFGIYYCLQYYSSPPAFGPGTRLEIK

SEQ ID NO: 468 - DNA, 753 bp, Artificial Sequence
  MGD41 heavy chain nuc
gaggtgcagctggtggagtcgggcccaggactggtgaagccttcggggaccctgtccgtcacctgcactatctctggtggctccatcaacactgataagtggtggacttgggtccgccagcccccagggaagggccttgagtgggtaggggaagtccttcatactgggagcaccaactacaacccgtccctgaggggtcgagtcaccatatcagtggacaagtccaagaaccagttctccctgaggctgagttctgtgaccgccgcggacacggccgtatattattgtgcgactatttctacattgaggcctcagcgggacatcgaagatctgcccagaccctccctctcggctgagccaggcaccgtggtccccctggggagccatgtgactttcgtgtgccggggcccggttggggttcaaacattccgcctggagagggagagcagatccacatacaatgatactgaagatgtgtctcaacctagtccatttgagtcagaggccagatttcgcattgactcagtaagtgaaggaaatgccgggccttatcgctgcatctattataagtcccctaaatggtctgaccagagtgactacgtggagttgctggtgaaaggtgaggacgtcacctgggccccgccccagtctcagctcgcccctcgagcttgtccccagggagaactccgcactagcaccgatattttctccatgaacgtctggggcaaagggaccacggtcaccgtctcctca

SEQ ID NO: 469 - DNA, 322 bp, Artificial Sequence
  MGD41 light chain nuc
gccatccggatgacccagtctccatcctcactgtctgcatttacaggagacagagtcaccatctcttgccgggcgagtcaggatattggtaattacttagattggtatcaccaaaagccaggaagagcccctaagctcctgatccatggtgcatccactttgctgactggggtcccatcacgattcagcggcagtggatccggaacagatttcactctcaacatcacctgcctgcagtctggagattttggaatttattactgtctacaatattacagttcccctccggccttcggcccagggacacggctggagattaaga

SEQ ID NO: 470 - protein, 9 aa, Artificial Sequence
  MGD47 CDRH1 aa
GGSISGYKW

SEQ ID NO: 471 - protein, 7 aa, Artificial Sequence
  MGD47 CDRH2 aa
VYDDGDT

SEQ ID NO: 472 - protein, 144 aa, Artificial Sequence
  MGD47 CDRH3 aa
ATISPLRPQSDTGDLPRPSISAEPGTAIPLGSQVTFVCRGPIGVQTFRLERESRALYNDSEDVSQVSPSASEARFRIDSVSEGNAGPYRCIYYKARRWSDQSDYLELLVKGEDVTWALPQSQLAPRACPQEDLNISTDIFSTNV

SEQ ID NO: 473 - protein, 6 aa, Artificial Sequence
  MGD47 CDRL1 aa
QDVGNY

SEQ ID NO: 474 - skipped

SEQ ID NO: 475 - protein, 9 aa, Artificial Sequence
  MGD47 CDRL2 long aa
LIHGASTLQ

SEQ ID NO: 476 - protein, 9 aa, Artificial Sequence
  MGD47 CDRL3 aa
QQYYTSPPV

SEQ ID NO: 477 - DNA, 27 bp, Artificial Sequence
  MGD47 CDRH1 nuc
ggtggctccatcagtggttacaagtgg

SEQ ID NO: 478 - DNA, 21 bp, Artificial Sequence
  MGD47 CDRH2 nuc
gtctatgatgatggcgacacc

SEQ ID NO: 479 - DNA, 432 bp, Artificial Sequence
  MGD47 CDRH3 nuc
gcgacaatttctccactgaggcctcagagtgacaccggagatctgcccagaccctccatctcggctgagccaggcaccgcgatccccctggggagccaagtgactttcgtgtgccggggcccaattggggttcaaacattccgcctggagagggagagtcgcgccttatataatgattctgaagatgtgtctcaagttagtccatctgcgtcagaggccagattccgcattgactcagtaagtgaaggcaatgccgggccttatcgctgtatctattataaggcccgcagatggtctgaccagagtgactatttggagttgttggtgaaaggtgaggacgtcacctgggccctgccccagtctcagctcgcccctcgcgcttgtccccaggaagatttgaacattagtaccgatattttctctacgaacgtc

SEQ ID NO: 480 - DNA, 18 bp, Artificial Sequence
  MGD47 CDRL1 nuc
caggatgttggaaattat

SEQ ID NO: 481 - skipped

SEQ ID NO: 482 - DNA, 27 bp, Artificial Sequence
  MGD47 CDRL2 long nuc
ctcatccacggtgcgtccactttgcaa

SEQ ID NO: 483 - DNA, 27 bp, Artificial Sequence
  MGD47 CDRL3 nuc
caacaatattacacttcccctccggtc

SEQ ID NO: 484 - protein, 251 aa, Artificial Sequence
  MGD47 heavy chain aa
QVRLQESGPGLVKPSGTLSLTCTVSGGSISGYKWWSWVRQAPGKGLEWIGQVYDDGDTNYNPDLKGRVALSVDKSKSRFSLSLASVTAADTAIYFCATISPLRPQSDTGDLPRPSISAEPGTAIPLGSQVTFVCRGPIGVQTFRLERESRALYNDSEDVSQVSPSASEARFRIDSVSEGNAGPYRCIYYKARRWSDQSDYLELLVKGEDVTWALPQSQLAPRACPQEDLNISTDIFSTNVWGKGTTVTVSS

SEQ ID NO: 485 - protein, 107 aa, Artificial Sequence
  MGD47 light chain aa
AIRMTQSPASLSASVGDRVTITCRTSQDVGNYLDWYQQKPGKAPKLLIHGASTLQAGVPSRFNGSGSGTDFTLGISCVQSGDFAIYYCQQYYTSPPVFGQGTRLEIK

SEQ ID NO: 486 - DNA, 753 bp, Artificial Sequence
  MGD47 heavy chain nuc
caggtgcggctgcaggagtcgggcccaggactggtgaagccttcggggaccctgtccctcacctgcactgtctcgggtggctccatcagtggttacaagtggtggagttgggtccgccaggccccaggcaagggcctggagtggattggacaggtctatgatgatggcgacaccaactacaatccggacctgaagggtcgagtcgccctgtcagtggacaagtccaagagtcgattttccctcagcctagcgtctgtgaccgccgcggacacggccatatacttctgtgcgacaatttctccactgaggcctcagagtgacaccggagatctgcccagaccctccatctcggctgagccaggcaccgcgatccccctggggagccaagtgactttcgtgtgccggggcccaattggggttcaaacattccgcctggagagggagagtcgcgccttatataatgattctgaagatgtgtctcaagttagtccatctgcgtcagaggccagattccgcattgactcagtaagtgaaggcaatgccgggccttatcgctgtatctattataaggcccgcagatggtctgaccagagtgactatttggagttgttggtgaaaggtgaggacgtcacctgggccctgccccagtctcagctcgcccctcgcgcttgtccccaggaagatttgaacattagtaccgatattttctctacgaacgtctggggcaaagggacaacggtcaccgtctcttca

SEQ ID NO: 487 - DNA, 321 bp, Artificial Sequence
  MGD47 light chain nuc
gccatccggatgacccagtctccagcgtccctgtctgcatctgtaggagacagagtcaccatcacttgtcggacgagtcaggatgttggaaattatttagattggtatcaacaaaaaccaggaaaagcccctaaactcctcatccacggtgcgtccactttgcaagctggggtcccatcacgtttcaacggcagtggatccgggacagatttcactctcggcatcagttgtgtgcagtctggagatttcgcgatctattactgtcaacaatattacacttcccctccggtcttcggccaagggacacgactggagattaaa

SEQ ID NO: 488 - protein, 9 aa, Artificial Sequence
  MGD55 CDRH1 aa
GGSISAYKW

SEQ ID NO: 489 - protein, 7 aa, Artificial Sequence
  MGD55 CDRH2 aa
VYHNGNT

SEQ ID NO: 490 - protein, 144 aa, Artificial Sequence
  MGD55 CDRH3 aa
ATISPLRPQSDTDDLPRPSISAEPGTVIPLGSHVTFVCRGPIGVQTFRLERESRSLYSDTEDVSQVSPFASEARFRIDSVSEGNAGPYRCIYYKDRKWSDQSDYLELLVKGEDVTWALPQSQLAPRACPQEELNISTDIFSMNV

SEQ ID NO: 491 - protein, 6 aa, Artificial Sequence
  MGD55 CDRL1 aa
QDVGNY

SEQ ID NO: 492 - skipped

SEQ ID NO: 493 - protein, 9 aa, Artificial Sequence
  MGD55 CDRL2 long aa
LIHGASTLQ

SEQ ID NO: 494 - protein, 9 aa, Artificial Sequence
  MGD55 CDRL3 aa
QQYYSSPPG

SEQ ID NO: 495 - DNA, 27 bp, Artificial Sequence
  MGD55 CDRH1 nuc
ggtggctccatcagtgcttataagtgg

SEQ ID NO: 496 - DNA, 21 bp, Artificial Sequence
  MGD55 CDRH2 nuc
gtctatcataatggcaacacc

SEQ ID NO: 497 - DNA, 432 bp, Artificial Sequence
  MGD55 CDRH3 nuc
gcgacaatttctccactgaggcctcagagtgacaccgacgatctgcccagaccctccatctcggctgagccaggcaccgtgatccccctggggagccatgtgactttcgtgtgccggggcccaattggggttcaaacattccgcctggagagggagagtagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtc

SEQ ID NO: 498 - DNA, 18 bp, Artificial Sequence
  MGD55 CDRL1 nuc
caggatgttggtaattat

SEQ ID NO: 499 - skipped

SEQ ID NO: 500 - DNA, 27 bp, Artificial Sequence
  MGD55 CDRL2 long nuc
ctgatccacggtgcgtccactttgcaa

SEQ ID NO: 501 - DNA, 27 bp, Artificial Sequence
  MGD55 CDRL3 nuc
caacaatattacagttcccctccgggc

SEQ ID NO: 502 - protein, 251 aa, Artificial Sequence
  MGD55 heavy chain aa
QVQLQESGPGLVKPSGTLSLTCTVSGGSISAYKWWSWVRQAPGKGLEWIGQVYHNGNTNYNPSLKGRVAMSVDKSKNRFSLRLASVTAADTAVYYCATISPLRPQSDTDDLPRPSISAEPGTVIPLGSHVTFVCRGPIGVQTFRLERESRSLYSDTEDVSQVSPFASEARFRIDSVSEGNAGPYRCIYYKDRKWSDQSDYLELLVKGEDVTWALPQSQLAPRACPQEELNISTDIFSMNVWGKGTTVTVSS

SEQ ID NO: 503 - protein, 107 aa, Artificial Sequence
  MGD55 light chain aa
AIRMTQSPASLSASIGDRVTITCRTSQDVGNYLDWYQQKPGKAPKLLIHGASTLQTGVPSRFSGSGAGTDFTLNITCLQSGDFAMYYCQQYYSSPPGFGQGTRLEIK

SEQ ID NO: 504 - DNA, 753 bp, Artificial Sequence
  MGD55 heavy chain nuc
caggtgcagctgcaggagtcgggcccaggactggtgaagccttcggggaccctgtccctcacctgcactgtctctggtggctccatcagtgcttataagtggtggagttgggtccgccaggccccaggcaagggcctggagtggattggacaggtctatcataatggcaacaccaactacaacccgtccctcaagggtcgagtcgccatgtcagtggacaagtccaagaatcgattttccctgagactagcgtctgtgaccgccgcggacacggccgtgtattactgtgcgacaatttctccactgaggcctcagagtgacaccgacgatctgcccagaccctccatctcggctgagccaggcaccgtgatccccctggggagccatgtgactttcgtgtgccggggcccaattggggttcaaacattccgcctggagagggagagtagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtctggggcaaagggaccacggtcaccgtctcctca

SEQ ID NO: 505 - DNA, 322 bp, Artificial Sequence
  MGD55 light chain nuc
gccatccggatgacccagtctccagcgtctctgtctgcatctataggagacagagtcaccatcacttgtcggacgagtcaggatgttggtaattatttagattggtatcaacaaaaaccaggaaaagcccctaaactcctgatccacggtgcgtccactttgcaaactggggtcccatcacggttcagcggcagtggagccgggacagatttcactctcaacatcacctgcctgcagtctggagatttcgcaatgtattactgtcaacaatattacagttcccctccgggcttcggccaagggacacgactggaaattaaga

SEQ ID NO: 506 - protein, 9 aa, Artificial Sequence
  MGD56 CDRH1 aa
GGSITTNNW

SEQ ID NO: 507 - protein, 7 aa, Artificial Sequence
  MGD56 CDRH2 aa
IFRSGTT

SEQ ID NO: 508 - protein, 144 aa, Artificial Sequence
  MGD56 CDRH3 aa
ATASPFKSQRDTKDLPRPSLSAEPGTVIPLGSHVTFVCRGPVGVQTFRLQRESRSLYNDTEDVSHPSPSESEARFRIDSVSEGNAGPYRCVYYKSSKWSEESDCLELLVKTEDVTWARPQPQLDPRACPRGDLRISTDVYYMDV

SEQ ID NO: 509 - protein, 6 aa, Artificial Sequence
  MGD56 CDRL1 aa
QAITSY

SEQ ID NO: 510 - skipped

SEQ ID NO: 511 - protein, 9 aa, Artificial Sequence
  MGD56 CDRL2 long aa
LIYNASTLQ

SEQ ID NO: 512 - protein, 9 aa, Artificial Sequence
  MGD56 CDRL3 aa
QHYYTYPPA

SEQ ID NO: 513 - DNA, 27 bp, Artificial Sequence
  MGD56 CDRH1 nuc
ggtggctccatcactactaataattgg

SEQ ID NO: 514 - DNA, 21 bp, Artificial Sequence
  MGD56 CDRH2 nuc
atctttcgtagtgggaccacc

SEQ ID NO: 515 - DNA, 432 bp, Artificial Sequence
  MGD56 CDRH3 nuc
gcgacagcctctccattcaagtctcagagggacaccaaagatttgcccagaccctccctctcggctgagccaggcaccgtgatccccctggggagtcatgtgactttcgtgtgccggggcccggttggggttcagacattccgcctgcagagggagagtagatccctttacaatgatactgaagatgtgtctcatcctagtccatctgagtcagaggccagattccgcattgactcagtgagtgagggaaatgccgggccttatcgctgcgtctattataagtcctctaaatggtccgaggagagtgactgcctggagctgctggtcaaaactgaggacgtcacctgggcccggccccagcctcagctcgaccctcgagcttgtccccggggggacctccgcattagcaccgatgtttactacatggacgtc

SEQ ID NO: 516 - DNA, 18 bp, Artificial Sequence
  MGD56 CDRL1 nuc
caggctattaccagttat

SEQ ID NO: 517 - skipped

SEQ ID NO: 518 - DNA, 27 bp, Artificial Sequence
  MGD56 CDRL2 long nuc
ctgatctataatgcttccactttgcaa

SEQ ID NO: 519 - DNA, 27 bp, Artificial Sequence
  MGD56 CDRL3 nuc
caacactattatacttaccctccggcc

SEQ ID NO: 520 - protein, 251 aa, Artificial Sequence
  MGD56 heavy chain aa
QVQLQESGPGLVKPSGTLSLTCAVSGGSITTNNWWSWVRQTPGKGLEWIGEIFRSGTTNYNPSLKSRVAISLDKSKNQFSLKLTSVTAADTAVYYCATASPFKSQRDTKDLPRPSLSAEPGTVIPLGSHVTFVCRGPVGVQTFRLQRESRSLYNDTEDVSHPSPSESEARFRIDSVSEGNAGPYRCVYYKSSKWSEESDCLELLVKTEDVTWARPQPQLDPRACPRGDLRISTDVYYMDVWGKGTTVTVSS

SEQ ID NO: 521 - protein, 107 aa, Artificial Sequence
  MGD56 light chain aa
AIRMTQSPSSLSASTGDRVTITCRASQAITSYLAWYRQKPGKAPDLLIYNASTLQSGVPSRFSASGSGTDFALTITGLQSEDFVIYFCQHYYTYPPAFGQGTRLEIK

SEQ ID NO: 522 - DNA, 753 bp, Artificial Sequence
  MGD56 heavy chain nuc
caggtgcagctgcaggagtcgggcccaggactggtgaagccttcggggaccctgtccctcacctgcgctgtctctggtggctccatcactactaataattggtggagttgggtccgtcagaccccaggaaaggggctggagtggattggagaaatctttcgtagtgggaccaccaactacaacccgtccctcaagagtcgggtcgccatttcattagacaagtccaagaaccagttctccctgaagttgacttctgtgaccgccgcggacacggccgtgtattactgtgcgacagcctctccattcaagtctcagagggacaccaaagatttgcccagaccctccctctcggctgagccaggcaccgtgatccccctggggagtcatgtgactttcgtgtgccggggcccggttggggttcagacattccgcctgcagagggagagtagatccctttacaatgatactgaagatgtgtctcatcctagtccatctgagtcagaggccagattccgcattgactcagtgagtgagggaaatgccgggccttatcgctgcgtctattataagtcctctaaatggtccgaggagagtgactgcctggagctgctggtcaaaactgaggacgtcacctgggcccggccccagcctcagctcgaccctcgagcttgtccccggggggacctccgcattagcaccgatgtttactacatggacgtctggggcaaagggaccacggtcaccgtctcctca

SEQ ID NO: 523 - DNA, 321 bp, Artificial Sequence
  MGD56 light chain nuc
gccatccggatgacccagtctccatcctcactctctgcatctacaggggacagagtcaccatcacttgtcgggcgagtcaggctattaccagttatttagcctggtatcggcagaaaccagggaaagcccctgacctcctgatctataatgcttccactttgcaaagtggggtcccatcaagattcagcgccagtggctctgggacagatttcgctctcaccatcaccggcctgcagtctgaggattttgtaatttatttctgccaacactattatacttaccctccggccttcggccaagggacacgactggagattaaa

SEQ ID NO: 524 - protein, 330 aa, Artificial Sequence
  IgG1 CH1-CH2-CH3 aa
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - protein, 107 aa, Artificial Sequence [sequence omitted]

SEQ ID NO: 526 - protein, 106 aa, Artificial Sequence
  IgG CL aa
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 527 - DNA, 990 bp, Artificial Sequence
  IgG1 CH1-CH2-CH3 nucl
gcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 528 - DNA, 321 bp, Artificial Sequence
  IgG CK nucl
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 529 - DNA, 318 bp, Artificial Sequence
  IgG CL nucl
ggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 530 - protein, 117 aa, Artificial Sequence
  N-terminal semi-conserved domain of a RIFIN
  misc_feature (2)..(3): Xaa can be any amino acid
  misc_feature (5)..(9): Xaa can be any amino acid
  misc_feature (11)..(11): Xaa can be any amino acid
  misc_feature (13)..(14): Xaa can be any amino acid
  misc_feature (17)..(17): Xaa can be any amino acid
  misc_feature (20)..(21): Xaa can be any amino acid
  misc_feature (23)..(25): Xaa can be any amino acid
  misc_feature (27)..(27): Xaa can be any amino acid
  misc_feature (36)..(40): Xaa can be any amino acid
  misc_feature (42)..(43): Xaa can be any amino acid
  misc_feature (46)..(47): Xaa can be any amino acid
  misc_feature (60)..(61): Xaa can be any amino acid
  misc_feature (65)..(67): Xaa can be any amino acid
  misc_feature (69)..(71): Xaa can be any amino acid
  misc_feature (73)..(73): Xaa can be any amino acid
  misc_feature (76)..(80): Xaa can be any amino acid
  misc_feature (85)..(85): Xaa can be any amino acid
  misc_feature (90)..(91): Xaa can be any amino acid
  misc_feature (94)..(94): Xaa can be any amino acid
  misc_feature (97)..(100): Xaa can be any amino acid
  misc_feature (102)..(104): Xaa can be any amino acid
  misc_feature (106)..(117): Xaa can be any amino acid
CXXYXXXXXDXDXXMKXVMXXFXXXTXQRFHEYDEXXXXXRXXCKXXCDKEIQKIILKDXXEKEXXXKXXXLXTDXXXXXIPTCXCEKSXXDKXEKXXXXCXXXLXXXXXXXXXXXX

SEQ ID NO: 531 - protein, 117 aa, Artificial Sequence
  N-terminal semi-conserved domain of a RIFIN
  misc_feature (2)..(3): Xaa can be any amino acid
  misc_feature (5)..(6): Xaa can be any amino acid
  misc_feature (8)..(9): Xaa can be any amino acid
  misc_feature (13)..(14): Xaa can be any amino acid
  misc_feature (17)..(17): Xaa can be any amino acid
  misc_feature (20)..(21): Xaa can be any amino acid
  misc_feature (23)..(25): Xaa can be any amino acid
  misc_feature (27)..(27): Xaa can be any amino acid
  misc_feature (36)..(40): Xaa can be any amino acid
  misc_feature (42)..(43): Xaa can be any amino acid
  misc_feature (46)..(47): Xaa can be any amino acid
  misc_feature (60)..(61): Xaa can be any amino acid
  misc_feature (65)..(67): Xaa can be any amino acid
  misc_feature (69)..(71): Xaa can be any amino acid
  misc_feature (73)..(73): Xaa can be any amino acid
  misc_feature (76)..(80): Xaa can be any amino acid
  misc_feature (85)..(85): Xaa can be any amino acid
  misc_feature (90)..(91): Xaa can be any amino acid
  misc_feature (94)..(94): Xaa can be any amino acid
  misc_feature (97)..(100): Xaa can be any amino acid
  misc_feature (102)..(104): Xaa can be any amino acid
  misc_feature (106)..(117): Xaa can be any amino acid
CXXYXXTXXDSDXXMKXVMXXFXXXTXQRFHEYDEXXXXXRXXCKXXCDKEIQKIILKDXXEKEXXXKXXXLXTDXXXXXIPTCXCEKSXXDKXEKXXXXCXXXLXXXXXXXXXXXX

SEQ ID NO: 532 - protein, 117 aa, Artificial Sequence
  N-terminal semi-conserved domain of a RIFIN
  misc_feature (23)..(25): Xaa can be any amino acid
  misc_feature (27)..(27): Xaa can be any amino acid
  misc_feature (36)..(40): Xaa can be any amino acid
  misc_feature (42)..(43): Xaa can be any amino acid
  misc_feature (46)..(47): Xaa can be any amino acid
  misc_feature (60)..(61): Xaa can be any amino acid
  misc_feature (65)..(67): Xaa can be any amino acid
  misc_feature (69)..(71): Xaa can be any amino acid
  misc_feature (73)..(73): Xaa can be any amino acid
  misc_feature (76)..(80): Xaa can be any amino acid
  misc_feature (85)..(85): Xaa can be any amino acid
  misc_feature (90)..(91): Xaa can be any amino acid
  misc_feature (94)..(94): Xaa can be any amino acid
  misc_feature (97)..(100): Xaa can be any amino acid
  misc_feature (102)..(104): Xaa can be any amino acid
  misc_feature (106)..(117): Xaa can be any amino acid
CELYSPTNYDSDPEMKRVMQQFXXXTXQRFHEYDEXXXXXRXXCKXXCDKEIQKIILKDXXEKEXXXKXXXLXTDXXXXXIPTCXCEKSXXDKXEKXXXXCXXXLXXXXXXXXXXXX

SEQ ID NO: 533 - protein, 117 aa, Artificial Sequence
  N-terminal semi-conserved domain of a RIFIN
  misc_feature (23)..(23): Xaa can be any amino acid
  misc_feature (36)..(40): Xaa can be any amino acid
  misc_feature (42)..(43): Xaa can be any amino acid
  misc_feature (46)..(46): Xaa can be any amino acid
  misc_feature (60)..(61): Xaa can be any amino acid
  misc_feature (65)..(67): Xaa can be any amino acid
  misc_feature (69)..(70): Xaa can be any amino acid
  misc_feature (73)..(73): Xaa can be any amino acid
  misc_feature (77)..(80): Xaa can be any amino acid
  misc_feature (94)..(94): Xaa can be any amino acid
  misc_feature (97)..(97): Xaa can be any amino acid
  misc_feature (100)..(100): Xaa can be any amino acid
  misc_feature (102)..(104): Xaa can be any amino acid
  misc_feature (108)..(108): Xaa can be any amino acid
  misc_feature (110)..(113): Xaa can be any amino acid
  misc_feature (115)..(115): Xaa can be any amino acid
CELYSPTNYDSDPEMKRVMQQFXDRTTQRFHEYDEXXXXXRXXCKXQCDKEIQKIILKDXXEKEXXXKXXTLXTDIXXXXIPTCVCEKSLADKXEKXCLXCXXXLGGXVXXXXGXLG

SEQ ID NO: 534 - protein, 117 aa, Plasmodium falciparum
CELYSPTNYDSDPEMKRVMQQFVDRTTQRFHEYDESLQSKRKQCKDQCDKEIQKIILKDKIEKEFTEKLSTLQTDITTKDIPTCVCEKSLADKMEKVCLKCAQNLGGIVAPSTGVLG

SEQ ID NO: 535 - protein, 117 aa, Plasmodium falciparum
CELYSPTNYDSDPEMKRVMQQFHDRTTQRFHEYDERMKTTRQECKEQCDKEIQKIILKDRLEKELMDKFATLHTDIQSDAIPTCVCEKSLADKTEKFCLNCGVQLGGGVLQASGLLG

SEQ ID NO: 536 - protein, 367 aa, Plasmodium falciparum
MKDHYINILLFALPLNILVYNQRNYYITPRHTETNRSLCECELYSPTNYDSDPEMKRVMQQFVDRTTQRFHEYDESLQSKRKQCKDQCDKEIQKIILKDKIEKEFTEKLSTLQTDITTKDIPTCVCEKSLADKMEKVCLKCAQNLGGIVAPSTGVLGEIAALAVNAWKTTALKNAIAAAQKAGDAAGKIAGESKGVETIIGILEQYYSIYELKGTPLKSFFATTHYTDISNIATVIDTELNTSCGLNSLANQAICGLRTKLGLVAKPGQVMVTQKEAITKMITNVVHKSEITAEAAKTEVAATKTAAAIKMNTEAIEAATTPYYTPIIASIVAIVVIVLIMVIIYLILRYRRKKKMKKKLQYIKLLN

SEQ ID NO: 537 - DNA, 1104 bp, Plasmodium falciparum
atgaaagaccattatattaatatattattgtttgctcttccattaaatatattggtatataatcaaaggaactattacattacaccacgtcatacagaaaccaacagatctttatgtgaatgtgaattatattcacctacgaactatgatagtgatcccgaaatgaaaagggtaatgcaacaatttgtggatcgtacaacacaacgatttcacgaatatgatgaaagtttgcaaagtaaacgaaagcaatgcaaagatcaatgcgataaagaaatccaaaaaattatattaaaagataaaatcgaaaaggaatttacagaaaaattatcaacattacaaacagatataacgactaaagacatacccacctgtgtttgcgaaaaatccttggcggacaaaatggaaaaagtatgcttgaaatgtgcacaaaatttgggaggtattgttgcaccctctacaggagtattaggcgaaattgctgcacttgctgtaaatgcctggaaaactacggcacttaagaacgctattgcggcagctcaaaaagcaggtgatgcggccggtaaaattgcgggggaatccaagggtgttgaaacaattattggaatattagaacaatattactctatatatgagttaaaaggaacaccattgaaatccttttttgctacaacgcattatactgatatctcaaatattgctactgttattgatacggaattgaatacgtcttgtgggttgaattccttagctaatcaggctatttgcggtcttcgtacgaaattaggtcttgttgcaaaacctggtcaagttatggttacacagaaagaagctataacaaagatgataaccaacgttgttcataaatctgaaattactgctgaagctgcaaagactgaggtggctgcaactaaaacagcagcagctataaagatgaacacagaagctatagaagctgcaactactccttactatactcctataatagcatccatcgttgcaatagtggtcatagttttaattatggtgataatttatttgattttacgttatcgaagaaaaaaaaaaatgaagaaaaaactccaatatataaaattattaaattaa

SEQ ID NO: 538 - protein, 378 aa, Plasmodium falciparum
MKFNYTNIILFSLSLNILLLSSRVYNKRNHKSIILHTSNENPIKTHRSLCECELYSPTNYDSDPEMKRVMQQFHDRTTQRFHEYDERMKTTRQECKEQCDKEIQKIILKDRLEKELMDKFATLHTDIQSDAIPTCVCEKSLADKTEKFCLNCGVQLGGGVLQASGLLGGIGQLGLDAWKAAALVTAKELAEKAGAAAGLKAGDIHGMKIVIEGLKALKVDTLKSGIFNSFVNNSHYTEVTGLAIAIDTEMNEVCSATYIGIHPICVVREKLGVIPKAGGTMVKQKDAITNVLKQALEKATQSAEALSETTAEDVAAKLTAQKTGAINTIFMSNQTAIIASIVAIVVIVLIMVIIYLILRYRRKKKMKKKLQYIKLLEE

SEQ ID NO: 539 - DNA, 1137 bp, Plasmodium falciparum
atgaagttcaattacactaatataatattattttccctttcattaaatatattgttattatcatcacgggtatacaataaaaggaatcataaaagcattatacttcatacatcaaacgaaaacccaataaaaacacatagatcattatgcgaatgcgaattatattcacctacgaactatgatagtgatcccgaaatgaaaagggtaatgcaacaatttcatgatcgtacaacacaacgatttcacgaatacgacgaaaggatgaaaactacacgccaagaatgtaaagaacaatgcgataaagaaatacaaaaaattattttaaaagacagattagaaaaagaattaatggacaaatttgccacactacacacagatatacaaagtgatgctattccaacatgtgtttgcgaaaagtcgttagcagataaaacagaaaaattttgtctgaactgtggggtgcaactaggaggtggtgtgttgcaagcttcgggtttattaggaggaattggtcaacttgggctagatgcatggaaagcagccgcgttggtaactgctaaggaacttgccgaaaaagccggtgctgcagcaggtcttaaagcaggtgatatccatggtatgaaaatagttattgaaggattaaaagcattgaaagtagatacattaaaatctggaatatttaattcctttgttaataacagccattatactgaagtcacagggcttgctattgctattgatactgaaatgaatgaggtgtgttcagcgacgtatattggtattcatcctatctgcgttgttcgtgagaaattaggtgtaataccaaaggctggtggaacaatggttaaacagaaagatgctataacaaatgtgttaaagcaagctcttgaaaaagctacacaaagtgctgaagcactttctgagactactgctgaagacgttgctgctaaactcacagctcaaaagacgggtgcgataaatactatatttatgagtaatcagactgctattattgcttccatcgttgcaatagtagttatagttttaattatggtgataatatatttaattttacgttatcgacgaaaaaaaaaaatgaagaaaaaactccaatatatcaaattattagaagaatag

SEQ ID NO: 540 - protein, 351 aa, Plasmodium falciparum
TGTGAATTATATTCACCTACGAACTATGATAGTGATCCCGAAATGAAAAGGGTAATGCAACAATTTGTGGATCGTACAACACAACGATTTCACGAATATGATGAAAGTTTGCAAAGTAAACGAAAGCAATGCAAAGATCAATGCGATAAAGAAATCCAAAAAATTATATTAAAAGATAAAATCGAAAAGGAATTTACAGAAAAATTATCAACATTACAAACAGATATAACGACTAAAGACATACCCACCTGTGTTTGCGAAAAATCCTTGGCGGACAAAATGGAAAAAGTATGCTTGAAATGTGCACAAAATTTGGGAGGTATTGTTGCACCCTCTACAGGAGTATTAGGC

SEQ ID NO: 541 - DNA, 357 bp, Plasmodium falciparum
tgcgaatgcgaattatattcacctacgaactatgatagtgatcccgaaatgaaaagggtaatgcaacaatttcatgatcgtacaacacaacgatttcacgaatacgacgaaaggatgaaaactacacgccaagaatgtaaagaacaatgcgataaagaaatacaaaaaattattttaaaagacagattagaaaaagaattaatggacaaatttgccacactacacacagatatacaaagtgatgctattccaacatgtgtttgcgaaaagtcgttagcagataaaacagaaaaattttgtctgaactgtggggtgcaactaggaggtggtgtgttgcaagcttcgggtttattagga

SEQ ID NO: 542 - protein, 250 aa, Artificial Sequence
  FI499V_DexinDJ aa
QVQPVQSGAEVKEPGSSVKVSCKTSGGLIRKSAVSWVRQAPGQGLEWMGGISALFNTKDYAEKFQGRLTITADESTATAYMELSSLTSEDTAIYYCATASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 543 - DNA, 750 bp, Artificial Sequence
  FI499V_DexinDJ nucl
caggtgcagcccgtccagtctggagcagaggtgaaggaacctggcagctccgtgaaggtctcttgcaaaacaagtggcgggctgatccgcaaaagtgccgtgtcatgggtccgacaggctcctggacagggactggaatggatgggaggcatcagcgcactgttcaacactaaggactacgccgaaaaatttcagggccggctgactattaccgccgatgagagtacagccactgcttatatggaactgtctagtctgaccagcgaggacacagctatctactattgcgcaaccgcctcaccactgaagtcccagagagacaccgacctgccaagaccttccatctctgcagaacctggcacagtgattccactggggtcccacgtgactttcgtctgtaggggaccagtgggcgtccagacctttcgcctggagcgggaaagaaattacctgtattccgacactgaggacgtgagccagaccagtccctcagagagcgaagctaggttccgcatcgattccgtgaacgctgggaatgcaggactgtttagatgcatctactataagtctaggaaatggagcgagcagtccgactacctggaactggtggtcaaaggggaggatgtgacatgggctctgtcccagtctcaggacgatccaagagcatgtccccagggcgagctgcccatctctactgacatctactatgtggatgtctggggcaacgggaccacagtgaccgtctcaagc

SEQ ID NO: 544 - protein, 248 aa, Artificial Sequence
  FI499VJ_DexinD aa
QVQPVQSGAEVKEPGSSVKVSCKTSGGLIRKSAVSWVRQAPGQGLEWMGGISALFNTKDYAEKFQGRLTITADESTATAYMELSSLTSEDTAIYYCATASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIFDYWGQGTLVTVSS

SEQ ID NO: 545 - DNA, 744 bp, Artificial Sequence
  FI499VJ_DexinD nucl
caggtgcagcccgtccagtctggagcagaggtgaaggaacctggcagctccgtgaaggtctcttgcaaaacaagtggcgggctgatccgcaaaagtgccgtgtcatgggtccgacaggctcctggacagggactggaatggatgggaggcatcagcgcactgttcaacactaaggactacgccgaaaaatttcagggccggctgaccattacagccgatgagagtactgccaccgcttatatggaactgtctagtctgaccagcgaggacacagctatctactattgcgcaaccgcctcaccactgaagtcccagagagacaccgacctgccaagaccttccatctctgcagaacctggcacagtgattccactggggtcccacgtgactttcgtctgtaggggaccagtgggcgtccagacctttcgcctggagcgggaaagaaattacctgtattccgacactgaggacgtgagccagaccagtccctcagagagcgaagctaggttccgcatcgattccgtgaacgctgggaatgcaggactgtttagatgcatctactataagtctaggaaatggagcgagcagtccgactacctggaactggtggtcaaaggggaggatgtgacttgggctctgtcccagtctcaggacgatccaagagcatgtccccagggcgagctgcccatctctaccgacattttcgattattggggccaggggacactggtgactgtctcaagc

SEQ ID NO: 546 - protein, 249 aa, Artificial Sequence
  MGD21_exin_longGS aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARGGGGSGGGGSDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALGGGGSGGGGSGGGGSGGGGSYYVDVWGNGTTVTVSS

SEQ ID NO: 547 - DNA, 747 bp, Artificial Sequence
  MGD21_exin_longGS nucl
gaagtgcagctggtggaaaccggccctggactgatgaagacctctggcacactgagtctgacatgcgctgtgagtggggactacgtcaacactaatcggagatggtcttgggtgcgacaggcaccaggaaaaggactggagtggatcggggaagtgcaccagagcggaaggaccaactataatcctagcctgaagtcccgcgtgacaatttcagtcgataagagcaaaaaccagttctccctgaaagtggactctgtcactgccgctgataccgcagtgtactattgtgccagaggcgggggaggctctgggggaggcgggagtgacctgcccaggcctagcatctccgctgaaccagggactgtgattcccctgggatctcacgtgaccttcgtctgcagaggccctgtgggggtccagacatttcgcctggagcgggaaagaaactacctgtattctgacaccgaggatgtgagtcagacatctcccagtgagtcagaagcaaggttccgcatcgattccgtcaacgccggaaatgctggcctgtttcgatgtatctactataagagccggaaatggagcgagcagtccgactacctggaactggtggtcaagggcgaggatgtgacctgggccctgggcgggggaggctctgggggaggcgggagtggaggcgggggatcaggtggaggcgggtcgtactatgtggacgtgtggggcaacgggaccacagtgaccgtcagctcc

SEQ ID NO: 548 - protein, 229 aa, Artificial Sequence
  MGD21_exin_short GS aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARGGGGSDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALGGGGSYYVDVWGNGTTVTVSS

SEQ ID NO: 549 - DNA, 687 bp, Artificial Sequence
  MGD21_exin_short GS nucl
gaagtgcagctggtggaaaccggccctggactgatgaagacctctggcacactgagtctgacatgcgctgtgagtggggactacgtcaacactaatcggagatggtcttgggtgcgacaggcaccaggaaaaggactggagtggatcggggaagtgcaccagagcggaaggaccaactataatcctagcctgaagtcccgcgtgacaatttcagtcgataagagcaaaaaccagttctccctgaaagtggactctgtcactgccgctgataccgcagtgtactattgtgccagagggggaggcgggagtgacctgcccaggcctagcatctccgctgaaccagggactgtgattcccctgggatctcacgtgaccttcgtctgcagaggccctgtgggggtccagacatttcgcctggagcgggaaagaaactacctgtattctgacaccgaggatgtgagtcagacatctcccagtgagtcagaagcaaggttccgcatcgattccgtcaacgccggaaatgctggcctgtttcgatgtatctactataagagccggaaatggagcgagcagtccgactacctggaactggtggtcaagggcgaggatgtgacctgggccctgggaggcgggggatcatactatgtggacgtgtggggcaacgggaccacagtgaccgtcagctcc

SEQ ID NO: 550 - protein, 133 aa, Artificial Sequence
  MGD21_NOexin aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTGELPISTDIYYVDVWGNGTTVTVSS

SEQ ID NO: 551 - DNA, 399 bp, Artificial Sequence
  MGD21_NOexin nucl
gaagtgcagctggtggaaaccggccctggactgatgaagacttcaggaaccctgagcctgacttgtgccgtgagcggcgactacgtcaacaccaatcggagatggagttgggtgcggcaggcaccaggaaaaggcctggagtggatcggcgaagtgcaccagtctgggcgaacaaactataatccctctctgaagagtagagtgactatttccgtggacaagtctaaaaaccagttcagcctgaaagtggactccgtcacagccgctgatactgccgtgtactattgtgcaagggccagtcccctgaagtcacagcgcgataccggggagctgcctatcagcacagacatctactatgtggatgtctgggggaatggaaccacagtgacagtcagctcc

SEQ ID NO: 552 - protein, 230 aa, Artificial Sequence
  MGD21_NOin aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGELPISTDIYYVDVWGNGTTVTVSS

SEQ ID NO: 553 - DNA, 690 bp, Artificial Sequence
  MGD21_NOin nucl
gaggtgcagctggtcgaaaccggcccagggctgatgaagacttccggaaccctgtctctgacatgcgccgtgtccggggactacgtcaacactaatcggagatggtcttgggtgaggcaggctcctggaaaaggcctggagtggatcggggaagtgcaccagtccggacggaccaactataatccatctctgaagagtagagtgacaattagtgtcgataagtcaaaaaaccagttctctctgaaagtggacagtgtcacagccgctgatactgcagtgtactattgtgcaagagcaagccccctgaagtcccagagagacaccgacctgcccaggccttctatcagtgctgaaccaggcactgtgattcccctggggtctcatgtgaccttcgtctgtagaggccccgtgggagtccagacttttcgcctggagagggaacgcaattacctgtattcagacaccgaggatgtgagccagacatcacctagcgagtccgaagcccgattccggatcgacagtgtgaacgctggaaatgcaggcctgtttcgctgtatctactataagagccgaaaatggtcagagcagagcgattacctggaactggtggtcaaaggcgagctgcctatcagcactgacatctactatgtggatgtctgggggaacggaaccacagtgaccgtcagctcc

SEQ ID NO: 554 - protein, 142 aa, Artificial Sequence
  MGD21_NOVD aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 555 - DNA, 426 bp, Artificial Sequence
  MGD21_NOVD nucl
gacctgccacgaccatctatttccgccgaacctgggactgtcattcctctggggagccacgtcacatttgtctgccggggacctgtcggggtgcagactttccggctggagcgggaaagaaactacctgtattctgacaccgaagatgtgagtcagacaagcccatccgagtctgaagctaggttccgcatcgactccgtcaacgccggcaatgctgggctgtttcgatgcatctactataagagcagaaaatggagcgagcagtccgactacctggaactggtggtcaagggagaggatgtcacctgggcactgagtcagtcacaggacgatccccgggcctgtcctcagggcgagctgcccatcagcactgatatctactatgtggatgtctgggggaatggcactactgtgaccgtctcaagc

SEQ ID NO: 556 - protein, 250 aa, Artificial Sequence
  MGD21GL_exinWT aa
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYMDVWGKGTTVTVSS

SEQ ID NO: 557 - DNA, 750 bp, Artificial Sequence
  MGD21GL_exinWT nucl
caggtccagctgcaggaaagcggcccaggactggtgaagcctagcggaacactgagtctgacttgtgccgtgagcggagggagcatcagctcctctaactggtggtcttgggtgaggcagccccctggcaagggactggagtggatcggcgaaatctaccacagcgggtccaccaactataatccttcactgaagagccgcgtgacaatcagtgtggacaagtcaaaaaatcagttcagcctgaaactgagttcagtgaccgccgctgatacagcagtctactattgcgcacgggccagcccactgaaatcccagcgagacactgatctgccacggccctctatcagtgctgaacccggaacagtgattcctctgggctcccatgtgactttcgtctgtcgcggaccagtgggcgtccagacctttcgactggagcgggaaagaaactacctgtattctgacactgaggatgtgagtcagacctcacccagcgagtccgaagccaggttccgcatcgacagcgtcaacgctgggaatgcaggactgtttagatgcatctactataagtccaggaaatggtccgagcagtctgactacctggaactggtggtcaagggggaggatgtgacatgggccctgtctcagagtcaggacgatcctagagcttgtccacagggcgagctgcccatttcaaccgatatctattacatggatgtctggggcaagggcaccaccgtgaccgtgagcagc

SEQ ID NO: 558 - protein, 251 aa, Artificial Sequence
  MGD21_wholeGL aa
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARASPLKSQRDTEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKGEDVTWALPQSQLDPRACPQGELPISTDIYYMDVWGKGTTVTVSS

SEQ ID NO: 559 - DNA, 767 bp, Artificial Sequence
  MGD21_wholeGL nucl
caggtgcagctgcaggaaagcggaccaggcctggtcaagccctcaggcactctgagcctgacctgcgctgtgagtggcgggtcaatcagctcctctaattggtggtcctgggtgaggcagccccctgggaaaggactggagtggatcggcgaaatctaccactctgggagtacaaactataatcccagcctgaagtcccgcgtgactatttccgtggacaagtctaaaaatcagttcagcctgaaactgagttcagtgacagccgctgatactgcagtctactattgcgcacgagccagtcctctgaagtcccagcgggacactgaggacctgcctagaccatcaatcagcgccgagcctggaactgtgattccactgggctctcatgtgaccttcgtctgtagaggaccagtgggagtccagaccttccggctggagagagaatcccgatctacctacaacgacacagaagatgtgagccaggctagtccatcagagagcgaagcacggtttagaatcgactccgtgtctgaggggaatgccggaccctacagatgcatctactataagccacccaaatggtctgagcagagtgactatctggaactgctggtgaaaggagaggatgtcacctgggcactgcctcagtctcagctggaccccagagcttgtcctcagggagagctgcctatcagcaccgacatctactatatggacgtgtggggcaaagggaccacagtgacagtcagctccgcgtcgacttcgca

SEQ ID NO: 560 - protein, 153 aa, Artificial Sequence
  MGD21_NOex aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTGEDVTWALSQSQDDPRACPQGELPISTDIYYVDVWGNGTTVTVSS

SEQ ID NO: 561 - DNA, 459 bp, Artificial Sequence
  MGD21_NOex nucl
gaagtgcagctggtggaaaccggccctggactgatgaagacttccggaaccctgtctctgacttgcgccgtgtctggcgactacgtcaacaccaatcggagatggagctgggtgcggcaggctccaggaaaaggcctggagtggatcggcgaagtgcaccagtccgggcgaacaaactataatccctcactgaagagcagagtgactattagtgtcgataagtcaaaaaaccagttctctctgaaagtggacagtgtcacagccgctgatactgccgtgtactattgcgcaagggccagccctctgaagtcccagagagacaccggggaggatgtgacatgggctctgtctcagagtcaggacgatccccgggcatgtcctcagggcgaactgccaatcagcaccgacatctactatgtggatgtctgggggaatggaaccacagtgacagtcagctcc

SEQ ID NO: 562 - protein, 243 aa, Artificial Sequence
  mIgG2b aa
AMVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 563 - DNA, 729 bp, Artificial Sequence
  mIgG2b nucl
gccatggttagatctcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 564 - protein, 232 aa, Artificial Sequence
  hIgG1 aa
AMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - DNA, 696 bp, Artificial Sequence
  hIgG1 nucl
gccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 566 - protein, 152 aa, Artificial Sequence
  DexinDJ variable part aa
ASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 567 - DNA, 456 bp, Artificial Sequence
  DexinDJ variable part nucl
gcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctca

SEQ ID NO: 568 - protein, 395 aa, Artificial Sequence
  DexinDJ-mIgG2b complete sequence aa
ASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSSAMVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 569 - DNA, 1185 bp, Artificial Sequence
  DexinDJ-mIgG2b complete sequence  nucl
gcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctcagccatggttagatctcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 570 - protein, 384 aa, Artificial Sequence
  DexinDJ-hIgG1 complete sequence aa
ASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSSAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 571 - DNA, 1152 bp, Artificial Sequence
  DexinDJ-hIgG1 complete sequence  nucl
gcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctcagccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 572 - protein, 142 aa, Artificial Sequence; identical to SEQ ID NO: 554
  exinDJ variable part aa

SEQ ID NO: 573 - DNA, 426 bp, Artificial Sequence
  exinDJ variable part nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctca

SEQ ID NO: 574 - protein, 374 aa, Artificial Sequence
  exinDJ-mIgG2b complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSSAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 575 - DNA, 1122 bp, Artificial Sequence
  exinDJ-mIgG2b complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctcagccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 576 - protein, 105 aa, Artificial Sequence
  exin variable part aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWAL

SEQ ID NO: 577 - DNA, 315 bp, Artificial Sequence
  exin variable part nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctg

SEQ ID NO: 578 - protein, 337 aa, Artificial Sequence
  exin-mIgG2b complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 579 - DNA, 1011 bp, Artificial Sequence
  exin-mIgG2b complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctggccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 580 - protein, 97 aa, Artificial Sequence; identical to SEQ ID NO: 34
  exon variable part aa

SEQ ID NO: 581 - DNA, 291 bp, Artificial Sequence; identical to SEQ ID NO: 35
  exon variable part nucl

SEQ ID NO: 582 - protein, 340 aa, Artificial Sequence
  ex-mIgG2b complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKAMVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 583 - DNA, 1020 bp, Artificial Sequence
  ex-mIgG2b complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaagccatggttagatctcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 584 - protein, 329 aa, Artificial Sequence
  ex-hIgG1 complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 585 - DNA, 987 bp, Artificial Sequence
  ex-hIgG1 complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaagccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 586 - DNA, 294 bp, Artificial Sequence
  LAIR1ex nucl
gaggacctgccaagacccagcatctccgcagaacctgggactgtgattccactgggctcccacgtgaccttcgtctgcagaggccccgtgggagtccagaccttccggctggagcgcgaatctcgaagtacctacaacgacacagaggacgtgagccaggcctcacccagcgagtccgaagctcggttcagaatcgactctgtcagtgaaggaaatgccggcccttacagatgcatctactataagccccctaaatggtcagagcagagcgattatctggaactgctggtgaag

SEQ ID NO: 587 - protein, 98 aa, Artificial Sequence
  LAIR1ex+L aa
EDLPRPSISAEPGTVIPLGSHVTFVCRGPVGVQTFRLERESRSLYNDTEDVSQASPSESEARFRIDSVSEGNAGPYRCIYYKPPKWSEQSDYLELLVK

SEQ ID NO: 588 - DNA, 294 bp, Artificial Sequence
  LAIR1ex+L nucl
gaggacctgccaagacccagcatctccgcagaacctgggaccgtgattccactgggctcccacgtgacattcgtctgcagaggccccgtgggagtccagacttttaggctggagcgcgaatctcgaagtctgtacaacgacacagaggacgtgagccaggcctcaccaagcgagtccgaagctcggttcagaatcgactctgtcagtgaaggaaatgccggcccttacagatgcatctactataagccccctaaatggtcagagcagagcgattatctggaactgctggtgaag

SEQ ID NO: 589 - protein, 98 aa, Artificial Sequence
  LAIR1ex+LR aa
EDLPRPSISAEPGTVIPLGSHVTFVCRGPVGVQTFRLERESRSLYNDTEDVSQASPSESEARFRIDSVSEGNAGPYRCIYYKPRKWSEQSDYLELLVK

SEQ ID NO: 590 - DNA, 294 bp, Artificial Sequence
  LAIR1ex+LR nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtacaacgacaccgaggacgtgagccaggcctcacctagcgagtccgaagctcggttcagaatcgactctgtcagtgaaggaaatgccggcccttacagatgcatctactataagccaagaaaatggtcagagcagagcgattatctggaactgctggtgaag

SEQ ID NO: 591 - protein, 98 aa, Artificial Sequence
  LAIR1ex+LS1 aa
EDLPRPSISAEPGTVIPLGSHVTFVCRGPVGVQTFRLERESRSLYSDTEDVSQASPSESEARFRIDSVSEGNAGPYRCIYYKPPKWSEQSDYLELLVK

SEQ ID NO: 592 - DNA, 294 bp, Artificial Sequence
  LAIR1ex+LS1 nucl
gaggacctgccaagacccagcatctccgcagaacctgggaccgtgattccactgggctcccacgtgacattcgtctgcagaggccccgtgggagtccagacttttaggctggagcgcgaatctcgaagtctgtactccgacacagaggacgtgagccaggcctcaccaagcgagtccgaagctcggttcagaatcgactctgtcagtgaaggaaacgccggcccttacagatgcatctactataagccccctaaatggtcagagcagagcgattatctggaactgctggtgaag

SEQ ID NO: 593 - protein, 98 aa, Artificial Sequence
  LAIR1ex+LS1R aa
EDLPRPSISAEPGTVIPLGSHVTFVCRGPVGVQTFRLERESRSLYSDTEDVSQASPSESEARFRIDSVSEGNAGPYRCIYYKPRKWSEQSDYLELLVK

SEQ ID NO: 594 - DNA, 294 bp, Artificial Sequence
  LAIR1ex+LS1R nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtactccgacaccgaggacgtgagccaggcctcacctagcgagtccgaagctcggttcagaatcgactctgtcagtgaaggaaacgccggcccttacagatgcatctactataagccaagaaaatggtcagagcagagcgattatctggaactgctggtgaag

SEQ ID NO: 595 - protein, 98 aa, Artificial Sequence
  LAIR1ex+LS1S2R aa
EDLPRPSISAEPGTVIPLGSHVTFVCRGPVGVQTFRLERESRSLYSDTEDVSQASPSESEARFRIDSVSEGNAGPYRCIYYKSRKWSEQSDYLELLVK

SEQ ID NO: 596 - DNA, 294 bp, Artificial Sequence
  LAIR1ex+LS1S2R nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtactccgacaccgaggacgtgagccaggcctcacctagcgagtccgaagctcggttcagaatcgactctgtcagtgaaggaaacgccggcccatacagatgcatctactataagagcagaaaatggtcagagcagagcgattatctggaactgctggtgaag

SEQ ID NO: 597 - protein, 98 aa, Artificial Sequence
  LAIR1ex+LS1T aa
EDLPRPSISAEPGTVIPLGSHVTFVCRGPVGVQTFRLERESRSLYSDTEDVSQTSPSESEARFRIDSVSEGNAGPYRCIYYKPPKWSEQSDYLELLVK

SEQ ID NO: 598 - DNA, 294 bp, Artificial Sequence
  LAIR1ex+LS1T nucl
gaggacctgccaagacccagcatctccgccgaacctgggactgtgattccactgggctcccacgtgaccttcgtctgcagaggccccgtgggagtccagaccttccggctggagcgcgaatctcgaagtctgtactccgacaccgaggacgtgagccagacatcacccagcgagtccgaagcccggttcagaatcgactctgtcagtgaaggaaacgctggcccttacagatgcatctactataagccccctaaatggtcagagcagagcgattatctggaactgctggtgaag

SEQ ID NO: 599 - protein, 98 aa, Artificial Sequence
  LAIR1ex+LS1TR aa
EDLPRPSISAEPGTVIPLGSHVTFVCRGPVGVQTFRLERESRSLYSDTEDVSQTSPSESEARFRIDSVSEGNAGPYRCIYYKPRKWSEQSDYLELLVK

SEQ ID NO: 600 - DNA, 294 bp, Artificial Sequence
  LAIR1ex+LS1TR nucl
gaggacctgcctagacctagcatctccgccgaaccagggactgtgattcccctgggctcccacgtgaccttcgtctgcagaggccccgtgggagtccagaccttccggctggagcgcgaatctcgaagtctgtactccgacaccgaggacgtgagccagacatcacctagcgagtccgaagcccggttcagaatcgactctgtcagtgaaggaaacgctggcccttacagatgcatctactataagccaagaaaatggtcagagcagagcgattatctggaactgctggtgaag

SEQ ID NO: 601 - protein, 98 aa, Artificial Sequence
  LAIR1ex+LS1TS2R aa
EDLPRPSISAEPGTVIPLGSHVTFVCRGPVGVQTFRLERESRSLYSDTEDVSQTSPSESEARFRIDSVSEGNAGPYRCIYYKSRKWSEQSDYLELLVK

SEQ ID NO: 602 - DNA, 294 bp, Artificial Sequence
  LAIR1ex+LS1TS2R nucl
gaggacctgcctagacctagcatctccgccgaaccagggactgtgattcccctgggctcccacgtgaccttcgtctgcagaggccccgtgggagtccagaccttccggctggagcgcgaatctcgaagtctgtactccgacaccgaggacgtgagccagacatcacctagcgagtccgaagcccggttcagaatcgactctgtcagtgaaggaaacgctggcccatacagatgcatctactataagagcagaaaatggtcagagcagagcgattatctggaactgctggtgaag

SEQ ID NO: 603 - protein, 98 aa, Artificial Sequence
  LAIR1ex+LS2R aa
EDLPRPSISAEPGTVIPLGSHVTFVCRGPVGVQTFRLERESRSLYNDTEDVSQASPSESEARFRIDSVSEGNAGPYRCIYYKSRKWSEQSDYLELLVK

SEQ ID NO: 604 - DNA, 294 bp, Artificial Sequence
  LAIR1ex+LS2R nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtacaacgacaccgaggacgtgagccaggcctcacctagcgagtccgaagctcggttcagaatcgactctgtcagtgaaggaaatgccggcccatacagatgcatctactataagtctagaaaatggtcagagcagagcgattatctggaactgctggtgaag

SEQ ID NO: 605 - protein, 98 aa, Artificial Sequence
  LAIR1ex+LT aa
EDLPRPSISAEPGTVIPLGSHVTFVCRGPVGVQTFRLERESRSLYNDTEDVSQTSPSESEARFRIDSVSEGNAGPYRCIYYKPPKWSEQSDYLELLVK

SEQ ID NO: 606 - DNA, 294 bp, Artificial Sequence
  LAIR1ex+LT nucl
gaggacctgccaagacccagcatctccgccgaacctgggactgtgattccactgggctcccacgtgaccttcgtctgcagaggccccgtgggagtccagaccttccggctggagcgcgaatctcgaagtctgtacaacgacaccgaggacgtgagccagacatcacccagcgagtccgaagcccggttcagaatcgactctgtcagtgaaggaaatgctggcccttacagatgcatctactataagccccctaaatggtcagagcagagcgattatctggaactgctggtgaag

SEQ ID NO: 607 - protein, 98 aa, Artificial Sequence
  LAIR1ex+R aa
EDLPRPSISAEPGTVIPLGSHVTFVCRGPVGVQTFRLERESRSTYNDTEDVSQASPSESEARFRIDSVSEGNAGPYRCIYYKPRKWSEQSDYLELLVK

SEQ ID NO: 608 - DNA, 294 bp, Artificial Sequence
  LAIR1ex+R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctacaacgacacagaggacgtgagccaggcctcacctagcgagtccgaagctcggttcagaatcgactctgtcagtgaaggaaatgccggcccttacagatgcatctactataagccaagaaaatggtcagagcagagcgattatctggaactgctggtgaag

SEQ ID NO: 609 - protein, 98 aa, Artificial Sequence
  LAIR1ex+S1 aa
EDLPRPSISAEPGTVIPLGSHVTFVCRGPVGVQTFRLERESRSTYSDTEDVSQASPSESEARFRIDSVSEGNAGPYRCIYYKPPKWSEQSDYLELLVK

SEQ ID NO: 610 - DNA, 294 bp, Artificial Sequence
  LAIR1ex+S1 nucl
gaggacctgccaagacccagcatctccgcagaacctgggactgtgattccactgggctcccacgtgaccttcgtctgcagaggccccgtgggagtccagaccttccggctggagcgcgaatctcgaagtacctactccgacacagaggacgtgagccaggcctcacccagcgagtccgaagctcggttcagaatcgactctgtcagtgaaggaaacgccggcccttacagatgcatctactataagccccctaaatggtcagagcagagcgattatctggaactgctggtgaag

SEQ ID NO: 611 - protein, 98 aa, Artificial Sequence
  LAIR1ex+S1R aa
EDLPRPSISAEPGTVIPLGSHVTFVCRGPVGVQTFRLERESRSTYSDTEDVSQASPSESEARFRIDSVSEGNAGPYRCIYYKPRKWSEQSDYLELLVK

SEQ ID NO: 612 - DNA, 294 bp, Artificial Sequence
  LAIR1ex+S1R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctactccgacacagaggacgtgagccaggcctcacctagcgagtccgaagctcggttcagaatcgactctgtcagtgaaggaaacgccggcccttacagatgcatctactataagccaagaaaatggtcagagcagagcgattatctggaactgctggtgaag

SEQ ID NO: 613 - protein, 98 aa, Artificial Sequence
  LAIR1ex+S1S2R aa
EDLPRPSISAEPGTVIPLGSHVTFVCRGPVGVQTFRLERESRSTYSDTEDVSQASPSESEARFRIDSVSEGNAGPYRCIYYKSRKWSEQSDYLELLVK

SEQ ID NO: 614 - DNA, 294 bp, Artificial Sequence
  LAIR1ex+S1S2R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctactccgacacagaggacgtgagccaggcctcacctagcgagtccgaagctcggttcagaatcgactctgtcagtgaaggaaacgccggcccatacagatgcatctactataagagcagaaaatggtcagagcagagcgattatctggaactgctggtgaag

SEQ ID NO: 615 - protein, 98 aa, Artificial Sequence
  LAIR1ex+S1T aa
EDLPRPSISAEPGTVIPLGSHVTFVCRGPVGVQTFRLERESRSTYSDTEDVSQTSPSESEARFRIDSVSEGNAGPYRCIYYKPPKWSEQSDYLELLVK

SEQ ID NO: 616 - DNA, 294 bp, Artificial Sequence
  LAIR1ex+S1T nucl
gaggacctgccaagacccagcatctccgccgaacctgggactgtgattccactgggctcccacgtgaccttcgtctgcagaggccccgtgggagtccagaccttccggctggagcgcgaatctcgaagtacctactccgacacagaggacgtgagccagacctcacccagcgagtccgaagcccggttcagaatcgactctgtcagtgaaggaaacgctggcccttacagatgcatctactataagccccctaaatggtcagagcagagcgattatctggaactgctggtgaag

SEQ ID NO: 617 - protein, 98 aa, Artificial Sequence
  LAIR1ex+S2 aa
EDLPRPSISAEPGTVIPLGSHVTFVCRGPVGVQTFRLERESRSTYNDTEDVSQASPSESEARFRIDSVSEGNAGPYRCIYYKSPKWSEQSDYLELLVK

SEQ ID NO: 618 - DNA, 294 bp, Artificial Sequence
  LAIR1ex+S2 nucl
gaggacctgcccagacctagcatctccgcagaaccagggactgtgattcccctgggctcccacgtgaccttcgtctgcagaggccccgtgggagtccagaccttccggctggagcgcgaatctcgaagtacctacaacgacacagaggacgtgagccaggcctcacctagcgagtccgaagctcggttcagaatcgactctgtcagtgaaggaaatgccggcccttacagatgcatctactataagtctccaaaatggtcagagcagagcgattatctggaactgctggtgaag

SEQ ID NO: 619 - protein, 98 aa, Artificial Sequence
  LAIR1ex+S2R aa
EDLPRPSISAEPGTVIPLGSHVTFVCRGPVGVQTFRLERESRSTYNDTEDVSQASPSESEARFRIDSVSEGNAGPYRCIYYKSRKWSEQSDYLELLVK

SEQ ID NO: 620 - DNA, 294 bp, Artificial Sequence
  LAIR1ex+S2R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctacaacgacacagaggacgtgagccaggcctcacctagcgagtccgaagctcggttcagaatcgactctgtcagtgaaggaaatgccggcccatacagatgcatctactataagtctagaaaatggtcagagcagagcgattatctggaactgctggtgaag

SEQ ID NO: 621 - protein, 98 aa, Artificial Sequence
  LAIR1ex+T aa
EDLPRPSISAEPGTVIPLGSHVTFVCRGPVGVQTFRLERESRSTYNDTEDVSQTSPSESEARFRIDSVSEGNAGPYRCIYYKPPKWSEQSDYLELLVK

SEQ ID NO: 622 - DNA, 294 bp, Artificial Sequence
  LAIR1ex+T nucl
gaggacctgccaagacccagcatctccgccgaacctgggactgtgattccactgggctcccacgtgaccttcgtctgcagaggccccgtgggagtccagaccttccggctggagcgcgaatctcgaagtacctacaacgacacagaggacgtgagccagacctcacccagcgagtccgaagcccggttcagaatcgactctgtcagtgaaggaaatgctggcccttacagatgcatctactataagccccctaaatggtcagagcagagcgattatctggaactgctggtgaag

SEQ ID NO: 623 - protein, 98 aa, Artificial Sequence
  LAIR1ex+TS2R aa
EDLPRPSISAEPGTVIPLGSHVTFVCRGPVGVQTFRLERESRSTYNDTEDVSQTSPSESEARFRIDSVSEGNAGPYRCIYYKSRKWSEQSDYLELLVK

SEQ ID NO: 624 - DNA, 294 bp, Artificial Sequence
  LAIR1ex+TS2R nucl
gaggacctgcctagacctagcatctccgccgaaccagggactgtgattcccctgggctcccacgtgaccttcgtctgcagaggccccgtgggagtccagaccttccggctggagcgcgaatctcgaagtacctacaacgacacagaggacgtgagccagacctcacctagcgagtccgaagcccggttcagaatcgactctgtcagtgaaggaaatgctggcccatacagatgcatctactataagtctagaaaatggtcagagcagagcgattatctggaactgctggtgaag

SEQ ID NO: 625 - protein, 15 aa, Artificial Sequence
  second variable (V2) domain of a RIFIN
  misc_feature (2)..(2): Xaa can be any amino acid
  misc_feature (4)..(8): Xaa can be any amino acid
  misc_feature (10)..(12): Xaa can be any amino acid
  misc_feature (14)..(14): Xaa can be any amino acid
HXTXXXXXAXXXDXE

SEQ ID NO: 626 - protein, 15 aa, Artificial Sequence
  second variable (V2) domain of a RIFIN
  misc_feature (4)..(8): Xaa can be any amino acid
  misc_feature (10)..(11): Xaa can be any amino acid
HYTXXXXXAXXIDTE

SEQ ID NO: 627 - protein, 19 aa, Artificial Sequence
  second variable (V2) domain of a RIFIN
  misc_feature (2)..(4): Xaa can be any amino acid
  misc_feature (6)..(7): Xaa can be any amino acid
  misc_feature (9)..(17): Xaa can be any amino acid
IXXXRXXLXXXXXXXXXMV

SEQ ID NO: 628 - protein, 19 aa, Artificial Sequence
  second variable (V2) domain of a RIFIN
  misc_feature (3)..(4): Xaa can be any amino acid
  misc_feature (6)..(7): Xaa can be any amino acid
  misc_feature (10)..(12): Xaa can be any amino acid
  misc_feature (14)..(14): Xaa can be any amino acid
  misc_feature (16)..(17): Xaa can be any amino acid
ICXXRXXLGXXXKXGXXMV

SEQ ID NO: 629 - protein, 48 aa, Artificial Sequence
  second variable (V2) domain of a RIFIN
  misc_feature (2)..(2): Xaa can be any amino acid
  misc_feature (4)..(8): Xaa can be any amino acid
  misc_feature (10)..(12): Xaa can be any amino acid
  misc_feature (14)..(14): Xaa can be any amino acid
  misc_feature (16)..(29): Xaa can be any amino acid
  misc_feature (31)..(33): Xaa can be any amino acid
  misc_feature (35)..(36): Xaa can be any amino acid
  misc_feature (38)..(46): Xaa can be any amino acid
HXTXXXXXAXXXDXEXXXXXXXXXXXXXXIXXXRXXLXXXXXXXXXMV

SEQ ID NO: 630 - protein, 48 aa, Artificial Sequence
  second variable (V2) domain of a RIFIN
  misc_feature (4)..(8): Xaa can be any amino acid
  misc_feature (10)..(11): Xaa can be any amino acid
  misc_feature (16)..(19): Xaa can be any amino acid
  misc_feature (21)..(29): Xaa can be any amino acid
  misc_feature (32)..(33): Xaa can be any amino acid
  misc_feature (35)..(36): Xaa can be any amino acid
  misc_feature (39)..(41): Xaa can be any amino acid
  misc_feature (43)..(43): Xaa can be any amino acid
  misc_feature (45)..(46): Xaa can be any amino acid
HYTXXXXXAXXIDTEXXXXCXXXXXXXXXICXXRXXLGXXXKXGXXMV

SEQ ID NO: 631 - protein, 15 aa, Artificial Sequence
  second variable (V2) domain of a RIFIN
  misc_feature (2)..(6): Xaa can be any amino acid
  misc_feature (8)..(12): Xaa can be any amino acid
  misc_feature (14)..(14): Xaa can be any amino acid
KXXXXXSXXXXXHXT

SEQ ID NO: 632 - protein, 16 aa, Artificial Sequence
  second variable (V2) domain of a RIFIN
  misc_feature (3)..(7): Xaa can be any amino acid
  misc_feature (10)..(13): Xaa can be any amino acid
LKXXXXXSFXXXXHYT

SEQ ID NO: 633 - protein, 23 aa, Artificial Sequence
  second variable (V2) domain of a RIFIN
  misc_feature (3)..(3): Xaa can be any amino acid
  misc_feature (5)..(7): Xaa can be any amino acid
  misc_feature (9)..(16): Xaa can be any amino acid
  misc_feature (18)..(22): Xaa can be any amino acid
MVXQXXXTXXXXXXXXKXXXXXE

SEQ ID NO: 634 - protein, 25 aa, Artificial Sequence
  second variable (V2) domain of a RIFIN
  misc_feature (3)..(3): Xaa can be any amino acid
  misc_feature (6)..(6): Xaa can be any amino acid
  misc_feature (10)..(17): Xaa can be any amino acid
  misc_feature (19)..(22): Xaa can be any amino acid
MVXQKXAITXXXXXXXXKXXXXAEA

SEQ ID NO: 635 - protein, 81 aa, Artificial Sequence
  second variable (V2) domain of a RIFIN
  misc_feature (2)..(6): Xaa can be any amino acid
  misc_feature (8)..(12): Xaa can be any amino acid
  misc_feature (14)..(14): Xaa can be any amino acid
  misc_feature (16)..(20): Xaa can be any amino acid
  misc_feature (22)..(24): Xaa can be any amino acid
  misc_feature (26)..(26): Xaa can be any amino acid
  misc_feature (28)..(41): Xaa can be any amino acid
  misc_feature (43)..(45): Xaa can be any amino acid
  misc_feature (47)..(48): Xaa can be any amino acid
  misc_feature (50)..(58): Xaa can be any amino acid
  misc_feature (61)..(61): Xaa can be any amino acid
  misc_feature (63)..(65): Xaa can be any amino acid
  misc_feature (67)..(74): Xaa can be any amino acid
  misc_feature (76)..(80): Xaa can be any amino acid
KXXXXXSXXXXXHXTXXXXXAXXXDXEXXXXXXXXXXXXXXIXXXRXXLXXXXXXXXXMVXQXXXTXXXXXXXXKXXXXXE

SEQ ID NO: 636 - protein, 84 aa, Artificial Sequence
  second variable (V2) domain of a RIFIN
  misc_feature (3)..(7): Xaa can be any amino acid
  misc_feature (10)..(13): Xaa can be any amino acid
  misc_feature (17)..(21): Xaa can be any amino acid
  misc_feature (23)..(24): Xaa can be any amino acid
  misc_feature (29)..(32): Xaa can be any amino acid
  misc_feature (34)..(42): Xaa can be any amino acid
  misc_feature (45)..(46): Xaa can be any amino acid
  misc_feature (48)..(49): Xaa can be any amino acid
  misc_feature (52)..(54): Xaa can be any amino acid
  misc_feature (56)..(56): Xaa can be any amino acid
  misc_feature (58)..(59): Xaa can be any amino acid
  misc_feature (62)..(62): Xaa can be any amino acid
  misc_feature (65)..(65): Xaa can be any amino acid
  misc_feature (69)..(76): Xaa can be any amino acid
  misc_feature (78)..(81): Xaa can be any amino acid
LKXXXXXSFXXXXHYTXXXXXAXXIDTEXXXXCXXXXXXXXXICXXRXXLGXXXKXGXXMVXQKXAITXXXXXXXXKXXXXAEA

SEQ ID NO: 637 - protein, 167 aa, Artificial Sequence
  second variable (V2) domain of a RIFIN
  misc_feature (2)..(3): Xaa can be any amino acid
  misc_feature (5)..(7): Xaa can be any amino acid
  misc_feature (11)..(12): Xaa can be any amino acid
  misc_feature (15)..(16): Xaa can be any amino acid
  misc_feature (18)..(20): Xaa can be any amino acid
  misc_feature (22)..(22): Xaa can be any amino acid
  misc_feature (26)..(26): Xaa can be any amino acid
  misc_feature (30)..(31): Xaa can be any amino acid
  misc_feature (34)..(36): Xaa can be any amino acid
  misc_feature (38)..(41): Xaa can be any amino acid
  misc_feature (43)..(53): Xaa can be any amino acid
  misc_feature (56)..(60): Xaa can be any amino acid
  misc_feature (63)..(66): Xaa can be any amino acid
  misc_feature (70)..(74): Xaa can be any amino acid
  misc_feature (76)..(77): Xaa can be any amino acid
  misc_feature (82)..(85): Xaa can be any amino acid
  misc_feature (87)..(95): Xaa can be any amino acid
  misc_feature (98)..(99): Xaa can be any amino acid
  misc_feature (101)..(102): Xaa can be any amino acid
  misc_feature (105)..(107): Xaa can be any amino acid
  misc_feature (109)..(109): Xaa can be any amino acid
  misc_feature (111)..(112): Xaa can be any amino acid
  misc_feature (115)..(115): Xaa can be any amino acid
  misc_feature (118)..(118): Xaa can be any amino acid
  misc_feature (122)..(129): Xaa can be any amino acid
  misc_feature (131)..(134): Xaa can be any amino acid
  misc_feature (138)..(142): Xaa can be any amino acid
  misc_feature (144)..(147): Xaa can be any amino acid
  misc_feature (149)..(154): Xaa can be any amino acid
  misc_feature (156)..(156): Xaa can be any amino acid
  misc_feature (159)..(166): Xaa can be any amino acid
IXXLXXXAWKXXALXXAXXXAXKAGXAAGXXAGXXXGXXXXIXXXXXXXXXXXLKXXXXXSFXXXXHYTXXXXXAXXIDTEXXXXCXXXXXXXXXICXXRXXLGXXXKXGXXMVXQKXAITXXXXXXXXKXXXXAEAXXXXXAXXXXAXXXXXXTXAIXXXXXXXXT

SEQ ID NO: 638 - protein, 167 aa, Plasmodium falciparum
IAALAVNAWKTTALKNAIAAAQKAGDAAGKIAGESKGVETIIGILEQYYSIYELKGTPLKSFFATTHYTDISNIATVIDTELNTSCGLNSLANQAICGLRTKLGLVAKPGQVMVTQKEAITKMITNVVHKSEITAEAAKTEVAATKTAAAIKMNTEAIEAATTPYYT

SEQ ID NO: 639 - protein, 166 aa, Plasmodium falciparum
IGQLGLDAWKAAALVTAKELAEKAGAAAGLKAGDIHGMKIVIEGLKALKVDTLKSGIFNSFVNNSHYTEVTGLAIAIDTEMNEVCSATYIGIHPICVVREKLGVIPKAGGTMVKQKDAITNVLKQALEKATQSAEALSETTAEDVAAKLTAQKTGAINTIFMSNQT